(19) United States
(12) Patent Application Publication
(10) Pub. No.: US 2014/0242080 A1
(43) Pub. Date: Aug. 28, 2014

(54) BISPECIFIC T CELL ACTIVATING ANTIGEN BINDING MOLECULES

(71) Applicant: ROCHE GLYCART AG, Schlieren (CH)

(72) Inventors: Christiane Jaeger, Niederweningen (CH); Christian Klein, Bonstetten (CH); Ekkehard Moessner, Kreuzlingen (CH); Pablo Umana, Wollerau (CH)

(73) Assignee: ROCHE GLYCART AG, Schlieren (CH)

(21) Appl. No.: 14/188,516

(22) Filed: Feb. 24, 2014

(30) Foreign Application Priority Data
Feb. 26, 2013 (EP) 13156673.9

(51) Int. Cl. C07K 16/28

(52) U.S. Cl. CPC C07K 16/2809
USPC 424/136.1; 530/387.3; 536/23.53; 435/320.1; 435/328; 435/69.6; 435/375; 435/419; 435/252.3; 435/254.2

(57) ABSTRACT

The present invention generally relates to novel bispecific antigen binding molecules for T cell activation and re-direction to specific target cells. In addition, the present invention relates to polynucleotides encoding such bispecific antigen binding molecules, and vectors and host cells comprising such polynucleotides. The invention further relates to methods for producing the bispecific antigen binding molecules of the invention, and to methods of using these bispecific antigen binding molecules in the treatment of disease.

### FIELD OF THE INVENTION

The present invention generally relates to bispecific antigen binding molecules for activating T cells. In addition, the present invention relates to polynucleotides encoding such bispecific antigen binding molecules, and vectors and host cells comprising such polynucleotides. The invention further relates to methods for producing the bispecific antigen binding molecules of the invention, and to methods of using these bispecific antigen binding molecules in the treatment of disease.

### BACKGROUND

The selective destruction of an individual cell or a specific cell type is often desirable in a variety of clinical settings. For example, it is a primary goal of cancer therapy to specifically destroy tumor cells, while leaving healthy cells and tissues intact and undamaged.

An attractive way of achieving this is by inducing an immune response against the tumor, to make immune effector cells such as natural killer (NK) cells or cytotoxic T lymphocytes (CTLs) attack and destroy tumor cells. CTLs constitute the most potent effector cells of the immune system, however they cannot be activated by the effector mechanism mediated by the Fc domain of conventional therapeutic antibodies.

In this regard, bispecific antibodies designed to bind with one “arm” to a surface antigen on target cells, and with the second “arm” to an activating, invariant component of the T cell receptor (TCR) complex, have become of interest in recent years. The simultaneous binding of such an antibody to both of its targets will force a temporary interaction between target cell and T cell, causing activation of any cytotoxic T cell and subsequent lysis of the target cell. Hence, the immune response is re-directed to the target cells and is independent of peptide antigen presentation by the target cell or the specificity of the T cell as would be relevant for normal MHC-restricted activation of CTLs. In this context it is crucial that CTLs are only activated when a target cell is presenting the bispecific antibody to them, i.e. the immunological synapse is mimicked. Particularly desirable are bispecific antibodies that do not require lymphocyte preconditioning or co-stimulation in order to elicit efficient lysis of target cells.

Several bispecific antibody formats have been developed and their suitability for T cell mediated immunotherapy investigated. Out of these, the so-called BiTE (bispecific T cell engager) molecules have been very well characterized and already shown some promise in the clinic (reviewed in Nagorsen and Bäuerle, Exp Cell Res 317, 1255-1260 (2011)). BiTEs are tandem scFv molecules wherein two scFv molecules are fused by a flexible linker. Further bispecific formats being evaluated for T cell engagement include diabodies (Holliger et al., Prot Eng 9, 299-305 (1996)) and derivatives thereof, such as tandem diabodies (Kipriyanov et al., J Mol Biol 293, 41-66 (1999)). A more recent development are the so-called DART (dual affinity retargeting) molecules, which are based on the diabody format but feature a C-terminal disulfide bridge for additional stabilization (Moore et al., Blood 117, 4542-51 (2011)). The so-called triomabs, which are whole hybrid mouse/rat IgG molecules and also currently being evaluated in clinical trials, represent a larger sized format (reviewed in Seimetz et al., Cancer Treat Rev 36, 458-467 (2010)).

The variety of formats that are being developed shows the great potential attributed to T cell re-direction and activation in immunotherapy. The task of generating bispecific antibodies suitable therefor is, however, by no means trivial, but involves a number of challenges that have to be met related to efficacy, toxicity, applicability and produceability of the antibodies.

Small constructs such as, for example, BiTE molecules—while being able to efficiently crosslink effector and target cells—have a very short serum half life requiring them to be administered to patients by continuous infusion. IgG-like formats on the other hand—while having the great benefit of a long half life—suffer from toxicity associated with the native effector functions inherent to IgG molecules. Their immunogenic potential constitutes another unfavorable feature of IgG-like bispecific antibodies, especially non-human formats, for successful therapeutic development. Finally, a major challenge in the general development of bispecific antibodies has been the production of bispecific antibody constructs at a clinically sufficient quantity and purity, due to the mispairing of antibody heavy and light chains of different specificities upon co-expression, which decreases the yield of the correctly assembled construct and results in a number of non-functional side products from which the desired bispecific antibody may be difficult to separate.

Given the difficulties and disadvantages associated with currently available bispecific antibodies for T cell mediated immunotherapy, there remains a need for novel, improved formats of such molecules. The present invention provides bispecific antigen binding molecules designed for T cell activation and re-direction that combine good efficacy and produceability with low toxicity and favorable pharmacokinetic properties.

### SUMMARY OF THE INVENTION

In a first aspect the present invention provides a T cell activating bispecific antigen binding molecule comprising a first and a second antigen binding moiety, one of which is a Fab molecule capable of specific binding to an activating T cell antigen and the other one of which is a Fab molecule capable of specific binding to a target cell antigen, and an IgG4 Fc domain composed of a first and a second subunit capable of stable association; wherein the first antigen binding moiety is (a) a single chain Fab molecule wherein the Fab light chain and the Fab heavy chain are connected by a peptide linker, or (b) a crossover Fab molecule wherein either the variable or the constant regions of the Fab light chain and the Fab heavy chain are exchanged.

In a particular embodiment, not more than one antigen binding moiety capable of specific binding to an activating T cell antigen is present in the T cell activating bispecific antigen binding molecule (i.e. the T cell activating bispecific antigen binding molecule provides monovalent binding to the activating T cell antigen). In particular embodiments, the first antigen binding moiety is a crossover Fab molecule. In even more particular embodiments, the first antigen binding moiety is a crossover Fab molecule wherein the constant regions of the Fab light chain and the Fab heavy chain are exchanged.

In some embodiments, the first and the second antigen binding moiety of the T cell activating bispecific antigen binding molecule are fused to each other, optionally via a peptide linker. In one such embodiment, the second antigen binding moiety is fused at the C-terminus of the Fab heavy chain to the N-terminus of the Fab heavy chain of the first antigen binding moiety. In another such embodiment, the first antigen binding moiety is fused at the C-terminus of the Fab heavy chain to the N-terminus of the Fab heavy chain of the second antigen binding moiety. In yet another such embodiment, the second antigen binding moiety is fused at the C-terminus of the Fab light chain to the N-terminus of the Fab light chain of the first antigen binding moiety. In embodiments wherein the first antigen binding moiety is a crossover Fab molecule and wherein either (i) the second antigen binding moiety is fused at the C-terminus of the Fab heavy chain to the N-terminus of the Fab heavy chain of the first antigen binding moiety or (ii) the first antigen binding moiety is fused at the C-terminus of the Fab heavy chain to the N-terminus of the Fab heavy chain of the second antigen binding moiety, additionally the Fab light chain of the first antigen binding moiety and the Fab light chain of the second antigen binding moiety may be fused to each other, optionally via a peptide linker.

In one embodiment, the second antigen binding moiety of the T cell activating bispecific antigen binding molecule is fused at the C-terminus of the Fab heavy chain to the N-terminus of the first or the second subunit of the Fc domain. In another embodiment, the first antigen binding moiety is fused at the C-terminus of the Fab heavy chain to the N-terminus of the first or second subunit of the Fc domain.

In one embodiment, the first and the second antigen binding moiety of the T cell activating bispecific antigen binding molecule are each fused at the C-terminus of the Fab heavy chain to the N-terminus of one of the subunits of the Fc domain.

In certain embodiments, the T cell activating bispecific antigen binding molecule comprises a third antigen binding moiety which is a Fab molecule capable of specific binding to a target cell antigen. In one such embodiment, the third antigen binding moiety is fused at the C-terminus of the Fab heavy chain to the N-terminus of the first or second subunit of the Fc domain. In a particular embodiment, the second and the third antigen binding moiety of the T cell activating antigen binding molecule are each fused at the C-terminus of the Fab heavy chain to the N-terminus of one of the subunits of the Fc domain, and the first antigen binding moiety is fused at the C-terminus of the Fab heavy chain to the N-terminus of the Fab heavy chain of the second antigen binding moiety. In another particular embodiment, the first and the third antigen binding moiety of the T cell activating antigen binding molecule are each fused at the C-terminus of the Fab heavy chain to the N-terminus of one of the subunits of the Fc domain, and the second antigen binding moiety is fused at the C-terminus of the Fab heavy chain to the N-terminus of the Fab heavy chain of the first antigen binding moiety. The components of the T cell activating bispecific antigen binding molecule may be fused directly or through suitable peptide linkers. In one embodiment the second and the third antigen binding moiety and the Fc domain are part of an immunoglobulin molecule.

In a particular embodiment, the Fc domain is an IgG₄ Fc domain. In particular embodiments the Fc domain is a human IgG₄ Fc domain.

In particular embodiments the Fc domain comprises a modification promoting the association of the first and the second Fc domain subunit. In a specific such embodiment, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable.

In a particular embodiment the Fc domain exhibits reduced binding affinity to an Fc receptor and/or reduced effector function, as compared to a native IgG₄ Fc domain. In certain embodiments the Fc domain is engineered to have reduced binding affinity to an Fc receptor and/or reduced effector function, as compared to a non-engineered Fc domain. In one embodiment, the Fc domain comprises one or more amino acid substitution that reduces binding to an Fc receptor and/or effector function. In one embodiment, the one or more amino acid substitution in the Fc domain that reduces binding to an Fc receptor and/or effector function is at one or more position selected from the group of L235, S228, and P329 (Kabat numbering). In one embodiment, the Fc domain of the T cell activating bispecific antigen binding molecule comprises the amino acid substitutions L235E and S228P (SPLE). In one embodiment, the Fc domain of the T cell activating bispecific antigen binding molecule comprises the amino acid substitutions L235E and S228P and P329G.

In one embodiment the Fc receptor for which binding is reduced is an Fcγ receptor. In one embodiment the Fc receptor is a human Fc receptor. In one embodiment, the Fc receptor is an activating Fc receptor. In a specific embodiment, the Fc receptor is human FcγRIIa, FcγRI, and/or FcγRIIIa. In one embodiment, the effector function is antibody-dependent cell-mediated cytotoxicity (ADCC).

In a particular embodiment, the activating T cell antigen that the bispecific antigen binding molecule is capable of binding is CD3. In other embodiments, the target cell antigen that the bispecific antigen binding molecule is capable of binding is a tumor cell antigen. In one embodiment, the target cell antigen is selected from the group consisting of: Melanoma-associated Chondroitin Sulfate Proteoglycan (MCSP), Epidermal Growth Factor Receptor (EGFR), Carcinoembryonic Antigen (CEA), Fibroblast Activation Protein (FAP), CD19, CD20 and CD33.

In another embodiment, the T cell activating bispecific antigen binding molecule comprises a first antigen binding moiety capable of specific binding to CD3, a second antigen binding moiety capable of binding to MCSP, and an IgG₄ Fc domain. In one such embodiment, the antigen binding molecule comprises the amino acid sequences of SEQ ID NOs 278, 319, 369, and 370 or comprises the amino acid sequences of SEQ ID NOs 278, 319, 371, and 372.

According to another aspect of the invention there is provided an isolated polynucleotide encoding a T cell activating bispecific antigen binding molecule of the invention or a fragment thereof. The invention also encompasses polypeptides encoded by the polynucleotides of the invention. The invention further provides an expression vector comprising the isolated polynucleotide of the invention, and a host cell comprising the isolated polynucleotide or the expression vector of the invention. In some embodiments the host cell is a eukaryotic cell, particularly a mammalian cell.

In another aspect is provided a method of producing the T cell activating bispecific antigen binding molecule of the invention, comprising the steps of a) culturing the host cell of the invention under conditions suitable for the expression of the T cell activating bispecific antigen binding molecule and b) recovering the T cell activating bispecific antigen binding molecule. The invention also encompasses a T cell activating bispecific antigen binding molecule produced by the method of the invention.

The invention further provides a pharmaceutical composition comprising the T cell activating bispecific antigen binding molecule of the invention and a pharmaceutically acceptable carrier. Also encompassed by the invention are methods of using the T cell activating bispecific antigen binding molecule and pharmaceutical composition of the invention. In one aspect the invention provides a T cell activating bispecific antigen binding molecule or a pharmaceutical composition of the invention for use as a medicament. In one aspect is provided a T cell activating bispecific antigen binding molecule or a pharmaceutical composition according to the invention for use in the treatment of a disease in an individual in need thereof. In a specific embodiment the disease is cancer.

Also provided is the use of a T cell activating bispecific antigen binding molecule of the invention for the manufacture of a medicament for the treatment of a disease in an individual in need thereof; as well as a method of treating a disease in an individual, comprising administering to said individual a therapeutically effective amount of a composition comprising the T cell activating bispecific antigen binding molecule according to the invention in a pharmaceutically acceptable form. In a specific embodiment the disease is cancer. In any of the above embodiments the individual preferably is a mammal, particularly a human.

The invention also provides a method for inducing lysis of a target cell, particularly a tumor cell, comprising contacting a target cell with a T cell activating bispecific antigen binding molecule of the invention in the presence of a T cell, particularly a cytotoxic T cell.

### BRIEF DESCRIPTION OF THE DRAWINGS

FIG. 1. Exemplary configurations of the T cell activating bispecific antigen binding molecules of the invention. Illustration of (A) the “1+1 IgG scFab, one armed”, and (B) the “1+1 IgG scFab, one armed inverted” molecule. In the “1+1 IgG scFab, one armed” molecule the light chain of the T cell targeting Fab is fused to the heavy chain by a linker, while the “1+1 IgG scFab, one armed inverted” molecule has the linker in the tumor targeting Fab. (C) Illustration of the “2+1 IgG scFab” molecule. (D) Illustration of the “1+1 IgG scFab” molecule. (E) Illustration of the “1+1 IgG Crossfab” molecule. (F) Illustration of the “2+1 IgG Crossfab” molecule. (G) Illustration of the “2+1 IgG Crossfab” molecule with alternative order of Crossfab and Fab components (“inverted”). (H) Illustration of the “1+1 IgG Crossfab light chain (LC) fusion” molecule. (I) Illustration of the “1+1 CrossMab” molecule. (J) Illustration of the “2+1 IgG Crossfab, linked light chain” molecule. (K) Illustration of the “1+1 IgG Crossfab, linked light chain” molecule. (L) Illustration of the “2+1 IgG Crossfab, inverted, linked light chain” molecule. (M) Illustration of the “1+1 IgG Crossfab, inverted, linked light chain” molecule. Black dot: optional modification in the Fc domain promoting heterodimerization.

FIG. 2. SDS PAGE (4-12% Bis/Tris, NuPage Invitrogen, Coomassie-stained) of “1+1 IgG scFab, one armed” (anti-MCSP/anti-huCD3) (see SEQ ID NOs 1, 3, 5), non reduced (A) and reduced (B), and of “1+1 IgG scFab, one armed inverted” (anti-MCSP/anti-huCD3) (see SEQ ID NOs 7, 9, 11), non reduced (C) and reduced (D).

FIG. 3. Analytical size exclusion chromatography (Superdex 200 10/300 GL GE Healthcare; 2 mM MOPS pH 7.3, 150 mM NaCl, 0.02% (w/v) NaCl; 50 μg sample injected) of “1+1 IgG scFab, one armed” (anti-MCSP/anti-huCD3) (see SEQ ID NOs 1, 3, 5) (A) and “1+1 IgG scFab, one armed inverted” (anti-MCSP/anti-huCD3) (see SEQ ID NOs 7, 9, 11) (B).

FIG. 4. SDS PAGE (4-12% Bis/Tris, NuPage Invitrogen, Coomassie-stained) of “1+1 IgG scFab, one armed” (anti-EGFR/anti-huCD3) (see SEQ ID NOs 43, 45, 57), non reduced (A) and reduced (B), and of “1+1 IgG scFab, one armed inverted” (anti-EGFR/anti-huCD3) (see SEQ ID NOs 11, 49, 51), non reduced (C) and reduced (D).

FIG. 5. Analytical size exclusion chromatography (Superdex 200 10/300 GL GE Healthcare; 2 mM MOPS pH 7.3, 150 mM NaCl, 0.02% (w/v) NaCl; 50 μg sample injected) of “1+1 IgG scFab, one armed” (anti-EGFR/anti-huCD3) (see SEQ ID NOs 43, 45, 47) (A) and “1+1 IgG scFab, one armed inverted” (anti-EGFR/anti-huCD3) (see SEQ ID NOs 11, 49, 51) (B).

FIG. 6. (A, B) SDS PAGE (4-12% Bis/Tris, NuPage Invitrogen, Coomassie-stained) of “1+1 IgG scFab, one armed inverted” (anti-FAP/anti-huCD3) (see SEQ ID NOs 11, 51, 55), non reduced (A) and reduced (B). (C) Analytical size exclusion chromatography (Superdex 200 10/300 GL GE Healthcare; 2 mM MOPS pH 7.3, 150 mM NaCl, 0.02% (w/v) NaCl; 50 μg sample injected) of “1+1 IgG scFab, one armed inverted” (anti-FAP/anti-huCD3).

FIG. 7. SDS PAGE (4-12% Bis/Tris, NuPage Invitrogen, Coomassie-stained) of (A) “2+1 IgG scFab, P329G LALA” (anti-MCSP/anti-huCD3) (see SEQ ID NOs 5, 21, 23), non reduced (lane 2) and reduced (lane 3); of (B) “2+1 IgG scFab, LALA” (anti-MCSP/anti-huCD3) (see SEQ ID NOs 5, 17, 19), non reduced (lane 2) and reduced (lane 3); of (C) “2+1 IgG scFab, wt” (anti-MCSP/anti-huCD3) (see SEQ ID NOs 5, 13, 15), non reduced (lane 2) and reduced (lane 3); and of (D) “2+1 IgG scFab, P329G LALA N297D” (anti-MCSP/anti-huCD3) (see SEQ ID NOs 5, 25, 27), non reduced (lane 2) and reduced (lane 3).

FIG. 8. Analytical size exclusion chromatography (Superdex 200 10/300 GL GE Healthcare; 2 mM MOPS pH 7.3, 150 mM NaCl, 0.02% (w/v) NaCl; 50 μg sample injected) of (A) “2+1 IgG scFab, P329G LALA” (anti-MCSP/anti-huCD3) (see SEQ ID NOs 5, 21, 23); of (B) “2+1 IgG scFab, LALA” (anti-MCSP/anti-huCD3) (see SEQ ID NOs 5, 17, 19); of (C) “2+1 IgG scFab, wt” (anti-MCSP/anti-huCD3) (see SEQ ID NOs 5, 13, 15); and of (D) “2+1 IgG scFab, P329G LALA N297D” (anti-MCSP/anti-huCD3) (see SEQ ID NOs 5, 25, 27).

FIG. 9. (A, B) SDS PAGE (4-12% Bis/Tris, NuPage Invitrogen, Coomassie-stained) of “2+1 IgG scFab, P329G LALA” (anti-EGFR/anti-huCD3) (see SEQ ID NOs 45, 47, 53), non reduced (A) and reduced (B). (C) Analytical size exclusion chromatography (Superdex 200 10/300 GL GE Healthcare; 2 mM MOPS pH 7.3, 150 mM NaCl, 0.02% (w/v) NaCl; 50 μg sample injected) of “2+1 IgG scFab, P329G LALA” (anti-EGFR/anti-huCD3).

FIG. 10. (A, B) SDS PAGE (4-12% Bis/Tris, NuPage Invitrogen, Coomassie-stained) of “2+1 IgG scFab, P329G LALA” (anti-FAP/anti-huCD3) (see SEQ ID NOs 57, 59, 61), non reduced (A) and reduced (B). (C) Analytical size exclusion chromatography (Superdex 200 10/300 GL GE Healthcare; 2 mM MOPS pH 7.3, 150 mM NaCl, 0.02% (w/v) NaCl; 50 μg sample injected) of “2+1 IgG scFab, P329G LALA” (anti-FAP/anti-huCD3).

FIG. 11. (A, B) SDS PAGE (4-12% Tris-Acetate (A) or 4-12% Bis/Tris (B), NuPage Invitrogen, Coomassie-stained) of “1+1 IgG Crossfab, Fc(hole) P329G LALA/Fc(knob) wt” (anti-MCSP/anti-huCD3) (see SEQ ID NOs 5, 29, 31, 33), non reduced (A) and reduced (B). (C) Analytical size exclusion chromatography (Superdex 200 10/300 GL GE Healthcare; 2 mM MOPS pH 7.3, 150 mM NaCl, 0.02% (w/v) NaCl; 50 μg sample injected) of “1+1 IgG Crossfab, Fc(hole) P329G LALA/Fc(knob) wt” (anti-MCSP/anti-huCD3).

FIG. 12. (A, B) SDS PAGE (4-12% Bis/Tris, NuPage Invitrogen, Coomassie-stained) of “2+1 IgG Crossfab” (anti-MCSP/anti-huCD3) (see SEQ ID NOs 3, 5, 29, 33), non reduced (A) and reduced (B). (C) Analytical size exclusion chromatography (Superdex 200 10/300 GL GE Healthcare; 2 mM MOPS pH 7.3, 150 mM NaCl, 0.02% (w/v) NaCl; 50 μg sample injected) of “2+1 IgG Crossfab” (anti-MCSP/anti-huCD3).

FIG. 13. (A, B) SDS PAGE (4-12% Bis/Tris, NuPage Invitrogen, Coomassie-stained) of “2+1 IgG Crossfab” (anti-MCSP/anti-cyCD3) (see SEQ ID NOs 3, 5, 35, 37), non reduced (A) and reduced (B). (C) Analytical size exclusion chromatography (Superdex 200 10/300 GL GE Healthcare; 2 mM MOPS pH 7.3, 150 mM NaCl, 0.02% (w/v) NaCl; 50 μg sample injected) of “2+1 IgG Crossfab” (anti-MCSP/anti-cyCD3).

FIG. 14. (A, B) SDS PAGE (4-12% Bis/Tris, NuPage Invitrogen, Coomassie-stained) of “2+1 IgG Crossfab, inverted” (anti-CEA/anti-huCD3) (see SEQ ID NOs 33, 63, 65, 67), non reduced (A) and reduced (B). (C) Analytical size exclusion chromatography (Superdex 200 10/300 GL GE Healthcare; 2 mM MOPS pH 7.3, 150 mM NaCl, 0.02% (w/v) NaCl; 50 μg sample injected) of “2+1 IgG Crossfab, inverted” (anti-CEA/anti-huCD3).

FIG. 15. (A) Thermal stability of “(scFv)₂-Fc” and “(dsscFv)₂-Fc” (anti-MCSP (LC007)/anti-huCD3 (V9)). Dynamic Light Scattering, measured in a temperature ramp from 25-75° C. at 0.05° C./min. Black curve: “(scFv)₂-Fc”; grey curve: “(dsscFv)₂-Fc”. (B) Thermal stability of “2+1 IgG scFab” (see SEQ ID NOs 5, 21, 23) and “2+1 IgG Crossfab” (anti-MCSP/anti-huCD3) (see SEQ ID NOs 3, 5, 29, 33). Dynamic Light Scattering, measured in a temperature ramp from 25-75° C. at 0.05° C./min. Black curve: “2+1 IgG scFab”; grey curve: “2+1 IgG Crossfab”.

FIG. 16. Biacore assay setup for (A) determination of interaction of various Fc-mutants with human FcγRIIIa, and for (B) simultaneous binding of T cell bespecific constructs with tumor target and human CD3γ(G₄S)₅CD3ε-AcTev-Fc(knob)-Avi/Fc(hole).

FIG. 17. Simultaneous binding of T-cell bispecific constructs to the D3 domain of human MCSP and human CD3γ(G₄S)₅CD3ε-AcTev-Fc(knob)-Avi/Fc(hole). (A) “2+1 IgG Crossfab” (see SEQ ID NOs 3, 5, 29, 33), (B) “2+1 IgG scFab” (see SEQ ID NOs 5, 21, 23).

FIG. 18. Simultaneous binding of T-cell bispecific constructs to human EGFR and human CD3γ(G₄S)₅CD3ε-AcTev-Fc(knob)-Avi/Fc(hole). (A) “2+1 IgG scFab” (see SEQ ID NOs 45, 47, 53), (B) “1+1 IgG scFab, one armed” (see SEQ ID NOs 43, 45, 47), (C) “1+1 IgG scFab, one armed inverted” (see SEQ ID NOs 11, 49, 51), and (D) “1+1 IgG scFab” (see SEQ ID NOs 47, 53, 213).

FIG. 19. Binding of the “(scFv)₂” molecule (50 nM) to CD3 expressed on Jurkat cells (A), or to MCSP on Colo-38 cells (B) measured by FACS. Mean fluorescence intensity compared to untreated cells and cells stained with the secondary antibody only is depicted.

FIG. 20. Binding of the “2+1 IgG scFab, LALA” (see SEQ ID NOs 5, 17, 19) construct (50 nM) to CD3 expressed on Jurkat cells (A), or to MCSP on Colo-38 cells (B) measured by FACS. Mean fluorescence intensity compared to cells treated with the reference anti-CD3 IgG (as indicated), untreated cells, and cells stained with the secondary antibody only is depicted.

FIG. 21. Binding of the “1+1 IgG scFab, one armed” (see SEQ ID NOs 1, 3, 5) and “1+1 IgG scFab, one armed inverted” (see SEQ ID NOs 7, 9, 11) constructs (50 nM) to CD3 expressed on Jurkat cells (A), or to MCSP on Colo-38 cells (B) measured by FACS. Mean fluorescence intensity compared to cells treated with the reference anti-CD3 or anti-MCSP IgG (as indicated), untreated cells, and cells stained with the secondary antibody only is depicted.

FIG. 22. Dose dependent binding of the “2+1 IgG scFab, LALA” (see SEQ ID NOs 5, 17, 19) bispecific construct and the corresponding anti-MCSP IgG to MCSP on Colo-38 cells as measured by FACS.

FIG. 23. Surface expression level of different activation markers on human T cells after incubation with 1 nM of “2+1 IgG scFab, LALA” (see SEQ ID NOs 5, 17, 19) or “(scFv)₂” CD3-MCSP bispecific constructs in the presence or absence of Colo-38 tumor target cells, as indicated (E:T ratio of PBMCs to tumor cells=10:1). Depicted is the expression level of the early activation marker CD69 (A), or the late activation marker CD25 (B) on CD8⁺ T cells after 15 or 24 hours incubation, respectively.

FIG. 24. Surface expression level of the late activation marker CD25 on human T cells after incubation with 1 nM of “2+1 IgG scFab, LALA” (see SEQ ID NOs 5, 17, 19) or “(scFv)₂” CD3-MCSP bispecific constructs in the presence or absence of Colo-38 tumor target cells, as indicated (E:T ratio=5:1). Depicted is the expression level of the late activation marker CD25 on CD8⁺ T cells (A) or on CD4⁺ T cells (B) after 5 days incubation.

FIG. 25. Surface expression level of the late activation marker CD25 on cynomolgus CD8⁺ T cells from two different animals (cyno Nestor, cyno Nobu) after 43 hours incubation with the indicated concentrations of the “2+1 IgG Crossfab” bispecific construct (targeting cynomolgus CD3 and human MCSP; see SEQ ID NOs 3, 5, 35, 37), in the presence or absence of human MCSP-expressing MV-3 tumor target cells (E:T ratio=3:1). As controls, the reference IgGs (anti-cynomolgus CD3 IgG, anti-human MCSP IgG) or the unphysiologic stimulus PHA-M were used.

FIG. 26. IFN-γ levels, secreted by human pan T cells that were activated for 18.5 hours by the “2+1 IgG scFab, LALA” CD3-MCSP bispecific construct (see SEQ ID NOs 5, 17, 19) in the presence of U87MG tumor cells (E:T ratio=5:1). As controls, the corresponding anti-CD3 and anti-MCSP IgGs were administered.

FIG. 27. Killing (as measured by LDH release) of MDA-MB-435 tumor cells upon co-culture with human pan T cells (E:T ratio=5:1) and activation for 20 hours by different concentrations of the “2+1 IgG scFab” (see SEQ ID NOs 5, 21, 23), “2+1 IgG Crossfab” (see SEQ ID NOs 3, 5, 29, 33) and “(scFv)₂” bispecific molecules and corresponding IgGs.

FIG. 28. Killing (as measured by LDH release) of MDA-MB-435 tumor cells upon co-culture with human pan T cells (E:T ratio=5:1), and activation for 20 hours by different concentrations of the bispecific constructs and corresponding IgGs. “2+1 IgG scFab” constructs differing in their Fc-domain (having either a wild-type Fc domain (see SEQ ID NOs 5, 13, 15), or a Fc-domain mutated to abolish (NK) effector cell function: P329G LALA (see SEQ ID NOs 5, 21, 23), P329G LALA N297D (see SEQ ID NOs 5, 25, 27)) and the “2+1 IgG Crossfab” (see SEQ ID NOs 3, 5, 29, 33) construct were compared.

FIG. 29. Killing (as measured by LDH release) of Colo-38 tumor cells upon co-culture with human pan T cells (E:T ratio=5:1), treated with CD3-MCSP bispecific “2+1 IgG scFab, LALA” (see SEQ ID NOs 5, 17, 19) construct, “(scFv)₂” molecule or corresponding IgGs for 18.5 hours.

FIG. 30. Killing (as measured by LDH release) of Colo-38 tumor cells upon co-culture with human pan T cells (E:T ratio=5:1), treated with CD3-MCSP bispecific “2+1 IgG scFab, LALA” (see SEQ ID NOs 5, 17, 19) construct, the “(scFv)₂” molecule or corresponding IgGs for 18 hours.

FIG. 31. Killing (as measured by LDH release) of MDA-MB-435 tumor cells upon co-culture with human pan T cells (E:T ratio=5:1), and activation for 23.5 hours by different concentrations of the CD3-MCSP bispecific “2+1 IgG scFab, LALA” (see SEQ ID NOs 5, 17, 19) construct, “(scFv)₂” molecule or corresponding IgGs.

FIG. 32. Killing (as measured by LDH release) of Colo-38 tumor cells upon co-culture with human pan T cells (E:T ratio=5:1) and activation for 19 hours by different concentrations of the CD3-MCSP bispecific “1+1 IgG scFab, one armed” (see SEQ ID NOs 1, 3, 5), “1+1 IgG scFab, one armed inverted” (see SEQ ID NOs 7, 9, 11) or “(scFv)₂” constructs, or corresponding IgGs.

FIG. 33. Killing (as measured by LDH release) of Colo-38 tumor cells upon co-culture with human pan T cells (E:T ratio=5:1), treated with “1+1 IgG scFab” CD3-MCSP bispecific construct (see SEQ ID NOs 5, 21, 213) or “(scFv)₂” molecule for 20 hours.

FIG. 34. Killing (as measured by LDH release) of MDA-MB-435 tumor cells upon co-culture with human pan T cells (E:T ratio=5:1), and activation for 21 hours by different concentrations of the bispecific constructs and corresponding IgGs. The CD3-MCSP bispecific “2+1 IgG Crossfab” (see SEQ ID NOs 3, 5, 29, 33) and “1+1 IgG Crossfab” (see SEQ ID NOs 5, 29, 31, 33) constructs, the “(scFv)₂” molecule and corresponding IgGs were compared.

FIG. 35. Killing (as measured by LDH release) of different target cells (MCSP-positive Colo-38 tumor target cells, mesenchymal stem cells derived from bone marrow or adipose tissue, or pericytes from placenta; as indicated) induced by the activation of human T cells by 135 ng/ml or 1.35 ng/ml of the “2+1 IgG Crossfab” CD3-MCSP bispecific construct (see SEQ ID NOs 3, 5, 29, 33) (E:T ratio=25:1).

FIG. 36. Killing (as measured by LDH release) of Colo-38 tumor target cells, measured after an overnight incubation of 21 h, upon co-culture with human PBMCs and different CD3-MCSP bispecific constructs (“2+1 IgG scFab, LALA” (see SEQ ID NOs 5, 17, 19) and “(scFv)₂”) or a glycoengineered anti-MCSP IgG (GlycoMab). The effector to target cell ratio was fixed at 25:1 (A), or varied as depicted (B). PBMCs were isolated from fresh blood (A) or from a Buffy Coat (B).

FIG. 37. Time-dependent cytotoxic effect of the “2+1 IgG Crossfab” construct, targeting cynomolgus CD3 and human MCSP (see SEQ ID NOs 3, 5, 35, 37). Depicted is the LDH release from human MCSP-expressing MV-3 cells upon co-culture with primary cynomolgus PBMCs (E:T ratio=3:1) for 24 h or 43 h. As controls, the reference IgGs (anti-cyno CD3 IgG and anti-human MCSP IgG) were used at the same molarity. PHA-M served as a control for (unphysiologic) T cell activation.

FIG. 38. Killing (as measured by LDH release) of huMCSP-positive MV-3 melanoma cells upon co-culture with human PBMCs (E:T ratio=10:1), treated with different CD3-MCSP bispecific constructs (“2+1 IgG Crossfab” (see SEQ ID NOs 3, 5, 29, 33) and “(scFv)₂”) for ˜26 hours.

FIG. 39. Killing (as measured by LDH release) of EGFR-positive LS-174T tumor cells upon co-culture with human pan T cells (E:T ratio=5:1), treated with different CD3-EGFR bispecific constructs (“2+1 IgG scFab” (see SEQ ID NOs 45, 47, 53), “1+1 IgG scFab” (see SEQ ID NOs 47, 53, 213) and “(scFv)₂”) or reference IgGs for 18 hours.

FIG. 40. Killing (as measured by LDH release) of EGFR-positive LS-174T tumor cells upon co-culture with human pan T cells (E:T ratio=5:1), treated with different CD3-EGFR bispecific constructs (“1+1 IgG scFab, one armed” (see SEQ ID NOs 43, 45, 47), “1+1 IgG scFab, one armed inverted” (see SEQ ID NOs 11, 49, 51), “1+1 IgG scFab” (see SEQ ID NOs 47, 53, 213) and “(scFv)₂”) or reference IgGs for 21 hours.

FIG. 41. Killing (as measured by LDH release) of EGFR-positive LS-174T tumor cells upon co-culture with either human pan T cells (A) or human naive T cells (B), treated with different CD3-EGFR bispecific constructs (“1+1 IgG scFab, one armed” (see SEQ ID NOs 43, 45, 47), “1+1 IgG scFab, one armed inverted” (see SEQ ID NOs 11, 49, 51) and “(scFv)₂”) or reference IgGs for 16 hours. The effector to target cell ratio was 5:1.

FIG. 42. Killing (as measured by LDH release) of FAP-positive GM05389 fibroblasts upon co-culture with human pan T cells (E:T ratio=5:1), treated with different CD3-FAP bispecific constructs (“1+1 IgG scFab, one armed inverted” (see SEQ ID NOs 11, 51, 55), “1+1 IgG scFab” (see SEQ ID NOs 57, 61, 213), “2+1 IgG scFab” (see SEQ ID NOs 57, 59, 61) and “(scFv)₂”) for ˜18 hours.

FIG. 43. Flow cytrometric analysis of expression levels of CD107a/b, as well as perforin levels in CD8⁺ T cells that have been treated with different CD3-MCSP bispecific constructs (“2+1 IgG scFab, LALA” (see SEQ ID NOs 5, 17, 19) and “(scFv)₂”) or corresponding control IgGs in the presence (A) or absence (B) of target cells for 6 h. Human pan T cells were incubated with 9.43 nM of the different molecules in the presence or absence of Colo-38 tumor target cells at an effector to target ratio of 5:1. Monensin was added after the first hour of incubation to increase intracellular protein levels by preventing protein transport. Gates were set either on all CD107a/b positive, perforin-positive or double-positive cells, as depicted.

FIG. 44. Relative proliferation of either CD8⁺ (A) or CD4⁺ (B) human T cells upon incubation with 1 nM of different CD3-MCSP bispecific constructs (“2+1 IgG scFab, LALA” (see SEQ ID NOs 5, 17, 19) or “(scFv)₂”) or corresponding control IgGs in the presence or absence of Colo-38 tumor target cells at an effector to target cell ratio of 5:1. CFSE-labeled human pan T cells were characterized by FACS. The relative proliferation level was determined by setting a gate around the non-proliferating cells and using the cell number of this gate relative to the overall measured cell number as the reference.

FIG. 45. Levels of different cytokines measured in the supernatant of human PBMCs after treatment with 1 nM of different CD3-MCSP bispecific constructs (“2+1 IgG scFab, LALA” (see SEQ ID NOs 5, 17, 19) or “(scFv)₂”) or corresponding control IgGs in the presence (A) or absence (B) of Colo-38 tumor cells for 24 hours. The effector to target cell ratio was 10:1.

FIG. 46. Levels of different cytokines measured in the supernatant of whole blood after treatment with 1 nM of different CD3-MCSP bispecific constructs (“2+1 IgG scFab”, “2+1 IgG Crossfab” (see SEQ ID NOs 3, 5, 29, 33) or “(scFv)₂”) or corresponding control IgGs in the presence (A, B) or absence (C, D) of Colo-38 tumor cells for 24 hours. Among the bispecific constructs were different “2+1 IgG scFab” constructs having either a wild-type Fc domain (see SEQ ID NOs 5, 13, 15), or an Fc domain mutated to abolish (NK) effector cell function (LALA (see SEQ ID NOs 5, 17, 19), P329G LALA (see SEQ ID NOs 5, 2, 23) and P329G LALA N297D (see SEQ ID NOs 5, 25, 27)).

FIG. 47. CE-SDS analyses. Electropherogram shown as SDS PAGE of 2+1 IgG Crossfab, linked light chain (see SEQ ID NOs 3, 5, 29, 179). (lane 1: reduced, lane 2: non-reduced).

FIG. 48. Analytical size exclusion chromatography of 2+1 IgG Crossfab, linked light chain (see SEQ ID NOs 3, 5, 29, 179) (final product). 20 μg sample were injected.

FIG. 49. Killing (as measured by LDH release) of MCSP-positive MV-3 tumor cells upon co-culture by human PBMCs (E:T ratio=10:1), treated with different CD3-MCSP bispecific constructs for ˜44 hours (“2+1 IgG Crossfab” (see SEQ ID NOs 3, 5, 29, 33) and “2+1 IgG Crossfab, linked LC” (see SEQ ID NOs 3, 5, 29, 179)). Human PBMCs were isolated from fresh blood of healthy volunteers.

FIG. 50. Killing (as measured by LDH release) of MCSP-positive Colo-38 tumor cells upon co-culture by human PBMCs (E:T ratio=10:1), treated with different CD3-MCSP bispecific constructs for ˜22 hours (“2+1 IgG Crossfab” (see SEQ ID NOs 3, 5, 29, 33) and “2+1 IgG Crossfab, linked LC” (see SEQ ID NOs 3, 5, 29, 179)). Human PBMCs were isolated from fresh blood of healthy volunteers.

FIG. 51. Killing (as measured by LDH release) of MCSP-positive Colo-38 tumor cells upon co-culture by human PBMCs (E:T ratio=10:1), treated with different CD3-MCSP bispecific constructs for ˜22 hours (“2+1 IgG Crossfab” (see SEQ ID NOs 3, 5, 29, 33) and “2+1 IgG Crossfab, linked LC” (see SEQ ID NOs 3, 5, 29, 179)). Human PBMCs were isolated from fresh blood of healthy volunteers.

FIG. 52. Killing (as measured by LDH release) of MCSP-positive WM266-4 cells upon co-culture by human PBMCs (E:T ratio=10:1), treated with different CD3-MCSP bispecific constructs for ˜22 hours (“2+1 IgG Crossfab” (see SEQ ID NOs 3, 5, 29, 33) and “2+1 IgG Crossfab, linked LC” (see SEQ ID NOs 3, 5, 29, 179)). Human PBMCs were isolated from fresh blood of healthy volunteers.

FIG. 53. Surface expression level of the early activation marker CD69 (A) and the late activation marker CD25 (B) on human CD8⁺ T cells after 22 hours incubation with 10 nM, 80 pM or 3 pM of different CD3-MCSP bispecific constructs (“2+1 IgG Crossfab” (see SEQ ID NOs 3, 5, 29, 33) and “2+1 IgG Crossfab, linked LC” (see SEQ ID NOs 3, 5, 29, 179)) in the presence or absence of human MCSP-expressing Colo-38 tumor target cells (E:T ratio=10:1).

FIG. 54. CE-SDS analyses. (A) Electropherogram shown as SDS-PAGE of 1+1 IgG Crossfab; VL/VH exchange (LC007N9) (see SEQ ID NOs 5, 29, 33, 181): a) non-reduced, b) reduced. (B) Electropherogram shown as SDS-PAGE of 1+1 CrossMab; CL/CH1 exchange (LC007/V9) (see SEQ ID NOs 5, 23, 183, 185): a) reduced, b) non-reduced. (C) Electropherogram shown as SDS-PAGE of 2+1 IgG Crossfab, inverted; CL/CH1 exchange (LC007/V9) (see SEQ ID NOs 5, 23, 183, 187): a) reduced, b) non-reduced. (D) Electropherogram shown as SDS-PAGE of 2+1 IgG Crossfab; VL/VH exchange (M4-3 ML2/V9) (see SEQ ID NOs 33, 189, 191, 193): a) reduced, b) non-reduced. (E) Electropherogram shown as SDS-PAGE of 2+1 IgG Crossfab; CL/CH1 exchange (M4-3 ML2/V9) (see SEQ ID NOs 183, 189, 193, 195): a) reduced, b) non-reduced. (F) Electropherogram shown as SDS-PAGE of 2+1 IgG Crossfab, inverted; CL/CH1 exchange (CH1A1A/V9) (see SEQ ID NOs 65, 67, 183, 197): a) reduced, b) non-reduced. (G) Electropherogram shown as SDS-PAGE of 2+1 IgG Crossfab; CL/CH1 exchange (M4-3 ML2/H2C) (see SEQ ID NOs 189, 193, 199, 201): a) reduced, b) non-reduced. (H) Electropherogram shown as SDS-PAGE of 2+1 IgG Crossfab, inverted; CL/CH1 exchange (431/26/V9) (see SEQ ID NOs 183, 203, 205, 207): a) reduced, b) non-reduced. (I) Electropherogram shown as SDS-PAGE of “2+1 IgG Crossfab light chain fusion” (CH1A1A/V9) (see SEQ ID NOs 183, 209, 211, 213): a) reduced, b) non-reduced. (J) SDS PAGE (4-12% Bis/Tris, NuPage Invitrogen, Coomassie-stained) of “2+1 IgG Crossfab” (anti-MCSP/anti-huCD3) (see SEQ ID NOs 5, 23, 215, 217), non-reduced (left) and reduced (right). (K) Electropherogram shown as SDS-PAGE of “2+1 IgG Crossfab, inverted” (anti-MCSP/anti-huCD3) (see SEQ ID NOs 5, 23, 215, 219): a) reduced, b) non-reduced. (L) SDS PAGE (4-12% Bis/Tris, NuPage Invitrogen, Coomassie-stained) of “1+1 IgG Crossfab” (anti-CD33/anti-huCD3) (see SEQ ID NOs 33, 213, 221, 223), reduced (left) and non-reduced (right). (M) SDS PAGE (4-12% Bis/Tris, NuPage Invitrogen, Coomassie-stained) of “2+1 IgG Crossfab” (anti-CD33/anti-huCD3) (see SEQ ID NOs 33, 221, 223, 225), reduced (left) and non-reduced (right). (N) SDS PAGE (4-12% Bis/Tris, NuPage Invitrogen, Coomassie-stained) of “2+1 IgG Crossfab” (anti-CD20/anti-huCD3) (see SEQ ID NOs 33, 227, 229, 231), non-reduced.

FIG. 55. Binding of bispecific constructs (CEA/CD3 “2+1 IgG Crossfab, inverted (VL/VH)” (see SEQ ID NOs 33, 63, 65, 67) and “2+1 IgG Crossfab, inverted (CL/CH1)” (see SEQ ID NOs 65, 67, 183, 197)) to human CD3, expressed by Jurkat cells (A), or to human CEA, expressed by LS-174T cells (B) as determined by FACS. As a control, the equivalent maximum concentration of the reference IgGs and the background staining due to the labeled 2ndary antibody (goat anti-human FITC-conjugated AffiniPure F(ab′)2 Fragment, Fcγ Fragment-specific, Jackson Immuno Research Lab #109-096-098) were assessed as well.

FIG. 56. Binding of bispecific constructs (MCSP/CD3 “2+1 IgG Crossfab” (see SEQ ID NOs 3, 5, 29, 33) and “2+1 IgG Crossfab, inverted” (see SEQ ID NOs 5, 23, 183, 187)) to human CD3, expressed by Jurkat cells (A), or to human MCSP, expressed by WM266-4 tumor cells (B) as determined by FACS.

FIG. 57. Binding of the “1+1 IgG Crossfab light chain fusion” (see SEQ ID NOs 183, 209, 211, 213) to human CD3, expressed by Jurkat cells (A), or to human CEA, expressed by LS-174T cells (B) as determined by FACS.

FIG. 58. Binding of the “2+1 IgG Crossfab” (see SEQ ID NOs 5, 23, 215, 217) and the “2+1 IgG Crossfab, inverted” (see SEQ ID NOs 5, 23, 215, 219) constructs to human CD3, expressed by Jurkat cells (A), or human MCSP, expressed by WM266-4 tumor cells (B) as determined by FACS.

FIG. 59. Surface expression level of the early activation marker CD69 (A) or the late activation marker CD25 (B) on human CD4⁺ or CD8⁺ T cells after 24 hours incubation with the indicated concentrations of the CD3/MCSP “1+1 CrossMab” (see SEQ ID NOs 5, 23, 183, 185), “1+1 IgG Crossfab” (see SEQ ID NOs 5, 29, 33, 181) and “2+1 IgG Crossfab” (see SEQ ID NOs 3, 5, 29, 33) constructs. The assay was performed in the presence or absence of MV-3 target cells, as indicated.

FIG. 60. Surface expression level of the early activation marker CD25 on CD4⁺ or CD8⁺ T cells from two different cynomolgus monkeys (A and B) in the presence or absence of huMCSP-positive MV-3 tumor cells upon co-culture with cynomolgus PBMCs (E:T ratio=3:1, normalized to CD3⁺ numbers), treated with the “2+1 IgG Crossfab” (see SEQ ID NOs 5, 23, 215, 217) and the “2+1 IgG Crossfab, inverted” (see SEQ ID NOs 5, 23, 215, 219) for ˜41 hours.

FIG. 61. Killing (as measured by LDH release) of MKN-45 (A) or LS-174T (B) tumor cells upon co-culture with human PBMCs (E:T ratio=10:1) and activation for 28 hours by different concentrations of the “2+1 IgG Crossfab, inverted (VL/VH)” (see SEQ ID NOs 33, 63, 65, 67) versus the “2+1 IgG Crossfab, inverted (CL/CH1)” (see SEQ ID NOs 65, 67, 183, 197) construct.

FIG. 62. Killing (as measured by LDH release) of WM266-4 tumor cells upon co-culture with human PBMCs (E:T ratio=10:1) and activation for 26 hours by different concentrations of the “2+1 IgG Crossfab (VL/VH)” (see SEQ ID NOs 33, 189, 191, 193) versus the “2+1 IgG Crossfab (CL/CH1)” (see SEQ ID NOs 183, 189, 193, 195) construct.

FIG. 63. Killing (as measured by LDH release) of MV-3 tumor cells upon co-culture with human PBMCs (E:T ratio=10:1) and activation for 27 hours by different concentrations of the “2+1 IgG Crossfab (VH/VL)” (see SEQ ID NOs 33, 189, 191, 193) versus the “2+1 IgG Crossfab (CL/CH1)” (see SEQ ID NOs 183, 189, 193, 195) constructs.

FIG. 64. Killing (as measured by LDH release) of human MCSP-positive WM266-4 (A) or MV-3 (B) tumor cells upon co-culture with human PBMCs (E:T ratio=10:1) and activation for 21 hours by different concentrations of the “2+1 IgG Crossfab” (see SEQ ID NOs 3, 5, 29, 33), the “1+1 CrossMab” (see SEQ ID NOs 5, 23, 183, 185), and the “1+1 IgG Crossfab” (see SEQ ID NOs 5, 29, 33, 181), as indicated.

FIG. 65. Killing (as measured by LDH release) of MKN-45 (A) or LS-174T (B) tumor cells upon co-culture with human PBMCs (E:T ratio=10:1) and activation for 28 hours by different concentrations of the “1+1 IgG Crossfab LC fusion” (see SEQ ID NOs 183, 209, 211, 213).

FIG. 66. Killing (as measured by LDH release) of MC38-huCEA tumor cells upon co-culture with human PBMCs (E:T ratio=10:1) and activation for 24 hours by different concentrations of the “1+1 IgG Crossfab LC fusion” (see SEQ ID NOs 183, 209, 211, 213) versus an untargeted “2+1 IgG Crossfab” reference.

FIG. 67. Killing (as measured by LDH release) of human MCSP-positive MV-3 (A) or WM266-4 (B) tumor cells upon co-culture with human PBMCs (E:T ratio=10:1), treated with the “2+1 IgG Crossfab (V9)” (see SEQ ID NOs 3, 5, 29, 33) and the “2+1 IgG Crossfab, inverted (V9)” (see SEQ ID NOs 5, 23, 183, 187), the “2+1 IgG Crossfab (anti-CD3)” (see SEQ ID NOs 5, 23, 215, 217) and the “2+1 IgG Crossfab, inverted (anti-CD3)” (see SEQ ID NOs 5, 23, 215, 219) constructs.

FIG. 68. Alignment of affinity matured anti-MCSP clones compared to the non-matured parental clone (M4-3 ML2).

FIG. 69. Schematic drawing of the MCSP TCB (2+1 Crossfab-IgG P329G LALA inverted) molecule.

FIG. 70. CE-SDS analyses of MCSP TCB (2+1 Crossfab-IgG P329G LALA inverted, SEQ ID NOs: 278, 319, 320 and 321). Electropherogram shown as SDS-PAGE of MCSP TCB: A) non reduced, B) reduced.

FIG. 71. Schematic drawing of CEA TCB (2+1 Crossfab-IgG P329G LALA inverted) molecule.

FIG. 72. CE-SDS analyses of CEA TCB (2+1 Crossfab-IgG P329G LALA inverted, SEQ ID NOs: 288, 322, 323 and 324) molecule. Electropherogram shown as SDS-Page of CEA TCB: A) non reduced, B) reduced.

FIG. 73. Binding of MCSP TCB (SEQ ID NOs: 278, 319, 320 and 321) to A375 cells (MCSP⁺) (A) and Jurkat (CD3⁺ cells) (B). “Untargeted TCB”: bispecific antibody engaging CD3 but no second antigen (SEQ ID NOs: 325, 326, 327 and 328).

FIG. 74. T-cell killing induced by MCSP TCB antibody (SEQ ID NOs: 278, 319, 320 and 321) of A375 (high MCSP) (A), MV-3 (medium MCSP) (B), HCT-116 (low MCSP) (C) and LS180 (MCSP negative) (D) target cells (E:T=10:1, effectors human PBMCs, incubation time 24 h). “Untargeted TCB”: bispecific antibody engaging CD3 but no second antigen (SEQ ID NOs: 325, 326, 327 and 328).

FIG. 75. Upregulation of CD25 and CD69 on human CD8⁺ (A, B) and CD4⁺ (C, D) T cells after T cell-mediated killing of MV3 melanoma cells (E:T=10:1, 24 h incubation) induced by MCSP TCB antibody (SEQ ID NOs: 278, 319, 320 and 321). “Untargeted TCB”: bispecific antibody engaging CD3 but no second antigen (SEQ ID NOs: 325, 326, 327 and 328).

FIG. 76. Secretion of IL-2 (A), IFN-γ (B), TNFα (C), IL-4 (D), IL-10 (E) and Granzyme B (F) by human PBMCs after T cell mediated killing of MV3 melanoma cells (E:T=10:1, 24 h incubation) induced by MCSP TCB antibody (SEQ ID NOs: 278, 319, 320 and 321). “Untargeted TCB”: bispecific antibody engaging CD3 but no second antigen (SEQ ID NOs: 325, 326, 327 and 328).

FIG. 77. Binding of CEA TCB (SEQ ID NOs: 288, 322, 323 and 324) to CEA-expressing A549 lung adenocarcinoma cells (A) and CD3-expressing immortalized human and cynomolgus T lymphocyte lines (Jurkat (B) and HSC-F (C), respectively).

FIG. 78. T-cell killing induced by CEA TCB (SEQ ID NOs: 288, 322, 323 and 324) of HPAFII (high CEA) (A, E), BxPC-3 (medium CEA) (B, F), ASPC-1 (low CEA) (C, G) and HCT-116 cells (CEA negative) (D, H). E:T=10:1, effectors human PBMCs, incubation time 24 h (A-D) or 48 h (E-H). “Untargeted TCB”: bispecific antibody engaging CD3 but no second antigen (SEQ ID NOs: 325, 326, 327 and 328).

FIG. 79. Human CD8⁺ and CD4⁺ T cell proliferation (A-D) and upregulation of CD25 on human CD8⁺ and CD4 T cells (E-H) 5 days after T cell-mediated killing of HPAFII (high CEA) (A, E), BxPC-3 (medium CEA) (B, F), ASPC-1 (low CEA) (C, G) and HCT-116 cells (CEA negative) (D, H) induced by CEA TCB (SEQ ID NOs: 288, 322, 323 and 324). “DP47 TCB”: bispecific antibody engaging CD3 but no second antigen (SEQ ID NOs: 325, 326, 327 and 328).

FIG. 80. Secretion of IFN-γ (A), TNFα (B), Granzyme B (C), IL-2 (D), IL-6 (E) and IL-10 (F) after T cell mediated killing of MKN45 tumor cells (E:T=10:1, 48 h incubation) induced by CEA TCB (SEQ ID NOs: 288, 322, 323 and 324). “Untargeted TCB”: bispecific antibody engaging CD3 but no second antigen (SEQ ID NOs: 325, 326, 327 and 328).

FIG. 81. T cell-mediated killing of CEA-expressing LS180 tumor target cells induced by CEA TCB (SEQ ID NOs: 288, 322, 323 and 324) in presence of increasing concentrations of shed CEA (sCEA), detected 24 h (A) or 48 h (B) after incubation with the CEA TCB and sCEA.

FIG. 82. T cell-mediated killing of A549 (lung adenocarcinoma) cells overexpressing human CEA (A549-hCEA), assessed 21 h (A, B) and 40 h (C, D) after incubation with CEA TCB (SEQ ID NOs: 288, 322, 323 and 324) and human PBMCs (A, C) or cynomolgus PBMCs (B, D) as effector cells.

FIG. 83. T cell-mediated killing of CEA-expressing human colorectal cancer cell lines induced by CEA TCB (SEQ ID NOs: 288, 322, 323 and 324) at 0.8 nM (A), 4 nM (B) and 20 nM (C). (D) correlation between CEA expression and % specific lysis at 20 nM of CEA TCB, (E) correlation between CEA expression and EC₅₀ of CEA TCB.

FIG. 84. In vivo anti-tumor efficacy of CEA TCB (SEQ ID NOs: 288, 322, 323 and 324) in a LS174T-fluc2 human colon carcinoma co-grafted with human PBMC (E:T ratio 5:1). Results show average and SEM from 12 mice of tumor volume measured by caliper (A and C) and by bioluminescence (Total Flux, B and D) in the different study groups. (A, B) early treatment starting at day 1, (C, D) delayed treatment starting at day 7. The MCSP TCB (SEQ ID NOs: 278, 319, 320 and 321) was used as negative control.

FIG. 85. In vivo anti-tumor efficacy of CEA TCB (SEQ ID NOs: 288, 322, 323 and 324) in a LS174T-fluc2 human colon carcinoma co-grafted with human PBMC (E:T ratio 1:1). Results show average and SEM from 10 mice of tumor volume measured by caliper (A) and by bioluminescence (Total Flux, B) in the different study groups. The MCSP TCB (SEQ ID NOs: 278, 319, 320 and 321) was used as negative control.

FIG. 86. In vivo efficacy of murinized CEA TCB in a Panco2-huCEA orthotopic tumor model in immunocompetent huCD3ε/huCEA transgenic mice.

FIG. 87. Thermal stability of CEA TCB. Dynamic Light Scattering measured in a temperature ramp from 25-75° C. at 0.05° C./min. Duplicate is shown in grey.

FIG. 88. Thermal stability of MCSP TCB. Dynamic Light Scattering measured in a temperature ramp from 25-75° C. at 0.05° C./min. Duplicate is shown as grey line.

FIG. 89. T cell-mediated killing induced by MCSP TCB (SEQ ID NOs: 278, 319, 320 and 321) and MCSP 1+1 CrossMab TCB antibodies of (A) A375 (high MCSP), (B) MV-3 (medium MCSP) and (C) HCT-116 (low MCSP) tumor target cells. (D) LS180 (MCSP negative tumor cell line) was used as negative control. Tumor cell killing was assessed 24 h (A-D) and 48 h (E-H) post incubation of target cells with the antibodies and effector cells (human PBMCs).

FIG. 90. CD25 and CD69 upregulation on CD8⁺ and CD4⁺ T cells after T-cell killing of MCSP-expressing tumor cells (A375, A-D and MV-3, E-H) mediated by the MCSP TCB (SEQ ID NOs: 278, 319, 320 and 321) and MCSP 1+1 CrossMab TCB antibodies.

FIG. 91. CE-SDS analyses of DP47 GS TCB (2+1 Crossfab-IgG P329G LALA inverted=“Untargeted TCB” SEQ ID NOs: 325, 326, 327 and 328) containing DP47 GS as non binding antibody and humanized CH2527 as anti CD3 antibody. Electropherogram shown as SDS-PAGE of DP47 GS TCB: A) non reduced, B) reduced.

FIG. 92. Schematic drawing of the MCSP TCB hIgG4 S228P/L325E molecule.

FIG. 93. CE-SDS analyses of the MCSP TCB hIgG4 S228P/L325E molecule (SEQ ID NOs: 278, 319, 369 and 370). Electropherogram shown as SDS-PAGE of MCSP TCB hIgG4 S228P/L325E: (A) non reduced, (B) reduced.

FIG. 94. Binding of MCSP TCB hIgG4 S228P/L325E (SEQ ID NOs: 278, 319, 369 and 370) to MV-3 cells (MCSP+; EC50=2029 pM) (A) and Jurkat (CD3+) (B).

FIG. 95. T-cell killing induced by MCSP TCB hIgG4 S228P/L325E (SEQ ID NOs: 278, 319, 369 and 370) of MV-3 target cells with an incubation time of 24 h (EC50=14.9 pM) (A) and 48 h (EC50=0.24 pM) (B) (E:T=10:1, effectors human PBMCs).

### DETAILED DESCRIPTION OF THE INVENTION

### Definitions

Terms are used herein as generally used in the art, unless otherwise defined in the following. As used herein, the term “antigen binding molecule” refers in its broadest sense to a molecule that specifically binds an antigenic determinant. Examples of antigen binding molecules are immunoglobulins and derivatives, e.g. fragments, thereof.

The term “bispecific” means that the antigen binding molecule is able to specifically bind to at least two distinct antigenic determinants. Typically, a bispecific antigen binding molecule comprises two antigen binding sites, each of which is specific for a different antigenic determinant. In certain embodiments the bispecific antigen binding molecule is capable of simultaneously binding two antigenic determinants, particularly two antigenic determinants expressed on two distinct cells.

The term “valent” as used herein denotes the presence of a specified number of antigen binding sites in an antigen binding molecule. As such, the term “monovalent binding to an antigen” denotes the presence of one (and not more than one) antigen binding site specific for the antigen in the antigen binding molecule.

An “antigen binding site” refers to the site, i.e. one or more amino acid residues, of an antigen binding molecule which provides interaction with the antigen. For example, the antigen binding site of an antibody comprises amino acid residues from the complementarity determining regions (CDRs). A native immunoglobulin molecule typically has two antigen binding sites, a Fab molecule typically has a single antigen binding site.

As used herein, the term “antigen binding moiety” refers to a polypeptide molecule that specifically binds to an antigenic determinant. In one embodiment, an antigen binding moiety is able to direct the entity to which it is attached (e.g. a second antigen binding moiety) to a target site, for example to a specific type of tumor cell or tumor stroma bearing the antigenic determinant. In another embodiment an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

As used herein, the term “antigenic determinant” is synonymous with “antigen” and “epitope,” and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM). The proteins referred to as antigens herein (e.g. MCSP, FAP, CEA, EGFR, CD33, CD3) can be any native form the proteins from any vertebrate source, including mammals such as primates (e.g. humans) and rodents (e.g. mice and rats), unless otherwise indicated. In a particular embodiment the antigen is a human protein. Where reference is made to a specific protein herein, the term encompasses the “full-length”, unprocessed protein as well as any form of the protein that results from processing in the cell. The term also encompasses naturally occurring variants of the protein, e.g. splice variants or allelic variants. Exemplary human proteins useful as antigens include, but are not limited to: Melanoma-associated Chondroitin Sulfate Proteoglycan (MCSP), also known as Chondroitin Sulfate Proteoglycan 4 (CSPG4; UniProt no. Q6UVK1 (version 70), NCBI RefSeq no. NP_{—}001888.2); Fibroblast Activation Protein (FAP), also known as Seprase (Uni Prot nos. Q12884, Q86Z29, Q99998, NCBI Accession no. NP_{—}004451); Carcinoembroynic antigen (CEA), also known as Carcinoembryonic antigen-related cell adhesion molecule 5 (CEACAM5; UniProt no. P06731 (version 119), NCBI RefSeq no. NP_{—}004354.2); CD33, also known as gp67 or Siglec-3 (UniProt no. P20138, NCBI Accession nos. NP_{—}001076087, NP_{—}001171079); Epidermal Growth Factor Receptor (EGFR), also known as ErbB-1 or Her1 (UniProt no. P0053, NCBI Accession nos. NP_{—}958439, NP_{—}958440), and CD3, particularly the epsilon subunit of CD3 (see UniProt no. P07766 (version 130), NCBI RefSeq no. NP_{—}000724.1, SEQ ID NO: 265 for the human sequence; or UniProt no. Q95LI5 (version 49), NCBI GenBank no. BAB71849.1, SEQ ID NO: 266 for the cynomolgus [Macaca fascicularis] sequence). In certain embodiments the T cell activating bispecific antigen binding molecule of the invention binds to an epitope of an activating T cell antigen or a target cell antigen that is conserved among the activating T cell antigen or target antigen from different species.

In certain embodiments, the T cell activating bispecific antigen binding molecule of the invention binds to CD3 and CEA (CEACAM5), but does not bind to CEACAM1 or CEACAM6. By “specific binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antigen binding molecule comprising that antigen binding moiety, has a dissociation constant (K_{D}) of ≦1 μM, ≦100 nM, ≦10 nM, ≦1 nM, ≦0.1 nM, ≦0.01 nM, or ≦0.001 nM (e.g. 10⁻⁸M or less, e.g. from 10⁻⁸M to 10⁻¹³M, e.g., from 10⁻⁹M to 10⁻¹³ M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

“Reduced binding”, for example reduced binding to an Fc receptor, refers to a decrease in affinity for the respective interaction, as measured for example by SPR. For clarity the term includes also reduction of the affinity to zero (or below the detection limit of the analytic method), i.e. complete abolishment of the interaction. Conversely, “increased binding” refers to an increase in binding affinity for the respective interaction.

An “activating T cell antigen” as used herein refers to an antigenic determinant expressed on the surface of a T lymphocyte, particularly a cytotoxic T lymphocyte, which is capable of inducing T cell activation upon interaction with an antigen binding molecule. Specifically, interaction of an antigen binding molecule with an activating T cell antigen may induce T cell activation by triggering the signaling cascade of the T cell receptor complex. In a particular embodiment the activating T cell antigen is CD3.

“T cell activation” as used herein refers to one or more cellular response of a T lymphocyte, particularly a cytotoxic T lymphocyte, selected from: proliferation, differentiation, cytokine secretion, cytotoxic effector molecule release, cytotoxic activity, and expression of activation markers. The T cell activating bispecific antigen binding molecules of the invention are capable of inducing T cell activation. Suitable assays to measure T cell activation are known in the art described herein.

A “target cell antigen” as used herein refers to an antigenic determinant presented on the surface of a target cell, for example a cell in a tumor such as a cancer cell or a cell of the tumor stroma.

As used herein, the terms “first” and “second” with respect to antigen binding moieties etc., are used for convenience of distinguishing when there is more than one of each type of moiety. Use of these terms is not intended to confer a specific order or orientation of the T cell activating bispecific antigen binding molecule unless explicitly so stated.

A “Fab molecule” refers to a protein consisting of the VH and CH1 domain of the heavy chain (the “Fab heavy chain”) and the VL and CL domain of the light chain (the “Fab light chain”) of an immunoglobulin.

By “fused” is meant that the components (e.g. a Fab molecule and an Fc domain subunit) are linked by peptide bonds, either directly or via one or more peptide linkers.

As used herein, the term “single-chain” refers to a molecule comprising amino acid monomers linearly linked by peptide bonds. In certain embodiments, one of the antigen binding moieties is a single-chain Fab molecule, i.e. a Fab molecule wherein the Fab light chain and the Fab heavy chain are connected by a peptide linker to form a single peptide chain. In a particular such embodiment, the C-terminus of the Fab light chain is connected to the N-terminus of the Fab heavy chain in the single-chain Fab molecule.

By a “crossover” Fab molecule (also termed “Crossfab”) is meant a Fab molecule wherein either the variable regions or the constant regions of the Fab heavy and light chain are exchanged, i.e. the crossover Fab molecule comprises a peptide chain composed of the light chain variable region and the heavy chain constant region, and a peptide chain composed of the heavy chain variable region and the light chain constant region. For clarity, in a crossover Fab molecule wherein the variable regions of the Fab light chain and the Fab heavy chain are exchanged, the peptide chain comprising the heavy chain constant region is referred to herein as the “heavy chain” of the crossover Fab molecule. Conversely, in a crossover Fab molecule wherein the constant regions of the Fab light chain and the Fab heavy chain are exchanged, the peptide chain comprising the heavy chain variable region is referred to herein as the “heavy chain” of the crossover Fab molecule.

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable region (VH), also called a variable heavy domain or a heavy chain variable domain, followed by three constant domains (CH1, CH2, and CH3), also called a heavy chain constant region. Similarly, from N- to C-terminus, each light chain has a variable region (VL), also called a variable light domain or a light chain variable domain, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “antibody” herein is used in the broadest sense and encompasses various antibody structures, including but not limited to monoclonal antibodies, polyclonal antibodies, and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Plückthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “antigen binding domain” refers to the part of an antibody that comprises the area which specifically binds to and is complementary to part or all of an antigen. An antigen binding domain may be provided by, for example, one or more antibody variable domains (also called antibody variable regions). Particularly, an antigen binding domain comprises an antibody light chain variable region (VL) and an antibody heavy chain variable region (VH).

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen-binding specificity.

The term “hypervariable region” or “HVR”, as used herein, refers to each of the regions of an antibody variable domain which are hypervariable in sequence and/or form structurally defined loops (“hypervariable loops”). Generally, native four-chain antibodies comprise six HVRs; three in the VH (H1, H2, H3), and three in the VL (L1, L2, L3). HVRs generally comprise amino acid residues from the hypervariable loops and/or from the complementarity determining regions (CDRs), the latter being of highest sequence variability and/or involved in antigen recognition. With the exception of CDR1 in VH, CDRs generally comprise the amino acid residues that form the hypervariable loops. Hypervariable regions (HVRs) are also referred to as “complementarity determining regions” (CDRs), and these terms are used herein interchangeably in reference to portions of the variable region that form the antigen binding regions. This particular region has been described by Kabat et al., U.S. Dept. of Health and Human Services, Sequences of Proteins of Immunological Interest (1983) and by Chothia et al., J Mol Biol 196:901-917 (1987), where the definitions include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth below in Table 1 as a comparison. The exact residue numbers which encompass a particular CDR will vary depending on the sequence and size of the CDR. Those skilled in the art can routinely determine which residues comprise a particular CDR given the variable region amino acid sequence of the antibody.

TABLE 1
CDR Definitions¹

| CDR | Kabat | Chothia | AbM² |
|---|---|---|---|
| V_{H} CDR1 | 31-35 | 26-32 | 26-35 |
| V_{H} CDR2 | 50-65 | 52-58 | 50-58 |
| V_{H} CDR3 | 95-102 | 95-102 | 95-102 |
| V_{L} CDR1 | 24-34 | 26-32 | 24-34 |
| V_{L} CDR2 | 50-56 | 50-52 | 50-56 |
| V_{L} CDR3 | 89-97 | 91-96 | 89-97 |

¹Numbering of all CDR definitions in Table 1 is according to the numbering conventions set forth by Kabat et al. (see below).
²“AbM” with a lowercase “b” as used in Table 1 refers to the CDRs as defined by Oxford Molecular's “AbM” antibody modeling software.

Kabat et al. also defined a numbering system for variable region sequences that is applicable to any antibody. One of ordinary skill in the art can unambiguously assign this system of “Kabat numbering” to any variable region sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983). Unless otherwise specified, references to the numbering of specific amino acid residue positions in an antibody variable region are according to the Kabat numbering system.

The polypeptide sequences of the sequence listing are not numbered according to the Kabat numbering system. However, it is well within the ordinary skill of one in the art to convert the numbering of the sequences of the Sequence Listing to Kabat numbering.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following sequence in VH (or VL): FR1-H1(L1)-FR2-H2(L2)-FR3-H3 (L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991. A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

As used herein, the terms “engineer, engineered, engineering”, are considered to include any manipulation of the peptide backbone or the post-translational modifications of a naturally occurring or recombinant polypeptide or fragment thereof. Engineering includes modifications of the amino acid sequence, of the glycosylation pattern, or of the side chain group of individual amino acids, as well as combinations of these approaches.

The term “amino acid mutation” as used herein is meant to encompass amino acid substitutions, deletions, insertions, and modifications. Any combination of substitution, deletion, insertion, and modification can be made to arrive at the final construct, provided that the final construct possesses the desired characteristics, e.g., reduced binding to an Fc receptor, or increased association with another peptide. Amino acid sequence deletions and insertions include amino- and/or carboxy-terminal deletions and insertions of amino acids. Particular amino acid mutations are amino acid substitutions. For the purpose of altering e.g. the binding characteristics of an Fc region, non-conservative amino acid substitutions, i.e. replacing one amino acid with another amino acid having different structural and/or chemical properties, are particularly preferred. Amino acid substitutions include replacement by non-naturally occurring amino acids or by naturally occurring amino acid derivatives of the twenty standard amino acids (e.g. 4-hydroxyproline, 3-methylhistidine, ornithine, homoserine, 5-hydroxylysine). Amino acid mutations can be generated using genetic or chemical methods well known in the art. Genetic methods may include site-directed mutagenesis, PCR, gene synthesis and the like. It is contemplated that methods of altering the side chain group of an amino acid by methods other than genetic engineering, such as chemical modification, may also be useful. Various designations may be used herein to indicate the same amino acid mutation. For example, a substitution from proline at position 329 of the Fc domain to glycine can be indicated as 329G, G329, G₃₂₉, P329G, or Pro329Gly.

As used herein, term “polypeptide” refers to a molecule composed of monomers (amino acids) linearly linked by amide bonds (also known as peptide bonds). The term “polypeptide” refers to any chain of two or more amino acids, and does not refer to a specific length of the product. Thus, peptides, dipeptides, tripeptides, oligopeptides, “protein,” “amino acid chain,” or any other term used to refer to a chain of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis. A polypeptide of the invention may be of a size of about 3 or more, 5 or more, 10 or more, 20 or more, 25 or more, 50 or more, 75 or more, 100 or more, 200 or more, 500 or more, 1,000 or more, or 2,000 or more amino acids. Polypeptides may have a defined three-dimensional structure, although they do not necessarily have such structure. Polypeptides with a defined three-dimensional structure are referred to as folded, and polypeptides which do not possess a defined three-dimensional structure, but rather can adopt a large number of different conformations, and are referred to as unfolded.

By an “isolated” polypeptide or a variant, or derivative thereof is intended a polypeptide that is not in its natural milieu. No particular level of purification is required. For example, an isolated polypeptide can be removed from its native or natural environment. Recombinantly produced polypeptides and proteins expressed in host cells are considered isolated for the purpose of the invention, as are native or recombinant polypeptides which have been separated, fractionated, or partially or substantially purified by any suitable technique.

Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary. In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “polynucleotide” refers to an isolated nucleic acid molecule or construct, e.g. messenger RNA (mRNA), virally-derived RNA, or plasmid DNA (pDNA). A polynucleotide may comprise a conventional phosphodiester bond or a non-conventional bond (e.g. an amide bond, such as found in peptide nucleic acids (PNA). The term “nucleic acid molecule” refers to any one or more nucleic acid segments, e.g. DNA or RNA fragments, present in a polynucleotide.

By “isolated” nucleic acid molecule or polynucleotide is intended a nucleic acid molecule, DNA or RNA, which has been removed from its native environment. For example, a recombinant polynucleotide encoding a polypeptide contained in a vector is considered isolated for the purposes of the present invention. Further examples of an isolated polynucleotide include recombinant polynucleotides maintained in heterologous host cells or purified (partially or substantially) polynucleotides in solution. An isolated polynucleotide includes a polynucleotide molecule contained in cells that ordinarily contain the polynucleotide molecule, but the polynucleotide molecule is present extrachromosomally or at a chromosomal location that is different from its natural chromosomal location. Isolated RNA molecules include in vivo or in vitro RNA transcripts of the present invention, as well as positive and negative strand forms, and double-stranded forms. Isolated polynucleotides or nucleic acids according to the present invention further include such molecules produced synthetically. In addition, a polynucleotide or a nucleic acid may be or may include a regulatory element such as a promoter, ribosome binding site, or a transcription terminator.

By a nucleic acid or polynucleotide having a nucleotide sequence at least, for example, 95% “identical” to a reference nucleotide sequence of the present invention, it is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These alterations of the reference sequence may occur at the 5′ or 3′ terminal positions of the reference nucleotide sequence or anywhere between those terminal positions, interspersed either individually among residues in the reference sequence or in one or more contiguous groups within the reference sequence. As a practical matter, whether any particular polynucleotide sequence is at least 80%, 85%, 90%, 95%, 96%, 97%, 98% or 99% identical to a nucleotide sequence of the present invention can be determined conventionally using known computer programs, such as the ones discussed above for polypeptides (e.g. ALIGN-2).

The term “expression cassette” refers to a polynucleotide generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter. In certain embodiments, the expression cassette of the invention comprises polynucleotide sequences that encode bispecific antigen binding molecules of the invention or fragments thereof.

The term “vector” or “expression vector” is synonymous with “expression construct” and refers to a DNA molecule that is used to introduce and direct the expression of a specific gene to which it is operably associated in a target cell. The term includes the vector as a self-replicating nucleic acid structure as well as the vector incorporated into the genome of a host cell into which it has been introduced. The expression vector of the present invention comprises an expression cassette. Expression vectors allow transcription of large amounts of stable mRNA. Once the expression vector is inside the target cell, the ribonucleic acid molecule or protein that is encoded by the gene is produced by the cellular transcription and/or translation machinery. In one embodiment, the expression vector of the invention comprises an expression cassette that comprises polynucleotide sequences that encode bispecific antigen binding molecules of the invention or fragments thereof.

The terms “host cell”, “host cell line,” and “host cell culture” are used interchangeably and refer to cells into which exogenous nucleic acid has been introduced, including the progeny of such cells. Host cells include “transformants” and “transformed cells,” which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein. A host cell is any type of cellular system that can be used to generate the bispecific antigen binding molecules of the present invention. Host cells include cultured cells, e.g. mammalian cultured cells, such as CHO cells, BHK cells, NS0 cells, SP2/0 cells, YO myeloma cells, P3X63 mouse myeloma cells, PER cells, PER.C6 cells or hybridoma cells, yeast cells, insect cells, and plant cells, to name only a few, but also cells comprised within a transgenic animal, transgenic plant or cultured plant or animal tissue.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

Antibody-dependent cell-mediated cytotoxicity (ADCC) is an immune mechanism leading to the lysis of antibody-coated target cells by immune effector cells. The target cells are cells to which antibodies or derivatives thereof comprising an Fc region specifically bind, generally via the protein part that is N-terminal to the Fc region. As used herein, the term “reduced ADCC” is defined as either a reduction in the number of target cells that are lysed in a given time, at a given concentration of antibody in the medium surrounding the target cells, by the mechanism of ADCC defined above, and/or an increase in the concentration of antibody in the medium surrounding the target cells, required to achieve the lysis of a given number of target cells in a given time, by the mechanism of ADCC. The reduction in ADCC is relative to the ADCC mediated by the same antibody produced by the same type of host cells, using the same standard production, purification, formulation and storage methods (which are known to those skilled in the art), but that has not been engineered. For example the reduction in ADCC mediated by an antibody comprising in its Fc domain an amino acid substitution that reduces ADCC, is relative to the ADCC mediated by the same antibody without this amino acid substitution in the Fc domain. Suitable assays to measure ADCC are well known in the art (see e.g. PCT publication no. WO 2006/082515 or PCT publication no. WO 2012/130831).

An “effective amount” of an agent refers to the amount that is necessary to result in a physiological change in the cell or tissue to which it is administered.

A “therapeutically effective amount” of an agent, e.g. a pharmaceutical composition, refers to an amount effective, at dosages and for periods of time necessary, to achieve the desired therapeutic or prophylactic result. A therapeutically effective amount of an agent for example eliminates, decreases, delays, minimizes or prevents adverse effects of a disease.

An “individual” or “subject” is a mammal. Mammals include, but are not limited to, domesticated animals (e.g. cows, sheep, cats, dogs, and horses), primates (e.g. humans and non-human primates such as monkeys), rabbits, and rodents (e.g. mice and rats). Particularly, the individual or subject is a human.

The term “pharmaceutical composition” refers to a preparation which is in such form as to permit the biological activity of an active ingredient contained therein to be effective, and which contains no additional components which are unacceptably toxic to a subject to which the formulation would be administered.

A “pharmaceutically acceptable carrier” refers to an ingredient in a pharmaceutical composition, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, T cell activating bispecific antigen binding molecules of the invention are used to delay development of a disease or to slow the progression of a disease.

The term “package insert” is used to refer to instructions customarily included in commercial packages of therapeutic products, that contain information about the indications, usage, dosage, administration, combination therapy, contraindications and/or warnings concerning the use of such therapeutic products.

### DETAILED DESCRIPTION OF THE EMBODIMENTS

In a first aspect the invention provides a T cell activating bispecific antigen binding molecule comprising a first and a second antigen binding moiety, one of which is a Fab molecule capable of specific binding to an activating T cell antigen and the other one of which is a Fab molecule capable of specific binding to a target cell antigen, and an IgG₄ Fc domain composed of a first and a second subunit capable of stable association; wherein the first antigen binding moiety is
(a) a single chain Fab molecule wherein the Fab light chain and the Fab heavy chain are connected by a peptide linker, or
(b) a crossover Fab molecule wherein either the variable or the constant regions of the Fab light chain and the Fab heavy chain are exchanged.

In some embodiments, the activating T cell antigen is CD3.

In a particular embodiment, the first antigen binding moiety is a Fab molecule capable of specific binding to CD3, comprising at least one heavy chain complementarity determining region (CDR) selected from the group consisting of SEQ ID NO: 270, SEQ ID NO: 271 and SEQ ID NO: 272 and at least one light chain CDR selected from the group of SEQ ID NO: 274, SEQ ID NO: 275, SEQ ID NO: 276.

In one embodiment the first antigen binding moiety is a Fab molecule capable of specific binding to CD3 comprising a heavy chain variable region comprising an amino acid sequence selected from the group of: SEQ ID NO: 269, SEQ ID NO: 298 and SEQ ID NO: 299 and a light chain variable region comprising an amino acid sequence selected from the group of: SEQ ID NO: 273 and SEQ ID NO: 297.

In one embodiment the first antigen binding moiety is a Fab molecule capable of specific binding to CD3 comprising a heavy chain variable region comprising the amino acid sequence of SEQ ID NO: 269 and a light chain variable region comprising the amino acid sequence of SEQ ID NO: 273.

In a specific embodiment the second antigen binding moiety is capable of specific binding to CEA and comprises at least one heavy chain complementarity determining region (CDR) selected from the group consisting of SEQ ID NO: 290, SEQ ID NO: 291 and SEQ ID NO: 292 and at least one light chain CDR selected from the group of SEQ ID NO: 294, SEQ ID NO: 295 and SEQ ID NO: 296.

In another specific embodiment, the second antigen binding moiety is capable of specific binding to CEA and comprises a heavy chain variable region comprising the amino acid sequence of SEQ ID NO: 289 and a light chain variable region comprising the amino acid sequence of SEQ ID NO: 293.

In another specific embodiment, the second antigen binding moiety is capable of specific binding to MCSP and comprises at least one heavy chain complementarity determining region (CDR) selected from the group consisting of SEQ ID NO: 280, SEQ ID NO: 281, SEQ ID NO: 282, SEQ ID NO: 301, SEQ ID NO: 303, SEQ ID NO: 304 and SEQ ID NO: 306 and at least one light chain CDR selected from the group of SEQ ID NO: 284, SEQ ID NO: 285, SEQ ID NO: 286, SEQ ID NO: 310, SEQ ID NO: 311, SEQ ID NO: 314, SEQ ID NO: 315 and SEQ ID NO: 316.

In another specific embodiment, the second antigen binding moiety is capable of specific binding to MCSP and comprises at least one heavy chain complementarity determining region (CDR) selected from the group consisting of SEQ ID NO: 280, SEQ ID NO: 281 and SEQ ID NO: 282 and at least one light chain CDR selected from the group of SEQ ID NO: 284, SEQ ID NO: 285 and SEQ ID NO: 286.

In another specific embodiment, the second antigen binding moiety is capable of specific binding to MCSP and comprises a heavy chain variable region comprising an amino acid sequence selected from the group of SEQ ID NO: 279, SEQ ID NO: 300, SEQ ID NO: 302, SEQ ID NO: 305 and SEQ ID NO: 307 and a light chain variable region comprising an amino acid sequence selected from the group of SEQ ID NO: 283, SEQ ID NO: 309, SEQ ID NO: 312, SEQ ID NO: 313 and SEQ ID NO: 317.

In another specific embodiment, the second antigen binding moiety is capable of specific binding to MCSP and comprises a heavy chain variable region comprising the amino acid sequence of SEQ ID NO: 279 and a light chain variable region comprising the amino acid sequence of SEQ ID NO: 283.

In one embodiment the present invention provides a T cell activating bispecific antigen binding molecule comprising

(i) a first antigen binding moiety which is a Fab molecule capable of specific binding to CD3, comprising at least one heavy chain complementarity determining region (CDR) selected from the group consisting of SEQ ID NO: 270, SEQ ID NO: 271 and SEQ ID NO: 272 and at least one light chain CDR selected from the group of SEQ ID NO: 274, SEQ ID NO: 275, SEQ ID NO: 276;
(ii) a second antigen binding moiety which is a Fab molecule capable of specific binding to CEA comprising at least one heavy chain complementarity determining region (CDR) selected from the group consisting of SEQ ID NO: 290, SEQ ID NO: 291 and SEQ ID NO: 292 and at least one light chain CDR selected from the group of SEQ ID NO: 294, SEQ ID NO: 295 and SEQ ID NO: 296.

In one embodiment the present invention provides a T cell activating bispecific antigen binding molecule comprising

(i) a first antigen binding moiety which is a Fab molecule capable of specific binding to CD3 comprising a heavy chain variable region comprising the amino acid sequence of SEQ ID NO: 269 and a light chain variable region comprising the amino acid sequence of SEQ ID NO: 273.
(ii) a second antigen binding moiety which is a Fab molecule capable of specific binding CEA comprising a heavy chain variable region comprising the amino acid sequence of SEQ ID NO: 289 and a light chain variable region comprising the amino acid sequence of SEQ ID NO: 293.

In one embodiment the present invention provides a T cell activating bispecific antigen binding molecule comprising

(i) a first antigen binding moiety which is a Fab molecule capable of specific binding to CD3, comprising at least one heavy chain complementarity determining region (CDR) selected from the group consisting of SEQ ID NO: 270, SEQ ID NO: 271 and SEQ ID NO: 272 and at least one light chain CDR selected from the group of SEQ ID NO: 274, SEQ ID NO: 275, SEQ ID NO: 276;
(ii) a second antigen binding moiety which is a Fab molecule capable of specific binding MCSP comprising at least one heavy chain complementarity determining region (CDR) selected from the group consisting of SEQ ID NO: 280, SEQ ID NO: 281 and SEQ ID NO: 282 and at least one light chain CDR selected from the group of SEQ ID NO: 284, SEQ ID NO: 285 and SEQ ID NO: 286.

In one embodiment the present invention provides a T cell activating bispecific antigen binding molecule comprising

(i) a first antigen binding moiety which is a Fab molecule capable of specific binding to CD3 comprising a heavy chain variable region comprising the amino acid sequence of SEQ ID NO: 269 and a light chain variable region comprising the amino acid sequence of SEQ ID NO: 273.
(ii) a second antigen binding moiety which is a Fab molecule capable of specific binding MCSP comprising a heavy chain variable region comprising the amino acid sequence of SEQ ID NO: 279 and a light chain variable region comprising the amino acid sequence of SEQ ID NO: 283.

In a particular embodiment, the first antigen binding moiety is a crossover Fab molecule wherein either the variable or the constant regions of the Fab light chain and the Fab heavy chain are exchanged. In an even more particular embodiment, the first antigen binding moiety is a crossover Fab molecule wherein the constant regions of the Fab light chain and the Fab heavy chain are exchanged.

In one embodiment, the second antigen binding moiety is a conventional Fab molecule.

In a further particular embodiment, not more than one antigen binding moiety capable of specific binding to CD3 is present in the T cell activating bispecific antigen binding molecule (i.e. the T cell activating bispecific antigen binding molecule provides monovalent binding to CD3).

In one embodiment the present invention provides a T cell activating bispecific antigen binding molecule comprising

(i) a first antigen binding moiety which is a Fab molecule capable of specific binding to CD3, comprising the heavy chain complementarity determining region (CDR) 1 of SEQ ID NO: 270, the heavy chain CDR 2 of SEQ ID NO: 271, the heavy chain CDR 3 of SEQ ID NO: 272, the light chain CDR 1 of SEQ ID NO: 274, the light chain CDR 2 of SEQ ID NO: 275 and the light chain CDR 3 of SEQ ID NO: 276, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety each of which is a Fab molecule capable of specific binding to CEA comprising the heavy chain CDR 1 of SEQ ID NO: 290, the heavy chain CDR 2 of SEQ ID NO: 291, the heavy chain CDR 3 of SEQ ID NO: 292, the light chain CDR 1 of SEQ ID NO: 294, the light chain CDR 2 of SEQ ID NO: 295 and the light chain CDR3 of SEQ ID NO: 296; and
(iii) an IgG₄ Fc domain composed of a first and a second subunit capable of stable association.

In one embodiment the present invention provides a T cell activating bispecific antigen binding molecule comprising

(i) a first antigen binding moiety which is a Fab molecule capable of specific binding to CD3 comprising a heavy chain variable region comprising an amino acid sequence that is at least about 95%, 96%, 97%, 98%, 99% or 100% identical to the amino acid sequence of SEQ ID NO: 269 and a light chain variable region comprising an amino acid sequence that is at least about 95%, 96%, 97%, 98%, 99% or 100% identical to the amino acid sequence of SEQ ID NO: 273, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety each of which is a Fab molecule capable of specific binding to CEA comprising heavy chain variable region comprising an amino acid sequence that is at least about 95%, 96%, 97%, 98%, 99% or 100% identical to the amino acid sequence of SEQ ID NO: 289 and a light chain variable region comprising an amino acid sequence that is at least about 95%, 96%, 97%, 98%, 99% or 100% identical to the amino acid sequence of SEQ ID NO: 293; and
(iii) an IgG₄ Fc domain composed of a first and a second subunit capable of stable association.

In one embodiment the present invention provides a T cell activating bispecific antigen binding molecule comprising

(i) a first antigen binding moiety which is a Fab molecule capable of specific binding to CD3, comprising the heavy chain complementarity determining region (CDR) 1 of SEQ ID NO: 270, the heavy chain CDR 2 of SEQ ID NO: 271, the heavy chain CDR 3 of SEQ ID NO: 272, the light chain CDR 1 of SEQ ID NO: 274, the light chain CDR 2 of SEQ ID NO: 275 and the light chain CDR 3 of SEQ ID NO: 276, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety each of which is a Fab molecule capable of specific binding to MCSP comprising the heavy chain CDR 1 of SEQ ID NO: 280, the heavy chain CDR 2 of SEQ ID NO: 281, the heavy chain CDR 3 of SEQ ID NO: 282, the light chain CDR 1 of SEQ ID NO: 284, the light chain CDR 2 of SEQ ID NO: 285 and the light chain CDR3 of SEQ ID NO: 286; and
(iii) an IgG₄ Fc domain composed of a first and a second subunit capable of stable association.

In one embodiment the present invention provides a T cell activating bispecific antigen binding molecule comprising

(i) a first antigen binding moiety which is a Fab molecule capable of specific binding to CD3 comprising a heavy chain variable region comprising an amino acid sequence that is at least about 95%, 96%, 97%, 98%, 99% or 100% identical to the amino acid sequence of SEQ ID NO: 269 and a light chain variable region comprising an amino acid sequence that is at least about 95%, 96%, 97%, 98%, 99% or 100% identical to the amino acid sequence of SEQ ID NO: 273, wherein the first antigen binding moiety is a crossover Fab molecule wherein either the variable or the constant regions, particularly the constant regions, of the Fab light chain and the Fab heavy chain are exchanged;
(ii) a second and a third antigen binding moiety each of which is a Fab molecule capable of specific binding to MCSP comprising a heavy chain variable region comprising an amino acid sequence that is at least about 95%, 96%, 97%, 98%, 99% or 100% identical to the amino acid sequence of SEQ ID NO: 279 and a light chain variable region comprising an amino acid sequence that is at least about 95%, 96%, 97%, 98%, 99% or 100% identical to the amino acid sequence of SEQ ID NO: 283; and
(iii) an IgG₄ Fc domain composed of a first and a second subunit capable of stable association.

In the T cell activating bispecific antigen binding molecule according to any of the four above embodiments preferably the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the IgG₄ Fc domain, and the third antigen binding moiety is fused at the C-terminus of the Fab heavy chain to the N-terminus of the second subunit of the IgG₄ Fc domain.

### T Cell Activating Bispecific Antigen Binding Molecule Formats

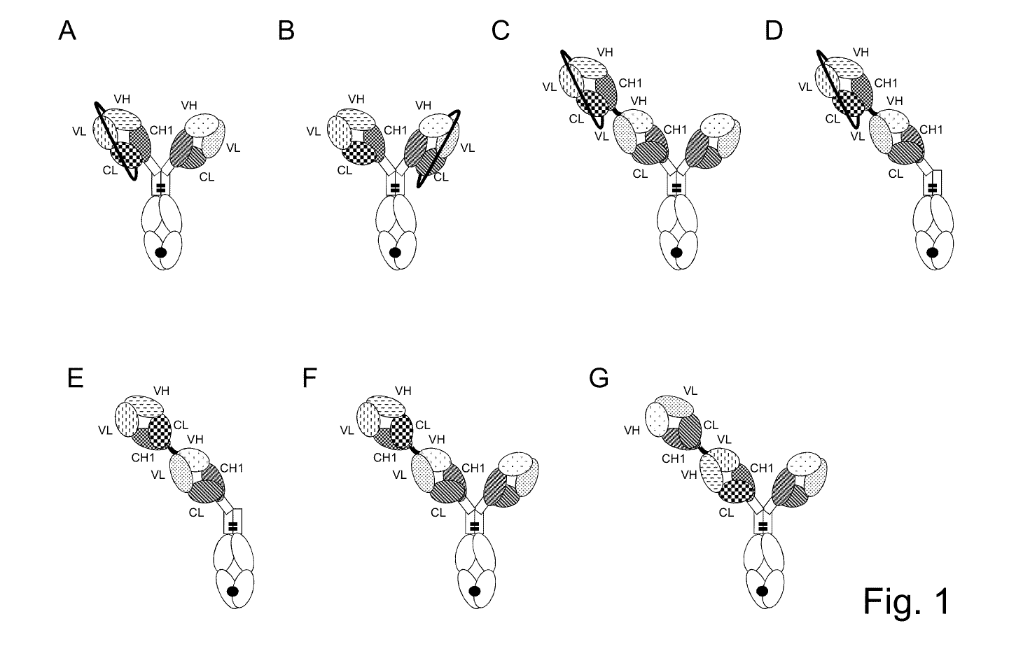

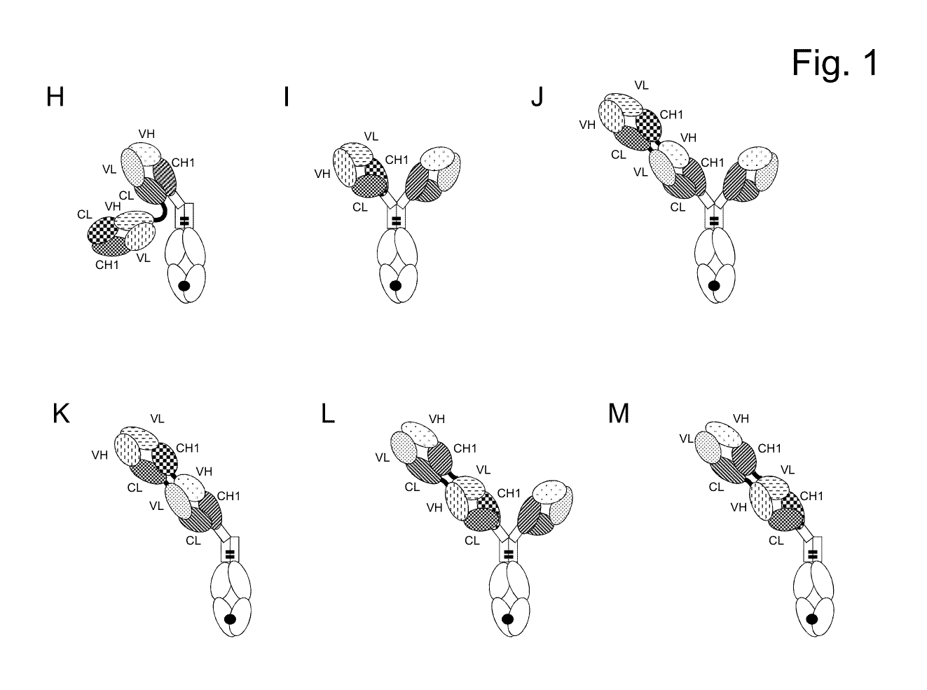

The components of the T cell activating bispecific antigen binding molecule can be fused to each other in a variety of configurations. Exemplary configurations are depicted in FIG. 1.

In some embodiments, the second antigen binding moiety is fused at the C-terminus of the Fab heavy chain to the N-terminus of the first or the second subunit of the Fc domain.

In a particular such embodiment, the first antigen binding moiety is fused at the C-terminus of the Fab heavy chain to the N-terminus of the Fab heavy chain of the second antigen binding moiety. In a specific such embodiment, the T cell activating bispecific antigen binding molecule essentially consists of a first and a second antigen binding moiety, an Fc domain composed of a first and a second subunit, and optionally one or more peptide linkers, wherein the first antigen binding moiety is fused at the C-terminus of the Fab heavy chain to the N-terminus of the Fab heavy chain of the second antigen binding moiety, and the second antigen binding moiety is fused at the C-terminus of the Fab heavy chain to the N-terminus of the first or the second subunit of the Fc domain. In an even more specific embodiment, the first antigen binding moiety is a single chain Fab molecule. Alternatively, in a particular embodiment, the first antigen binding moiety is a crossover Fab molecule. Optionally, if the first antigen binding moiety is a crossover Fab molecule, the Fab light chain of the first antigen binding moiety and the Fab light chain of the second antigen binding moiety may additionally be fused to each other.

In an alternative such embodiment, the first antigen binding moiety is fused at the C-terminus of the Fab heavy chain to the N-terminus of the first or second subunit of the Fc domain. In a specific such embodiment, the T cell activating bispecific antigen binding molecule essentially consists of a first and a second antigen binding moiety, an Fc domain composed of a first and a second subunit, and optionally one or more peptide linkers, wherein the first and the second antigen binding moiety are each fused at the C-terminus of the Fab heavy chain to the N-terminus of one of the subunits of the Fc domain. In an even more specific embodiment, the first antigen binding moiety is a single chain Fab molecule. Alternatively, in a particular embodiment, the first antigen binding moiety is a crossover Fab molecule.

In yet another such embodiment, the second antigen binding moiety is fused at the C-terminus of the Fab light chain to the N-terminus of the Fab light chain of the first antigen binding moiety. In a specific such embodiment, the T cell activating bispecific antigen binding molecule essentially consists of a first and a second antigen binding moiety, an Fc domain composed of a first and a second subunit, and optionally one or more peptide linkers, wherein the first antigen binding moiety is fused at the N-terminus of the Fab light chain to the C-terminus of the Fab light chain of the second antigen binding moiety, and the second antigen binding moiety is fused at the C-terminus of the Fab heavy chain to the N-terminus of the first or the second subunit of the Fc domain. In an even more specific embodiment, the first antigen binding moiety is a crossover Fab molecule.

In other embodiments, the first antigen binding moiety is fused at the C-terminus of the Fab heavy chain to the N-terminus of the first or second subunit of the Fc domain.

In a particular such embodiment, the second antigen binding moiety is fused at the C-terminus of the Fab heavy chain to the N-terminus of the Fab heavy chain of the first antigen binding moiety. In a specific such embodiment, the T cell activating bispecific antigen binding molecule essentially consists of a first and a second antigen binding moiety, an Fc domain composed of a first and a second subunit, and optionally one or more peptide linkers, wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first or the second subunit of the Fc domain. In an even more specific embodiment, the first antigen binding moiety is a crossover Fab molecule. Optionally, the Fab light chain of the first antigen binding moiety and the Fab light chain of the second antigen binding moiety may additionally be fused to each other.

In particular of these embodiments, the first antigen binding moiety is capable of specific binding to an activating T cell antigen. In other embodiments, the first antigen binding moiety is capable of specific binding to a target cell antigen.

The antigen binding moieties may be fused to the Fc domain or to each other directly or through a peptide linker, comprising one or more amino acids, typically about 2-20 amino acids. Peptide linkers are known in the art and are described herein. Suitable, non-immunogenic peptide linkers include, for example, (G₄S)ₙ, (SG₄)ₙ, (G₄S)ₙ or G₄(SG₄)ₙ peptide linkers. “n” is generally a number between 1 and 10, typically between 2 and 4. A particularly suitable peptide linker for fusing the Fab light chains of the first and the second antigen binding moiety to each other is (G₄S)₂. An exemplary peptide linker suitable for connecting the Fab heavy chains of the first and the second antigen binding moiety is EPKSC(D)-(G₄S)₂ (SEQ ID NOs 150 and 151). Additionally, linkers may comprise (a portion of) an immunoglobulin hinge region. Particularly where an antigen binding moiety is fused to the N-terminus of an Fc domain subunit, it may be fused via an immunoglobulin hinge region or a portion thereof, with or without an additional peptide linker.

A T cell activating bispecific antigen binding molecule with a single antigen binding moiety capable of specific binding to a target cell antigen (for example as shown in FIG. 1A, 1B, 1D, 1E, 1H, 1I, 1K or 1M) is useful, particularly in cases where internalization of the target cell antigen is to be expected following binding of a high affinity antigen binding moiety. In such cases, the presence of more than one antigen binding moiety specific for the target cell antigen may enhance internalization of the target cell antigen, thereby reducing its availability.

In many other cases, however, it will be advantageous to have a T cell activating bispecific antigen binding molecule comprising two or more antigen binding moieties specific for a target cell antigen (see examples in shown in FIG. 1C, 1F, 1G, 1J or 1L), for example to optimize targeting to the target site or to allow crosslinking of target cell antigens.

Accordingly, in certain embodiments, the T cell activating bispecific antigen binding molecule of the invention further comprises a third antigen binding moiety which is a Fab molecule capable of specific binding to a target cell antigen. In one embodiment, the third antigen binding moiety is capable of specific binding to the same target cell antigen as the first or second antigen binding moiety. In a particular embodiment, the first antigen binding moiety is capable of specific binding to an activating T cell antigen, and the second and third antigen binding moieties are capable of specific binding to a target cell antigen.

In one embodiment, the third antigen binding moiety is fused at the C-terminus of the Fab heavy chain to the N-terminus of the first or second subunit of the Fc domain. In a particular embodiment, the second and the third antigen binding moiety are each fused at the C-terminus of the Fab heavy chain to the N-terminus of one of the subunits of the Fc domain, and the first antigen binding moiety is fused at the C-terminus of the Fab heavy chain to the N-terminus of the Fab heavy chain of the second antigen binding moiety. In one such embodiment the first antigen binding moiety is a single chain Fab molecule. In a particular such embodiment the first antigen binding moiety is a crossover Fab molecule. Optionally, if the first antigen binding moiety is a crossover Fab molecule, the Fab light chain of the first antigen binding moiety and the Fab light chain of the second antigen binding moiety may additionally be fused to each other.

The second and the third antigen binding moiety may be fused to the Fc domain directly or through a peptide linker. In a particular embodiment the second and the third antigen binding moiety are each fused to the Fc domain through an immunoglobulin hinge region. In a specific embodiment, the immunoglobulin hinge region is a human IgG₁ hinge region. In one embodiment the second and the third antigen binding moiety and the Fc domain are part of an immunoglobulin molecule. In a particular embodiment the immunoglobulin molecule is an IgG class immunoglobulin. In an even more particular embodiment the immunoglobulin is an IgG₁ subclass immunoglobulin. In another embodiment the immunoglobulin is an IgG₄ subclass immunoglobulin. In a further particular embodiment the immunoglobulin is a human immunoglobulin. In other embodiments the immunoglobulin is a chimeric immunoglobulin or a humanized immunoglobulin. In one embodiment, the T cell activating bispecific antigen binding molecule essentially consists of an immunoglobulin molecule capable of specific binding to a target cell antigen, and an antigen binding moiety capable of specific binding to an activating T cell antigen wherein the antigen binding moiety is a single chain Fab molecule or a crossover Fab molecule, particularly a crossover Fab molecule, fused to the N-terminus of one of the immunoglobulin heavy chains, optionally via a peptide linker.

In an alternative embodiment, the first and the third antigen binding moiety are each fused at the C-terminus of the Fab heavy chain to the N-terminus of one of the subunits of the Fc domain, and the second antigen binding moiety is fused at the C-terminus of the Fab heavy chain to the N-terminus of the Fab heavy chain of the first antigen binding moiety. In a specific such embodiment, the T cell activating bispecific antigen binding molecule essentially consists of a first, a second and a third antigen binding moiety, an Fc domain composed of a first and a second subunit, and optionally one or more peptide linkers, wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain. In a particular such embodiment the first antigen binding moiety is a crossover Fab molecule. Optionally, the Fab light chain of the first antigen binding moiety and the Fab light chain of the second antigen binding moiety may additionally be fused to each other.

In some of the T cell activating bispecific antigen binding molecule of the invention, the Fab light chain of the first antigen binding moiety and the Fab light chain of the second antigen binding moiety are fused to each other, optionally via a linker peptide. Depending on the configuration of the first and the second antigen binding moiety, the Fab light chain of the first antigen binding moiety may be fused at its C-terminus to the N-terminus of the Fab light chain of the second antigen binding moiety, or the Fab light chain of the second antigen binding moiety may be fused at its C-terminus to the N-terminus of the Fab light chain of the first antigen binding moiety. Fusion of the Fab light chains of the first and the second antigen binding moiety further reduces mispairing of unmatched Fab heavy and light chains, and also reduces the number of plasmids needed for expression of some of the T cell activating bispecific antigen binding molecules of the invention.

In certain embodiments the T cell activating bispecific antigen binding molecule comprises a polypeptide wherein a first Fab light chain shares a carboxy-terminal peptide bond with a peptide linker, which in turn shares a carboxy-terminal peptide bond with a first Fab heavy chain, which in turn shares a carboxy-terminal peptide bond with an Fc domain subunit (VL-CL-linker-VH-CH1-CH2-CH2(—CH4)), and a polypeptide wherein a second Fab heavy chain shares a carboxy-terminal peptide bond with an Fc domain subunit (VH-CH1-CH2-CH3(—CH4)). In some embodiments the T cell activating bispecific antigen binding molecule further comprises a second Fab light chain polypeptide (VL-CL). In certain embodiments the polypeptides are covalently linked, e.g., by a disulfide bond.

In some embodiments, the T cell activating bispecific antigen binding molecule comprises a polypeptide wherein a first Fab light chain shares a carboxy-terminal peptide bond with a peptide linker, which in turn shares a carboxy-terminal peptide bond with a first Fab heavy chain, which in turn shares a carboxy-terminal peptide bond with a second Fab heavy chain, which in turn shares a carboxy-terminal peptide bond with an Fc domain subunit (VL-CL-linker-VH-CH1-VH-CH1-CH2-CH3(—CH4)). In one of these embodiments that T cell activating bispecific antigen binding molecule further comprises a second Fab light chain polypeptide (VL-CL). The T cell activating bispecific antigen binding molecule according to these embodiments may further comprise (i) an Fc domain subunit polypeptide (CH2-CH3(—CH4)), or (ii) a polypeptide wherein a third Fab heavy chain shares a carboxy-terminal peptide bond with an Fc domain subunit (VH-CH1-CH2-CH3(—CH4)) and a third Fab light chain polypeptide (VL-CL). In certain embodiments the polypeptides are covalently linked, e.g., by a disulfide bond.

In certain embodiments the T cell activating bispecific antigen binding molecule comprises a polypeptide wherein a first Fab light chain variable region shares a carboxy-terminal peptide bond with a first Fab heavy chain constant region (i.e. a crossover Fab heavy chain, wherein the heavy chain variable region is replaced by a light chain variable region), which in turn shares a carboxy-terminal peptide bond with an Fc domain subunit (VL-CH1-CH2-CH3(—CH4)), and a polypeptide wherein a second Fab heavy chain shares a carboxy-terminal peptide bond with an Fc domain subunit (VH-CH1-CH2-CH3(—CH4)). In some embodiments the T cell activating bispecific antigen binding molecule further comprises a polypeptide wherein a Fab heavy chain variable region shares a carboxy-terminal peptide bond with a Fab light chain constant region (VH-CL) and a Fab light chain polypeptide (VL-CL). In certain embodiments the polypeptides are covalently linked, e.g., by a disulfide bond.

In alternative embodiments the T cell activating bispecific antigen binding molecule comprises a polypeptide wherein a first Fab heavy chain variable region shares a carboxy-terminal peptide bond with a first Fab light chain constant region (i.e. a crossover Fab heavy chain, wherein the heavy chain constant region is replaced by a light chain constant region), which in turn shares a carboxy-terminal peptide bond with an Fc domain subunit (VH-CL-CH2-CH3(—CH4)), and a polypeptide wherein a second Fab heavy chain shares a carboxy-terminal peptide bond with an Fc domain subunit (VH-CH1-CH2-CH3(—CH4)). In some embodiments the T cell activating bispecific antigen binding molecule further comprises a polypeptide wherein a Fab light chain variable region shares a carboxy-terminal peptide bond with a Fab heavy chain constant region (VL-CH1) and a Fab light chain polypeptide (VL-CL). In certain embodiments the polypeptides are covalently linked, e.g., by a disulfide bond.

In some embodiments, the T cell activating bispecific antigen binding molecule comprises a polypeptide wherein a first Fab light chain variable region shares a carboxy-terminal peptide bond with a first Fab heavy chain constant region (i.e. a crossover Fab heavy chain, wherein the heavy chain variable region is replaced by a light chain variable region), which in turn shares a carboxy-terminal peptide bond with a second Fab heavy chain, which in turn shares a carboxy-terminal peptide bond with an Fc domain subunit (VL-CH1-VH-CH1-CH2-CH3(—CH4)). In other embodiments, the T cell activating bispecific antigen binding molecule comprises a polypeptide wherein a first Fab heavy chain variable region shares a carboxy-terminal peptide bond with a first Fab light chain constant region (i.e. a crossover Fab heavy chain, wherein the heavy chain constant region is replaced by a light chain constant region), which in turn shares a carboxy-terminal peptide bond with a second Fab heavy chain, which in turn shares a carboxy-terminal peptide bond with an Fc domain subunit (VH-CL-VH-CH1-CH2-CH3(—CH4)). In still other embodiments, the T cell activating bispecific antigen binding molecule comprises a polypeptide wherein a second Fab heavy chain shares a carboxy-terminal peptide bond with a first Fab light chain variable region which in turn shares a carboxy-terminal peptide bond with a first Fab heavy chain constant region (i.e. a crossover Fab heavy chain, wherein the heavy chain variable region is replaced by a light chain variable region), which in turn shares a carboxy-terminal peptide bond with an Fc domain subunit (VH-CH1-VL-CH1-CH2-CH3(—CH4)). In other embodiments, the T cell activating bispecific antigen binding molecule comprises a polypeptide wherein a second Fab heavy chain shares a carboxy-terminal peptide bond with a first Fab heavy chain variable region which in turn shares a carboxy-terminal peptide bond with a first Fab light chain constant region (i.e. a crossover Fab heavy chain, wherein the heavy chain constant region is replaced by a light chain constant region), which in turn shares a carboxy-terminal peptide bond with an Fc domain subunit (VH-CH1-VH-CL-CH2-CH3(—CH4)).

In some of these embodiments the T cell activating bispecific antigen binding molecule further comprises a crossover Fab light chain polypeptide, wherein a Fab heavy chain variable region shares a carboxy-terminal peptide bond with a Fab light chain constant region (VH-CL), and a Fab light chain polypeptide (VL-CL). In others of these embodiments the T cell activating bispecific antigen binding molecule further comprises a crossover Fab light chain polypeptide, wherein a Fab light chain variable region shares a carboxy-terminal peptide bond with a Fab heavy chain constant region (VL-CH1), and a Fab light chain polypeptide (VL-CL). In still others of these embodiments the T cell activating bispecific antigen binding molecule further comprises a polypeptide wherein a Fab light chain variable region shares a carboxy-terminal peptide bond with a Fab heavy chain constant region which in turn shares a carboxy-terminal peptide bond with a Fab light chain polypeptide (VL-CH1-VL-CL), a polypeptide wherein a Fab heavy chain variable region shares a carboxy-terminal peptide bond with a Fab light chain constant region which in turn shares a carboxy-terminal peptide bond with a Fab light chain polypeptide (VH-CL-VL-CL), a polypeptide wherein a Fab light chain polypeptide shares a carboxy-terminal peptide bond with a Fab light chain variable region which in turn shares a carboxy-terminal peptide bond with a Fab heavy chain constant region (VL-CL-VL-CH1), or a polypeptide wherein a Fab light chain polypeptide shares a carboxy-terminal peptide bond with a Fab heavy chain variable region which in turn shares a carboxy-terminal peptide bond with a Fab light chain constant region (VL-CL-VH-CL).

The T cell activating bispecific antigen binding molecule according to these embodiments may further comprise (i) an Fc domain subunit polypeptide (CH2-CH3(—CH4)), or (ii) a polypeptide wherein a third Fab heavy chain shares a carboxy-terminal peptide bond with an Fc domain subunit (VH-CH1-CH2-CH3(—CH4)) and a third Fab light chain polypeptide (VL-CL). In certain embodiments the polypeptides are covalently linked, e.g., by a disulfide bond.

In one embodiment, the T cell activating bispecific antigen binding molecule comprises a polypeptide wherein a second Fab light chain shares a carboxy-terminal peptide bond with a first Fab light chain variable region which in turn shares a carboxy-terminal peptide bond with a first Fab heavy chain constant region (i.e. a crossover Fab light chain, wherein the light chain constant region is replaced by a heavy chain constant region) (VL-CL-VL-CH1), a polypeptide wherein a second Fab heavy chain shares a carboxy-terminal peptide bond with an Fc domain subunit (VH-CH1-CH2-CH3(—CH4)), and a polypeptide wherein a first Fab heavy chain variable region shares a carboxy-terminal peptide bond with a first Fab light chain constant region (VH-CL). In another embodiment, the T cell activating bispecific antigen binding molecule comprises a polypeptide wherein a second Fab light chain shares a carboxy-terminal peptide bond with a first Fab heavy chain variable region which in turn shares a carboxy-terminal peptide bond with a first Fab light chain constant region (i.e. a crossover Fab light chain, wherein the light chain variable region is replaced by a heavy chain variable region) (VL-CL-VH-CL), a polypeptide wherein a second Fab heavy chain shares a carboxy-terminal peptide bond with an Fc domain subunit (VH-CH1-CH2-CH3(—CH4)), and a polypeptide wherein a first Fab light chain variable region shares a carboxy-terminal peptide bond with a first Fab heavy chain constant region (VL-CH1). The T cell activating bispecific antigen binding molecule according to these embodiments may further comprise (i) an Fc domain subunit polypeptide (CH2-CH3(—CH4)), or (ii) a polypeptide wherein a third Fab heavy chain shares a carboxy-terminal peptide bond with an Fc domain subunit (VH-CH1-CH2-CH3(—CH4)) and a third Fab light chain polypeptide (VL-CL). In certain embodiments the polypeptides are covalently linked, e.g., by a disulfide bond.

According to any of the above embodiments, components of the T cell activating bispecific antigen binding molecule (e.g. antigen binding moiety, Fc domain) may be fused directly or through various linkers, particularly peptide linkers comprising one or more amino acids, typically about 2-20 amino acids, that are described herein or are known in the art. Suitable, non-immunogenic peptide linkers include, for example, (G₄S)ₙ, (SG₄)ₙ, (G₄S)ₙ or G₄(SG₄)ₙ peptide linkers, wherein n is generally a number between 1 and 10, typically between 2 and 4.

### Fc Domain

The Fc domain of the T cell activating bispecific antigen binding molecule consists of a pair of polypeptide chains comprising heavy chain domains of an immunoglobulin molecule. For example, the Fc domain of an immunoglobulin G (IgG) molecule is a dimer, each subunit of which comprises the CH2 and CH3 IgG heavy chain constant domains. The two subunits of the Fc domain are capable of stable association with each other. In one embodiment the T cell activating bispecific antigen binding molecule of the invention comprises not more than one Fc domain.

In one embodiment according the invention the Fc domain of the T cell activating bispecific antigen binding molecule is an IgG Fc domain. In a particular embodiment the Fc domain is an IgG₁ Fc domain. An exemplary sequence of a human IgG₁ Fc region is given in SEQ ID NO: 149.

In another embodiment the Fc domain is an IgG₄ Fc domain. In one embodiment, the Fc domain is an IgG₄ Fc domain comprising an amino acid substitution at position 5228 (Kabat numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further particular embodiment the Fc domain is human. In another embodiment, the Fc domain is an IgG₄ Fc domain comprising an amino acid substitution at position L235 (Kabat numbering), particularly the amino acid substitution L235E. In another embodiment, the Fc domain is an IgG₄ Fc domain comprising both the amino acid substitution S228P and L235E (SPLE). In another embodiment, the Fc domain is an IgG₄ Fc domain comprising the amino acid substitution P329G (Kabat numbering). In another embodiment, the Fc domain is an IgG₄ Fc domain comprising the amino acid substitutions S228P, L235E and P329G.

### Fc Domain Modifications Promoting Heterodimerization

T cell activating bispecific antigen binding molecules according to the invention comprise different antigen binding moieties, fused to one or the other of the two subunits of the Fc domain, thus the two subunits of the Fc domain are typically comprised in two non-identical polypeptide chains. Recombinant co-expression of these polypeptides and subsequent dimerization leads to several possible combinations of the two polypeptides. To improve the yield and purity of T cell activating bispecific antigen binding molecules in recombinant production, it will thus be advantageous to introduce in the Fc domain of the T cell activating bispecific antigen binding molecule a modification promoting the association of the desired polypeptides.

Accordingly, in particular embodiments the Fc domain of the T cell activating bispecific antigen binding molecule according to the invention comprises a modification promoting the association of the first and the second subunit of the Fc domain. The site of most extensive protein-protein interaction between the two subunits of a human IgG Fc domain is in the CH3 domain of the Fc domain. Thus, in one embodiment said modification is in the CH3 domain of the Fc domain.

In a specific embodiment said modification is a so-called “knob-into-hole” modification, comprising a “knob” modification in one of the two subunits of the Fc domain and a “hole” modification in the other one of the two subunits of the Fc domain.

The knob-into-hole technology is described e.g. in U.S. Pat. No. 5,731,168; U.S. Pat. No. 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in a particular embodiment, in the CH3 domain of the first subunit of the Fc domain of the T cell activating bispecific antigen binding molecule an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

The protuberance and cavity can be made by altering the nucleic acid encoding the polypeptides, e.g. by site-specific mutagenesis, or by peptide synthesis.

In a specific embodiment, in the CH3 domain of the first subunit of the Fc domain the threonine residue at position 366 is replaced with a tryptophan residue (T366W), and in the CH3 domain of the second subunit of the Fc domain the tyrosine residue at position 407 is replaced with a valine residue (Y407V). In one embodiment, in the second subunit of the Fc domain additionally the threonine residue at position 366 is replaced with a serine residue (T366S) and the leucine residue at position 368 is replaced with an alanine residue (L368A).

In yet a further embodiment, in the first subunit of the Fc domain additionally the serine residue at position 354 is replaced with a cysteine residue (S354C), and in the second subunit of the Fc domain additionally the tyrosine residue at position 349 is replaced by a cysteine residue (Y349C). Introduction of these two cysteine residues results in formation of a disulfide bridge between the two subunits of the Fc domain, further stabilizing the dimer (Carter, J Immunol Methods 248, 7-15 (2001)).

In a particular embodiment the antigen binding moiety capable of binding to an activating T cell antigen is fused (optionally via the antigen binding moiety capable of binding to a target cell antigen) to the first subunit of the Fc domain (comprising the “knob” modification). Without wishing to be bound by theory, fusion of the antigen binding moiety capable of binding to an activating T cell antigen to the knob-containing subunit of the Fc domain will (further) minimize the generation of antigen binding molecules comprising two antigen binding moieties capable of binding to an activating T cell antigen (steric clash of two knob-containing polypeptides).

In an alternative embodiment a modification promoting association of the first and the second subunit of the Fc domain comprises a modification mediating electrostatic steering effects, e.g. as described in PCT publication WO 2009/089004. Generally, this method involves replacement of one or more amino acid residues at the interface of the two Fc domain subunits by charged amino acid residues so that homodimer formation becomes electrostatically unfavorable but heterodimerization electrostatically favorable.

Fc Domain Modifications Reducing Fc Receptor Binding and/or Effector Function

The Fc domain confers to the T cell activating bispecific antigen binding molecule favorable pharmacokinetic properties, including a long serum half-life which contributes to good accumulation in the target tissue and a favorable tissue-blood distribution ratio. At the same time it may, however, lead to undesirable targeting of the T cell activating bispecific antigen binding molecule to cells expressing Fc receptors rather than to the preferred antigen-bearing cells. Moreover, the co-activation of Fc receptor signaling pathways may lead to cytokine release which, in combination with the T cell activating properties and the long half-life of the antigen binding molecule, results in excessive activation of cytokine receptors and severe side effects upon systemic administration. Activation of (Fc receptor-bearing) immune cells other than T cells may even reduce efficacy of the T cell activating bispecific antigen binding molecule due to the potential destruction of T cells e.g. by NK cells.

Accordingly, in particular embodiments the Fc domain of the T cell activating bispecific antigen binding molecules according to the invention exhibits reduced binding affinity to an Fc receptor and/or reduced effector function, as compared to a native IgG₁ Fc domain. In one such embodiment the Fc domain (or the T cell activating bispecific antigen binding molecule comprising said Fc domain) exhibits less than 50%, preferably less than 20%, more preferably less than 10% and most preferably less than 5% of the binding affinity to an Fc receptor, as compared to a native IgG₁ Fc domain (or a T cell activating bispecific antigen binding molecule comprising a native IgG₁ Fc domain), and/or less than 50%, preferably less than 20%, more preferably less than 10% and most preferably less than 5% of the effector function, as compared to a native IgG₁ Fc domain (or a T cell activating bispecific antigen binding molecule comprising a native IgG₁ Fc domain). In one embodiment, the Fc domain (or the T cell activating bispecific antigen binding molecule comprising said Fc domain) does not substantially bind to an Fc receptor and/or induce effector function. In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of CDC, ADCC, ADCP, and cytokine secretion. In a particular embodiment the effector function is ADCC. In one embodiment the Fc domain exhibits substantially similar binding affinity to neonatal Fc receptor (FcRn), as compared to a native IgG₁ Fc domain. Substantially similar binding to FcRn is achieved when the Fc domain (or the T cell activating bispecific antigen binding molecule comprising said Fc domain) exhibits greater than about 70%, particularly greater than about 80%, more particularly greater than about 90% of the binding affinity of a native IgG₁ Fc domain (or the T cell activating bispecific antigen binding molecule comprising a native IgG₁ Fc domain) to FcRn.

In certain embodiments the Fc domain is engineered to have reduced binding affinity to an Fc receptor and/or reduced effector function, as compared to a non-engineered Fc domain. In particular embodiments, the Fc domain of the T cell activating bispecific antigen binding molecule comprises one or more amino acid mutation that reduces the binding affinity of the Fc domain to an Fc receptor and/or effector function. Typically, the same one or more amino acid mutation is present in each of the two subunits of the Fc domain. In one embodiment the amino acid mutation reduces the binding affinity of the Fc domain to an Fc receptor. In one embodiment the amino acid mutation reduces the binding affinity of the Fc domain to an Fc receptor by at least 2-fold, at least 5-fold, or at least 10-fold. In embodiments where there is more than one amino acid mutation that reduces the binding affinity of the Fc domain to the Fc receptor, the combination of these amino acid mutations may reduce the binding affinity of the Fc domain to an Fc receptor by at least 10-fold, at least 20-fold, or even at least 50-fold. In one embodiment the T cell activating bispecific antigen binding molecule comprising an engineered Fc domain exhibits less than 20%, particularly less than 10%, more particularly less than 5% of the binding affinity to an Fc receptor as compared to a T cell activating bispecific antigen binding molecule comprising a non-engineered Fc domain. In a particular embodiment the Fc receptor is an Fcγ receptor. In some embodiments the Fc receptor is a human Fc receptor. In some embodiments the Fc receptor is an activating Fc receptor. In a specific embodiment the Fc receptor is an activating human Fcγ receptor, more specifically human FcγRIIIa, FcγRI or FcγRIIa, most specifically human FcγRIIIa. Preferably, binding to each of these receptors is reduced. In some embodiments binding affinity to a complement component, specifically binding affinity to C1q, is also reduced. In one embodiment binding affinity to neonatal Fc receptor (FcRn) is not reduced. Substantially similar binding to FcRn, i.e. preservation of the binding affinity of the Fc domain to said receptor, is achieved when the Fc domain (or the T cell activating bispecific antigen binding molecule comprising said Fc domain) exhibits greater than about 70% of the binding affinity of a non-engineered form of the Fc domain (or the T cell activating bispecific antigen binding molecule comprising said non-engineered form of the Fc domain) to FcRn. The Fc domain, or T cell activating bispecific antigen binding molecules of the invention comprising said Fc domain, may exhibit greater than about 80% and even greater than about 90% of such affinity. In certain embodiments the Fc domain of the T cell activating bispecific antigen binding molecule is engineered to have reduced effector function, as compared to a non-engineered Fc domain. The reduced effector function can include, but is not limited to, one or more of the following: reduced complement dependent cytotoxicity (CDC), reduced antibody-dependent cell-mediated cytotoxicity (ADCC), reduced antibody-dependent cellular phagocytosis (ADCP), reduced cytokine secretion, reduced immune complex-mediated antigen uptake by antigen-presenting cells, reduced binding to NK cells, reduced binding to macrophages, reduced binding to monocytes, reduced binding to polymorphonuclear cells, reduced direct signaling inducing apoptosis, reduced crosslinking of target-bound antibodies, reduced dendritic cell maturation, or reduced T cell priming. In one embodiment the reduced effector function is one or more selected from the group of reduced CDC, reduced ADCC, reduced ADCP, and reduced cytokine secretion. In a particular embodiment the reduced effector function is reduced ADCC. In one embodiment the reduced ADCC is less than 20% of the ADCC induced by a non-engineered Fc domain (or a T cell activating bispecific antigen binding molecule comprising a non-engineered Fc domain).

In one embodiment the amino acid mutation that reduces the binding affinity of the Fc domain to an Fc receptor and/or effector function is an amino acid substitution. In one embodiment the Fc domain comprises an amino acid substitution at a position selected from the group of E233, L234, L235, N297, P331 and P329. In a more specific embodiment the Fc domain comprises an amino acid substitution at a position selected from the group of L234, L235 and P329. In some embodiments the Fc domain comprises the amino acid substitutions L234A and L235A. In one such embodiment, the Fc domain is an IgG₁ Fc domain, particularly a human IgG₁ Fc domain. In one embodiment the Fc domain comprises an amino acid substitution at position P329. In a more specific embodiment the amino acid substitution is P329A or P329G, particularly P329G. In one embodiment the Fc domain comprises an amino acid substitution at position P329 and a further amino acid substitution at a position selected from E233, L234, L235, N297 and P331. In a more specific embodiment the further amino acid substitution is E233P, L234A, L235A, L235E, N297A, N297D or P331 S. In particular embodiments the Fc domain comprises amino acid substitutions at positions P329, L234 and L235. In more particular embodiments the Fc domain comprises the amino acid mutations L234A, L235A and P329G (“P329G LALA”). In one such embodiment, the Fc domain is an IgG₁ Fc domain, particularly a human IgG₁ Fc domain. The “P329G LALA” combination of amino acid substitutions almost completely abolishes Fcγreceptor binding of a human IgG₁ Fc domain, as described in PCT publication no. WO 2012/130831, incorporated herein by reference in its entirety. WO 2012/130831 also describes methods of preparing such mutant Fc domains and methods for determining its properties such as Fc receptor binding or effector functions.

IgG₄ antibodies exhibit reduced binding affinity to Fc receptors and reduced effector functions as compared to IgG₁ antibodies. Hence, in some embodiments the Fc domain of the T cell activating bispecific antigen binding molecules of the invention is an IgG₄ Fc domain, particularly a human IgG₄ Fc domain. In one embodiment the IgG₄ Fc domain comprises amino acid substitutions at position 5228, specifically the amino acid substitution S228P. To further reduce its binding affinity to an Fc receptor and/or its effector function, in one embodiment the IgG₄ Fc domain comprises an amino acid substitution at position L235, specifically the amino acid substitution L235E. In another embodiment, the IgG₄ Fc domain comprises an amino acid substitution at position P329, specifically the amino acid substitution P329G. In a particular embodiment, the IgG₄ Fc domain comprises amino acid substitutions at positions S228, L235 and P329, specifically amino acid substitutions S228P, L235E and P329G. Such IgG₄ Fc domain mutants and their Fcγ receptor binding properties are described in PCT patent application no. WO 2012/130831, incorporated herein by reference in its entirety.

In a particular embodiment the Fc domain exhibiting reduced binding affinity to an Fc receptor and/or reduced effector function, as compared to a native IgG₁ Fc domain, is a human IgG₁ Fc domain comprising the amino acid substitutions L234A, L235A and optionally P329G, or a human IgG₄ Fc domain comprising the amino acid substitutions S228P, L235E and optionally P329G.

In certain embodiments N-glycosylation of the Fc domain has been eliminated. In one such embodiment the Fc domain comprises an amino acid mutation at position N297, particularly an amino acid substitution replacing asparagine by alanine (N297A) or aspartic acid (N297D).

In addition to the Fc domains described hereinabove and in PCT publication no. WO 2012/130831, Fc domains with reduced Fc receptor binding and/or effector function also include those with substitution of one or more of Fc domain residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Mutant Fc domains can be prepared by amino acid deletion, substitution, insertion or modification using genetic or chemical methods well known in the art. Genetic methods may include site-specific mutagenesis of the encoding DNA sequence, PCR, gene synthesis, and the like. The correct nucleotide changes can be verified for example by sequencing.

Binding to Fc receptors can be easily determined e.g. by ELISA, or by Surface Plasmon Resonance (SPR) using standard instrumentation such as a BIAcore instrument (GE Healthcare), and Fc receptors such as may be obtained by recombinant expression. A suitable such binding assay is described herein. Alternatively, binding affinity of Fc domains or cell activating bispecific antigen binding molecules comprising an Fc domain for Fc receptors may be evaluated using cell lines known to express particular Fc receptors, such as human NK cells expressing FcγIIIa receptor.

Effector function of an Fc domain, or a T cell activating bispecific antigen binding molecule comprising an Fc domain, can be measured by methods known in the art. A suitable assay for measuring ADCC is described herein. Other examples of in vitro assays to assess ADCC activity of a molecule of interest are described in U.S. Pat. No. 5,500,362; Hellstrom et al. Proc Natl Acad Sci USA 83, 7059-7063 (1986) and Hellstrom et al., Proc Natl Acad Sci USA 82, 1499-1502 (1985); U.S. Pat. No. 5,821,337; Bruggemann et al., J Exp Med 166, 1351-1361 (1987). Alternatively, non-radioactive assays methods may be employed (see, for example, ACTI™ non-radioactive cytotoxicity assay for flow cytometry (CellTechnology, Inc. Mountain View, Calif.); and CytoTox 96® non-radioactive cytotoxicity assay (Promega, Madison, Wis.)). Useful effector cells for such assays include peripheral blood mononuclear cells (PBMC) and Natural Killer (NK) cells. Alternatively, or additionally, ADCC activity of the molecule of interest may be assessed in vivo, e.g. in a animal model such as that disclosed in Clynes et al., Proc Natl Acad Sci USA 95, 652-656 (1998).

In some embodiments, binding of the Fc domain to a complement component, specifically to C1q, is reduced. Accordingly, in some embodiments wherein the Fc domain is engineered to have reduced effector function, said reduced effector function includes reduced CDC. C1q binding assays may be carried out to determine whether the T cell activating bispecific antigen binding molecule is able to bind C1q and hence has CDC activity. See e.g., C1q and C3c binding ELISA in WO 2006/029879 and WO 2005/100402. To assess complement activation, a CDC assay may be performed (see, for example, Gazzano-Santoro et al., J Immunol Methods 202, 163 (1996); Cragg et al., Blood 101, 1045-1052 (2003); and Cragg and Glennie, Blood 103, 2738-2743 (2004)).

### Antigen Binding Moieties

The antigen binding molecule of the invention is bispecific, i.e. it comprises at least two antigen binding moieties capable of specific binding to two distinct antigenic determinants. According to the invention, the antigen binding moieties are Fab molecules (i.e. antigen binding domains composed of a heavy and a light chain, each comprising a variable and a constant region). In one embodiment said Fab molecules are human. In another embodiment said Fab molecules are humanized. In yet another embodiment said Fab molecules comprise human heavy and light chain constant regions.

At least one of the antigen binding moieties is a single chain Fab molecule or a crossover Fab molecule. Such modifications prevent mispairing of heavy and light chains from different Fab molecules, thereby improving the yield and purity of the T cell activating bispecific antigen binding molecule of the invention in recombinant production. In a particular single chain Fab molecule useful for the T cell activating bispecific antigen binding molecule of the invention, the C-terminus of the Fab light chain is connected to the N-terminus of the Fab heavy chain by a peptide linker. The peptide linker allows arrangement of the Fab heavy and light chain to form a functional antigen binding moiety. Peptide linkers suitable for connecting the Fab heavy and light chain include, for example, (G₄S)₆-GG (SEQ ID NO: 152) or (SG₃)₂-(SEG₃)₄-(SG₃)-SG (SEQ ID NO: 153). In a particular crossover Fab molecule useful for the T cell activating bispecific antigen binding molecule of the invention, the constant regions of the Fab light chain and the Fab heavy chain are exchanged. In another crossover Fab molecule useful for the T cell activating bispecific antigen binding molecule of the invention, the variable regions of the Fab light chain and the Fab heavy chain are exchanged.

In a particular embodiment according to the invention, the T cell activating bispecific antigen binding molecule is capable of simultaneous binding to a target cell antigen, particularly a tumor cell antigen, and an activating T cell antigen. In one embodiment, the T cell activating bispecific antigen binding molecule is capable of crosslinking a T cell and a target cell by simultaneous binding to a target cell antigen and an activating T cell antigen. In an even more particular embodiment, such simultaneous binding results in lysis of the target cell, particularly a tumor cell. In one embodiment, such simultaneous binding results in activation of the T cell. In other embodiments, such simultaneous binding results in a cellular response of a T lymphocyte, particularly a cytotoxic T lymphocyte, selected from the group of: proliferation, differentiation, cytokine secretion, cytotoxic effector molecule release, cytotoxic activity, and expression of activation markers. In one embodiment, binding of the T cell activating bispecific antigen binding molecule to the activating T cell antigen without simultaneous binding to the target cell antigen does not result in T cell activation.

In one embodiment, the T cell activating bispecific antigen binding molecule is capable of re-directing cytotoxic activity of a T cell to a target cell. In a particular embodiment, said re-direction is independent of MHC-mediated peptide antigen presentation by the target cell and and/or specificity of the T cell.

Particularly, a T cell according to any of the embodiments of the invention is a cytotoxic T cell. In some embodiments the T cell is a CD4⁺ or a CD8⁺ T cell, particularly a CD8⁺ T cell.

### Activating T Cell Antigen Binding Moiety

The T cell activating bispecific antigen binding molecule of the invention comprises at least one antigen binding moiety capable of binding to an activating T cell antigen (also referred to herein as an “activating T cell antigen binding moiety”). In a particular embodiment, the T cell activating bispecific antigen binding molecule comprises not more than one antigen binding moiety capable of specific binding to an activating T cell antigen. In one embodiment the T cell activating bispecific antigen binding molecule provides monovalent binding to the activating T cell antigen. The activating T cell antigen binding moiety can either be a conventional Fab molecule or a modified Fab molecule, i.e. a single chain or crossover Fab molecule. In embodiments where there is more than one antigen binding moiety capable of specific binding to a target cell antigen comprised in the T cell activating bispecific antigen binding molecule, the antigen binding moiety capable of specific binding to an activating T cell antigen preferably is a modified Fab molecule.

In a particular embodiment the activating T cell antigen is CD3, particularly human CD3 (SEQ ID NO: 265) or cynomolgus CD3 (SEQ ID NO: 266), most particularly human CD3. In a particular embodiment the activating T cell antigen binding moiety is cross-reactive for (i.e. specifically binds to) human and cynomolgus CD3. In some embodiments, the activating T cell antigen is the epsilon subunit of CD3.

In one embodiment, the activating T cell antigen binding moiety can compete with monoclonal antibody H2C (described in PCT publication no. WO2008/119567) for binding an epitope of CD3. In another embodiment, the activating T cell antigen binding moiety can compete with monoclonal antibody V9 (described in Rodrigues et al., Int J Cancer Suppl 7, 45-50 (1992) and U.S. Pat. No. 6,054,297) for binding an epitope of CD3. In yet another embodiment, the activating T cell antigen binding moiety can compete with monoclonal antibody FN18 (described in Nooij et al., Eur J Immunol 19, 981-984 (1986)) for binding an epitope of CD3. In a particular embodiment, the activating T cell antigen binding moiety can compete with monoclonal antibody SP34 (described in Pessano et al., EMBO J 4, 337-340 (1985)) for binding an epitope of CD3. In one embodiment, the activating T cell antigen binding moiety binds to the same epitope of CD3 as monoclonal antibody SP34. In one embodiment, the activating T cell antigen binding moiety comprises the heavy chain CDR1 of SEQ ID NO: 163, the heavy chain CDR2 of SEQ ID NO: 165, the heavy chain CDR3 of SEQ ID NO: 167, the light chain CDR1 of SEQ ID NO: 171, the light chain CDR2 of SEQ ID NO: 173, and the light chain CDR3 of SEQ ID NO: 175. In a further embodiment, the activating T cell antigen binding moiety comprises a heavy chain variable region sequence that is at least about 80%, 85%, 90%, 95%, 96%, 97%, 98%, 99% or 100% identical to SEQ ID NO: 169 and a light chain variable region sequence that is at least about 80%, 85%, 90%, 95%, 96%, 97%, 98%, 99% or 100% identical to SEQ ID NO: 177, or variants thereof that retain functionality.

In a particular embodiment, the activating T cell antigen binding moiety comprises the heavy chain CDR1 of SEQ ID NO: 249, the heavy chain CDR2 of SEQ ID NO: 251, the heavy chain CDR3 of SEQ ID NO: 253, the light chain CDR1 of SEQ ID NO: 257, the light chain CDR2 of SEQ ID NO: 259, and the light chain CDR3 of SEQ ID NO: 261. In one embodiment, the activating T cell antigen binding moiety can compete for binding an epitope of CD3 with an antigen binding moiety comprising the heavy chain CDR1 of SEQ ID NO: 249, the heavy chain CDR2 of SEQ ID NO: 251, the heavy chain CDR3 of SEQ ID NO: 253, the light chain CDR1 of SEQ ID NO: 257, the light chain CDR2 of SEQ ID NO: 259, and the light chain CDR3 of SEQ ID NO: 261. In one embodiment, the activating T cell antigen binding moiety binds to the same epitope of CD3 as an antigen binding moiety comprising the heavy chain CDR1 of SEQ ID NO: 249, the heavy chain CDR2 of SEQ ID NO: 251, the heavy chain CDR3 of SEQ ID NO: 253, the light chain CDR1 of SEQ ID NO: 257, the light chain CDR2 of SEQ ID NO: 259, and the light chain CDR3 of SEQ ID NO: 261. In a further embodiment, the activating T cell antigen binding moiety comprises a heavy chain variable region sequence that is at least about 80%, 85%, 90%, 95%, 96%, 97%, 98%, 99% or 100% identical to SEQ ID NO: 255 and a light chain variable region sequence that is at least about 80%, 85%, 90%, 95%, 96%, 97%, 98%, 99% or 100% identical to SEQ ID NO: 263, or variants thereof that retain functionality. In one embodiment, the activating T cell antigen binding moiety can compete for binding an epitope of CD3 with an antigen binding moiety comprising the heavy chain variable region sequence of SEQ ID NO: 255 and the light chain variable region sequence of SEQ ID NO: 263. In one embodiment, the activating T cell antigen binding moiety binds to the same epitope of CD3 as an antigen binding moiety comprising the heavy chain variable region sequence of SEQ ID NO: 255 and the light chain variable region sequence of SEQ ID NO: 263. In another embodiment, the activating T cell antigen binding moiety comprises a humanized version of the heavy chain variable region sequence of SEQ ID NO: 255 and a humanized version of the light chain variable region sequence of SEQ ID NO: 263. In one embodiment, the activating T cell antigen binding moiety comprises the heavy chain CDR1 of SEQ ID NO: 249, the heavy chain CDR2 of SEQ ID NO: 251, the heavy chain CDR3 of SEQ ID NO: 253, the light chain CDR1 of SEQ ID NO: 257, the light chain CDR2 of SEQ ID NO: 259, the light chain CDR3 of SEQ ID NO: 261, and human heavy and light chain variable region framework sequences.

In one embodiment the CD3 antigen binding moiety comprises at least one heavy chain complementarity determining region (CDR) selected from the group consisting of SEQ ID NO: 270, SEQ ID NO: 271 and SEQ ID NO: 272 and at least one light chain CDR selected from the group of SEQ ID NO: 274, SEQ ID NO: 275 and SEQ ID NO: 276.

In one embodiment the CD3 antigen binding moiety comprises the heavy chain CDR1 of SEQ ID NO: 270, the heavy chain CDR2 of SEQ ID NO: 271, the heavy chain CDR3 of SEQ ID NO: 272, the light chain CDR1 of SEQ ID NO: 274, the light chain CDR2 of SEQ ID NO: 275, and the light chain CDR3 of SEQ ID NO: 276.

In one embodiment the CD3 antigen binding moiety comprises a heavy chain variable region sequence that is at least about 95%, 96%, 97%, 98%, 99% or 100% identical to an amino acid sequence selected from the group of: SEQ ID NO: 269, SEQ ID NO: 298 and SEQ ID NO: 299, and a light chain variable region sequence that is at least about 95%, 96%, 97%, 98%, 99% or 100% identical to an amino acid sequence selected from the group of: SEQ ID NO: 273 and SEQ ID NO: 297.

In one embodiment the CD3 antigen binding moiety comprises a heavy chain variable region comprising an amino acid sequence selected from the group of: SEQ ID NO: 269, SEQ ID NO: 298 and SEQ ID NO: 299 and a light chain variable region comprising an amino acid sequence selected from the group of: SEQ ID NO: 273 and SEQ ID NO: 297.

In one embodiment the CD3 antigen binding moiety comprises heavy chain variable region comprising the amino acid sequence of SEQ ID NO: 269 and a light chain variable region comprising the amino acid sequence of SEQ ID NO: 273.

In one embodiment the CD3 antigen binding moiety comprises the heavy chain variable region sequence of SEQ ID NO: 269 and the light chain variable region sequence of SEQ ID NO: 273.

In one embodiment the CD3 antigen binding moiety comprises a heavy chain variable region sequence that is at least about 95%, 96%, 97%, 98%, 99% or 100% identical to SEQ ID NO: 269 and a light chain variable region sequence that is at least about 95%, 96%, 97%, 98%, 99% or 100% identical to SEQ ID NO: 273.

### Target Cell Antigen Binding Moiety

The T cell activating bispecific antigen binding molecule of the invention comprises at least one antigen binding moiety capable of binding to a target cell antigen (also referred to herein as an “target cell antigen binding moiety”). In certain embodiments, the T cell activating bispecific antigen binding molecule comprises two antigen binding moieties capable of binding to a target cell antigen. In a particular such embodiment, each of these antigen binding moieties specifically binds to the same antigenic determinant. In one embodiment, the T cell activating bispecific antigen binding molecule comprises an immunoglobulin molecule capable of specific binding to a target cell antigen. In one embodiment the T cell activating bispecific antigen binding molecule comprises not more than two antigen binding moieties capable of binding to a target cell antigen.

The target cell antigen binding moiety is generally a Fab molecule that binds to a specific antigenic determinant and is able to direct the T cell activating bispecific antigen binding molecule to a target site, for example to a specific type of tumor cell that bears the antigenic determinant.

In certain embodiments the target cell antigen binding moiety is directed to an antigen associated with a pathological condition, such as an antigen presented on a tumor cell or on a virus-infected cell. Suitable antigens are cell surface antigens, for example, but not limited to, cell surface receptors. In particular embodiments the antigen is a human antigen. In a specific embodiment the target cell antigen is selected from the group of Fibroblast Activation Protein (FAP), Melanoma-associated Chondroitin Sulfate Proteoglycan (MCSP), Epidermal Growth Factor Receptor (EGFR), Carcinoembryonic Antigen (CEA), CD19, CD20 and CD33.

In particular embodiments the T cell activating bispecific antigen binding molecule comprises at least one antigen binding moiety that is specific for Melanoma-associated Chondroitin Sulfate Proteoglycan (MCSP). In one embodiment the T cell activating bispecific antigen binding molecule comprises at least one, typically two or more antigen binding moieties that can compete with monoclonal antibody LC007 (see SEQ ID NOs 75 and 83, and European patent application no. EP 11178393.2, incorporated herein by reference in its entirety) for binding to an epitope of MCSP. In one embodiment, the antigen binding moiety that is specific for MCSP comprises the heavy chain CDR1 of SEQ ID NO: 69, the heavy chain CDR2 of SEQ ID NO: 71, the heavy chain CDR3 of SEQ ID NO: 73, the light chain CDR1 of SEQ ID NO: 77, the light chain CDR2 of SEQ ID NO: 79, and the light chain CDR3 of SEQ ID NO: 81. In a further embodiment, the antigen binding moiety that is specific for MCSP comprises a heavy chain variable region sequence that is at least about 80%, 85%, 90%, 95%, 96%, 97%, 98%, 99% or 100% identical to SEQ ID NO: 75 and a light chain variable region sequence that is at least about 80%, 85%, 90%, 95%, 96%, 97%, 98%, 99% or 100% identical to SEQ ID NO: 83, or variants thereof that retain functionality. In particular embodiments the T cell activating bispecific antigen binding molecule comprises at least one, typically two or more antigen binding moieties that can compete with monoclonal antibody M4-3 ML2 (see SEQ ID NOs 239 and 247, and European patent application no. EP 11178393.2, incorporated herein by reference in its entirety) for binding to an epitope of MCSP. In one embodiment, the antigen binding moiety that is specific for MCSP binds to the same epitope of MCSP as monoclonal antibody M4-3 ML2. In one embodiment, the antigen binding moiety that is specific for MCSP comprises the heavy chain CDR1 of SEQ ID NO: 233, the heavy chain CDR2 of SEQ ID NO: 235, the heavy chain CDR3 of SEQ ID NO: 237, the light chain CDR1 of SEQ ID NO: 241, the light chain CDR2 of SEQ ID NO: 243, and the light chain CDR3 of SEQ ID NO: 245. In a further embodiment, the antigen binding moiety that is specific for MCSP comprises a heavy chain variable region sequence that is at least about 80%, 85%, 90%, 95%, 96%, 97%, 98%, 99% or 100%, particularly about 98%, 99% or 100%, identical to SEQ ID NO: 239 and a light chain variable region sequence that is at least about 80%, 85%, 90%, 95%, 96%, 97%, 98%, 99% or 100%, particularly about 98%, 99% or 100%, identical to SEQ ID NO: 247, or variants thereof that retain functionality. In one embodiment, the antigen binding moiety that is specific for MCSP comprises the heavy and light chain variable region sequences of an affinity matured version of monoclonal antibody M4-3 ML2 (SEQ ID NO: 239 and 247). In one embodiment, the antigen binding moiety that is specific for MCSP binds to MCSP with a K_{D} of ≦5×10⁻⁹M, ≦2×10⁻⁹M, ≦1×10⁻⁹M, ≦5×10⁻¹° M, ≦2×10⁻⁹M, ≦1×10⁻¹⁰ M, ≦5×10⁻¹¹ M, ≦1×10⁻¹¹ M, ≦5×10⁻¹² M, ≦1×10⁻¹² M, or less. In one embodiment, the antigen binding moiety that is specific for MCSP has an increased affinity of at least 1.5-fold, 2-fold, 2.5-fold, 3-fold, 4-fold, 5-fold, 10-fold, 20-fold or greater as compared to the anti-MCSP antibody M4-3/ML2.

In one embodiment, the antigen binding moiety that is specific for MCSP comprises the heavy chain variable region sequence of SEQ ID NO: 239 with one, two, three, four, five, six or seven, particularly two, three, four or five, amino acid substitutions; and the light chain variable region sequence of SEQ ID NO: 247 with one, two, three, four, five, six or seven, particularly two, three, four or five, amino acid substitutions. Any amino acid residue within the variable region sequences may be substituted by a different amino acid, including amino acid residues within the CDR regions, provided that binding to MCSP, particularly human MCSP, is preserved. Preferred variants are those having a binding affinity for MCSP at least equal (or stronger) to the binding affinity of the antigen binding moiety comprising the unsubstituted variable region sequences.

In one embodiment the T cell activating bispecific antigen binding molecule comprises the polypeptide sequence of SEQ ID NO: 1, the polypeptide sequence of SEQ ID NO: 3 and the polypeptide sequence of SEQ ID NO: 5, or variants thereof that retain functionality. In a further embodiment the T cell activating bispecific antigen binding molecule comprises the polypeptide sequence of SEQ ID NO: 7, the polypeptide sequence of SEQ ID NO: 9 and the polypeptide sequence of SEQ ID NO: 11, or variants thereof that retain functionality. In yet another embodiment the T cell activating bispecific antigen binding molecule comprises the polypeptide sequence of SEQ ID NO: 13, the polypeptide sequence of SEQ ID NO: 15 and the polypeptide sequence of SEQ ID NO: 5, or variants thereof that retain functionality. In yet another embodiment the T cell activating bispecific antigen binding molecule comprises the polypeptide sequence of SEQ ID NO: 17, the polypeptide sequence of SEQ ID NO: 19 and the polypeptide sequence of SEQ ID NO: 5, or variants thereof that retain functionality. In another embodiment the T cell activating bispecific antigen binding molecule comprises the polypeptide sequence of SEQ ID NO: 21, the polypeptide sequence of SEQ ID NO: 23 and the polypeptide sequence of SEQ ID NO: 5, or variants thereof that retain functionality. In still another embodiment the T cell activating bispecific antigen binding molecule comprises the polypeptide sequence of SEQ ID NO: 25, the polypeptide sequence of SEQ ID NO: 27 and the polypeptide sequence of SEQ ID NO: 5, or variants thereof that retain functionality. In another embodiment the T cell activating bispecific antigen binding molecule comprises the polypeptide sequence of SEQ ID NO: 29, the polypeptide sequence of SEQ ID NO: 31, the polypeptide sequence of SEQ ID NO: 33, and the polypeptide sequence of SEQ ID NO: 5, or variants thereof that retain functionality. In another embodiment the T cell activating bispecific antigen binding molecule comprises the polypeptide sequence of SEQ ID NO: 29, the polypeptide sequence of SEQ ID NO: 3, the polypeptide sequence of SEQ ID NO: 33, and the polypeptide sequence of SEQ ID NO: 5, or variants thereof that retain functionality. In another embodiment the T cell activating bispecific antigen binding molecule comprises the polypeptide sequence of SEQ ID NO: 35, the polypeptide sequence of SEQ ID NO: 3, the polypeptide sequence of SEQ ID NO: 37, and the polypeptide sequence of SEQ ID NO: 5, or variants thereof that retain functionality. In another embodiment the T cell activating bispecific antigen binding molecule comprises the polypeptide sequence of SEQ ID NO: 39, the polypeptide sequence of SEQ ID NO: 3, the polypeptide sequence of SEQ ID NO: 41, and the polypeptide sequence of SEQ ID NO: 5, or variants thereof that retain functionality. In yet another embodiment the T cell activating bispecific antigen binding molecule comprises the polypeptide sequence of SEQ ID NO: 29, the polypeptide sequence of SEQ ID NO: 3, the polypeptide sequence of SEQ ID NO: 5 and the polypeptide sequence of SEQ ID NO: 179, or variants thereof that retain functionality. In one embodiment the T cell activating bispecific antigen binding molecule comprises the polypeptide sequence of SEQ ID NO: 5, the polypeptide sequence of SEQ ID NO: 29, the polypeptide sequence of SEQ ID NO: 33 and the polypeptide sequence of SEQ ID NO: 181, or variants thereof that retain functionality. In one embodiment the T cell activating bispecific antigen binding molecule comprises the polypeptide sequence of SEQ ID NO: 5, the polypeptide sequence of SEQ ID NO: 23, the polypeptide sequence of SEQ ID NO: 183 and the polypeptide sequence of SEQ ID NO: 185, or variants thereof that retain functionality. In one embodiment the T cell activating bispecific antigen binding molecule comprises the polypeptide sequence of SEQ ID NO: 5, the polypeptide sequence of SEQ ID NO: 23, the polypeptide sequence of SEQ ID NO: 183 and the polypeptide sequence of SEQ ID NO: 187, or variants thereof that retain functionality. In one embodiment the T cell activating bispecific antigen binding molecule comprises the polypeptide sequence of SEQ ID NO: 33, the polypeptide sequence of SEQ ID NO: 189, the polypeptide sequence of SEQ ID NO: 191 and the polypeptide sequence of SEQ ID NO: 193, or variants thereof that retain functionality. In one embodiment the T cell activating bispecific antigen binding molecule comprises the polypeptide sequence of SEQ ID NO: 183, the polypeptide sequence of SEQ ID NO: 189, the polypeptide sequence of SEQ ID NO: 193 and the polypeptide sequence of SEQ ID NO: 195, or variants thereof that retain functionality. In one embodiment the T cell activating bispecific antigen binding molecule comprises the polypeptide sequence of SEQ ID NO: 189, the polypeptide sequence of SEQ ID NO: 193, the polypeptide sequence of SEQ ID NO: 199 and the polypeptide sequence of SEQ ID NO: 201, or variants thereof that retain functionality. In one embodiment the T cell activating bispecific antigen binding molecule comprises the polypeptide sequence of SEQ ID NO: 5, the polypeptide sequence of SEQ ID NO: 23, the polypeptide sequence of SEQ ID NO: 215 and the polypeptide sequence of SEQ ID NO: 217, or variants thereof that retain functionality. In one embodiment the T cell activating bispecific antigen binding molecule comprises the polypeptide sequence of SEQ ID NO: 5, the polypeptide sequence of SEQ ID NO: 23, the polypeptide sequence of SEQ ID NO: 215 and the polypeptide sequence of SEQ ID NO: 219, or variants thereof that retain functionality.

In one embodiment, the antigen binding moiety that is specific for MCSP comprises at least one heavy chain complementarity determining region (CDR) selected from the group consisting of SEQ ID NO: 280, SEQ ID NO: 281, SEQ ID NO: 282, SEQ ID NO: 301, SEQ ID NO: 303, SEQ ID NO: 304 and SEQ ID NO: 306 and at least one light chain CDR selected from the group of SEQ ID NO: 284, SEQ ID NO: 285, SEQ ID NO: 286, SEQ ID NO: 310, SEQ ID NO: 311, SEQ ID NO: 314, SEQ ID NO: 315, and SEQ ID NO: 316.

In one embodiment, the antigen binding moiety that is specific for MCSP comprises at least one heavy chain complementarity determining region (CDR) selected from the group consisting of SEQ ID NO: 280, SEQ ID NO: 281 and SEQ ID NO: 282 and at least one light chain CDR selected from the group of SEQ ID NO: 284, SEQ ID NO: 285 and SEQ ID NO: 286.

In one embodiment, the antigen binding moiety that is specific for MCSP comprises the heavy chain CDR1 of SEQ ID NO: 280, the heavy chain CDR2 of SEQ ID NO: 281, the heavy chain CDR3 of SEQ ID NO: 282, the light chain CDR1 of SEQ ID NO: 284, the light chain CDR2 of SEQ ID NO: 285 and the light chain CDR3 of SEQ ID NO: 286.

In a further embodiment, the antigen binding moiety that is specific for MCSP comprises a heavy chain variable region sequence that is at least about 95%, 96%, 97%, 98%, 99% or 100% identical to an amino acid sequence selected from the group of SEQ ID NO: 279, SEQ ID NO: 300, SEQ ID NO: 302, SEQ ID NO: 305 and SEQ ID NO: 307 and a light chain variable region sequence that is at least about 95%, 96%, 97%, 98%, 99% or 100% identical to an amino acid sequence selected from the group of SEQ ID NO: 283, SEQ ID NO: 309, SEQ ID NO: 312, SEQ ID NO: 313 and SEQ ID NO: 317.

In a further embodiment, the antigen binding moiety that is specific for MCSP comprises a heavy chain variable region comprising an amino acid sequence selected from the group of SEQ ID NO: 279, SEQ ID NO: 300, SEQ ID NO: 302, SEQ ID NO: 305 and SEQ ID NO: 307 and a light chain variable region comprising an amino acid sequence selected from the group of SEQ ID NO: 283, SEQ ID NO: 309, SEQ ID NO: 312, SEQ ID NO: 313 and SEQ ID NO: 317.

In one embodiment, the antigen binding moiety that is specific for MCSP comprises a heavy chain variable region comprising the amino acid sequence of SEQ ID NO: 279 and a light chain variable region comprising the amino acid sequence of SEQ ID NO: 283.

In one embodiment, the antigen binding moiety that is specific for MCSP comprises the heavy chain variable region sequence of SEQ ID NO: 279 and the light chain variable region sequence of SEQ ID NO: 283.

In a further embodiment, the antigen binding moiety that is specific for MCSP comprises a heavy chain variable region sequence that is at least about 95%, 96%, 97%, 98%, 99% or 100% identical to the amino acid sequence of SEQ ID NO: 279 and a light chain variable region sequence that is at least about 95%, 96%, 97%, 98%, 99% or 100% identical to the amino acid sequence of SEQ ID NO: 283, or variants thereof that retain functionality.

In one embodiment the T cell activating bispecific antigen binding molecule comprises a polypeptide sequence that is at least about 95%, 96%, 97%, 98%, 99% or 100% identical to SEQ ID NO: 278, a polypeptide sequence that is at least about 95%, 96%, 97%, 98%, 99% or 100% identical to SEQ ID NO: 319, a polypeptide sequence that is at least about 95%, 96%, 97%, 98%, 99% or 100% identical to SEQ ID NO: 320, and a polypeptide sequence that is at least about 95%, 96%, 97%, 98%, 99% or 100% identical to SEQ ID NO: 321.

In one embodiment the T cell activating bispecific antigen binding molecule comprises a polypeptide sequence that is at least about 95%, 96%, 97%, 98%, 99% or 100% identical to SEQ ID NO: 278, a polypeptide sequence that is at least about 95%, 96%, 97%, 98%, 99% or 100% identical to SEQ ID NO: 319, a polypeptide sequence that is at least about 95%, 96%, 97%, 98%, 99% or 100% identical to SEQ ID NO: 369, and a polypeptide sequence that is at least about 95%, 96%, 97%, 98%, 99% or 100% identical to SEQ ID NO: 370.

In one embodiment the T cell activating bispecific antigen binding molecule comprises a polypeptide sequence that is at least about 95%, 96%, 97%, 98%, 99% or 100% identical to SEQ ID NO: 278, a polypeptide sequence that is at least about 95%, 96%, 97%, 98%, 99% or 100% identical to SEQ ID NO: 319, a polypeptide sequence that is at least about 95%, 96%, 97%, 98%, 99% or 100% identical to SEQ ID NO: 371, and a polypeptide sequence that is at least about 95%, 96%, 97%, 98%, 99% or 100% identical to SEQ ID NO: 372.

In a specific embodiment the T cell activating bispecific antigen binding molecule comprises a polypeptide sequence encoded by a polynucleotide sequence that is at least about 80%, 85%, 90%, 95%, 96%, 97%, 98%, 99% or 100% identical to a sequence selected from the group of SEQ ID NO: 70, SEQ ID NO: 72, SEQ ID NO: 74, SEQ ID NO: 76, SEQ ID NO: 78, SEQ ID NO: 80, SEQ ID NO: 82, SEQ ID NO: 84, SEQ ID NO: 234, SEQ ID NO: 236, SEQ ID NO: 238, SEQ ID NO: 240, SEQ ID NO: 242, SEQ ID NO: 244, SEQ ID NO: 246, SEQ ID NO: 248, SEQ ID NO: 2, SEQ ID NO: 4, SEQ ID NO: 6, SEQ ID NO: 8, SEQ ID NO: 10, SEQ ID NO: 12, SEQ ID NO: 14, SEQ ID NO: 16, SEQ ID NO: 18, SEQ ID NO: 20, SEQ ID NO: 22, SEQ ID NO: 24, SEQ ID NO: 26, SEQ ID NO: 28, SEQ ID NO: 30, SEQ ID NO: 32, SEQ ID NO: 34, SEQ ID NO: 36, SEQ ID NO: 38, SEQ ID NO: 40, SEQ ID NO: 42, SEQ ID NO: 180, SEQ ID NO: 182, SEQ ID NO: 184, SEQ ID NO: 186, SEQ ID NO: 188, SEQ ID NO: 190, SEQ ID NO: 192, SEQ ID NO: 194, SEQ ID NO: 196, SEQ ID NO: 200, SEQ ID NO: 202, SEQ ID NO: 216, SEQ ID NO: 218 and SEQ ID NO: 220.

In one embodiment the T cell activating bispecific antigen binding molecule comprises at least one antigen binding moiety that is specific for Epidermal Growth Factor Receptor (EGFR). In another embodiment the T cell activating bispecific antigen binding molecule comprises at least one, typically two or more antigen binding moieties that can compete with monoclonal antibody GA201 for binding to an epitope of EGFR. See PCT publication WO 2006/082515, incorporated herein by reference in its entirety. In one embodiment, the antigen binding moiety that is specific for EGFR comprises the heavy chain CDR1 of SEQ ID NO: 85, the heavy chain CDR2 of SEQ ID NO: 87, the heavy chain CDR3 of SEQ ID NO: 89, the light chain CDR1 of SEQ ID NO: 93, the light chain CDR2 of SEQ ID NO: 95, and the light chain CDR3 of SEQ ID NO: 97. In a further embodiment, the antigen binding moiety that is specific for EGFR comprises a heavy chain variable region sequence that is at least about 80%, 85%, 90%, 95%, 96%, 97%, 98%, 99% or 100% identical to SEQ ID NO: 91 and a light chain variable region sequence that is at least about 80%, 85%, 90%, 95%, 96%, 97%, 98%, 99% or 100% identical to SEQ ID NO: 99, or variants thereof that retain functionality.

In yet another embodiment the T cell activating bispecific antigen binding molecule comprises the polypeptide sequence of SEQ ID NO: 43, the polypeptide sequence of SEQ ID NO: 45 and the polypeptide sequence of SEQ ID NO: 47, or variants thereof that retain functionality. In a further embodiment the T cell activating bispecific antigen binding molecule comprises the polypeptide sequence of SEQ ID NO: 49, the polypeptide sequence of SEQ ID NO: 51 and the polypeptide sequence of SEQ ID NO: 11, or variants thereof that retain functionality. In yet another embodiment the T cell activating bispecific antigen binding molecule comprises the polypeptide sequence of SEQ ID NO: 53, the polypeptide sequence of SEQ ID NO: 45 and the polypeptide sequence of SEQ ID NO: 47, or variants thereof that retain functionality.

In a specific embodiment the T cell activating bispecific antigen binding molecule comprises a polypeptide sequence encoded by a polynucleotide sequence that is at least about 80%, 85%, 90%, 95%, 96%, 97%, 98%, 99% or 100% identical to a sequence selected from the group of SEQ ID NO: 86, SEQ ID NO: 88, SEQ ID NO: 90, SEQ ID NO: 92, SEQ ID NO: 94, SEQ ID NO: 96, SEQ ID NO: 98, SEQ ID NO: 100, SEQ ID NO: 44, SEQ ID NO: 46, SEQ ID NO: 48, SEQ ID NO: 50, SEQ ID NO: 52, SEQ ID NO: 54 and SEQ ID NO: 12.

In one embodiment the T cell activating bispecific antigen binding molecule comprises at least one antigen binding moiety that is specific for Fibroblast Activation Protein (FAP). In another embodiment the T cell activating bispecific antigen binding molecule comprises at least one, typically two or more antigen binding moieties that can compete with monoclonal antibody 3F2 for binding to an epitope of FAP. See PCT publication WO 2012/020006, incorporated herein by reference in its entirety. In one embodiment, the antigen binding moiety that is specific for FAP comprises the heavy chain CDR1 of SEQ ID NO: 101, the heavy chain CDR2 of SEQ ID NO: 103, the heavy chain CDR3 of SEQ ID NO: 105, the light chain CDR1 of SEQ ID NO: 109, the light chain CDR2 of SEQ ID NO: 111, and the light chain CDR3 of SEQ ID NO: 113. In a further embodiment, the antigen binding moiety that is specific for FAP comprises a heavy chain variable region sequence that is at least about 80%, 85%, 90%, 95%, 96%, 97%, 98%, 99% or 100% identical to SEQ ID NO: 107 and a light chain variable region sequence that is at least about 80%, 85%, 90%, 95%, 96%, 97%, 98%, 99% or 100% identical to SEQ ID NO: 115, or variants thereof that retain functionality.

In yet another embodiment the T cell activating bispecific antigen binding molecule comprises the polypeptide sequence of SEQ ID NO: 55, the polypeptide sequence of SEQ ID NO: 51 and the polypeptide sequence of SEQ ID NO: 11, or variants thereof that retain functionality. In a further embodiment the T cell activating bispecific antigen binding molecule comprises the polypeptide sequence of SEQ ID NO: 57, the polypeptide sequence of SEQ ID NO: 59 and the polypeptide sequence of SEQ ID NO: 61, or variants thereof that retain functionality.

In a specific embodiment the T cell activating bispecific antigen binding molecule comprises a polypeptide sequence encoded by a polynucleotide sequence that is at least about 80%, 85%, 90%, 95%, 96%, 97%, 98%, 99% or 100% identical to a sequence selected from the group of SEQ ID NO: 102, SEQ ID NO: 104, SEQ ID NO: 106, SEQ ID NO: 108, SEQ ID NO: 110, SEQ ID NO: 112, SEQ ID NO: 114, SEQ ID NO: 116, SEQ ID NO: 56, SEQ ID NO: 58, SEQ ID NO: 60, SEQ ID NO: 62, SEQ ID NO: 52 and SEQ ID NO: 12.

In particular embodiments the T cell activating bispecific antigen binding molecule comprises at least one antigen binding moiety that is specific for Carcinoembryonic Antigen (CEA). In one embodiment the T cell activating bispecific antigen binding molecule comprises at least one, typically two or more antigen binding moieties that can compete with monoclonal antibody BW431/26 (described in European patent no. EP 160 897, and Bosslet et al., Int J Cancer 36, 75-84 (1985)) for binding to an epitope of CEA. In one embodiment the T cell activating bispecific antigen binding molecule comprises at least one, typically two or more antigen binding moieties that can compete with monoclonal antibody CH1A1A (see SEQ ID NOs 123 and 131) for binding to an epitope of CEA. See PCT patent publication number WO 2011/023787, incorporated herein by reference in its entirety. In one embodiment, the antigen binding moiety that is specific for CEA binds to the same epitope of CEA as monoclonal antibody CH1A1A. In one embodiment, the antigen binding moiety that is specific for CEA comprises the heavy chain CDR1 of SEQ ID NO: 117, the heavy chain CDR2 of SEQ ID NO: 119, the heavy chain CDR3 of SEQ ID NO: 121, the light chain CDR1 of SEQ ID NO: 125, the light chain CDR2 of SEQ ID NO: 127, and the light chain CDR3 of SEQ ID NO: 129. In a further embodiment, the antigen binding moiety that is specific for CEA comprises a heavy chain variable region sequence that is at least about 80%, 85%, 90%, 95%, 96%, 97%, 98%, 99% or 100%, particularly about 98%, 99% or 100%, identical to SEQ ID NO: 123 and a light chain variable region sequence that is at least about 80%, 85%, 90%, 95%, 96%, 97%, 98%, 99% or 100%, particularly about 98%, 99% or 100%, identical to SEQ ID NO: 131, or variants thereof that retain functionality. In one embodiment, the antigen binding moiety that is specific for CEA comprises the heavy and light chain variable region sequences of an affinity matured version of monoclonal antibody CH1A1A. In one embodiment, the antigen binding moiety that is specific for CEA comprises the heavy chain variable region sequence of SEQ ID NO: 123 with one, two, three, four, five, six or seven, particularly two, three, four or five, amino acid substitutions; and the light chain variable region sequence of SEQ ID NO: 131 with one, two, three, four, five, six or seven, particularly two, three, four or five, amino acid substitutions. Any amino acid residue within the variable region sequences may be substituted by a different amino acid, including amino acid residues within the CDR regions, provided that binding to CEA, particularly human CEA, is preserved. Preferred variants are those having a binding affinity for CEA at least equal (or stronger) to the binding affinity of the antigen binding moiety comprising the unsubstituted variable region sequences.

In one embodiment the T cell activating bispecific antigen binding molecule comprises the polypeptide sequence of SEQ ID NO: 63, the polypeptide sequence of SEQ ID NO: 65, the polypeptide sequence of SEQ ID NO: 67 and the polypeptide sequence of SEQ ID NO: 33, or variants thereof that retain functionality. In one embodiment the T cell activating bispecific antigen binding molecule comprises the polypeptide sequence of SEQ ID NO: 65, the polypeptide sequence of SEQ ID NO: 67, the polypeptide sequence of SEQ ID NO: 183 and the polypeptide sequence of SEQ ID NO: 197, or variants thereof that retain functionality. In one embodiment the T cell activating bispecific antigen binding molecule comprises the polypeptide sequence of SEQ ID NO: 183, the polypeptide sequence of SEQ ID NO: 203, the polypeptide sequence of SEQ ID NO: 205 and the polypeptide sequence of SEQ ID NO: 207, or variants thereof that retain functionality. In one embodiment the T cell activating bispecific antigen binding molecule comprises the polypeptide sequence of SEQ ID NO: 183, the polypeptide sequence of SEQ ID NO: 209, the polypeptide sequence of SEQ ID NO: 211 and the polypeptide sequence of SEQ ID NO: 213, or variants thereof that retain functionality.

In a specific embodiment the T cell activating bispecific antigen binding molecule comprises a polypeptide sequence encoded by a polynucleotide sequence that is at least about 80%, 85%, 90%, 95%, 96%, 97%, 98%, 99% or 100% identical to a sequence selected from the group of SEQ ID NO: 118, SEQ ID NO: 120, SEQ ID NO: 122, SEQ ID NO: 124, SEQ ID NO: 126, SEQ ID NO: 128, SEQ ID NO: 130, SEQ ID NO: 132, SEQ ID NO: 64, SEQ ID NO: 66, SEQ ID NO: 68, SEQ ID NO: 34, SEQ ID NO: 184, SEQ ID NO: 198, SEQ ID NO: 204, SEQ ID NO: 206, SEQ ID NO: 208, SEQ ID NO: 210, SEQ ID NO: 212 and SEQ ID NO: 214.

In one embodiment, the antigen binding moiety that is specific for CEA comprises at least one heavy chain complementarity determining region (CDR) selected from the group consisting of SEQ ID NO: 290, SEQ ID NO: 291 and SEQ ID NO: 292 and at least one light chain CDR selected from the group of SEQ ID NO: 294, SEQ ID NO: 295 and SEQ ID NO: 296.

In one embodiment, the antigen binding moiety that is specific for CEA comprises the heavy chain CDR1 of SEQ ID NO: 290, the heavy chain CDR2 of SEQ ID NO: 291, the heavy chain CDR3 of SEQ ID NO: 292, the light chain CDR1 of SEQ ID NO: 294, the light chain CDR2 of SEQ ID NO: 295 and the light chain CDR3 of SEQ ID NO: 296.

In one embodiment, the antigen binding moiety that is specific for CEA comprises a heavy chain variable region comprising the amino acid sequence of SEQ ID NO: 289 and a light chain variable region comprising the amino acid sequence of SEQ ID NO: 293.

In one embodiment, the antigen binding moiety that is specific for CEA comprises the heavy chain variable region sequence of SEQ ID NO: 289 and the light chain variable region sequence of SEQ ID NO: 293.

In a further embodiment, the antigen binding moiety that is specific for CEA comprises a heavy chain variable region sequence that is at least about 95%, 96%, 97%, 98%, 99% or 100% identical to the amino acid sequence of SEQ ID NO: 289 and a light chain variable region sequence that is at least about 95%, 96%, 97%, 98%, 99% or 100% identical to the amino acid sequence of SEQ ID NO: 293, or variants thereof that retain functionality.

In one embodiment the T cell activating bispecific antigen binding molecule comprises a polypeptide sequence that is at least about 95%, 96%, 97%, 98%, 99% or 100% identical to SEQ ID NO: 288, a polypeptide sequence that is at least about 95%, 96%, 97%, 98%, 99% or 100% identical to SEQ ID NO: 322, a polypeptide sequence that is at least about 95%, 96%, 97%, 98%, 99% or 100% identical to SEQ ID NO: 323, and a polypeptide sequence that is at least about 95%, 96%, 97%, 98%, 99% or 100% identical to SEQ ID NO: 324.

In one embodiment the T cell activating bispecific antigen binding molecule comprises at least one antigen binding moiety that is specific for CD33. In one embodiment, the antigen binding moiety that is specific for CD33 comprises the heavy chain CDR1 of SEQ ID NO: 133, the heavy chain CDR2 of SEQ ID NO: 135, the heavy chain CDR3 of SEQ ID NO: 137, the light chain CDR1 of SEQ ID NO: 141, the light chain CDR2 of SEQ ID NO: 143, and the light chain CDR3 of SEQ ID NO: 145. In a further embodiment, the antigen binding moiety that is specific for CD33 comprises a heavy chain variable region sequence that is at least about 80%, 85%, 90%, 95%, 96%, 97%, 98%, 99% or 100% identical to SEQ ID NO: 139 and a light chain variable region sequence that is at least about 80%, 85%, 90%, 95%, 96%, 97%, 98%, 99% or 100% identical to SEQ ID NO: 147, or variants thereof that retain functionality.

In one embodiment the T cell activating bispecific antigen binding molecule comprises the polypeptide sequence of SEQ ID NO: 33, the polypeptide sequence of SEQ ID NO: 213, the polypeptide sequence of SEQ ID NO: 221 and the polypeptide sequence of SEQ ID NO: 223, or variants thereof that retain functionality. In one embodiment the T cell activating bispecific antigen binding molecule comprises the polypeptide sequence of SEQ ID NO: 33, the polypeptide sequence of SEQ ID NO: 221, the polypeptide sequence of SEQ ID NO: 223 and the polypeptide sequence of SEQ ID NO: 225, or variants thereof that retain functionality.

In a specific embodiment the T cell activating bispecific antigen binding molecule comprises a polypeptide sequence encoded by a polynucleotide sequence that is at least about 80%, 85%, 90%, 95%, 96%, 97%, 98%, 99% or 100% identical to a sequence selected from the group of SEQ ID NO: 134, SEQ ID NO: 136, SEQ ID NO: 138, SEQ ID NO: 140, SEQ ID NO: 142, SEQ ID NO: 144, SEQ ID NO: 146, SEQ ID NO: 148, SEQ ID NO: 34, SEQ ID NO: 214, SEQ ID NO: 222, SEQ ID NO: 224 and SEQ ID NO: 226.

### Polynucleotides

The invention further provides isolated polynucleotides encoding a T cell activating bispecific antigen binding molecule as described herein or a fragment thereof. In some embodiments, said fragment is an antigen binding fragment.

Polynucleotides of the invention include those that are at least about 80%, 85%, 90%, 95%, 96%, 97%, 98%, 99%, or 100% identical to the sequences set forth in SEQ ID NOs 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262, 264, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 373, and 374 including functional fragments or variants thereof.

The polynucleotides encoding T cell activating bispecific antigen binding molecules of the invention may be expressed as a single polynucleotide that encodes the entire T cell activating bispecific antigen binding molecule or as multiple (e.g., two or more) polynucleotides that are co-expressed. Polypeptides encoded by polynucleotides that are co-expressed may associate through, e.g., disulfide bonds or other means to form a functional T cell activating bispecific antigen binding molecule. For example, the light chain portion of an antigen binding moiety may be encoded by a separate polynucleotide from the portion of the T cell activating bispecific antigen binding molecule comprising the heavy chain portion of the antigen binding moiety, an Fc domain subunit and optionally (part of) another antigen binding moiety. When co-expressed, the heavy chain polypeptides will associate with the light chain polypeptides to form the antigen binding moiety. In another example, the portion of the T cell activating bispecific antigen binding molecule comprising one of the two Fc domain subunits and optionally (part of) one or more antigen binding moieties could be encoded by a separate polynucleotide from the portion of the T cell activating bispecific antigen binding molecule comprising the other of the two Fc domain subunits and optionally (part of) an antigen binding moiety. When co-expressed, the Fc domain subunits will associate to form the Fc domain.

In certain embodiments, an isolated polynucleotide of the invention encodes a fragment of a T cell activating bispecific antigen binding molecule comprising a first and a second antigen binding moiety, and an Fc domain consisting of two subunits, wherein the first antigen binding moiety is a single chain Fab molecule. In one embodiment, an isolated polynucleotide of the invention encodes the first antigen binding moiety and a subunit of the Fc domain. In a more specific embodiment the isolated polynucleotide encodes a polypeptide wherein a single chain Fab molecule shares a carboxy-terminal peptide bond with an Fc domain subunit. In another embodiment, an isolated polynucleotide of the invention encodes the heavy chain of the second antigen binding moiety and a subunit of the Fc domain. In a more specific embodiment the isolated polynucleotide encodes a polypeptide wherein a Fab heavy chain shares a carboxy terminal peptide bond with an Fc domain subunit. In yet another embodiment, an isolated polynucleotide of the invention encodes the first antigen binding moiety, the heavy chain of the second antigen binding moiety and a subunit of the Fc domain. In a more specific embodiment, the isolated polynucleotide encodes a polypeptide wherein a single chain Fab molecule shares a carboxy-terminal peptide bond with a Fab heavy chain, which in turn shares a carboxy-terminal peptide bond with an Fc domain subunit.

In certain embodiments, an isolated polynucleotide of the invention encodes a fragment of a T cell activating bispecific antigen binding molecule comprising a first and a second antigen binding moiety, and an Fc domain consisting of two subunits, wherein the first antigen binding moiety is a crossover Fab molecule. In one embodiment, an isolated polynucleotide of the invention encodes the heavy chain of the first antigen binding moiety and a subunit of the Fc domain. In a more specific embodiment the isolated polynucleotide encodes a polypeptide wherein Fab light chain variable region shares a carboxy terminal peptide bond with a Fab heavy chain constant region, which in turn shares a carboxy-terminal peptide bond with an Fc domain subunit. In another specific embodiment the isolated polynucleotide encodes a polypeptide wherein Fab heavy chain variable region shares a carboxy terminal peptide bond with a Fab light chain constant region, which in turn shares a carboxy-terminal peptide bond with an Fc domain subunit. In another embodiment, an isolated polynucleotide of the invention encodes the heavy chain of the second antigen binding moiety and a subunit of the Fc domain. In a more specific embodiment the isolated polynucleotide encodes a polypeptide wherein a Fab heavy chain shares a carboxy terminal peptide bond with an Fc domain subunit. In yet another embodiment, an isolated polynucleotide of the invention encodes the heavy chain of the first antigen binding moiety, the heavy chain of the second antigen binding moiety and a subunit of the Fc domain. In a more specific embodiment, the isolated polynucleotide encodes a polypeptide wherein a Fab light chain variable region shares a carboxy-terminal peptide bond with a Fab heavy chain constant region, which in turn shares a carboxy-terminal peptide bond with a Fab heavy chain, which in turn shares a carboxy-terminal peptide bond with an Fc domain subunit. In another specific embodiment, the isolated polynucleotide encodes a polypeptide wherein a Fab heavy chain variable region shares a carboxy-terminal peptide bond with a Fab light chain constant region, which in turn shares a carboxy-terminal peptide bond with a Fab heavy chain, which in turn shares a carboxy-terminal peptide bond with an Fc domain subunit. In yet another specific embodiment the isolated polynucleotide encodes a polypeptide wherein a Fab heavy chain shares a carboxy-terminal peptide bond with a Fab light chain variable region, which in turn shares a carboxy-terminal peptide bond with a Fab heavy chain constant region, which in turn shares a carboxy-terminal peptide bond with an Fc domain subunit. In still another specific embodiment the isolated polynucleotide encodes a polypeptide wherein a Fab heavy chain shares a carboxy-terminal peptide bond with a Fab heavy chain variable region, which in turn shares a carboxy-terminal peptide bond with a Fab light chain constant region, which in turn shares a carboxy-terminal peptide bond with an Fc domain subunit.

In further embodiments, an isolated polynucleotide of the invention encodes the heavy chain of a third antigen binding moiety and a subunit of the Fc domain. In a more specific embodiment the isolated polynucleotide encodes a polypeptide wherein a Fab heavy chain shares a carboxy terminal peptide bond with an Fc domain subunit.

In further embodiments, an isolated polynucleotide of the invention encodes the light chain of an antigen binding moiety. In some embodiments, the isolated polynucleotide encodes a polypeptide wherein a Fab light chain variable region shares a carboxy-terminal peptide bond with a Fab heavy chain constant region. In other embodiments, the isolated polynucleotide encodes a polypeptide wherein a Fab heavy chain variable region shares a carboxy-terminal peptide bond with a Fab light chain constant region. In still other embodiments, an isolated polynucleotide of the invention encodes the light chain of the first antigen binding moiety and the light chain of the second antigen binding moiety. In a more specific embodiment, the isolated polynucleotide encodes a polypeptide wherein a Fab heavy chain variable region shares a carboxy-terminal peptide bond with a Fab light chain constant region, which in turn shares a carboxy-terminal peptide bond with a Fab light chain. In another specific embodiment the isolated polynucleotide encodes a polypeptide wherein a Fab light chain shares a carboxy-terminal peptide bond with a Fab heavy chain variable region, which in turn shares a carboxy-terminal peptide bond with a Fab light chain constant region. In yet another specific embodiment, the isolated polynucleotide encodes a polypeptide wherein a Fab light chain variable region shares a carboxy-terminal peptide bond with a Fab heavy chain constant region, which in turn shares a carboxy-terminal peptide bond with a Fab light chain. In yet another specific embodiment the isolated polynucleotide encodes a polypeptide wherein a Fab light chain shares a carboxy-terminal peptide bond with a Fab light chain variable region, which in turn shares a carboxy-terminal peptide bond with a Fab heavy chain constant region.

In another embodiment, the present invention is directed to an isolated polynucleotide encoding a T cell activating bispecific antigen binding molecule of the invention or a fragment thereof, wherein the polynucleotide comprises a sequence that encodes a variable region sequence as shown in SEQ ID NOs 75, 83, 91, 99, 107, 115, 123, 131, 139, 147, 169, 177, 239, 247, 255 and 263. In another embodiment, the present invention is directed to an isolated polynucleotide encoding a T cell activating bispecific antigen binding molecule or fragment thereof, wherein the polynucleotide comprises a sequence that encodes a polypeptide sequence as shown in SEQ ID NOs 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 179, 181, 183, 185, 187, 189, 191, 193, 195, 197, 199, 201, 203, 205, 207, 209, 211, 213, 215, 217, 219, 221, 223, 225, 227, 229 and 231. In another embodiment, the invention is further directed to an isolated polynucleotide encoding a T cell activating bispecific antigen binding molecule of the invention or a fragment thereof, wherein the polynucleotide comprises a sequence that is at least about 80%, 85%, 90%, 95%, 96%, 97%, 98%, or 99% identical to a nucleotide sequence shown in SEQ ID NOs 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262 or 264. In another embodiment, the invention is directed to an isolated polynucleotide encoding a T cell activating bispecific antigen binding molecule of the invention or a fragment thereof, wherein the polynucleotide comprises a nucleic acid sequence shown in SEQ ID NOs 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 136, 138, 140, 142, 144, 146, 148, 164, 166, 168, 170, 172, 174, 176, 178, 180, 182, 184, 186, 188, 190, 192, 194, 196, 198, 200, 202, 204, 206, 208, 210, 212, 214, 216, 218, 220, 222, 224, 226, 228, 230, 232, 234, 236, 238, 240, 242, 244, 246, 248, 250, 252, 254, 256, 258, 260, 262 or 264. In another embodiment, the invention is directed to an isolated polynucleotide encoding a T cell activating bispecific antigen binding molecule of the invention or a fragment thereof, wherein the polynucleotide comprises a sequence that encodes a variable region sequence that is at least about 80%, 85%, 90%, 95%, 96%, 97%, 98%, or 99% identical to an amino acid sequence in SEQ ID NOs 75, 83, 91, 99, 107, 115, 123, 131, 139, 147, 169, 177, 239, 247, 255 or 263. In another embodiment, the invention is directed to an isolated polynucleotide encoding a T cell activating bispecific antigen binding molecule or fragment thereof, wherein the polynucleotide comprises a sequence that encodes a polypeptide sequence that is at least 80%, 85%, 90%, 95%, 96%, 97%, 98%, or 99% identical to an amino acid sequence in SEQ ID NOs 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 179, 181, 183, 185, 187, 189, 191, 193, 195, 197, 199, 201, 203, 205, 207, 209, 211, 213, 215, 217, 219, 221, 223, 225, 227, 229 or 231. The invention encompasses an isolated polynucleotide encoding a T cell activating bispecific antigen binding molecule of the invention or a fragment thereof, wherein the polynucleotide comprises a sequence that encodes the variable region sequence of SEQ ID NOs 75, 83, 91, 99, 107, 115, 123, 131, 139, 147, 169, 177, 239, 247, 255 or 263 with conservative amino acid substitutions.

In another embodiment, the present invention is directed to an isolated polynucleotide encoding a T cell activating bispecific antigen binding molecule of the invention or a fragment thereof, wherein the polynucleotide comprises a sequence that encodes a variable region sequence as shown in SEQ ID NOs 269, 273, 279, 283, 289, 293, 297, 298, 299, 300, 302, 305, 307, 309, 312, 313 or 317. In another embodiment, the present invention is directed to an isolated polynucleotide encoding a T cell activating bispecific antigen binding molecule or fragment thereof, wherein the polynucleotide comprises a sequence that encodes a polypeptide sequence as shown in SEQ ID NOs 288, 322, 323, 324, 278, 319, 320, 321, 369, 370, 371 or 372. In another embodiment, the invention is further directed to an isolated polynucleotide encoding a T cell activating bispecific antigen binding molecule of the invention or a fragment thereof, wherein the polynucleotide comprises a sequence that is at least about 80%, 85%, 90%, 95%, 96%, 97%, 98%, or 99% identical to a nucleotide sequence shown in SEQ ID NOs 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 364, 364, 373 or 374. In another embodiment, the invention is directed to an isolated polynucleotide encoding a T cell activating bispecific antigen binding molecule of the invention or a fragment thereof, wherein the polynucleotide comprises a nucleic acid sequence shown in SEQ ID NOs 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 373 or 374. In another embodiment, the invention is directed to an isolated polynucleotide encoding a T cell activating bispecific antigen binding molecule of the invention or a fragment thereof, wherein the polynucleotide comprises a sequence that encodes a variable region sequence that is at least about 80%, 85%, 90%, 95%, 96%, 97%, 98%, or 99% identical to the amino acid sequence of SEQ ID NOs 269, 273, 279, 283, 289, 293, 297, 298, 299, 300, 302, 305, 307, 309, 312, 313 or 317. In another embodiment, the invention is directed to an isolated polynucleotide encoding a T cell activating bispecific antigen binding molecule or fragment thereof, wherein the polynucleotide comprises a sequence that encodes a polypeptide sequence that is at least 80%, 85%, 90%, 95%, 96%, 97%, 98%, or 99% identical to the amino acid sequence of SEQ ID NOs 288, 322, 323, 324, 278, 319, 320, 321, 369, 370, 371 or 372.

The invention encompasses an isolated polynucleotide encoding a T cell activating bispecific antigen binding molecule of the invention or a fragment thereof, wherein the polynucleotide comprises a sequence that encodes the variable region sequence of SEQ ID NOs 269, 273, 279, 283, 289, 293, 297, 298, 299, 300, 302, 305, 307, 309, 312, 313 or 317 with conservative amino acid substitutions. The invention also encompasses an isolated polynucleotide encoding a T cell activating bispecific antigen binding molecule of the invention or fragment thereof, wherein the polynucleotide comprises a sequence that encodes the polypeptide sequence of SEQ ID NOs 288, 322, 323, 324, 278, 319, 320 or 321 with conservative amino acid substitutions.

The invention also encompasses an isolated polynucleotide encoding a T cell activating bispecific antigen binding molecule of the invention or fragment thereof, wherein the polynucleotide comprises a sequence that encodes the polypeptide sequence of SEQ ID NOs 278, 319, 369 or 370 with conservative amino acid substitutions.

The invention also encompasses an isolated polynucleotide encoding a T cell activating bispecific antigen binding molecule of the invention or fragment thereof, wherein the polynucleotide comprises a sequence that encodes the polypeptide sequence of SEQ ID NOs 278, 319, 371 or 372 with conservative amino acid substitutions.

The invention also encompasses an isolated polynucleotide encoding a T cell activating bispecific antigen binding molecule of the invention or fragment thereof, wherein the polynucleotide comprises a sequence that encodes the polypeptide sequence of SEQ ID NOs 1, 3, 5, 7, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 179, 181, 183, 185, 187, 189, 191, 193, 195, 197, 199, 201, 203, 205, 207, 209, 211, 213, 215, 217, 219, 221, 223, 225, 227, 229, 231, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 364, 364, 373 or 374 with conservative amino acid substitutions.

In certain embodiments the polynucleotide or nucleic acid is DNA. In other embodiments, a polynucleotide of the present invention is RNA, for example, in the form of messenger RNA (mRNA). RNA of the present invention may be single stranded or double stranded.

### Recombinant Methods

T cell activating bispecific antigen binding molecules of the invention may be obtained, for example, by solid-state peptide synthesis (e.g. Merrifield solid phase synthesis) or recombinant production. For recombinant production one or more polynucleotide encoding the T cell activating bispecific antigen binding molecule (fragment), e.g., as described above, is isolated and inserted into one or more vectors for further cloning and/or expression in a host cell. Such polynucleotide may be readily isolated and sequenced using conventional procedures. In one embodiment a vector, preferably an expression vector, comprising one or more of the polynucleotides of the invention is provided. Methods which are well known to those skilled in the art can be used to construct expression vectors containing the coding sequence of a T cell activating bispecific antigen binding molecule (fragment) along with appropriate transcriptional/translational control signals. These methods include in vitro recombinant DNA techniques, synthetic techniques and in vivo recombination/genetic recombination. See, for example, the techniques described in Maniatis et al., MOLECULAR CLONING: A LABORATORY MANUAL, Cold Spring Harbor Laboratory, N.Y. (1989); and Ausubel et al., CURRENT PROTOCOLS IN MOLECULAR BIOLOGY, Greene Publishing Associates and Wiley Interscience, N.Y (1989). The expression vector can be part of a plasmid, virus, or may be a nucleic acid fragment. The expression vector includes an expression cassette into which the polynucleotide encoding the T cell activating bispecific antigen binding molecule (fragment) (i.e. the coding region) is cloned in operable association with a promoter and/or other transcription or translation control elements. As used herein, a “coding region” is a portion of nucleic acid which consists of codons translated into amino acids. Although a “stop codon” (TAG, TGA, or TAA) is not translated into an amino acid, it may be considered to be part of a coding region, if present, but any flanking sequences, for example promoters, ribosome binding sites, transcriptional terminators, introns, 5′ and 3′ untranslated regions, and the like, are not part of a coding region. Two or more coding regions can be present in a single polynucleotide construct, e.g. on a single vector, or in separate polynucleotide constructs, e.g. on separate (different) vectors. Furthermore, any vector may contain a single coding region, or may comprise two or more coding regions, e.g. a vector of the present invention may encode one or more polypeptides, which are post- or co-translationally separated into the final proteins via proteolytic cleavage. In addition, a vector, polynucleotide, or nucleic acid of the invention may encode heterologous coding regions, either fused or unfused to a polynucleotide encoding the T cell activating bispecific antigen binding molecule (fragment) of the invention, or variant or derivative thereof. Heterologous coding regions include without limitation specialized elements or motifs, such as a secretory signal peptide or a heterologous functional domain. An operable association is when a coding region for a gene product, e.g. a polypeptide, is associated with one or more regulatory sequences in such a way as to place expression of the gene product under the influence or control of the regulatory sequence(s). Two DNA fragments (such as a polypeptide coding region and a promoter associated therewith) are “operably associated” if induction of promoter function results in the transcription of mRNA encoding the desired gene product and if the nature of the linkage between the two DNA fragments does not interfere with the ability of the expression regulatory sequences to direct the expression of the gene product or interfere with the ability of the DNA template to be transcribed. Thus, a promoter region would be operably associated with a nucleic acid encoding a polypeptide if the promoter was capable of effecting transcription of that nucleic acid. The promoter may be a cell-specific promoter that directs substantial transcription of the DNA only in predetermined cells. Other transcription control elements, besides a promoter, for example enhancers, operators, repressors, and transcription termination signals, can be operably associated with the polynucleotide to direct cell-specific transcription. Suitable promoters and other transcription control regions are disclosed herein. A variety of transcription control regions are known to those skilled in the art. These include, without limitation, transcription control regions, which function in vertebrate cells, such as, but not limited to, promoter and enhancer segments from cytomegaloviruses (e.g. the immediate early promoter, in conjunction with intron-A), simian virus 40 (e.g. the early promoter), and retroviruses (such as, e.g. Rous sarcoma virus). Other transcription control regions include those derived from vertebrate genes such as actin, heat shock protein, bovine growth hormone and rabbit d-globin, as well as other sequences capable of controlling gene expression in eukaryotic cells. Additional suitable transcription control regions include tissue-specific promoters and enhancers as well as inducible promoters (e.g. promoters inducible tetracycline). Similarly, a variety of translation control elements are known to those of ordinary skill in the art. These include, but are not limited to ribosome binding sites, translation initiation and termination codons, and elements derived from viral systems (particularly an internal ribosome entry site, or IRES, also referred to as a CITE sequence). The expression cassette may also include other features such as an origin of replication, and/or chromosome integration elements such as retroviral long terminal repeats (LTRs), or adeno-associated viral (AAV) inverted terminal repeats (ITRs).

Polynucleotide and nucleic acid coding regions of the present invention may be associated with additional coding regions which encode secretory or signal peptides, which direct the secretion of a polypeptide encoded by a polynucleotide of the present invention. For example, if secretion of the T cell activating bispecific antigen binding molecule is desired, DNA encoding a signal sequence may be placed upstream of the nucleic acid encoding a T cell activating bispecific antigen binding molecule of the invention or a fragment thereof. According to the signal hypothesis, proteins secreted by mammalian cells have a signal peptide or secretory leader sequence which is cleaved from the mature protein once export of the growing protein chain across the rough endoplasmic reticulum has been initiated. Those of ordinary skill in the art are aware that polypeptides secreted by vertebrate cells generally have a signal peptide fused to the N-terminus of the polypeptide, which is cleaved from the translated polypeptide to produce a secreted or “mature” form of the polypeptide. In certain embodiments, the native signal peptide, e.g. an immunoglobulin heavy chain or light chain signal peptide is used, or a functional derivative of that sequence that retains the ability to direct the secretion of the polypeptide that is operably associated with it. Alternatively, a heterologous mammalian signal peptide, or a functional derivative thereof, may be used. For example, the wild-type leader sequence may be substituted with the leader sequence of human tissue plasminogen activator (TPA) or mouse β-glucuronidase. Exemplary amino acid and polynucleotide sequences of secretory signal peptides are given in SEQ ID NOs 154-162.

DNA encoding a short protein sequence that could be used to facilitate later purification (e.g. a histidine tag) or assist in labeling the T cell activating bispecific antigen binding molecule may be included within or at the ends of the T cell activating bispecific antigen binding molecule (fragment) encoding polynucleotide.

In a further embodiment, a host cell comprising one or more polynucleotides of the invention is provided. In certain embodiments a host cell comprising one or more vectors of the invention is provided. The polynucleotides and vectors may incorporate any of the features, singly or in combination, described herein in relation to polynucleotides and vectors, respectively. In one such embodiment a host cell comprises (e.g. has been transformed or transfected with) a vector comprising a polynucleotide that encodes (part of) a T cell activating bispecific antigen binding molecule of the invention. As used herein, the term “host cell” refers to any kind of cellular system which can be engineered to generate the T cell activating bispecific antigen binding molecules of the invention or fragments thereof. Host cells suitable for replicating and for supporting expression of T cell activating bispecific antigen binding molecules are well known in the art. Such cells may be transfected or transduced as appropriate with the particular expression vector and large quantities of vector containing cells can be grown for seeding large scale fermenters to obtain sufficient quantities of the T cell activating bispecific antigen binding molecule for clinical applications. Suitable host cells include prokaryotic microorganisms, such as E. coli, or various eukaryotic cells, such as Chinese hamster ovary cells (CHO), insect cells, or the like. For example, polypeptides may be produced in bacteria in particular when glycosylation is not needed. After expression, the polypeptide may be isolated from the bacterial cell paste in a soluble fraction and can be further purified. In addition to prokaryotes, eukaryotic microbes such as filamentous fungi or yeast are suitable cloning or expression hosts for polypeptide-encoding vectors, including fungi and yeast strains whose glycosylation pathways have been “humanized”, resulting in the production of a polypeptide with a partially or fully human glycosylation pattern. See Gerngross, Nat Biotech 22, 1409-1414 (2004), and Li et al., Nat Biotech 24, 210-215 (2006).

Suitable host cells for the expression of (glycosylated) polypeptides are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. See e.g. U.S. Pat. Nos. 5,959,177, 6,040,498, 6,420,548, 7,125,978, and 6,417,429 (describing PLANTIBODIES™ technology for producing antibodies in transgenic plants). Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham et al., J Gen Virol 36, 59 (1977)), baby hamster kidney cells (BHK), mouse sertoli cells (TM4 cells as described, e.g., in Mather, Biol Reprod 23, 243-251 (1980)), monkey kidney cells (CV1), African green monkey kidney cells (VERO-76), human cervical carcinoma cells (HELA), canine kidney cells (MDCK), buffalo rat liver cells (BRL 3A), human lung cells (W138), human liver cells (Hep G2), mouse mammary tumor cells (MMT 060562), TRI cells (as described, e.g., in Mather et al., Annals N.Y. Acad Sci 383, 44-68 (1982)), MRC 5 cells, and FS4 cells. Other useful mammalian host cell lines include Chinese hamster ovary (CHO) cells, including dhfr⁻ CHO cells (Urlaub et al., Proc Natl Acad Sci USA 77, 4216 (1980)); and myeloma cell lines such as YO, NS0, P3X63 and Sp2/0. For a review of certain mammalian host cell lines suitable for protein production, see, e.g., Yazaki and Wu, Methods in Molecular Biology, Vol. 248 (B. K. C. Lo, ed., Humana Press, Totowa, N.J.), pp. 255-268 (2003). Host cells include cultured cells, e.g., mammalian cultured cells, yeast cells, insect cells, bacterial cells and plant cells, to name only a few, but also cells comprised within a transgenic animal, transgenic plant or cultured plant or animal tissue. In one embodiment, the host cell is a eukaryotic cell, preferably a mammalian cell, such as a Chinese Hamster Ovary (CHO) cell, a human embryonic kidney (HEK) cell or a lymphoid cell (e.g., Y0, NS0, Sp20 cell).

Standard technologies are known in the art to express foreign genes in these systems. Cells expressing a polypeptide comprising either the heavy or the light chain of an antigen binding domain such as an antibody, may be engineered so as to also express the other of the antibody chains such that the expressed product is an antibody that has both a heavy and a light chain.

In one embodiment, a method of producing a T cell activating bispecific antigen binding molecule according to the invention is provided, wherein the method comprises culturing a host cell comprising a polynucleotide encoding the T cell activating bispecific antigen binding molecule, as provided herein, under conditions suitable for expression of the T cell activating bispecific antigen binding molecule, and recovering the T cell activating bispecific antigen binding molecule from the host cell (or host cell culture medium).

The components of the T cell activating bispecific antigen binding molecule are genetically fused to each other. T cell activating bispecific antigen binding molecule can be designed such that its components are fused directly to each other or indirectly through a linker sequence. The composition and length of the linker may be determined in accordance with methods well known in the art and may be tested for efficacy. Examples of linker sequences between different components of T cell activating bispecific antigen binding molecules are found in the sequences provided herein. Additional sequences may also be included to incorporate a cleavage site to separate the individual components of the fusion if desired, for example an endopeptidase recognition sequence.

In certain embodiments the one or more antigen binding moieties of the T cell activating bispecific antigen binding molecules comprise at least an antibody variable region capable of binding an antigenic determinant. Variable regions can form part of and be derived from naturally or non-naturally occurring antibodies and fragments thereof. Methods to produce polyclonal antibodies and monoclonal antibodies are well known in the art (see e.g. Harlow and Lane, “Antibodies, a laboratory manual”, Cold Spring Harbor Laboratory, 1988). Non-naturally occurring antibodies can be constructed using solid phase-peptide synthesis, can be produced recombinantly (e.g. as described in U.S. Pat. No. 4,186,567) or can be obtained, for example, by screening combinatorial libraries comprising variable heavy chains and variable light chains (see e.g. U.S. Pat. No. 5,969,108 to McCafferty).

Any animal species of antibody, antibody fragment, antigen binding domain or variable region can be used in the T cell activating bispecific antigen binding molecules of the invention. Non-limiting antibodies, antibody fragments, antigen binding domains or variable regions useful in the present invention can be of murine, primate, or human origin. If the T cell activating bispecific antigen binding molecule is intended for human use, a chimeric form of antibody may be used wherein the constant regions of the antibody are from a human. A humanized or fully human form of the antibody can also be prepared in accordance with methods well known in the art (see e. g. U.S. Pat. No. 5,565,332 to Winter). Humanization may be achieved by various methods including, but not limited to (a) grafting the non-human (e.g., donor antibody) CDRs onto human (e.g. recipient antibody) framework and constant regions with or without retention of critical framework residues (e.g. those that are important for retaining good antigen binding affinity or antibody functions), (b) grafting only the non-human specificity-determining regions (SDRs or a-CDRs; the residues critical for the antibody-antigen interaction) onto human framework and constant regions, or (c) transplanting the entire non-human variable domains, but “cloaking” them with a human-like section by replacement of surface residues. Humanized antibodies and methods of making them are reviewed, e.g., in Almagro and Fransson, Front Biosci 13, 1619-1633 (2008), and are further described, e.g., in Riechmann et al., Nature 332, 323-329 (1988); Queen et al., Proc Natl Acad Sci USA 86, 10029-10033 (1989); U.S. Pat. Nos. 5,821,337, 7,527,791, 6,982,321, and U.S. Pat. No. 7,087,409; Jones et al., Nature 321, 522-525 (1986); Morrison et al., Proc Natl Acad Sci 81, 6851-6855 (1984); Morrison and Oi, Adv Immunol 44, 65-92 (1988); Verhoeyen et al., Science 239, 1534-1536 (1988); Padlan, Molec Immun 31(3), 169-217 (1994); Kashmiri et al., Methods 36, 25-34 (2005) (describing SDR (a-CDR) grafting); Padlan, Mol Immunol 28, 489-498 (1991) (describing “resurfacing”); Dall'Acqua et al., Methods 36, 43-60 (2005) (describing “FR shuffling”); and Osbourn et al., Methods 36, 61-68 (2005) and Klimka et al., Br J Cancer 83, 252-260 (2000) (describing the “guided selection” approach to FR shuffling). Human antibodies and human variable regions can be produced using various techniques known in the art. Human antibodies are described generally in van Dijk and van de Winkel, Curr Opin Pharmacol 5, 368-74 (2001) and Lonberg, Curr Opin Immunol 20, 450-459 (2008). Human variable regions can form part of and be derived from human monoclonal antibodies made by the hybridoma method (see e.g. Monoclonal Antibody Production Techniques and Applications, pp. 51-63 (Marcel Dekker, Inc., New York, 1987)). Human antibodies and human variable regions may also be prepared by administering an immunogen to a transgenic animal that has been modified to produce intact human antibodies or intact antibodies with human variable regions in response to antigenic challenge (see e.g. Lonberg, Nat Biotech 23, 1117-1125 (2005). Human antibodies and human variable regions may also be generated by isolating Fv clone variable region sequences selected from human-derived phage display libraries (see e.g., Hoogenboom et al. in Methods in Molecular Biology 178, 1-37 (O'Brien et al., ed., Human Press, Totowa, N.J., 2001); and McCafferty et al., Nature 348, 552-554; Clackson et al., Nature 352, 624-628 (1991)). Phage typically display antibody fragments, either as single-chain Fv (scFv) fragments or as Fab fragments.

In certain embodiments, the antigen binding moieties useful in the present invention are engineered to have enhanced binding affinity according to, for example, the methods disclosed in U.S. Pat. Appl. Publ. No. 2004/0132066, the entire contents of which are hereby incorporated by reference. The ability of the T cell activating bispecific antigen binding molecule of the invention to bind to a specific antigenic determinant can be measured either through an enzyme-linked immunosorbent assay (ELISA) or other techniques familiar to one of skill in the art, e.g. surface plasmon resonance technique (analyzed on a BIACORE T100 system) (Liljeblad, et al., Glyco J 17, 323-329 (2000)), and traditional binding assays (Heeley, Endocr Res 28, 217-229 (2002)). Competition assays may be used to identify an antibody, antibody fragment, antigen binding domain or variable domain that competes with a reference antibody for binding to a particular antigen, e.g. an antibody that competes with the V9 antibody for binding to CD3. In certain embodiments, such a competing antibody binds to the same epitope (e.g. a linear or a conformational epitope) that is bound by the reference antibody. Detailed exemplary methods for mapping an epitope to which an antibody binds are provided in Morris (1996) “Epitope Mapping Protocols,” in Methods in Molecular Biology vol. 66 (Humana Press, Totowa, N.J.). In an exemplary competition assay, immobilized antigen (e.g. CD3) is incubated in a solution comprising a first labeled antibody that binds to the antigen (e.g. V9 antibody) and a second unlabeled antibody that is being tested for its ability to compete with the first antibody for binding to the antigen. The second antibody may be present in a hybridoma supernatant. As a control, immobilized antigen is incubated in a solution comprising the first labeled antibody but not the second unlabeled antibody. After incubation under conditions permissive for binding of the first antibody to the antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to the antigen. See Harlow and Lane (1988) Antibodies: A Laboratory Manual ch.14 (Cold Spring Harbor Laboratory, Cold Spring Harbor, N.Y.).

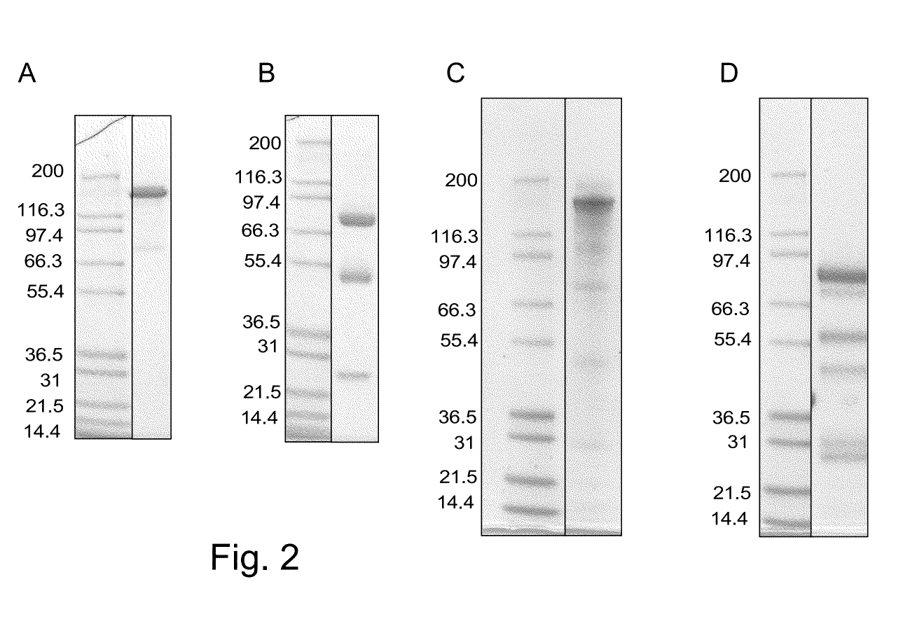

T cell activating bispecific antigen binding molecules prepared as described herein may be purified by art-known techniques such as high performance liquid chromatography, ion exchange chromatography, gel electrophoresis, affinity chromatography, size exclusion chromatography, and the like. The actual conditions used to purify a particular protein will depend, in part, on factors such as net charge, hydrophobicity, hydrophilicity etc., and will be apparent to those having skill in the art. For affinity chromatography purification an antibody, ligand, receptor or antigen can be used to which the T cell activating bispecific antigen binding molecule binds. For example, for affinity chromatography purification of T cell activating bispecific antigen binding molecules of the invention, a matrix with protein A or protein G may be used. Sequential Protein A or G affinity chromatography and size exclusion chromatography can be used to isolate a T cell activating bispecific antigen binding molecule essentially as described in the Examples. The purity of the T cell activating bispecific antigen binding molecule can be determined by any of a variety of well known analytical methods including gel electrophoresis, high pressure liquid chromatography, and the like. For example, the heavy chain fusion proteins expressed as described in the Examples were shown to be intact and properly assembled as demonstrated by reducing SDS-PAGE (see e.g. FIG. 2). Three bands were resolved at approximately Mr 25,000, Mr 50,000 and Mr 75,000, corresponding to the predicted molecular weights of the T cell activating bispecific antigen binding molecule light chain, heavy chain and heavy chain/light chain fusion protein.

### Assays

T cell activating bispecific antigen binding molecules provided herein may be identified, screened for, or characterized for their physical/chemical properties and/or biological activities by various assays known in the art.

### Affinity Assays

The affinity of the T cell activating bispecific antigen binding molecule for an Fc receptor or a target antigen can be determined in accordance with the methods set forth in the Examples by surface plasmon resonance (SPR), using standard instrumentation such as a BIAcore instrument (GE Healthcare), and receptors or target proteins such as may be obtained by recombinant expression. Alternatively, binding of T cell activating bispecific antigen binding molecules for different receptors or target antigens may be evaluated using cell lines expressing the particular receptor or target antigen, for example by flow cytometry (FACS). A specific illustrative and exemplary embodiment for measuring binding affinity is described in the following and in the Examples below.

According to one embodiment, K_{D} is measured by surface plasmon resonance using a BIACORE® T100 machine (GE Healthcare) at 25° C.

To analyze the interaction between the Fc-portion and Fc receptors, His-tagged recombinant Fc-receptor is captured by an anti-Penta His antibody (Qiagen) immobilized on CM5 chips and the bispecific constructs are used as analytes. Briefly, carboxymethylated dextran biosensor chips (CM5, GE Healthcare) are activated with N-ethyl-N′-(3-dimethylaminopropyl)-carbodiimide hydrochloride (EDC) and N-hydroxysuccinimide (NHS) according to the supplier's instructions. Anti Penta-His antibody is diluted with 10 mM sodium acetate, pH 5.0, to 40 μg/ml before injection at a flow rate of 5 μl/min to achieve approximately 6500 response units (RU) of coupled protein. Following the injection of the ligand, 1 M ethanolamine is injected to block unreacted groups. Subsequently the Fc-receptor is captured for 60 s at 4 or 10 nM. For kinetic measurements, four-fold serial dilutions of the bispecific construct (range between 500 nM and 4000 nM) are injected in HBS-EP (GE Healthcare, 10 mM HEPES, 150 mM NaCl, 3 mM EDTA, 0.05% Surfactant P20, pH 7.4) at 25° C. at a flow rate of 30 μl/min for 120 s.

To determine the affinity to the target antigen, bispecific constructs are captured by an anti human Fab specific antibody (GE Healthcare) that is immobilized on an activated CM5-sensor chip surface as described for the anti Penta-His antibody. The final amount of coupled protein is is approximately 12000 RU. The bispecific constructs are captured for 90 s at 300 nM. The target antigens are passed through the flow cells for 180 s at a concentration range from 250 to 1000 nM with a flowrate of 30 μl/min. The dissociation is monitored for 180 s.

Bulk refractive index differences are corrected for by subtracting the response obtained on reference flow cell. The steady state response was used to derive the dissociation constant K_{D} by non-linear curve fitting of the Langmuir binding isotherm. Association rates (kₒₙ) and dissociation rates (k_{off}) are calculated using a simple one-to-one Langmuir binding model (BIACORE® T100 Evaluation Software version 1.1.1) by simultaneously fitting the association and dissociation sensorgrams. The equilibrium dissociation constant (K_{D}) is calculated as the ratio k_{off}/kₒₙ. See, e.g., Chen et al., J Mol Biol 293, 865-881 (1999).

### Activity Assays

Biological activity of the T cell activating bispecific antigen binding molecules of the invention can be measured by various assays as described in the Examples. Biological activities may for example include the induction of proliferation of T cells, the induction of signaling in T cells, the induction of expression of activation markers in T cells, the induction of cytokine secretion by T cells, the induction of lysis of target cells such as tumor cells, and the induction of tumor regression and/or the improvement of survival.

### Compositions, Formulations, and Routes of Administration

In a further aspect, the invention provides pharmaceutical compositions comprising any of the T cell activating bispecific antigen binding molecules provided herein, e.g., for use in any of the below therapeutic methods. In one embodiment, a pharmaceutical composition comprises any of the T cell activating bispecific antigen binding molecules provided herein and a pharmaceutically acceptable carrier. In another embodiment, a pharmaceutical composition comprises any of the T cell activating bispecific antigen binding molecules provided herein and at least one additional therapeutic agent, e.g., as described below.

Further provided is a method of producing a T cell activating bispecific antigen binding molecule of the invention in a form suitable for administration in vivo, the method comprising (a) obtaining a T cell activating bispecific antigen binding molecule according to the invention, and (b) formulating the T cell activating bispecific antigen binding molecule with at least one pharmaceutically acceptable carrier, whereby a preparation of T cell activating bispecific antigen binding molecule is formulated for administration in vivo.

Pharmaceutical compositions of the present invention comprise a therapeutically effective amount of one or more T cell activating bispecific antigen binding molecule dissolved or dispersed in a pharmaceutically acceptable carrier. The phrases “pharmaceutical or pharmacologically acceptable” refers to molecular entities and compositions that are generally non-toxic to recipients at the dosages and concentrations employed, i.e. do not produce an adverse, allergic or other untoward reaction when administered to an animal, such as, for example, a human, as appropriate. The preparation of a pharmaceutical composition that contains at least one T cell activating bispecific antigen binding molecule and optionally an additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards or corresponding authorities in other countries. Preferred compositions are lyophilized formulations or aqueous solutions. As used herein, “pharmaceutically acceptable carrier” includes any and all solvents, buffers, dispersion media, coatings, surfactants, antioxidants, preservatives (e.g. antibacterial agents, antifungal agents), isotonic agents, absorption delaying agents, salts, preservatives, antioxidants, proteins, drugs, drug stabilizers, polymers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The composition may comprise different types of carriers depending on whether it is to be administered in solid, liquid or aerosol form, and whether it need to be sterile for such routes of administration as injection. T cell activating bispecific antigen binding molecules of the present invention (and any additional therapeutic agent) can be administered intravenously, intradermally, intraarterially, intraperitoneally, intralesionally, intracranially, intraarticularly, intraprostatically, intrasplenically, intrarenally, intrapleurally, intratracheally, intranasally, intravitreally, intravaginally, intrarectally, intratumorally, intramuscularly, intraperitoneally, subcutaneously, subconjunctivally, intravesicularlly, mucosally, intrapericardially, intraumbilically, intraocularally, orally, topically, locally, by inhalation (e.g. aerosol inhalation), injection, infusion, continuous infusion, localized perfusion bathing target cells directly, via a catheter, via a lavage, in cremes, in lipid compositions (e.g. liposomes), or by other method or any combination of the forgoing as would be known to one of ordinary skill in the art (see, for example, Remington's Pharmaceutical Sciences, 18th Ed. Mack Printing Company, 1990, incorporated herein by reference). Parenteral administration, in particular intravenous injection, is most commonly used for administering polypeptide molecules such as the T cell activating bispecific antigen binding molecules of the invention.

Parenteral compositions include those designed for administration by injection, e.g. subcutaneous, intradermal, intralesional, intravenous, intraarterial intramuscular, intrathecal or intraperitoneal injection. For injection, the T cell activating bispecific antigen binding molecules of the invention may be formulated in aqueous solutions, preferably in physiologically compatible buffers such as Hanks' solution, Ringer's solution, or physiological saline buffer. The solution may contain formulatory agents such as suspending, stabilizing and/or dispersing agents. Alternatively, the T cell activating bispecific antigen binding molecules may be in powder form for constitution with a suitable vehicle, e.g., sterile pyrogen-free water, before use. Sterile injectable solutions are prepared by incorporating the T cell activating bispecific antigen binding molecules of the invention in the required amount in the appropriate solvent with various of the other ingredients enumerated below, as required. Sterility may be readily accomplished, e.g., by filtration through sterile filtration membranes. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein. Suitable pharmaceutically acceptable carriers include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Aqueous injection suspensions may contain compounds which increase the viscosity of the suspension, such as sodium carboxymethyl cellulose, sorbitol, dextran, or the like. Optionally, the suspension may also contain suitable stabilizers or agents which increase the solubility of the compounds to allow for the preparation of highly concentrated solutions. Additionally, suspensions of the active compounds may be prepared as appropriate oily injection suspensions. Suitable lipophilic solvents or vehicles include fatty oils such as sesame oil, or synthetic fatty acid esters, such as ethyl cleats or triglycerides, or liposomes.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences (18th Ed. Mack Printing Company, 1990). Sustained-release preparations may be prepared. Suitable examples of sustained-release preparations include semipermeable matrices of solid hydrophobic polymers containing the polypeptide, which matrices are in the form of shaped articles, e.g. films, or microcapsules. In particular embodiments, prolonged absorption of an injectable composition can be brought about by the use in the compositions of agents delaying absorption, such as, for example, aluminum monostearate, gelatin or combinations thereof.

In addition to the compositions described previously, the T cell activating bispecific antigen binding molecules may also be formulated as a depot preparation. Such long acting formulations may be administered by implantation (for example subcutaneously or intramuscularly) or by intramuscular injection. Thus, for example, the T cell activating bispecific antigen binding molecules may be formulated with suitable polymeric or hydrophobic materials (for example as an emulsion in an acceptable oil) or ion exchange resins, or as sparingly soluble derivatives, for example, as a sparingly soluble salt.

Pharmaceutical compositions comprising the T cell activating bispecific antigen binding molecules of the invention may be manufactured by means of conventional mixing, dissolving, emulsifying, encapsulating, entrapping or lyophilizing processes. Pharmaceutical compositions may be formulated in conventional manner using one or more physiologically acceptable carriers, diluents, excipients or auxiliaries which facilitate processing of the proteins into preparations that can be used pharmaceutically. Proper formulation is dependent upon the route of administration chosen.

The T cell activating bispecific antigen binding molecules may be formulated into a composition in a free acid or base, neutral or salt form. Pharmaceutically acceptable salts are salts that substantially retain the biological activity of the free acid or base. These include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Pharmaceutical salts tend to be more soluble in aqueous and other protic solvents than are the corresponding free base forms.

### Therapeutic Methods and Compositions

Any of the T cell activating bispecific antigen binding molecules provided herein may be used in therapeutic methods. T cell activating bispecific antigen binding molecules of the invention can be used as immunotherapeutic agents, for example in the treatment of cancers.

For use in therapeutic methods, T cell activating bispecific antigen binding molecules of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners.

In one aspect, T cell activating bispecific antigen binding molecules of the invention for use as a medicament are provided. In further aspects, T cell activating bispecific antigen binding molecules of the invention for use in treating a disease are provided. In certain embodiments, T cell activating bispecific antigen binding molecules of the invention for use in a method of treatment are provided. In one embodiment, the invention provides a T cell activating bispecific antigen binding molecule as described herein for use in the treatment of a disease in an individual in need thereof. In certain embodiments, the invention provides a T cell activating bispecific antigen binding molecule for use in a method of treating an individual having a disease comprising administering to the individual a therapeutically effective amount of the T cell activating bispecific antigen binding molecule. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. In further embodiments, the invention provides a T cell activating bispecific antigen binding molecule as described herein for use in inducing lysis of a target cell, particularly a tumor cell. In certain embodiments, the invention provides a T cell activating bispecific antigen binding molecule for use in a method of inducing lysis of a target cell, particularly a tumor cell, in an individual comprising administering to the individual an effective amount of the T cell activating bispecific antigen binding molecule to induce lysis of a target cell. An “individual” according to any of the above embodiments is a mammal, preferably a human.

In a further aspect, the invention provides for the use of a T cell activating bispecific antigen binding molecule of the invention in the manufacture or preparation of a medicament. In one embodiment the medicament is for the treatment of a disease in an individual in need thereof. In a further embodiment, the medicament is for use in a method of treating a disease comprising administering to an individual having the disease a therapeutically effective amount of the medicament. In certain embodiments the disease to be treated is a proliferative disorder. In a particular embodiment the disease is cancer. In one embodiment, the method further comprises administering to the individual a therapeutically effective amount of at least one additional therapeutic agent, e.g., an anti-cancer agent if the disease to be treated is cancer. In a further embodiment, the medicament is for inducing lysis of a target cell, particularly a tumor cell. In still a further embodiment, the medicament is for use in a method of inducing lysis of a target cell, particularly a tumor cell, in an individual comprising administering to the individual an effective amount of the medicament to induce lysis of a target cell. An “individual” according to any of the above embodiments may be a mammal, preferably a human.

In a further aspect, the invention provides a method for treating a disease. In one embodiment, the method comprises administering to an individual having such disease a therapeutically effective amount of a T cell activating bispecific antigen binding molecule of the invention. In one embodiment a composition is administered to said individual, comprising the T cell activating bispecific antigen binding molecule of the invention in a pharmaceutically acceptable form. In certain embodiments the disease to be treated is a proliferative disorder. In a particular embodiment the disease is cancer. In certain embodiments the method further comprises administering to the individual a therapeutically effective amount of at least one additional therapeutic agent, e.g., an anti-cancer agent if the disease to be treated is cancer. An “individual” according to any of the above embodiments may be a mammal, preferably a human.

In a further aspect, the invention provides a method for inducing lysis of a target cell, particularly a tumor cell. In one embodiment the method comprises contacting a target cell with a T cell activating bispecific antigen binding molecule of the invention in the presence of a T cell, particularly a cytotoxic T cell. In a further aspect, a method for inducing lysis of a target cell, particularly a tumor cell, in an individual is provided. In one such embodiment, the method comprises administering to the individual an effective amount of a T cell activating bispecific antigen binding molecule to induce lysis of a target cell. In one embodiment, an “individual” is a human.

In certain embodiments the disease to be treated is a proliferative disorder, particularly cancer. Non-limiting examples of cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, skin cancer, squamous cell carcinoma, bone cancer, and kidney cancer. Other cell proliferation disorders that can be treated using a T cell activating bispecific antigen binding molecule of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, spleen, thoracic region, and urogenital system. Also included are pre-cancerous conditions or lesions and cancer metastases. In certain embodiments the cancer is chosen from the group consisting of renal cell cancer, skin cancer, lung cancer, colorectal cancer, breast cancer, brain cancer, head and neck cancer. A skilled artisan readily recognizes that in many cases the T cell activating bispecific antigen binding molecule may not provide a cure but may only provide partial benefit. In some embodiments, a physiological change having some benefit is also considered therapeutically beneficial. Thus, in some embodiments, an amount of T cell activating bispecific antigen binding molecule that provides a physiological change is considered an “effective amount” or a “therapeutically effective amount”. The subject, patient, or individual in need of treatment is typically a mammal, more specifically a human.

In some embodiments, an effective amount of a T cell activating bispecific antigen binding molecule of the invention is administered to a cell. In other embodiments, a therapeutically effective amount of a T cell activating bispecific antigen binding molecule of the invention is administered to an individual for the treatment of disease.

For the prevention or treatment of disease, the appropriate dosage of a T cell activating bispecific antigen binding molecule of the invention (when used alone or in combination with one or more other additional therapeutic agents) will depend on the type of disease to be treated, the route of administration, the body weight of the patient, the type of T cell activating bispecific antigen binding molecule, the severity and course of the disease, whether the T cell activating bispecific antigen binding molecule is administered for preventive or therapeutic purposes, previous or concurrent therapeutic interventions, the patient's clinical history and response to the T cell activating bispecific antigen binding molecule, and the discretion of the attending physician. The practitioner responsible for administration will, in any event, determine the concentration of active ingredient(s) in a composition and appropriate dose(s) for the individual subject. Various dosing schedules including but not limited to single or multiple administrations over various time-points, bolus administration, and pulse infusion are contemplated herein.

The T cell activating bispecific antigen binding molecule is suitably administered to the patient at one time or over a series of treatments. Depending on the type and severity of the disease, about 1 μg/kg to 15 mg/kg (e.g. 0.1 mg/kg-10 mg/kg) of T cell activating bispecific antigen binding molecule can be an initial candidate dosage for administration to the patient, whether, for example, by one or more separate administrations, or by continuous infusion. One typical daily dosage might range from about 1 μg/kg to 100 mg/kg or more, depending on the factors mentioned above. For repeated administrations over several days or longer, depending on the condition, the treatment would generally be sustained until a desired suppression of disease symptoms occurs. One exemplary dosage of the T cell activating bispecific antigen binding molecule would be in the range from about 0.005 mg/kg to about 10 mg/kg. In other non-limiting examples, a dose may also comprise from about 1 microgram/kg body weight, about 5 microgram/kg body weight, about 10 microgram/kg body weight, about 50 microgram/kg body weight, about 100 microgram/kg body weight, about 200 microgram/kg body weight, about 350 microgram/kg body weight, about 500 microgram/kg body weight, about 1 milligram/kg body weight, about 5 milligram/kg body weight, about 10 milligram/kg body weight, about 50 milligram/kg body weight, about 100 milligram/kg body weight, about 200 milligram/kg body weight, about 350 milligram/kg body weight, about 500 milligram/kg body weight, to about 1000 mg/kg body weight or more per administration, and any range derivable therein. In non-limiting examples of a derivable range from the numbers listed herein, a range of about 5 mg/kg body weight to about 100 mg/kg body weight, about 5 microgram/kg body weight to about 500 milligram/kg body weight, etc., can be administered, based on the numbers described above. Thus, one or more doses of about 0.5 mg/kg, 2.0 mg/kg, 5.0 mg/kg or 10 mg/kg (or any combination thereof) may be administered to the patient. Such doses may be administered intermittently, e.g. every week or every three weeks (e.g. such that the patient receives from about two to about twenty, or e.g. about six doses of the T cell activating bispecific antigen binding molecule). An initial higher loading dose, followed by one or more lower doses may be administered. However, other dosage regimens may be useful. The progress of this therapy is easily monitored by conventional techniques and assays.

The T cell activating bispecific antigen binding molecules of the invention will generally be used in an amount effective to achieve the intended purpose. For use to treat or prevent a disease condition, the T cell activating bispecific antigen binding molecules of the invention, or pharmaceutical compositions thereof, are administered or applied in a therapeutically effective amount. Determination of a therapeutically effective amount is well within the capabilities of those skilled in the art, especially in light of the detailed disclosure provided herein.

For systemic administration, a therapeutically effective dose can be estimated initially from in vitro assays, such as cell culture assays. A dose can then be formulated in animal models to achieve a circulating concentration range that includes the IC₅₀ as determined in cell culture. Such information can be used to more accurately determine useful doses in humans.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the T cell activating bispecific antigen binding molecules which are sufficient to maintain therapeutic effect. Usual patient dosages for administration by injection range from about 0.1 to 50 mg/kg/day, typically from about 0.5 to 1 mg/kg/day. Therapeutically effective plasma levels may be achieved by administering multiple doses each day. Levels in plasma may be measured, for example, by HPLC.

In cases of local administration or selective uptake, the effective local concentration of the T cell activating bispecific antigen binding molecules may not be related to plasma concentration. One having skill in the art will be able to optimize therapeutically effective local dosages without undue experimentation.

A therapeutically effective dose of the T cell activating bispecific antigen binding molecules described herein will generally provide therapeutic benefit without causing substantial toxicity. Toxicity and therapeutic efficacy of a T cell activating bispecific antigen binding molecule can be determined by standard pharmaceutical procedures in cell culture or experimental animals. Cell culture assays and animal studies can be used to determine the LD₅₀ (the dose lethal to 50% of a population) and the ED₅₀ (the dose therapeutically effective in 50% of a population). The dose ratio between toxic and therapeutic effects is the therapeutic index, which can be expressed as the ratio LD₅₀/ED₅₀. T cell activating bispecific antigen binding molecules that exhibit large therapeutic indices are preferred. In one embodiment, the T cell activating bispecific antigen binding molecule according to the present invention exhibits a high therapeutic index. The data obtained from cell culture assays and animal studies can be used in formulating a range of dosages suitable for use in humans. The dosage lies preferably within a range of circulating concentrations that include the ED₅₀ with little or no toxicity. The dosage may vary within this range depending upon a variety of factors, e.g., the dosage form employed, the route of administration utilized, the condition of the subject, and the like. The exact formulation, route of administration and dosage can be chosen by the individual physician in view of the patient's condition (see, e.g., Fingl et al., 1975, in: The Pharmacological Basis of Therapeutics, Ch. 1, p. 1, incorporated herein by reference in its entirety).

The attending physician for patients treated with T cell activating bispecific antigen binding molecules of the invention would know how and when to terminate, interrupt, or adjust administration due to toxicity, organ dysfunction, and the like. Conversely, the attending physician would also know to adjust treatment to higher levels if the clinical response were not adequate (precluding toxicity). The magnitude of an administered dose in the management of the disorder of interest will vary with the severity of the condition to be treated, with the route of administration, and the like. The severity of the condition may, for example, be evaluated, in part, by standard prognostic evaluation methods. Further, the dose and perhaps dose frequency will also vary according to the age, body weight, and response of the individual patient.

### Other Agents and Treatments

The T cell activating bispecific antigen binding molecules of the invention may be administered in combination with one or more other agents in therapy. For instance, a T cell activating bispecific antigen binding molecule of the invention may be co-administered with at least one additional therapeutic agent. The term “therapeutic agent” encompasses any agent administered to treat a symptom or disease in an individual in need of such treatment. Such additional therapeutic agent may comprise any active ingredients suitable for the particular indication being treated, preferably those with complementary activities that do not adversely affect each other. In certain embodiments, an additional therapeutic agent is an immunomodulatory agent, a cytostatic agent, an inhibitor of cell adhesion, a cytotoxic agent, an activator of cell apoptosis, or an agent that increases the sensitivity of cells to apoptotic inducers. In a particular embodiment, the additional therapeutic agent is an anti-cancer agent, for example a microtubule disruptor, an antimetabolite, a topoisomerase inhibitor, a DNA intercalator, an alkylating agent, a hormonal therapy, a kinase inhibitor, a receptor antagonist, an activator of tumor cell apoptosis, or an antiangiogenic agent.

Such other agents are suitably present in combination in amounts that are effective for the purpose intended. The effective amount of such other agents depends on the amount of T cell activating bispecific antigen binding molecule used, the type of disorder or treatment, and other factors discussed above. The T cell activating bispecific antigen binding molecules are generally used in the same dosages and with administration routes as described herein, or about from 1 to 99% of the dosages described herein, or in any dosage and by any route that is empirically/clinically determined to be appropriate.

Such combination therapies noted above encompass combined administration (where two or more therapeutic agents are included in the same or separate compositions), and separate administration, in which case, administration of the T cell activating bispecific antigen binding molecule of the invention can occur prior to, simultaneously, and/or following, administration of the additional therapeutic agent and/or adjuvant. T cell activating bispecific antigen binding molecules of the invention can also be used in combination with radiation therapy.

### Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is a T cell activating bispecific antigen binding molecule of the invention. The label or package insert indicates that the composition is used for treating the condition of choice. Moreover, the article of manufacture may comprise (a) a first container with a composition contained therein, wherein the composition comprises a T cell activating bispecific antigen binding molecule of the invention; and (b) a second container with a composition contained therein, wherein the composition comprises a further cytotoxic or otherwise therapeutic agent. The article of manufacture in this embodiment of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The following are examples of methods and compositions of the invention. It is understood that various other embodiments may be practiced, given the general description provided above.

### General Methods

### Recombinant DNA Techniques

Standard methods were used to manipulate DNA as described in Sambrook et al., Molecular cloning: A laboratory manual; Cold Spring Harbor Laboratory Press, Cold Spring Harbor, N.Y., 1989. The molecular biological reagents were used according to the manufacturers' instructions. General information regarding the nucleotide sequences of human immunoglobulins light and heavy chains is given in: Kabat, E. A. et al., (1991) Sequences of Proteins of Immunological Interest, 5^{th} ed., NIH Publication No. 91-3242.

### DNA Sequencing

DNA sequences were determined by double strand sequencing.

### Gene Synthesis

Desired gene segments where required were either generated by PCR using appropriate templates or were synthesized by Geneart AG (Regensburg, Germany) from synthetic oligonucleotides and PCR products by automated gene synthesis. In cases where no exact gene sequence was available, oligonucleotide primers were designed based on sequences from closest homologues and the genes were isolated by RT-PCR from RNA originating from the appropriate tissue. The gene segments flanked by singular restriction endonuclease cleavage sites were cloned into standard cloning/sequencing vectors. The plasmid DNA was purified from transformed bacteria and concentration determined by UV spectroscopy. The DNA sequence of the subcloned gene fragments was confirmed by DNA sequencing. Gene segments were designed with suitable restriction sites to allow sub-cloning into the respective expression vectors. All constructs were designed with a 5′-end DNA sequence coding for a leader peptide which targets proteins for secretion in eukaryotic cells. SEQ ID NOs 154-162 give exemplary leader peptides and polynucleotide sequences encoding them, respectively.

Isolation of Primary Human Pan T Cells from PBMCs

Peripheral blood mononuclear cells (PBMCs) were prepared by Histopaque density centrifugation from enriched lymphocyte preparations (buffy coats) obtained from local blood banks or from fresh blood from healthy human donors. Briefly, blood was diluted with sterile PBS and carefully layered over a Histopaque gradient (Sigma, H8889). After centrifugation for 30 minutes at 450×g at room temperature (brake switched off), part of the plasma above the PBMC containing interphase was discarded. The PBMCs were transferred into new 50 ml Falcon tubes and tubes were filled up with PBS to a total volume of 50 ml. The mixture was centrifuged at room temperature for 10 minutes at 400×g (brake switched on). The supernatant was discarded and the PBMC pellet washed twice with sterile PBS (centrifugation steps at 4° C. for 10 minutes at 350×g). The resulting PBMC population was counted automatically (ViCell) and stored in RPMI1640 medium, containing 10% FCS and 1% L-alanyl-L-glutamine (Biochrom, K0302) at 37° C., 5% CO₂ in the incubator until assay start.

T cell enrichment from PBMCs was performed using the Pan T Cell Isolation Kit II (Miltenyi Biotec #130-091-156), according to the manufacturer's instructions. Briefly, the cell pellets were diluted in 40 μl cold buffer per 10 million cells (PBS with 0.5% BSA, 2 mM EDTA, sterile filtered) and incubated with 10 μl Biotin-Antibody Cocktail per 10 million cells for 10 min at 4° C. 30 μl cold buffer and 20 μl Anti-Biotin magnetic beads per 10 million cells were added, and the mixture incubated for another 15 min at 4° C. Cells were washed by adding 10-20x the current volume and a subsequent centrifugation step at 300×g for 10 min. Up to 100 million cells were resuspended in 500 μl buffer. Magnetic separation of unlabeled human pan T cells was performed using LS columns (Miltenyi Biotec #130-042-401) according to the manufacturer's instructions. The resulting T cell population was counted automatically (ViCell) and stored in AIM-V medium at 37° C., 5% CO₂ in the incubator until assay start (not longer than 24 h).

Isolation of Primary Human Naive T Cells from PBMCs

Peripheral blood mononuclar cells (PBMCs) were prepared by Histopaque density centrifugation from enriched lymphocyte preparations (buffy coats) obtained from local blood banks or from fresh blood from healthy human donors. T-cell enrichment from PBMCs was performed using the Naive CD8⁺ T cell isolation Kit from Miltenyi Biotec (#130-093-244), according to the manufacturer's instructions, but skipping the last isolation step of CD8⁺ T cells (also see description for the isolation of primary human pan T cells).

Isolation of Murine Pan T Cells from Splenocytes

Spleens were isolated from C57BL/6 mice, transferred into a GentleMACS C-tube (Miltenyi Biotech #130-093-237) containing MACS buffer (PBS+0.5% BSA+2 mM EDTA) and dissociated with the GentleMACS Dissociator to obtain single-cell suspensions according to the manufacturer's instructions. The cell suspension was passed through a pre-separation filter to remove remaining undissociated tissue particles. After centrifugation at 400×g for 4 min at 4° C., ACK Lysis Buffer was added to lyse red blood cells (incubation for 5 min at room temperature). The remaining cells were washed with MACS buffer twice, counted and used for the isolation of murine pan T cells. The negative (magnetic) selection was performed using the Pan T Cell Isolation Kit from Miltenyi Biotec (#130-090-861), following the manufacturer's instructions. The resulting T cell population was automatically counted (ViCell) and immediately used for further assays.

Isolation of Primary Cynomolgus PBMCs from Heparinized Blood

Peripheral blood mononuclar cells (PBMCs) were prepared by density centrifugation from fresh blood from healthy cynomolgus donors, as follows: Heparinized blood was diluted 1:3 with sterile PBS, and Lymphoprep medium (Axon Lab #1114545) was diluted to 90% with sterile PBS. Two volumes of the diluted blood were layered over one volume of the diluted density gradient and the PBMC fraction was separated by centrifugation for 30 min at 520×g, without brake, at room temperature. The PBMC band was transferred into a fresh 50 ml Falcon tube and washed with sterile PBS by centrifugation for 10 min at 400×g at 4° C. One low-speed centrifugation was performed to remove the platelets (15 min at 150×g, 4° C.), and the resulting PBMC population was automatically counted (ViCell) and immediately used for further assays.

### Target Cells

For the assessment of MCSP-targeting bispecific antigen binding molecules, the following tumor cell lines were used: the human melanoma cell line WM266-4 (ATCC #CRL-1676), derived from a metastatic site of a malignant melanoma and expressing high levels of human MCSP; the human melanoma cell line MV-3 (a kind gift from The Radboud University Nijmegen Medical Centre), expressing medium levels of human MCSP; the human malignant melanoma (primary tumour) cell line A375 (ECACC #88113005) expressing high levels of MCSP; the human colon carcinoma cell line HCT-116 (ATCC #CCL-247) that does not express MCSP; and the human Caucasian colon adenocarcinoma cell line LS180 (ECACC #87021202) that does not express MCSP.

For the assessment of CEA-targeting bispecific antigen binding molecules, the following tumor cell lines were used: the human gastric cancer cell line MKN45 (DSMZ #ACC 409), expressing very high levels of human CEA; the human pancreas adenocarcinoma cell line HPAF-II (kind gift of Roche Nutley), expressing high levels of human CEA; the human primary pancreatic adenocarcinoma cell line BxPC-3 (ECACC #93120816) expressing medium levels of human CEA; the human female Caucasian colon adenocarcinoma cell line LS-174T (ECACC #87060401), expressing medium levels of human CEA; the human pancreas adenocarcinoma cell line ASPC-1 (ECACC #96020930) expressing low levels of human CEA; the human epithelioid pancreatic carcinoma cell line Panc-1 (ATCC #CRL-1469), expressing (very) low levels of human CEA; the human colon carcinoma cell line HCT-116 (ATCC #CCL-247) that does not express CEA; a human adenocarcinomic alveolar basal epithelial cell line A549-huCEA that was stably transfected in-house to express human CEA; and a murine colon carcinoma cell line MC38-huCEA, that was engineered in-house to stably express human CEA.

In addition, a human T cell leukaemia cell line, Jurkat (ATCC #TIB-152), was used to assess binding of different bispecific constructs to human CD3 on cells.

### Example 1

### Preparation, Purification and Characterization of Bispecific Antigen Binding Molecules

The heavy and light chain variable region sequences were subcloned in frame with either the constant heavy chain or the constant light chain pre-inserted into the respective recipient mammalian expression vector. The antibody expression was driven by an MPSV promoter and a synthetic polyA signal sequence is located at the 3′ end of the CDS. In addition each vector contained an EBV OriP sequence.

The molecules were produced by co-transfecting HEK293 EBNA cells with the mammalian expression vectors. Exponentially growing HEK293 EBNA cells were transfected using the calcium phosphate method. Alternatively, HEK293 EBNA cells growing in suspension were transfected using polyethylenimine (PEI). For preparation of “1+1 IgG scFab, one armed/one armed inverted” constructs, cells were transfected with the corresponding expression vectors in a 1:1:1 ratio (“vector heavy chain”:“vector light chain”:“vector heavy chain-scFab”). For preparation of “2+1 IgG scFab” constructs, cells were transfected with the corresponding expression vectors in a 1:2:1 ratio (“vector heavy chain”:“vector light chain”:“vector heavy chain-scFab”). For preparation of “1+1 IgG Crossfab” constructs, cells were transfected with the corresponding expression vectors in a 1:1:1:1 ratio (“vector second heavy chain”:“vector first light chain”:“vector light chain Crossfab”:“vector first heavy chain-heavy chain Crossfab”). For preparation of “2+1 IgG Crossfab” constructs cells were transfected with the corresponding expression vectors in a 1:2:1:1 ratio (“vector second heavy chain”:“vector light chain”:“vector first heavy chain-heavy chain Crossfab)”:“vector light chain Crossfab”. For preparation of the “2+1 IgG Crossfab, linked light chain” construct, cells were transfected with the corresponding expression vectors in a 1:1:1:1 ratio (“vector heavy chain”:“vector light chain”:“vector heavy chain (CrossFab-Fab-Fc)”:“vector linked light chain”). For preparation of the “1+1 CrossMab” construct, cells were transfected with the corresponding expression vectors in a 1:1:1:1 ratio (“vector first heavy chain”:“vector second heavy chain”:“vector first light chain”:“vector second light chain”). For preparation of the “1+1 IgG Crossfab light chain fusion” construct, cells were transfected with the corresponding expression vectors in a 1:1:1:1 ratio (“vector first heavy chain”:“vector second heavy chain”:“vector light chain Crossfab”:“vector second light chain”).

For transfection using calcium phosphate cells were grown as adherent monolayer cultures in T-flasks using DMEM culture medium supplemented with 10% (v/v) FCS, and transfected when they were between 50 and 80% confluent. For the transfection of a T150 flask, 15 million cells were seeded 24 hours before transfection in 25 ml DMEM culture medium supplemented with FCS (at 10% v/v final), and cells were placed at 37° C. in an incubator with a 5% CO₂ atmosphere overnight. For each T150 flask to be transfected, a solution of DNA, CaCl₂ and water was prepared by mixing 94 μg total plasmid vector DNA divided in the corresponding ratio, water to a final volume of 469 μl and 469 μl of a 1 M CaCl₂ solution. To this solution, 938 μl of a 50 mM HEPES, 280 mM NaCl, 1.5 mM Na₂HPO₄ solution at pH 7.05 were added, mixed immediately for 10 s and left to stand at room temperature for 20 s. The suspension was diluted with 10 ml of DMEM supplemented with 2% (v/v) FCS, and added to the T150 in place of the existing medium. Subsequently, additional 13 ml of transfection medium were added. The cells were incubated at 37° C., 5% CO₂ for about 17 to 20 hours, then medium was replaced with 25 ml DMEM, 10% FCS. The conditioned culture medium was harvested approximately 7 days post-media exchange by centrifugation for 15 min at 210×g, sterile filtered (0.22·m filter), supplemented with sodium azide to a final concentration of 0.01% (w/v), and kept at 4° C.

For transfection using polyethylenimine (PEI) HEK293 EBNA cells were cultivated in suspension in serum free CD CHO culture medium. For the production in 500 ml shake flasks, 400 million HEK293 EBNA cells were seeded 24 hours before transfection. For transfection cells were centrifuged for 5 min at 210×g, and supernatant was replaced by 20 ml pre-warmed CD CHO medium. Expression vectors were mixed in 20 ml CD CHO medium to a final amount of 200 μg DNA. After addition of 540 μl PEI, the mixture was vortexed for 15 s and subsequently incubated for 10 min at room temperature. Afterwards cells were mixed with the DNA/PEI solution, transferred to a 500 ml shake flask and incubated for 3 hours at 37° C. in an incubator with a 5% CO₂ atmosphere. After the incubation time 160 ml F17 medium was added and cells were cultivated for 24 hours. One day after transfection 1 mM valproic acid and 7% Feed 1 (Lonza) were added. After a cultivation of 7 days, supernatant was collected for purification by centrifugation for 15 min at 210×g, the solution was sterile filtered (0.22 μm filter), supplemented with sodium azide to a final concentration of 0.01% w/v, and kept at 4° C.

The secreted proteins were purified from cell culture supernatants by Protein A affinity chromatography, followed by a size exclusion chromatography step.

For affinity chromatography supernatant was loaded on a HiTrap Protein A HP column (CV=5 ml, GE Healthcare) equilibrated with 25 ml 20 mM sodium phosphate, 20 mM sodium citrate, pH 7.5 or 40 ml 20 mM sodium phosphate, 20 mM sodium citrate, 0.5 M sodium chloride, pH 7.5. Unbound protein was removed by washing with at least ten column volumes 20 mM sodium phosphate, 20 mM sodium citrate, 0.5 M sodium chloride pH 7.5, followed by an additional wash step using six column volumes 10 mM sodium phosphate, 20 mM sodium citrate, 0.5 M sodium chloride pH 5.45. Subsequently, the column was washed with 20 ml 10 mM MES, 100 mM sodium chloride, pH 5.0, and target protein was eluted in six column volumes 20 mM sodium citrate, 100 mM sodium chloride, 100 mM glycine, pH 3.0. Alternatively, target protein was eluted using a gradient over 20 column volumes from 20 mM sodium citrate, 0.5 M sodium chloride, pH 7.5 to 20 mM sodium citrate, 0.5 M sodium chloride, pH 2.5. The protein solution was neutralized by adding 1/10 of 0.5 M sodium phosphate, pH 8. The target protein was concentrated and filtrated prior to loading on a HiLoad Superdex 200 column (GE Healthcare) equilibrated with 25 mM potassium phosphate, 125 mM sodium chloride, 100 mM glycine solution of pH 6.7. For the purification of 1+1 IgG Crossfab the column was equilibrated with 20 mM histidine, 140 mM sodium chloride solution of pH 6.0.

The protein concentration of purified protein samples was determined by measuring the optical density (OD) at 280 nm, using the molar extinction coefficient calculated on the basis of the amino acid sequence. Purity and molecular weight of the bispecific constructs were analyzed by SDS-PAGE in the presence and absence of a reducing agent (5 mM 1,4-dithiotreitol) and staining with Coomassie (SimpleBlue™ SafeStain from Invitrogen) using the NuPAGE® Pre-Cast gel system (Invitrogen, USA) was used according to the manufacturer's instructions (4-12% Tris-Acetate gels or 4-12% Bis-Tris). Alternatively, purity and molecular weight of molecules were analyzed by CE-SDS analyses in the presence and absence of a reducing agent, using the Caliper LabChip GXII system (Caliper Lifescience) according to the manufacturer's instructions.

The aggregate content of the protein samples was analyzed using a Superdex 200 10/300GL analytical size-exclusion chromatography column (GE Healthcare) in 2 mM MOPS, 150 mM NaCl, 0.02% (w/v) NaN₃, pH 7.3 running buffer at 25° C. Alternatively, the aggregate content of antibody samples was analyzed using a TSKgel G3000 SW XL analytical size-exclusion column (Tosoh) in 25 mM K₂HPO₄, 125 mM NaCl, 200 mM L-arginine monohydrocloride, 0.02% (w/v) NaN₃, pH 6.7 running buffer at 25° C.

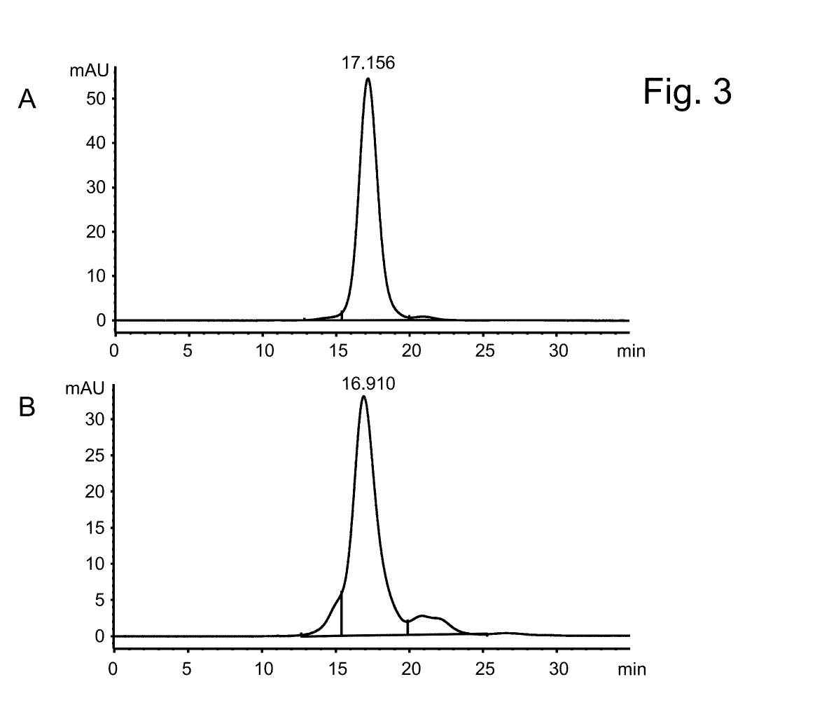

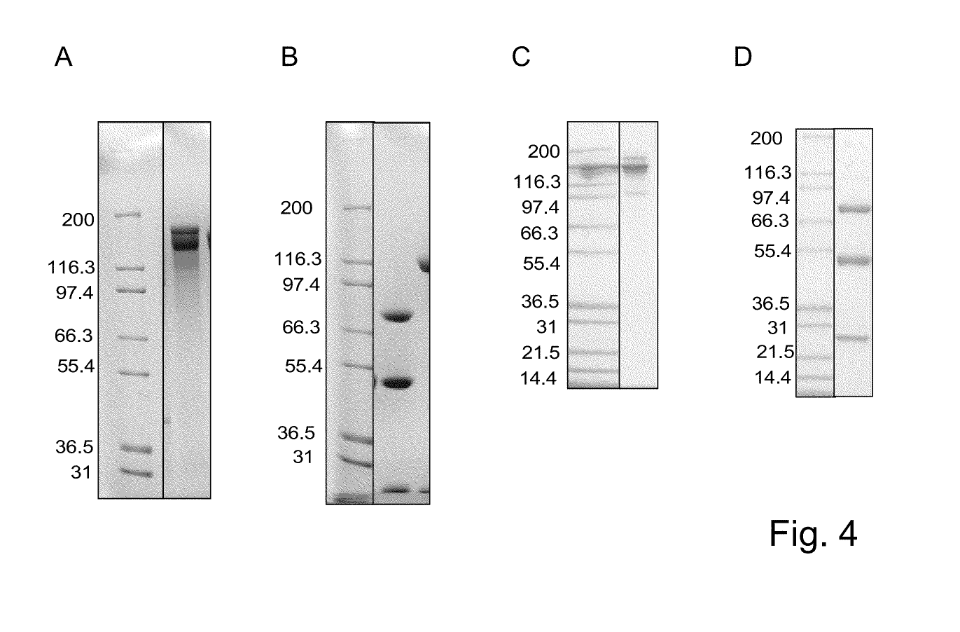

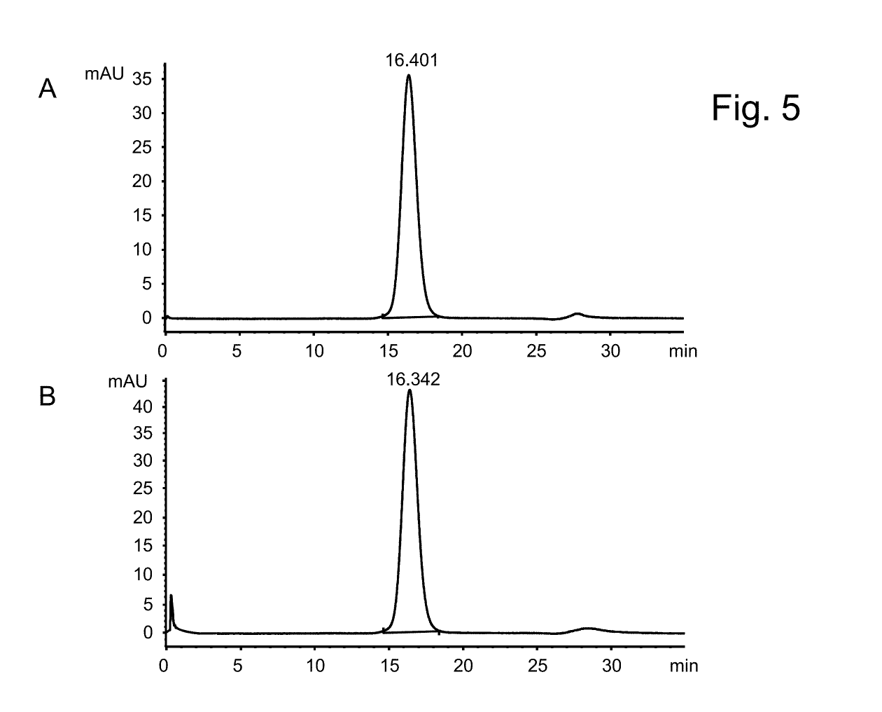

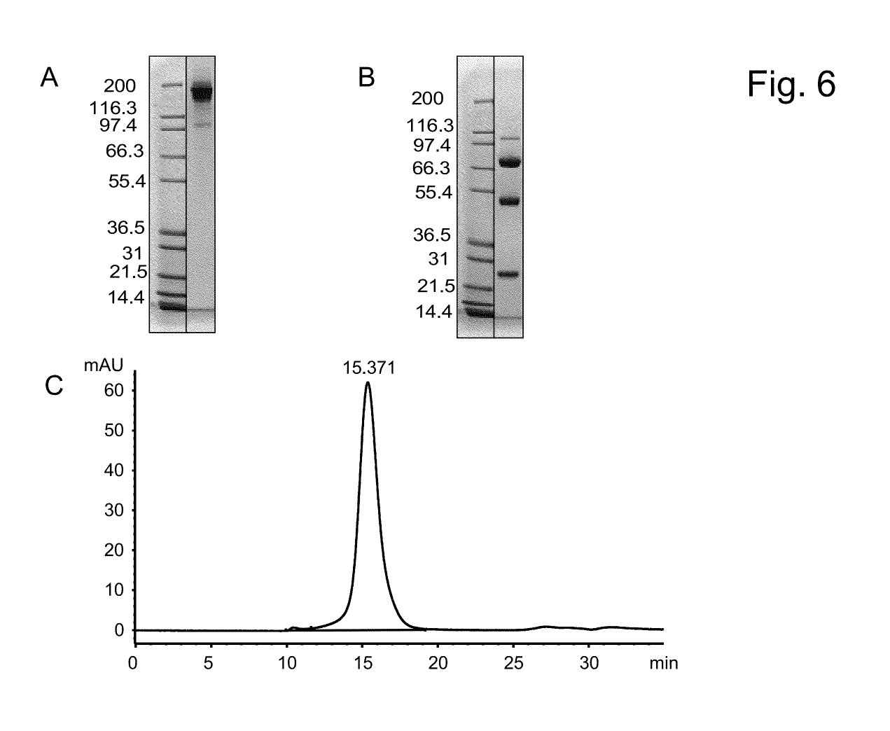

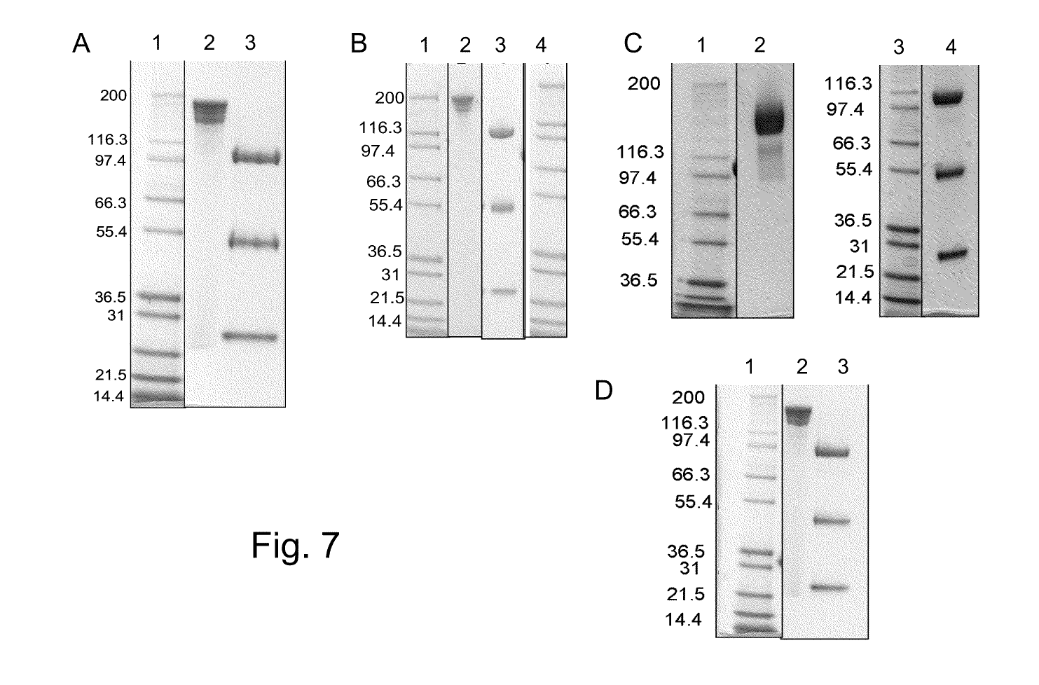

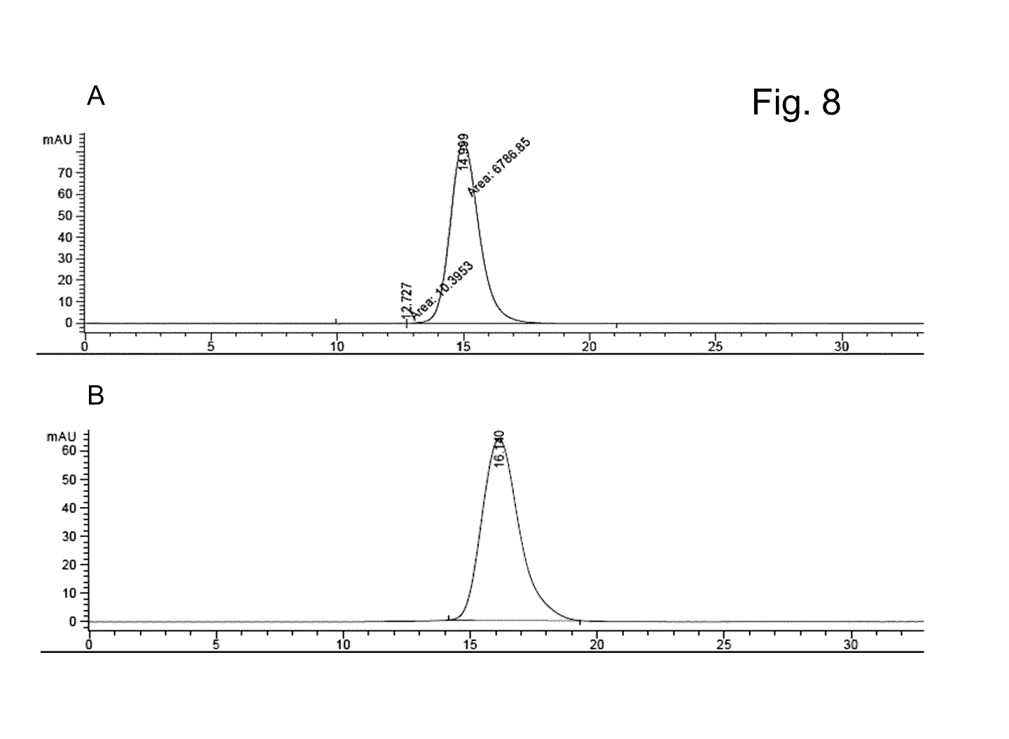

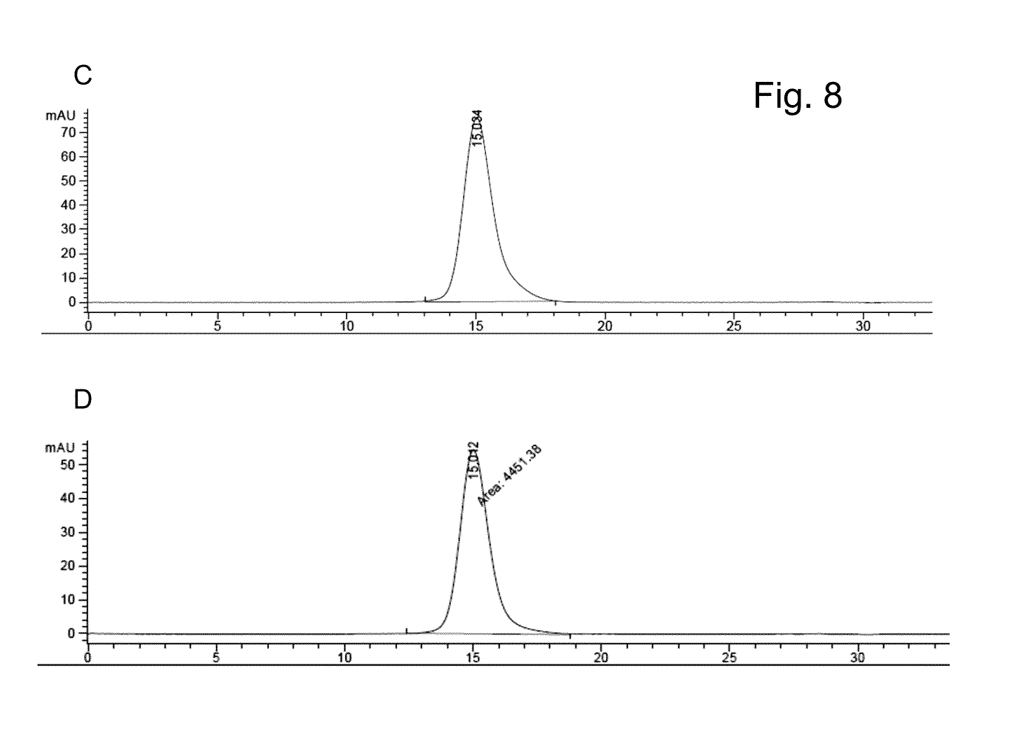

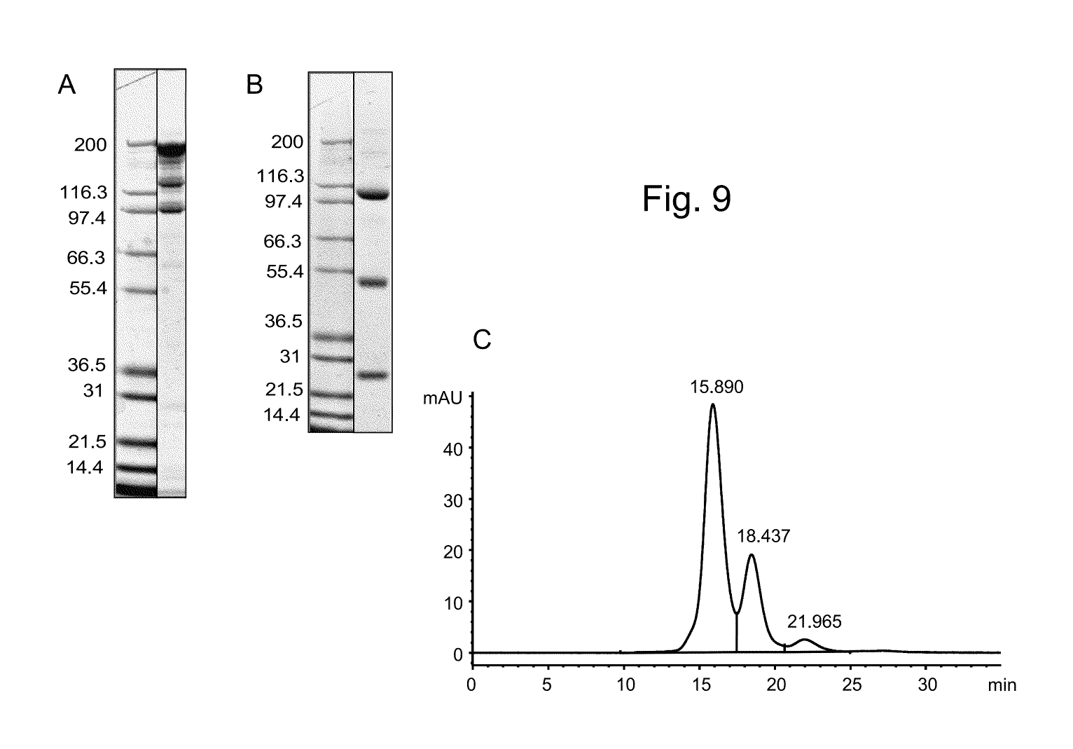

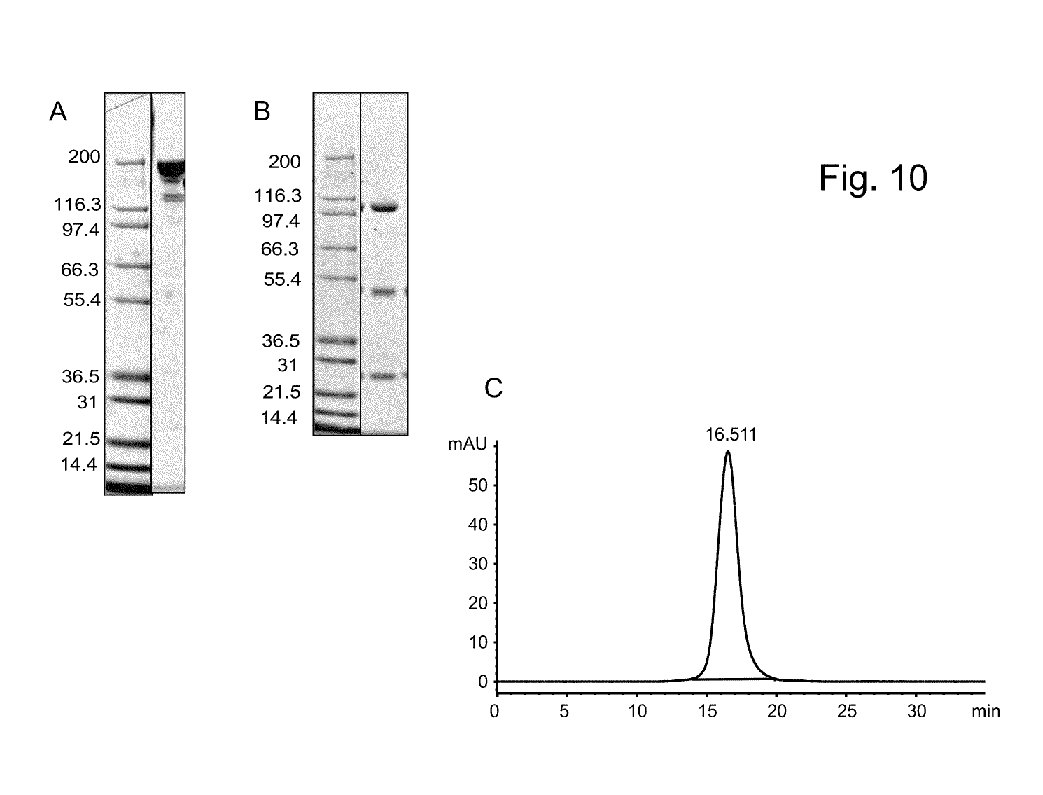

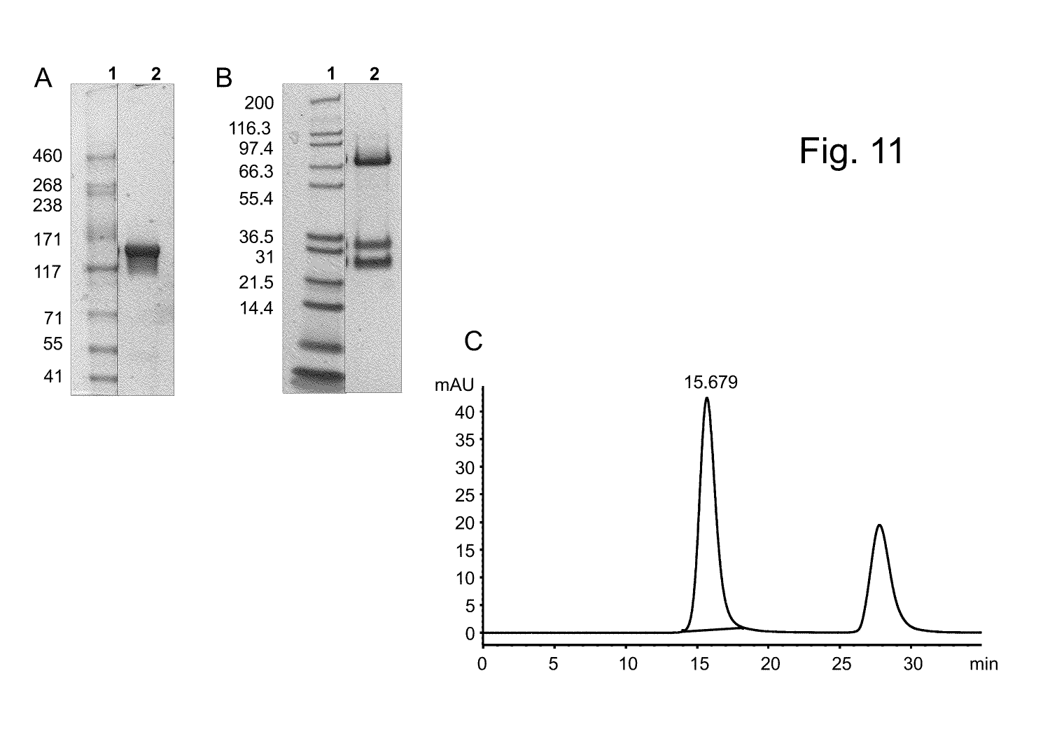

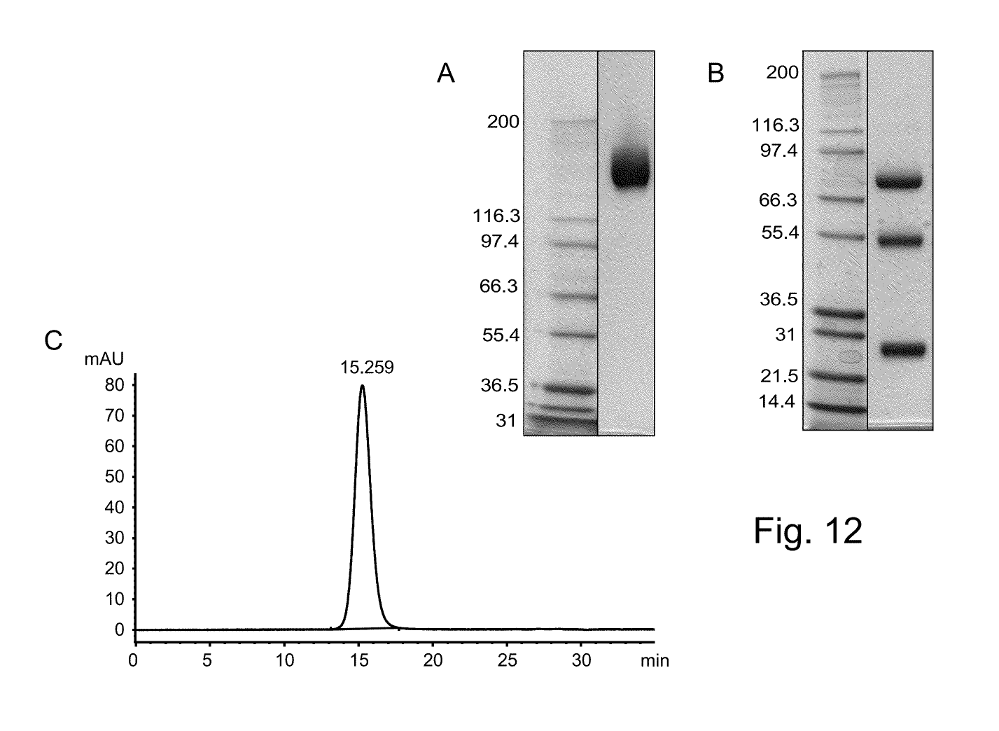

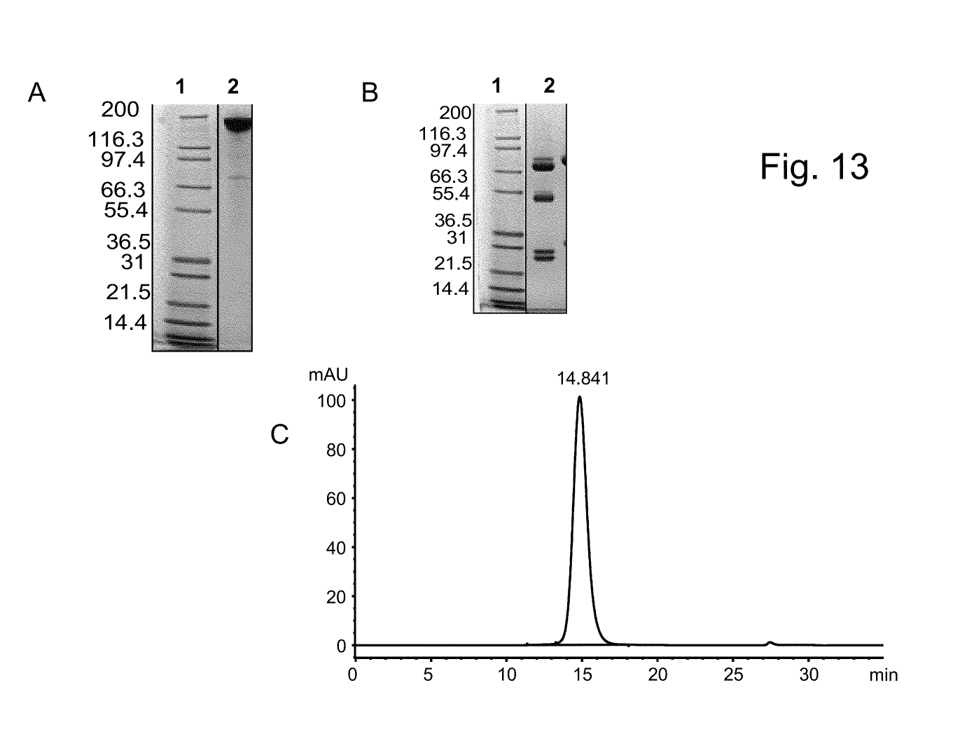

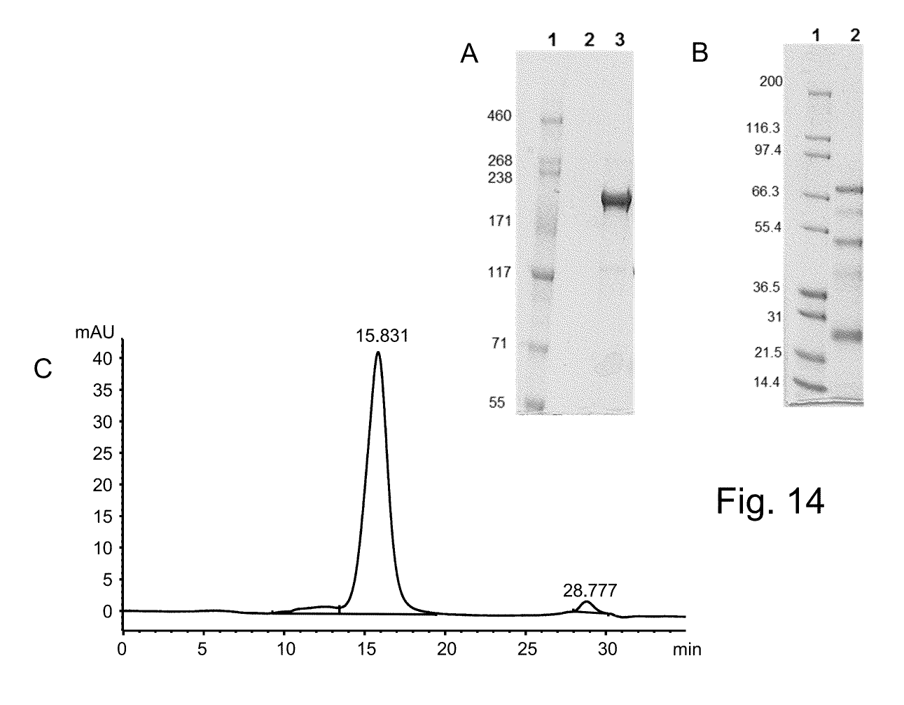

FIGS. 2-14 show the results of the SDS PAGE and analytical size exclusion chromatography and Table 2A shows the yields, aggregate content after Protein A, and final monomer content of the preparations of the different bispecific constructs.

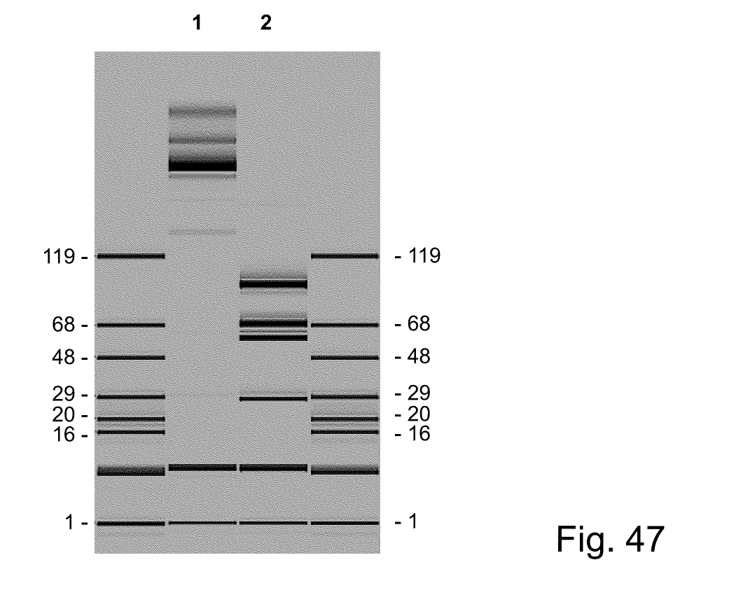

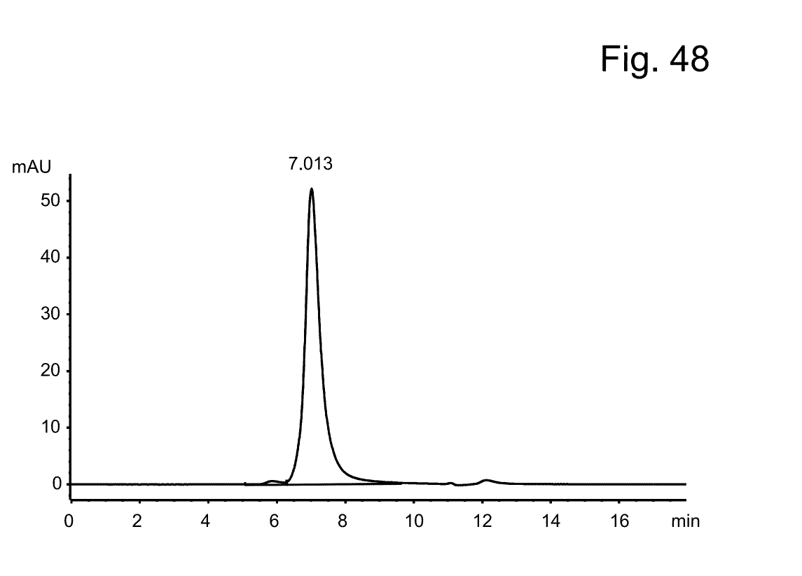

FIG. 47 shows the result of the CE-SDS analyses of the anti-CD3/anti-MCSP bispecific “2+1 IgG Crossfab, linked light chain” construct (see SEQ ID NOs 3, 5, 29 and 179). 2 μg sample was used for analyses. FIG. 48 shows the result of the analytical size exclusion chromatography of the final product (20 μg sample injected).

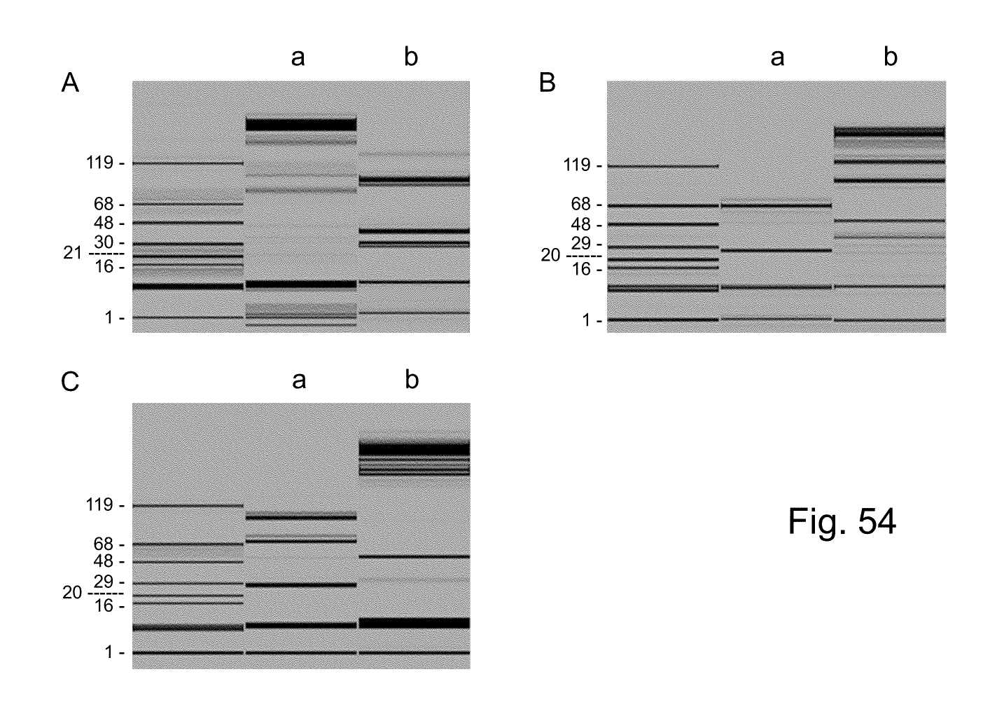

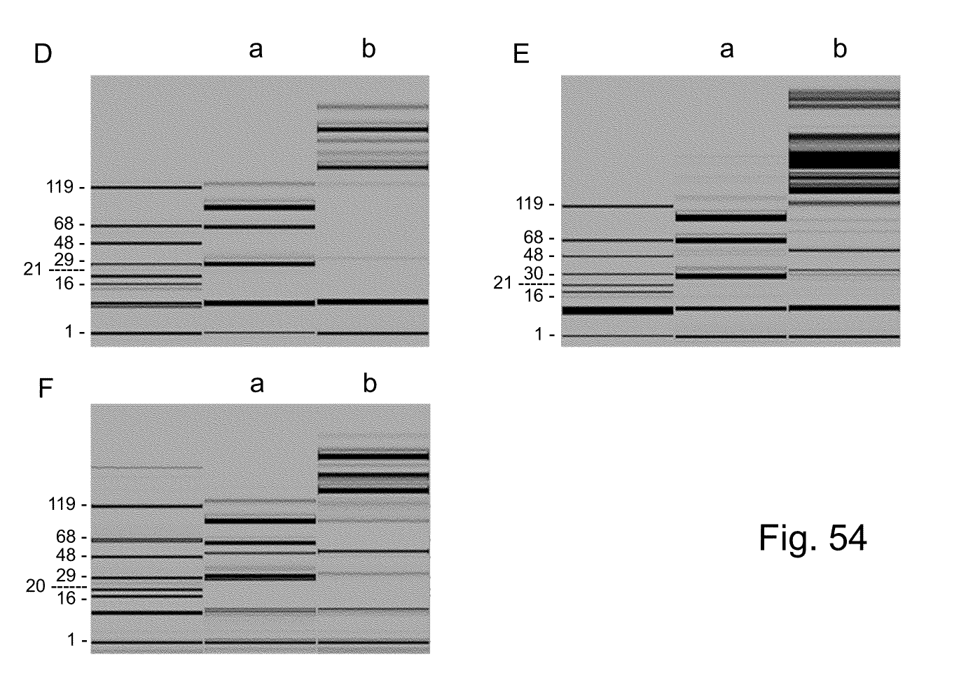

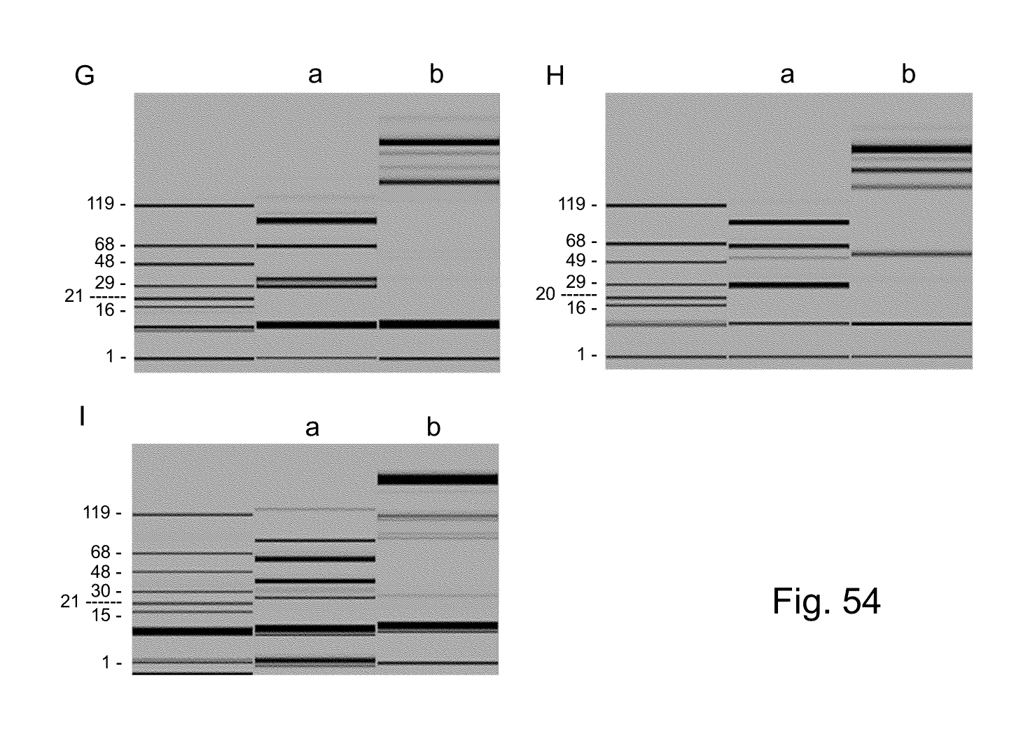

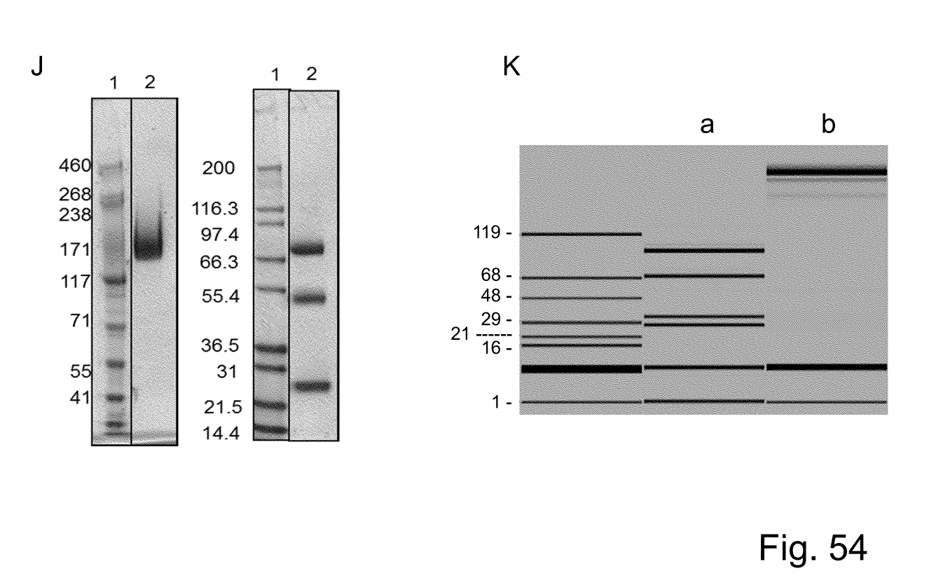

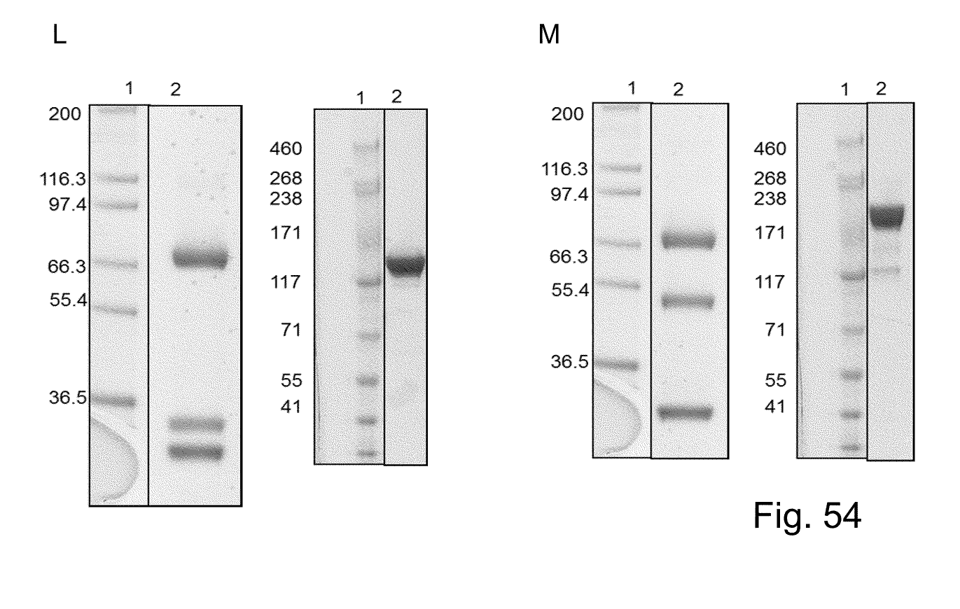

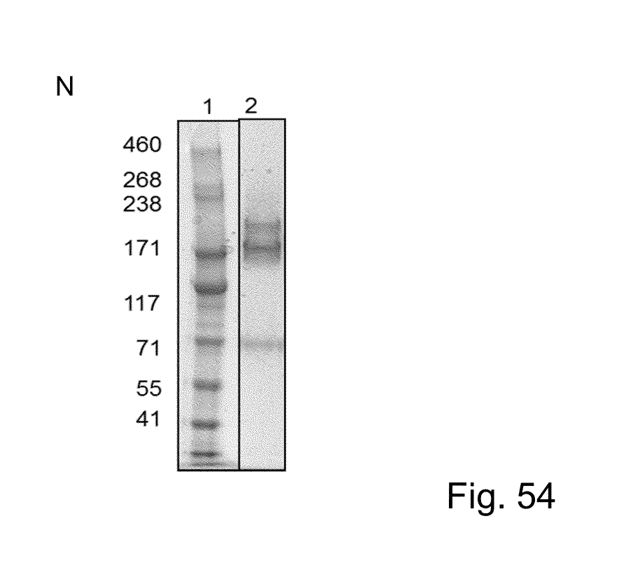

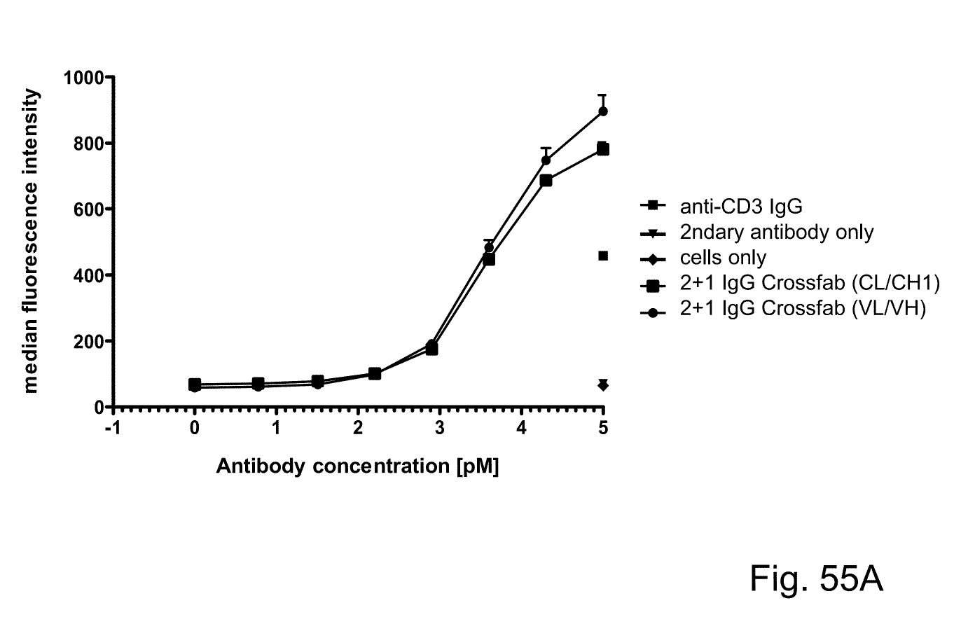

FIG. 54 shows the results of the CE-SDS and SDS PAGE analyses of various constructs, and Table 2A shows the yields, aggregate content after Protein A and final monomer content of the preparations of the different bispecific constructs.

TABLE 2A
Yields, aggregate content after Protein A and final monomer content.

| | | Aggre- | | | |
|---|---|---|---|---|---|
| | | gate | | | |
| | | content | | | |
| | | after | | | Mon- |
| | Yield | Protein | HMW | LMW | omer |
| Construct | [mg/l] | A [%] | [%] | [%] | [%] |
| MCSP | | | | | |
| 2 + 1 IgG Crossfab; VH/VL | 12.8 | 2.2 | 0 | 0 | 100 |
| exchange (LC007/V9) | | | | | |
| (SEQ ID NOs 3, 5, 29, 33) | | | | | |
| 2 + 1 IgG Crossfab; VH/VL | 3.2 | 5.7 | 0.4 | 0 | 99.6 |
| exchange (LC007/FN18) | | | | | |
| (SEQ ID NOs 3, 5, 35, 37) | | | | | |
| 2 + 1 IgG scFab, P329G | 11.9 | 23 | 0.3 | 0 | 99.7 |
| LALA (SEQ ID NOs 5, | | | | | |
| 21, 23) | | | | | |
| 2 + 1 IgG scFab, LALA | 9 | 23 | 0 | 0 | 100 |
| (SEQ ID NOs 5, 17, 19) | | | | | |
| 2 + 1 IgG scFab, P329G | 12.9 | 32.7 | 0 | 0 | 100 |
| LALA N297D (SEQ ID | | | | | |
| NOs 5, 25, 27) | | | | | |
| 2 + 1 IgG scFab, wt | 15.5 | 31.8 | 0 | 0 | 100 |
| (SEQ ID NOs 5, 13, 15) | | | | | |
| 1 + 1 IgG scFab | 7 | 24.5 | 0 | 0 | 100 |
| (SEQ ID NOs 5, 21, 213) | | | | | |
| 1 + 1 IgG scFab “one armed” | 7.6 | 43.7 | 2.3 | 0 | 97.7 |
| (SEQ ID NOs 1, 3, 5) | | | | | |
| 1 + 1 IgG scFab “one armed | 1 | 27 | 7.1 | 9.1 | 83.8 |
| inverted” (SEQ ID NOs 7, | | | | | |
| 9, 11) | | | | | |
| 1 + 1 IgG Crossfab; VH/VL | 9.8 | 0 | 0 | 0 | 100 |
| exchange (LC007/V9) | | | | | |
| (SEQ ID NOs 5, 29, 31, 33) | | | | | |
| 2 + 1 IgG Crossfab, linked | 0.54 | 40 | 1.4 | 0 | 98.6 |
| light chain; VL/VH | | | | | |
| exchange (LC007/V9) | | | | | |
| (SEQ ID NOs 3, 5, 29, 179) | | | | | |
| 1 + 1 IgG Crossfab; VL/VH | 6.61 | 8.5 | 0 | 0 | 100 |
| exchange (LC007/V9) | | | | | |
| (SEQ ID NOs 5, 29, | | | | | |
| 33, 181) | | | | | |
| 1 + 1 CrossMab; CL/CH1 | 6.91 | 10.5 | 1.3 | 1.7 | 97 |
| exchange (LC00/V9) | | | | | |
| (SEQ ID NOs 5, 23, | | | | | |
| 183, 185) | | | | | |
| 2 + 1 IgG Crossfab, inverted; | 9.45 | 6.1 | 0.8 | 0 | 99.2 |
| CL/CH1 exchange | | | | | |
| (LC007/V9) | | | | | |
| (SEQ ID NOs 5, 23, | | | | | |
| 183, 187) | | | | | |
| 2 + 1 IgG Crossfab; VL/VH | 36.6 | 0 | 9.5 | 35.3 | 55.2 |
| exchange (M4-3 ML2/V9) | | | | | |
| (SEQ ID NOs 33, 189, | | | | | |
| 191, 193) | | | | | |
| 2 + 1 IgG Crossfab; CL/CH1 | 2.62 | 12 | 2.8 | 0 | 97.2 |
| exchange (M4-3 ML2/V9) | | | | | |
| (SEQ ID NOs 183, 189, | | | | | |
| 193, 195) | | | | | |
| 2 + 1 IgG Crossfab; CL/CH1 | 29.75 | 0 | 0 | 0 | 100 |
| exchange (M4-3 ML2/H2C) | | | | | |
| (SEQ ID NOs 189, 193, | | | | | |
| 199, 201) | | | | | |
| 2 + 1 IgG Crossfab; | 1.2 | 0 | 1.25 | 1.65 | 97.1 |
| CL/CH1 exchange | | | | | |
| (LC007/anti-CD3) | | | | | |
| (SEQ ID NOs 5, 23, | | | | | |
| 215, 217) | | | | | |
| 2 + 1 IgG Crossfab, | 7.82 | 0.5 | 0 | 0 | 100 |
| inverted; CL/CH1 | | | | | |
| exchange (LC007/ | | | | | |
| anti-CD3) | | | | | |
| (SEQ ID NOs 5, 23, | | | | | |
| 215, 219) | | | | | |
| EGFR | | | | | |
| 2 + 1 IgG scFab | 5.2 | 53 | 0 | 30 | 70 |
| (SEQ ID NOs 45, 47, 53) | | | | | |
| 1 + 1 IgG scFab | 3.4 | 66.6 | 0 | 1.6 | 98.4 |
| (SEQ ID NOs 47, 53, 213) | | | | | |
| 1 + 1 IgG scFab “one armed” | 9.05 | 60.8 | 0 | 0 | 100 |
| (SEQ ID NOs 43, 45, 47) | | | | | |
| 1 + 1 IgG scFab “one armed | 3.87 | 58.8 | 0 | 0 | 100 |
| inverted” (SEQ ID NOs 11, | | | | | |
| 49, 51) | | | | | |
| FAP | | | | | |
| 2 + 1 IgG scFab | 12.57 | 53 | 0 | 0 | 100 |
| (SEQ ID NOs 57, 59, 61) | | | | | |
| 1 + 1 IgG scFab | 17.95 | 41 | 0.4 | 0 | 99.6 |
| (SEQ ID NOs 57, 61, 213) | | | | | |
| 1 + 1 IgG scFab “one armed | 2.44 | 69 | 0.6 | 0 | 99.4 |
| inverted” (SEQ ID NOs 11, | | | | | |
| 51, 55) | | | | | |
| CEA | | | | | |
| 2 + 1 IgG Crossfab, inverted; | 0.34 | 13 | 4.4 | 0 | 95.6 |
| VL/VH exchange | | | | | |
| (CH1A1A/V9) | | | | | |
| (SEQ ID NOs 33, 63, | | | | | |
| 65, 67) | | | | | |
| 2 + 1 IgG Crossfab, inverted; | 12.7 | 43 | 0 | 0 | 100 |
| CL/CH1 exchange | | | | | |
| (CH1A1A/V9) | | | | | |
| (SEQ ID NOs 65, 67, | | | | | |
| 183, 197) | | | | | |
| 2 + 1 IgG Crossfab, inverted; | 7.1 | 20 | 0 | 0 | 100 |
| CL/CH1 exchange | | | | | |
| (431/26/V9) | | | | | |
| (SEQ ID NOs 183, 203, | | | | | |
| 205, 207) | | | | | |
| 1 + 1 IgG-Crossfab light | 7.85 | 27 | 4.3 | 3.2 | 92.5 |
| chain fusion (CH1A1A/V9) | | | | | |
| (SEQ ID NOs 183, 209, | | | | | |
| 211, 213) | | | | | |

As controls, bispecific antigen binding molecules were generated in the prior art tandem scFv format (“(scFv)₂”) and by fusing a tandem scFv to an Fc domain (“(scFv)₂-Fc”). The molecules were produced in HEK293-EBNA cells and purified by Protein A affinity chromatography followed by a size exclusion chromatographic step in an analogous manner as described above for the bispecific antigen binding molecules of the invention. Due to high aggregate formation, some of the samples had to be further purified by applying eluted and concentrated samples from the HiLoad Superdex 200 column (GE Healthcare) to a Superdex 10/300 GL column (GE Healthcare) equilibrated with 20 mM histidine, 140 mM sodium chloride, pH 6.7 in order to obtain protein with high monomer content. Subsequently, protein concentration, purity and molecular weight, and aggregate content were determined as described above.

Yields, aggregate content after the first purification step, and final monomer content for the control molecules is shown in Table 2B. Comparison of the aggregate content after the first purification step (Protein A) indicates the superior stability of the IgG Crossfab and IgG scFab constructs compared to the “(scFv)₂-Fc” and the disulfide bridge-stabilized “(dsscFv)₂-Fc” molecules.

TABLE 2B
Yields, aggregate content after Protein A and final monomer content.

| | | Aggregates | | | |
|---|---|---|---|---|---|
| | | after | Final | | |
| | Yield | Protein | HMW | LMW | Monomer |
| Construct | [mg/l] | A [%] | [%] | [%] | [%] |
| (scFv)₂-Fc | 76.5 | 40 | 0.5 | 0 | 99.5 |
| (antiMCSP/anti huCD3) | | | | | |
| (dsscFv)₂-Fc | 2.65 | 48 | 7.3 | 8.0 | 84.7 |
| (antiMCSP/anti huCD3) | | | | | |

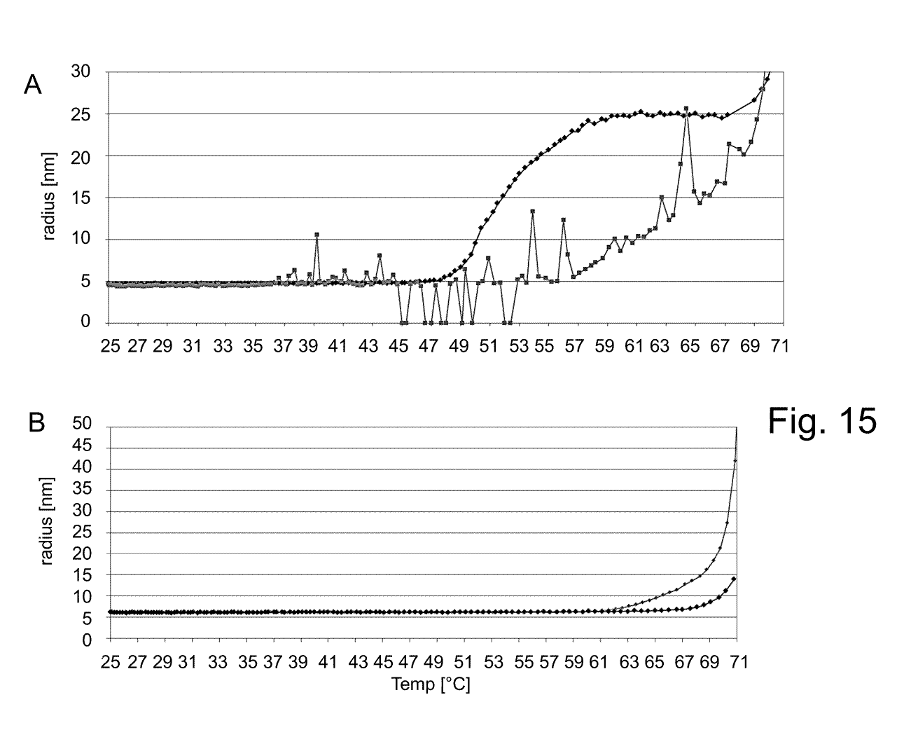

Thermal stability of the proteins was monitored by Dynamic Light Scattering (DLS). 30·g of filtered protein sample with a protein concentration of 1 mg/ml was applied in duplicate to a Dynapro plate reader (Wyatt Technology Corporation; USA). The temperature was ramped from 25 to 75° C. at 0.05° C./min, with the radius and total scattering intensity being collected. The results are shown in FIG. 15 and Table 2C. For the “(scFv)2-Fc” (antiMCSP/anti huCD3) molecule two aggregation points were observed, at 49° C. and 68° C. The “(dsscFv)₂-Fc” construct has an increased aggregation temperature (57° C.) as a result of the introduced disulfide bridge (FIG. 15A, Table 2C). Both, the “2+1 IgG scFab” and the “2+1 IgG Crossfab” constructs are aggregating at temperatures higher than 60° C., demonstrating their superior thermal stability as compared to the “(scFv)₂-Fc” and “(dsscFv)₂-Fc” formats (FIG. 15B, Table 2C).

TABLE 2C
Thermal stability determined by
dynamic light scattering.

| Construct | T_{agg} [° C.] |
|---|---|
| 2 + 1 IgG scFab (LC007/V9) | 68 |
| 2 + 1 IgG Crossfab (LC007/V9) | 65 |
| Fc-(scFv)2 (LC007/V9) | 49/68 |
| Fc-(dsscFv)2 (LC007/V9) | 57 |

### Example 2

### Surface Plasmon Resonance Analysis of Fc Receptor and Target Antigen Binding

### Method

All surface plasmon resonance (SPR) experiments are performed on a Biacore T100 at 25° C. with HBS-EP as running buffer (0.01 M HEPES pH 7.4, 0.15 M NaCl, 3 mM EDTA, 0.005% Surfactant P20, Biacore, Freiburg/Germany).

### Analysis of FcR Binding of Different Fc-Variants

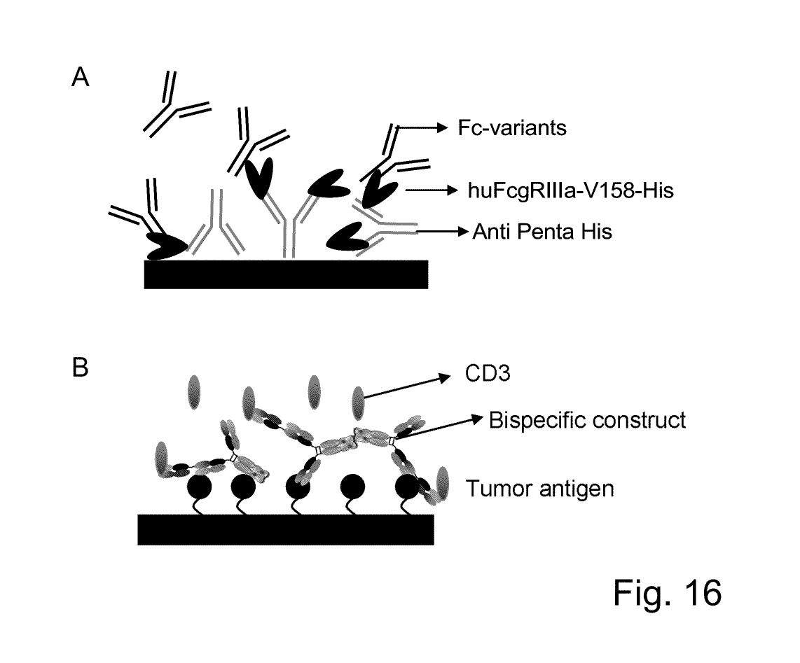

The assay setup is shown in FIG. 16A. For analyzing interaction of different Fc-variants with human FcγRIIIa-V158 and murine FcγRIV direct coupling of around 6,500 resonance units (RU) of the anti-Penta His antibody (Qiagen) is performed on a CM5 chip at pH 5.0 using the standard amine coupling kit (Biacore, Freiburg/Germany). HuFcγRIIIa-V158-K6H6 and muFcγRIV-aviHis-biotin are captured for 60 s at 4 and 10 nM respectively.

Constructs with different Fc-mutations are passed through the flow cells for 120 s at a concentration of 1000 nM with a flow rate of 30 μl/min. The dissociation is monitored for 220 s. Bulk refractive index differences are corrected for by subtracting the response obtained in a reference flow cell. Here, the Fc-variants are flown over a surface with immobilized anti-Penta His antibody but on which HBS-EP has been injected rather than HuFcγRIIIa-V158-K6H6 or muFcγRIV-aviHis-biotin. Affinity for human FcγRIIIa-V158 and murine FcγRIV was determined for wild-type Fc using a concentration range from 500-4000 nM.

The steady state response was used to derive the dissociation constant K_{D} by non-linear curve fitting of the Langmuir binding isotherm. Kinetic constants were derived using the Biacore T100 Evaluation Software (vAA, Biacore AB, Uppsala/Sweden), to fit rate equations for 1:1 Langmuir binding by numerical integration.

### Result

The interaction of Fc variants with human FcγRIIIa and murine FcγRIV was monitored by surface plasmon resonance. Binding to captured huFcγRIIIa-V158-K6H6 and muFcγRIV-aviHis-biotin is significantly reduced for all analyzed Fc mutants as compared to the construct with a wild-type (wt) Fc domain.

The Fc mutants with the lowest binding to the human Fcγ-receptor were P329G L234A L235A (LALA) and P329G LALA N297D. The LALA mutation alone was not enough to abrogate binding to huFcγRIIIa-V158-K6H6. The Fc variant carrying only the LALA mutation had a residual binding affinity to human FcγRIIIa of 2.100 nM, while the wt Fc bound the human FcγRIIIa receptor with an affinity of 600 nM (Table 3). Both K_{D} values were derived by 1:1 binding model, using a single concentration.

Affinity to human FcγRIIIa-V158 and murine FcγRIV could only be analyzed for wt Fc. K_{D} values are listed in Table 3. Binding to the murine FcγRIV was almost completely eliminated for all analyzed Fc mutants.

TABLE 3
Affinity of Fc-variants to the human
FcγRIIIa-V158 and murine FcγRIV.

| | human | | murine | |
|---|---|---|---|---|
| K_{D} in nM | FcγRIIIa-V158 | | FcγRIV | |
| T = 25° C. | kinetic | steady state | kinetic | steady state |
| Fc-wt | 600* (1200) | 3470 | 576 | 1500 |
| (SEQ ID NOs 5, | | | | |
| 13, 15) | | | | |
| Fc-LALA | 2130* | n.d. | n.d. | |
| (SEQ ID NOs 5, | | | | |
| 17, 19) | | | | |
| Fc-P329G LALA | n.d. | | n.d. | |
| (SEQ ID NOs 5, | | | | |
| 21, 23) | | | | |
| Fc-P329G LALA | n.d. | | n.d. | |
| N297D (SEQ ID NOs | | | | |
| 5, 25, 27) | | | | |

*determined using one concentration (1000 nM)

### Analysis of Simultaneous Binding to Tumor Antigen and CD3

Analysis of simultaneous binding of the T-cell bispecific constructs to the tumor antigen and the human CD3c was performed by direct coupling of 1650 resonance units (RU) of biotinylated D3 domain of MCSP on a sensor chip SA using the standard coupling procedure. Human EGFR was immobilized using standard amino coupling procedure. 8000 RU were immobilized on a CM5 sensor chip at pH 5.5. The assay setup is shown in FIG. 16B.

Different T-cell bispecific constructs were captured for 60 s at 200 nM. Human CD3γ(G₄S)₅CD3ε-AcTev-Fc(knob)-Avi/Fc(hole) was subsequently passed at a concentration of 2000 nM and a flow rate of 40 μl/min for 60 s. Bulk refractive index differences were corrected for by subtracting the response obtained on a reference flow cell where the recombinant CD3c was flown over a surface with immobilized D3 domain of MCSP or EGFR without captured T-cell bispecific constructs.

### Result

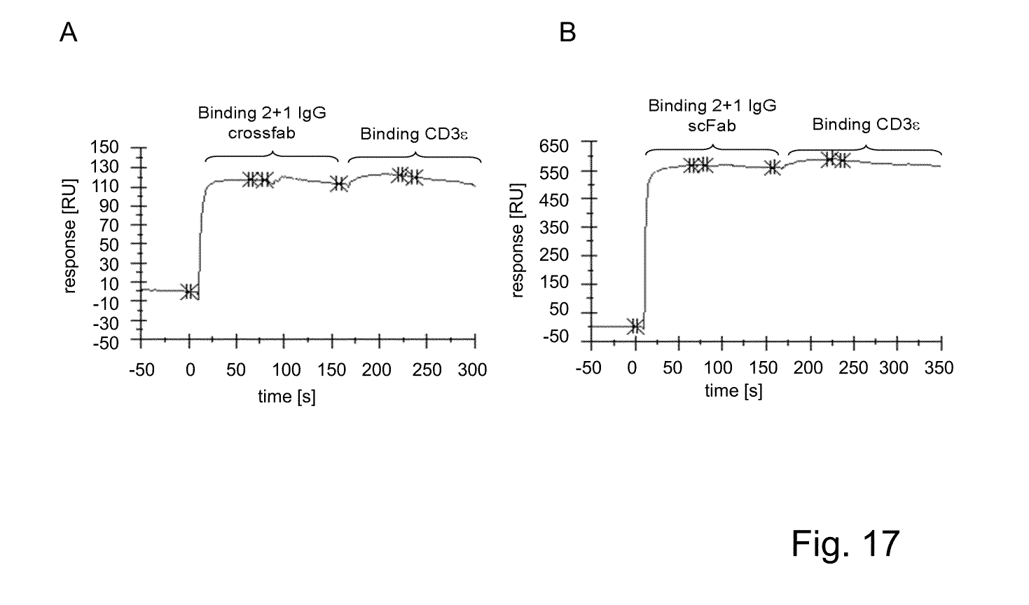

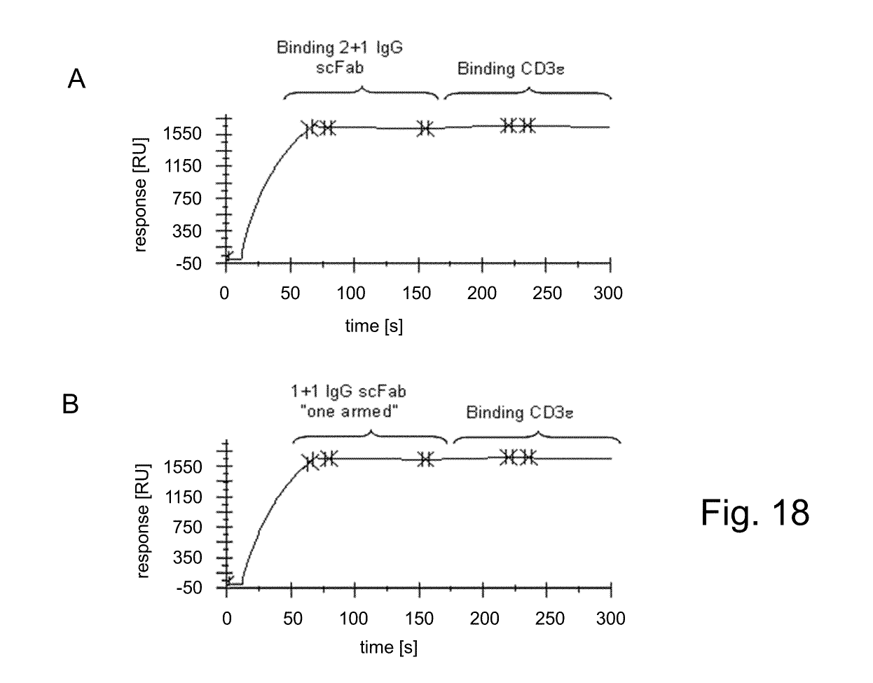

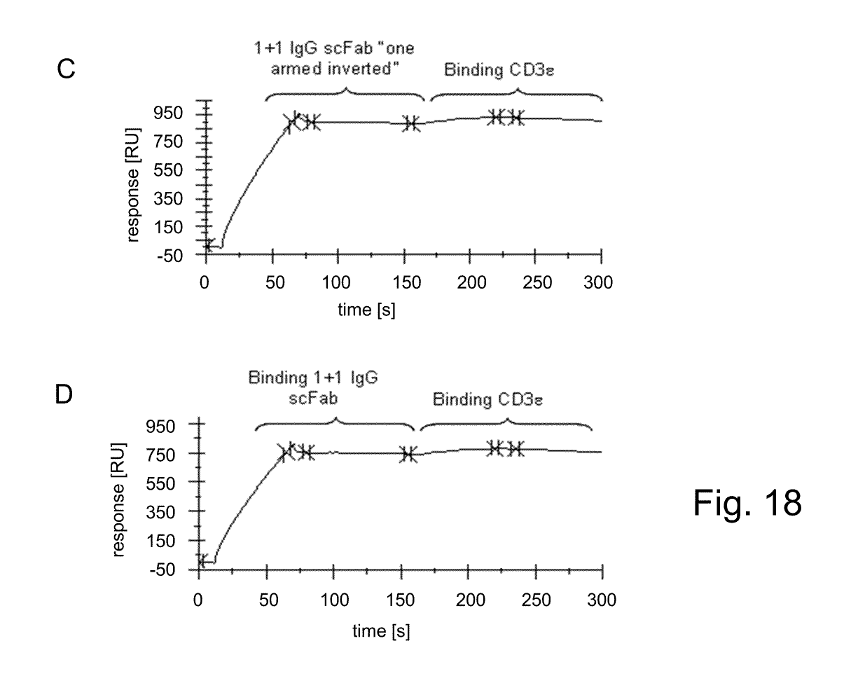

Simultaneous binding to both tumor antigen and human CD3c was analyzed by surface plasmon resonance (FIG. 17, FIG. 18). All constructs were able to bind the tumor antigen and the CD3 simultaneously. For most of the constructs the binding level (RU) after injection of human CD3c was higher than the binding level achieved after injection of the construct alone reflecting that both tumor antigen and the human CD3c were bound to the construct.

### Example 3

### Binding of Bispecific Constructs to the Respective Target Antigen on Cells

Binding of the different bispecific constructs to CD3 on Jurkat cells (ATCC #TIB-152), and the respective tumor antigen on target cells, was determined by FACS. Briefly, cells were harvested, counted and checked for viability. 0.15-0.2 million cells per well (in PBS containing 0.1% BSA; 90 μl) were plated in a round-bottom 96-well plate and incubated with the indicated concentration of the bispecific constructs and corresponding IgG controls (10 μl) for 30 min at 4° C. For a better comparison, all constructs and IgG controls were normalized to same molarity. After the incubation, cells were centrifuged (5 min, 350×g), washed with 150 μl PBS containing 0.1% BSA, resuspended and incubated for further 30 min at 4° C. with 12 μl/well of a FITC- or PE-conjugated secondary antibody. Bound constructs were detected using a FACSCantoII (Software FACS Diva). The “(scFv)₂” molecule was detected using a FITC-conjugated anti-His antibody (Lucerna, #RHIS-45F-Z). For all other molecules, a FITC- or PE-conjugated AffiniPure F(ab′)2 Fragment goat anti-human IgG Fcγ Fragment Specific (Jackson Immuno Research Lab #109-096-098/working solution 1:20, or #109-116-170/working solution 1:80, respectively) was used. Cells were washed by addition of 120 μl/well PBS containing 0.1% BSA and centrifugation at 350×g for 5 min. A second washing step was performed with 150 μl/well PBS containing 0.1% BSA. Unless otherwise indicated, cells were fixed with 100 μl/well fixation buffer (BD #554655) for 15 min at 4° C. in the dark, centrifuged for 6 min at 400×g and kept in 200 μl/well PBS containing 0.1% BSA until the samples were measured with FACS CantoII. EC50 values were calculated using the GraphPad Prism software.

In a first experiment, different bispecific constructs targeting human MCSP and human CD3 were analyzed by flow cytometry for binding to human CD3 expressed on Jurkat, human T cell leukaemia cells, or to human MCSP on Colo-38 human melanoma cells.

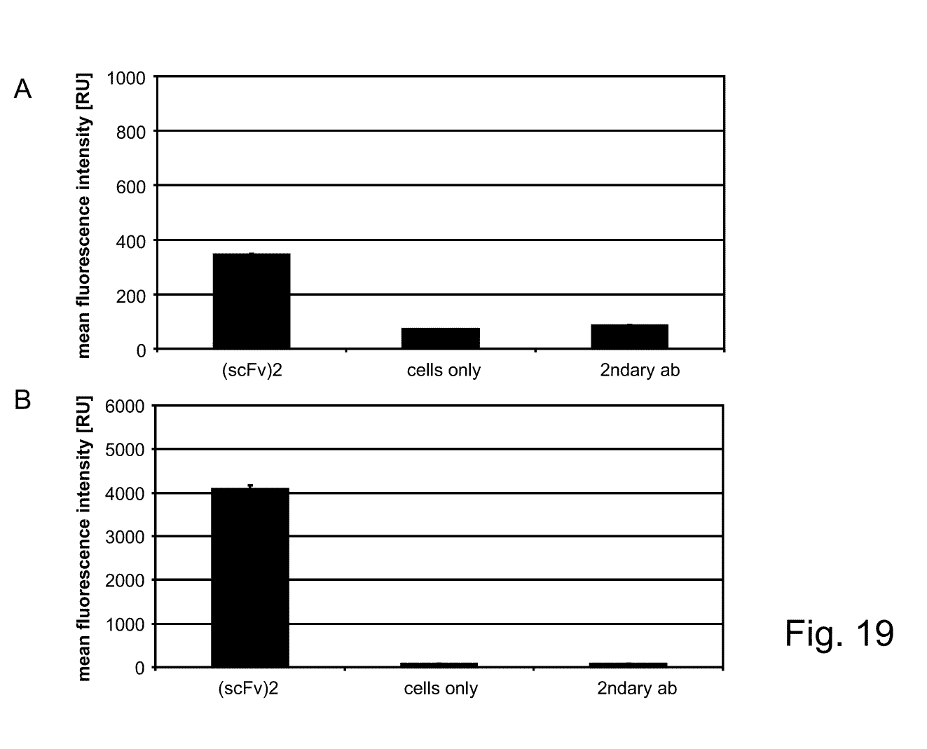

Results are presented in FIG. 19-21, which show the mean fluorescence intensity of cells that were incubated with the bispecific molecule, control IgG, the secondary antibody only, or left untreated.

As shown in FIG. 19, for both antigen binding moieties of the “(scFv)₂” molecule, i.e. CD3 (FIG. 191A) and MCSP (FIG. 19B), a clear binding signal is observed compared to the control samples.

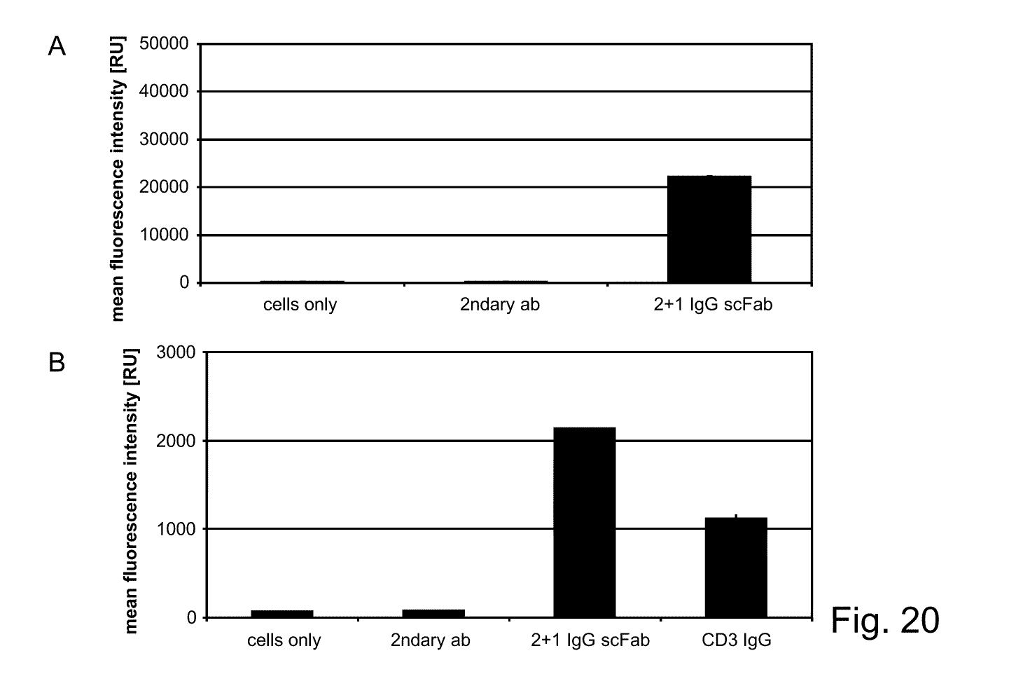

The “2+1 IgG scFab” molecule (SEQ ID NOs 5, 17, 19) shows good binding to huMCSP on Colo-38 cells (FIG. 20A). The CD3 moiety binds CD3 slightly better than the reference anti-human CD3 IgG (FIG. 20B).

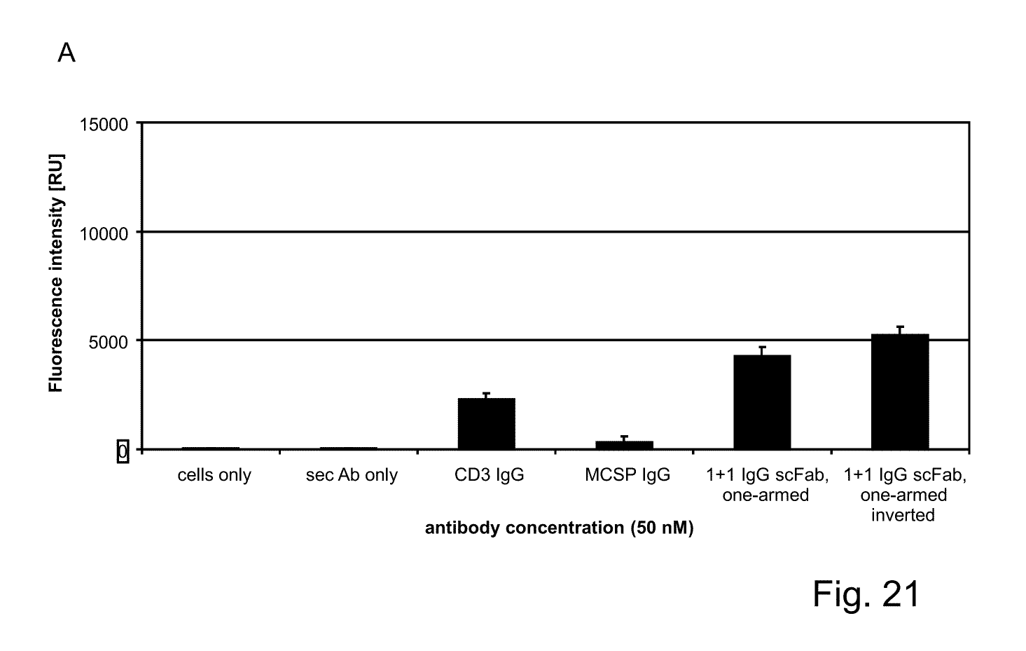

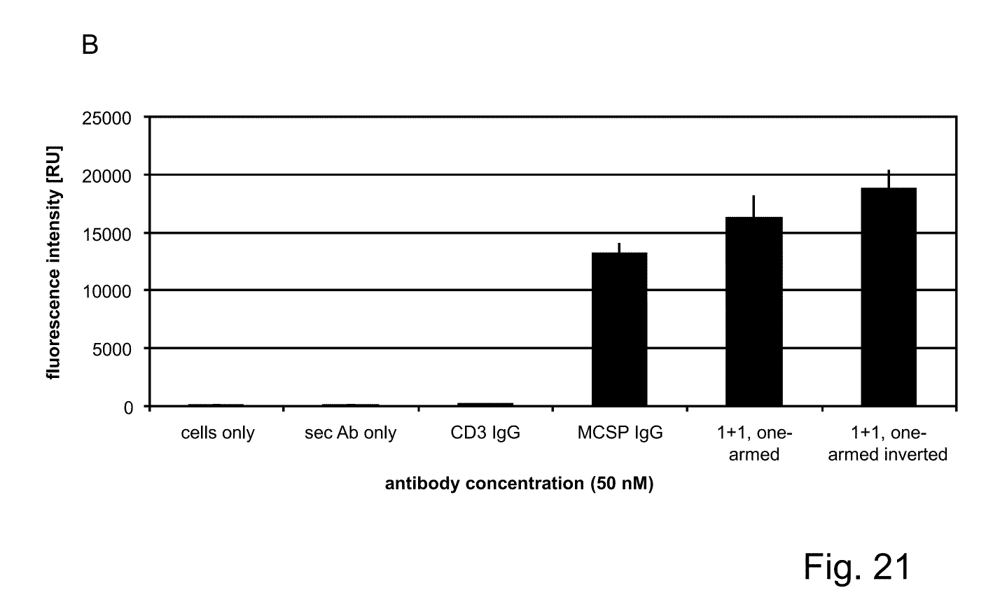

As depicted in FIG. 21A, the two “1+1” constructs show comparable binding signals to human CD3 on cells. The reference anti-human CD3 IgG gives a slightly weaker signal. In addition, both constructs tested (“1+1 IgG scFab, one-armed” (SEQ ID NOs 1, 3, 5) and “1+1 IgG scFab, one-armed inverted” (SEQ ID NOs 7, 9, 11)) show comparable binding to human MCSP on cells (FIG. 21B). The binding signal obtained with the reference anti-human MCSP IgG is slightly weaker.

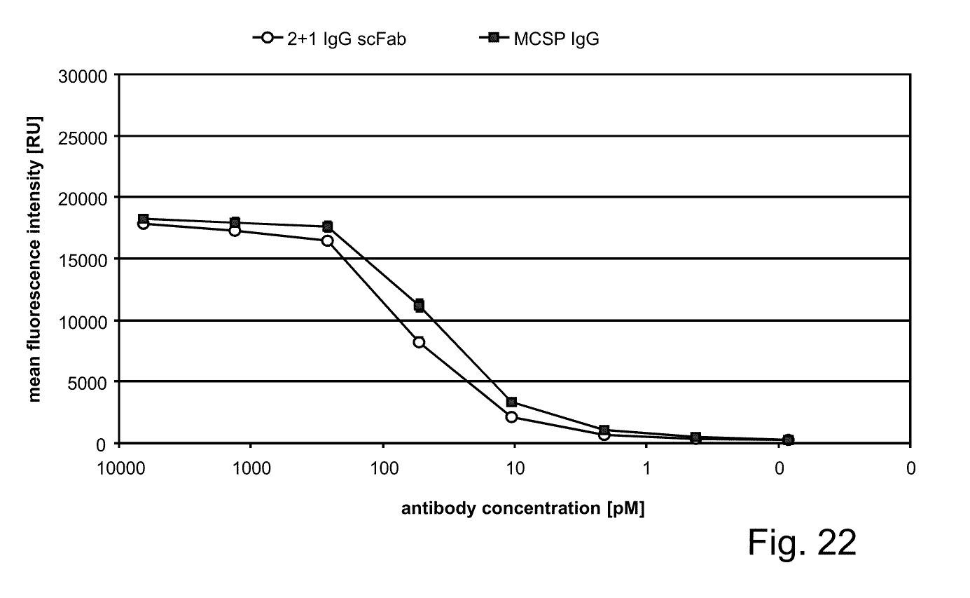

In another experiment, the purified “2+1 IgG scFab” bispecific construct (SEQ ID NOs 5, 17, 19) and the corresponding anti human MCSP IgG were analyzed by flow cytometry for dose-dependent binding to human MCSP on Colo-38 human melanoma cells, to determine whether the bispecific construct binds to MCSP via one or both of its “arms”. As depicted in FIG. 22, the “2+1 IgG scFab” construct shows the same binding pattern as the MCSP IgG.

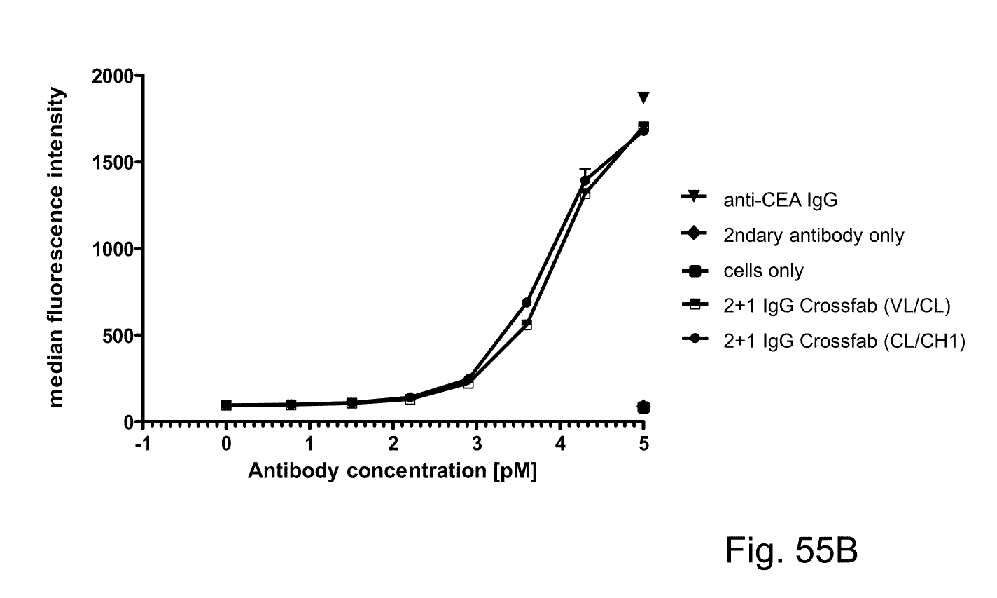

In yet another experiment, the binding of CD3/CEA “2+1 IgG Crossfab, inverted” bispecific constructs with either a VL/VH (see SEQ ID NOs 33, 63, 65, 67) or a CL/CH1 exchange (see SEQ ID NOs 66, 67, 183, 197) in the Crossfab fragment to human CD3, expressed by Jurkat cells, or to human CEA, expressed by LS-174T cells, was assessed. As a control, the equivalent maximum concentration of the corresponding IgGs and the background staining due to the labeled 2ndary antibody (goat anti-human FITC-conjugated AffiniPure F(ab′)2 Fragment, FcγFragment-specific, Jackson Immuno Research Lab #109-096-098) were assessed as well. As illustrated in FIG. 55, both constructs show good binding to human CEA, as well as to human CD3 on cells. The calculated EC50 values were 4.6 and 3.9 nM (CD3), and 9.3 and 6.7 nM (CEA) for the “2+1 IgG Crossfab, inverted (VL/VH)” and the “2+1 IgG Crossfab, inverted (CL/CH1)” constructs, respectively.

In another experiment, the binding of CD3/MCSP “2+1 IgG Crossfab” (see SEQ ID NOs 3, 5, 29, 33) and “2+1 IgG Crossfab, inverted” (see SEQ ID NOs 5, 23, 183, 187) constructs to human

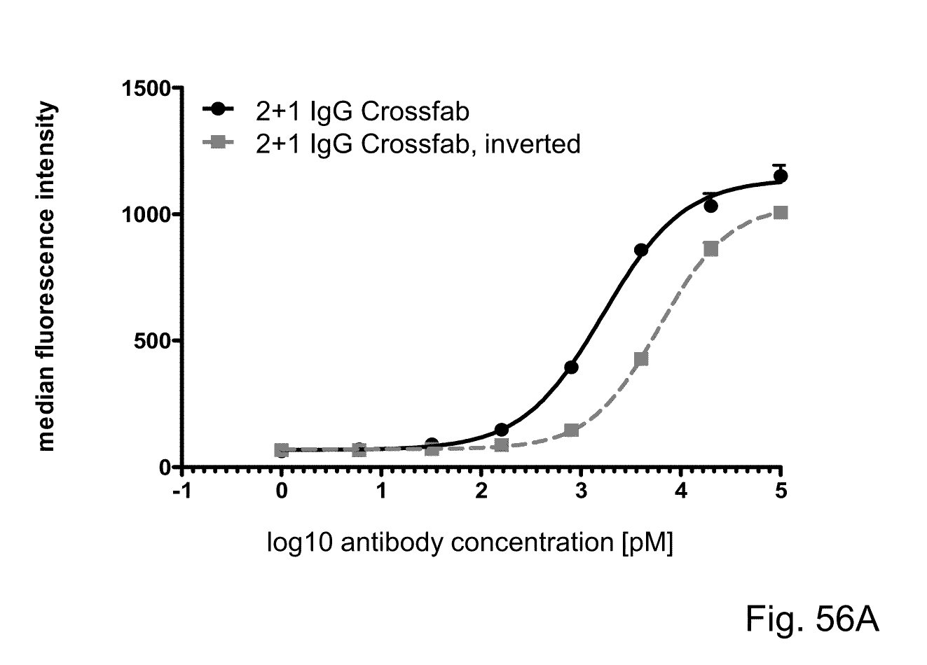

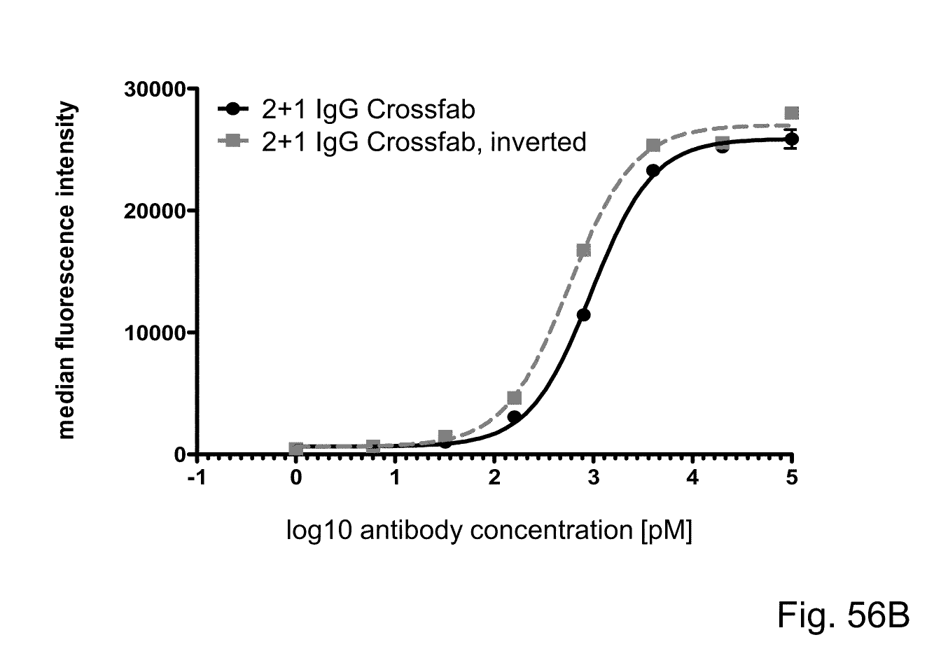

CD3, expressed by Jurkat cells, or to human MCSP, expressed by WM266-4 cells, was assessed. FIG. 56 shows that, while binding of both constructs to MCSP on cells was comparably good, the binding of the “inverted” construct to CD3 was reduced compared to the other construct. The calculated EC50 values were 6.1 and 1.66 nM (CD3), and 0.57 and 0.95 nM (MCSP) for the “2+1 IgG Crossfab, inverted” and the “2+1 IgG Crossfab” constructs, respectively.

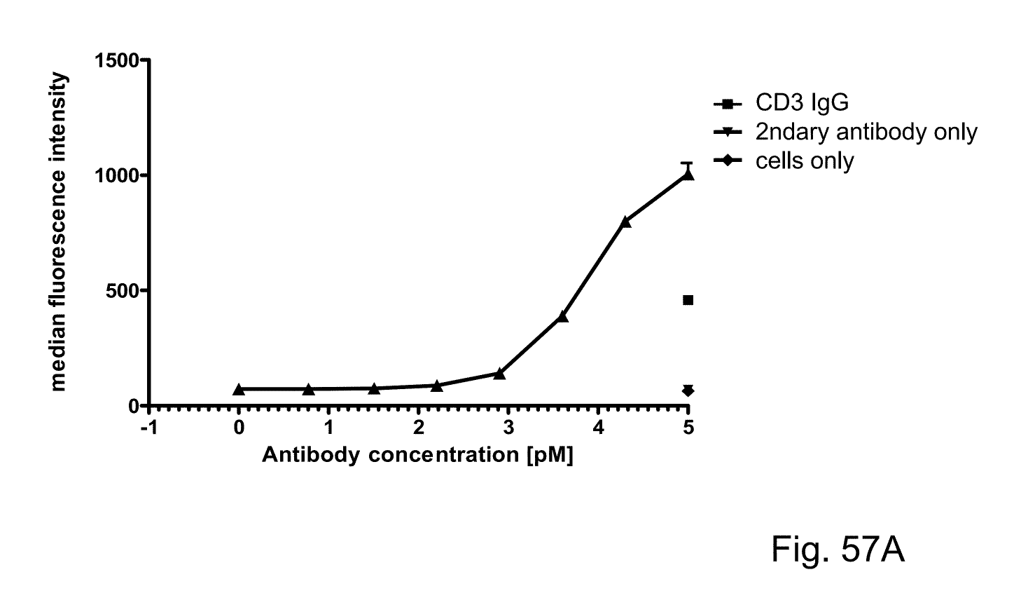

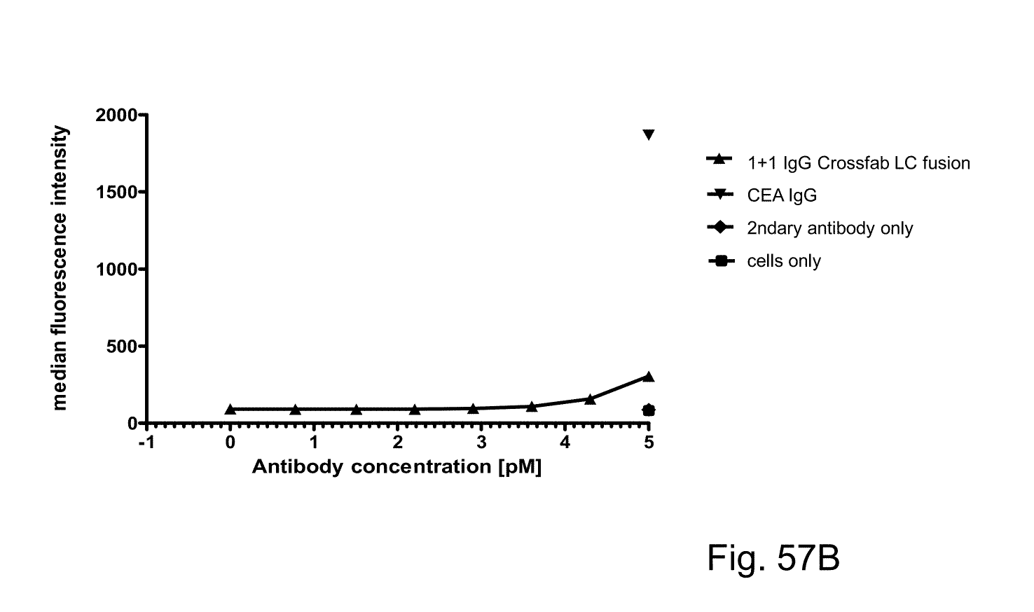

In a further experiment, binding of the “1+1 IgG Crossfab light chain (LC) fusion” construct (SEQ ID NOs 183, 209, 211, 213) to human CD3, expressed by Jurkat cells, and to human CEA, expressed by LS-174T cells was determined. As a control, the equivalent maximum concentration of the corresponding anti-CD3 and anti-CEA IgGs and the background staining due to the labeled 2ndary antibody (goat anti-human FITC-conjugated AffiniPure F(ab′)2 Fragment, Fcγ Fragment-specific, Jackson Immuno Research Lab #109-096-098) were assessed as well. As depicted in FIG. 57, the binding of the “1+1 IgG Crossfab LC fusion” to CEA appears to be greatly reduced, whereas the binding to CD3 was at least comparable to the reference IgG.

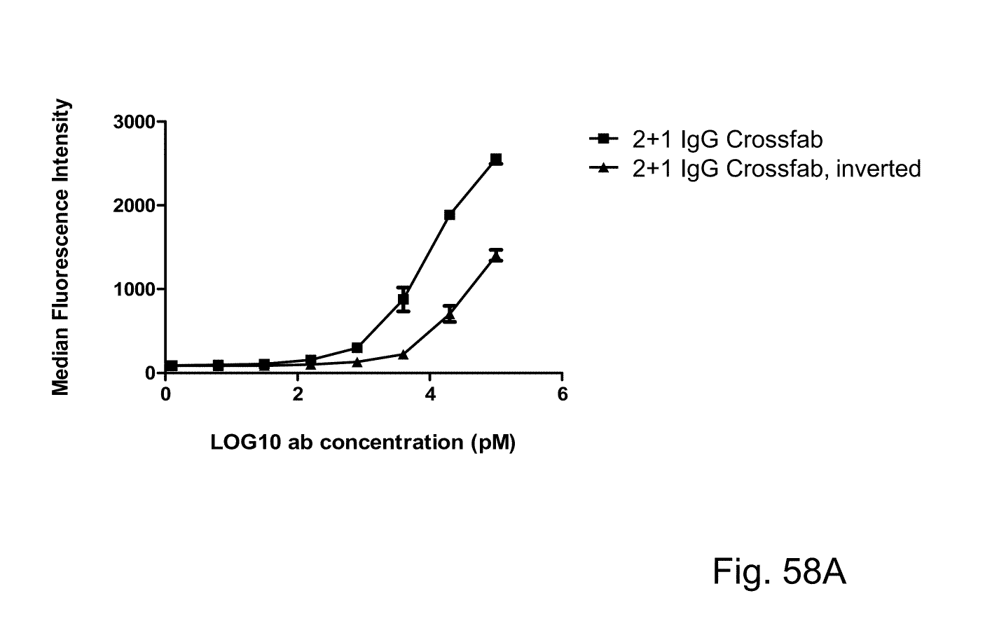

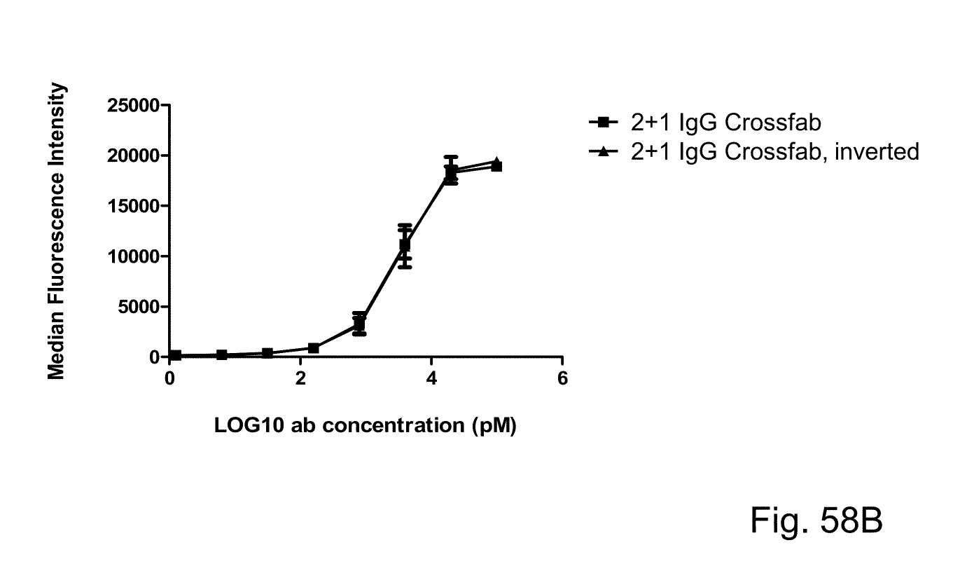

In a final experiment, binding of the “2+1 IgG Crossfab” (SEQ ID NOs 5, 23, 215, 217) and the “2+1 IgG Crossfab, inverted” (SEQ ID NOs 5, 23, 215, 219) constructs to human CD3, expressed by Jurkat cells, and to human MCSP, expressed by WM266-4 tumor cells was determined. As depicted in FIG. 58 the binding to human CD3 was reduced for the “2+1 IgG Crossfab, inverted” compared to the other construct, but the binding to human MCSP was comparably good. The calculated EC50 values were 10.3 and 32.0 nM (CD3), and 3.1 and 3.4 nM (MCSP) for the “2+1 IgG Crossfab” and the “2+1 IgG Crossfab, inverted” construct, respectively.

### Example 4

### FACS Analysis of Surface Activation Markers on Primary Human T Cells Upon Engagement of Bispecific Constructs

The purified huMCSP-huCD3-targeting bispecific “2+1 IgG scFab” (SEQ ID NOs 5, 17, 19) and “(scFv)₂” molecules were tested by flow cytometry for their potential to up-regulate the early surface activation marker CD69, or the late activation marker CD25 on CD8⁺ T cells in the presence of human MCSP-expressing tumor cells.

Briefly, MCSP-positive Colo-38 cells were harvested with Cell Dissociation buffer, counted and checked for viability. Cells were adjusted to 0.3×10⁶ (viable) cells per ml in AIM-V medium, 100 μl of this cell suspension per well were pipetted into a round-bottom 96-well plate (as indicated). 50 μl of the (diluted) bispecific construct were added to the cell-containing wells to obtain a final concentration of 1 nM. Human PBMC effector cells were isolated from fresh blood of a healthy donor and adjusted to 6×10⁶ (viable) cells per ml in AIM-V medium. 50 μl of this cell suspension was added per well of the assay plate (see above) to obtain a final E:T ratio of 10:1. To analyze whether the bispecific constructs are able to activate T cells exclusively in the presence of target cells expressing the tumor antigen huMCSP, wells were included that contained 1 nM of the respective bispecific molecules, as well as PBMCs, but no target cells.

After incubation for 15 h (CD69), or 24 h (CD25) at 37° C., 5% CO₂, cells were centrifuged (5 min, 350×g) and washed twice with 150 μl/well PBS containing 0.1% BSA. Surface staining for CD8 (mouse IgG1, κ; clone HIT8a; BD #555635), CD69 (mouse IgG1; clone L78; BD #340560) and CD25 (mouse IgG1, κ; clone M-A251; BD #555434) was performed at 4° C. for 30 min, according to the supplier's suggestions. Cells were washed twice with 150 μl/well PBS containing 0.1% BSA and fixed for 15 min at 4° C., using 100 μl/well fixation buffer (BD #554655). After centrifugation, the samples were resuspended in 200 μl/well PBS with 0.1% BSA and analyzed using a FACS CantoII machine (Software FACS Diva).

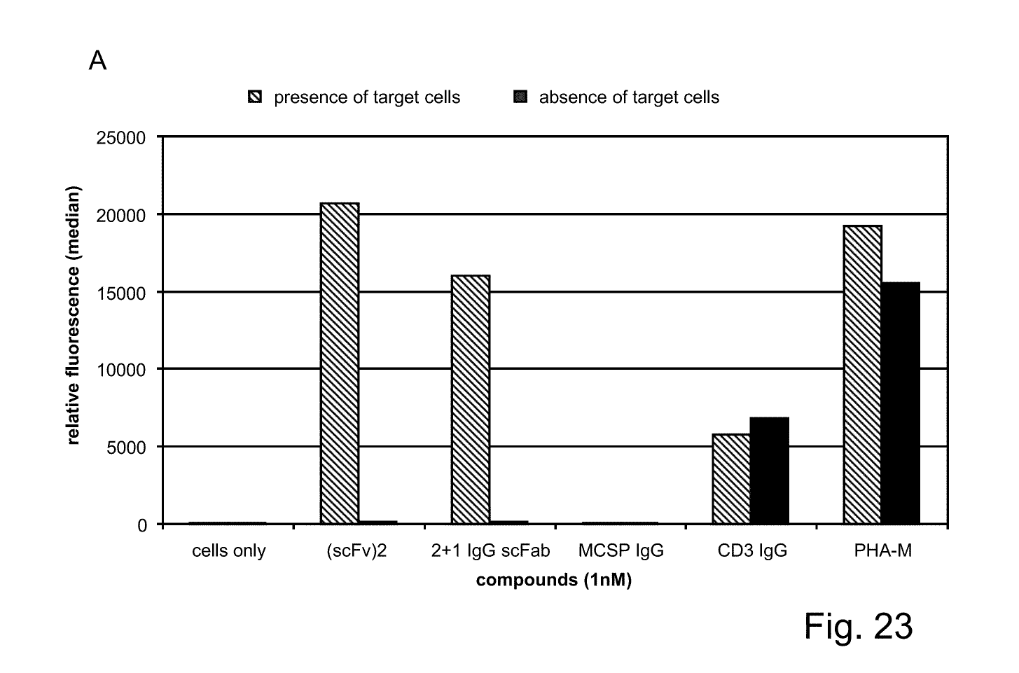

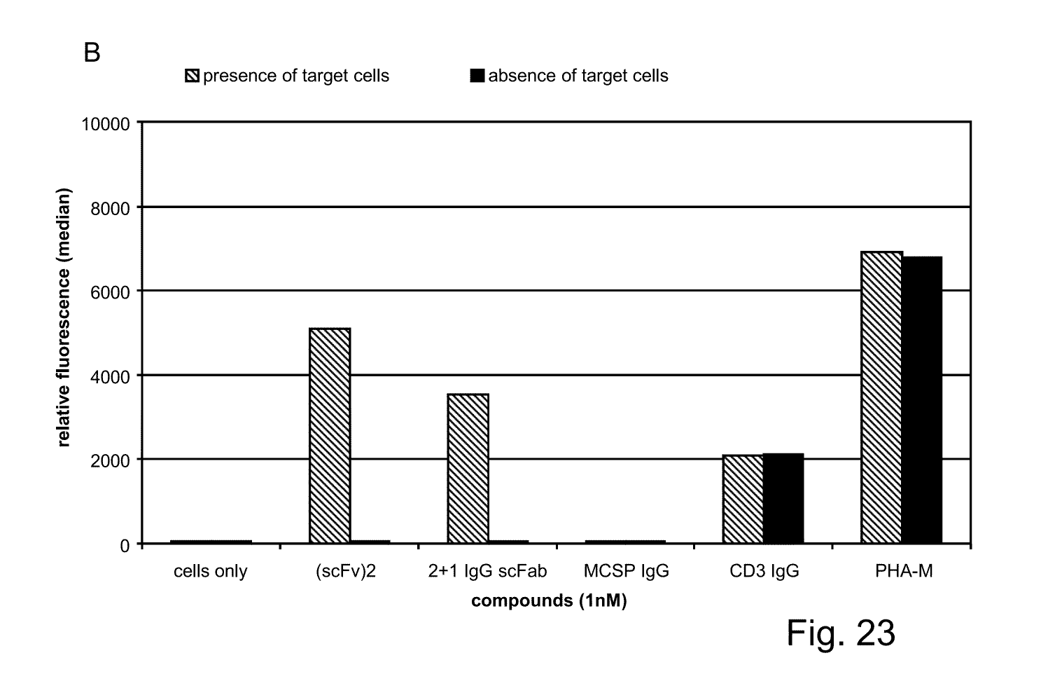

FIG. 23 depicts the expression level of the early activation marker CD69 (A), or the late activation marker CD25 (B) on CD8⁺ T cells after 15 hours or 24 hours incubation, respectively. Both constructs induce up-regulation of both activation markers exclusively in the presence of target cells. The “(scFv)₂” molecule seems to be slightly more active in this assay than the “2+1 IgG scFab” construct.

The purified huMCSP-huCD3-targeting bispecific “2+1 IgG scFab” and “(scFv)₂” molecules were further tested by flow cytometry for their potential to up-regulate the late activation marker CD25 on CD8⁺ T cells or CD4⁺ T cells in the presence of human MCSP-expressing tumor cells. Experimental procedures were as described above, using human pan T effector cells at an E:T ratio of 5:1 and an incubation time of five days.

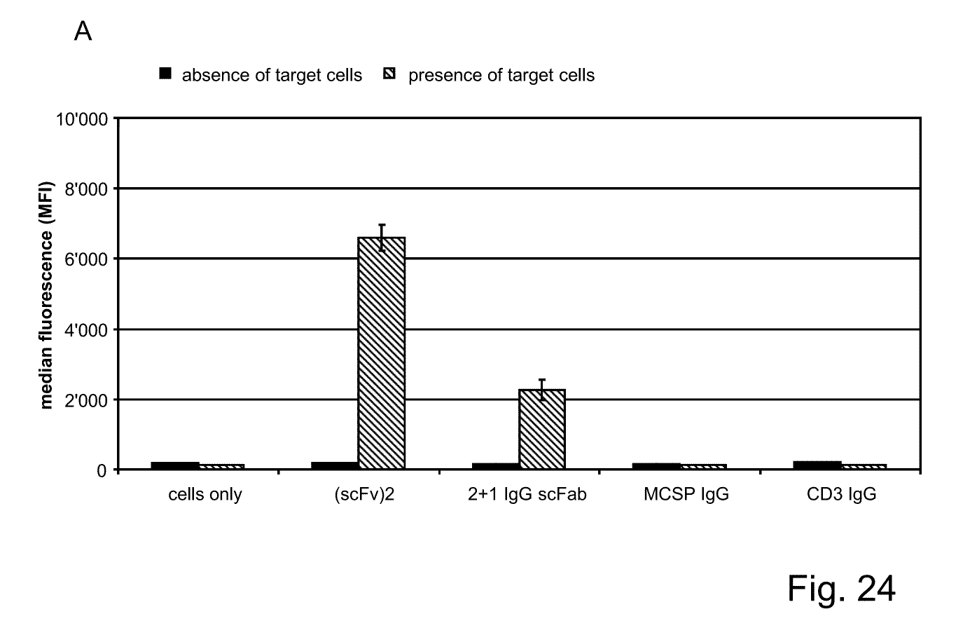

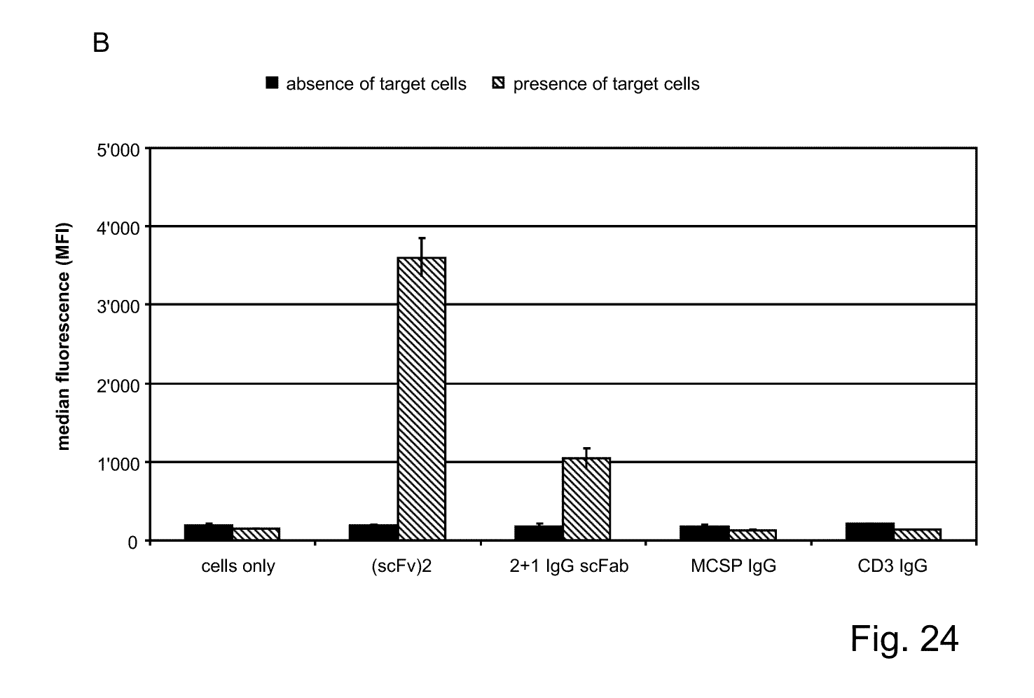

FIG. 24 shows that both constructs induce up-regulation of CD25 exclusively in the presence of target cells on both, CD8⁺ (A) as well as CD4⁺ (B) T cells. The “2+1 IgG scFab” construct seems to induce less up-regulation of CD25 in this assay, compared to the “(scFv)₂” molecule. In general, the up-regulation of CD25 is more pronounced on CD8⁺ than on CD4⁺ T cells.

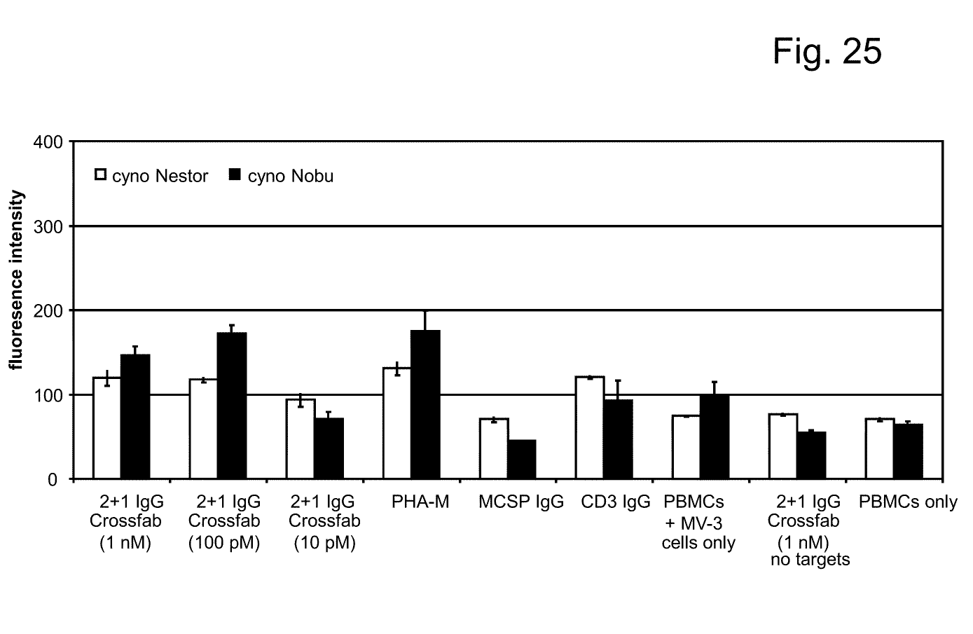

In another experiment, purified “2+1 IgG Crossfab” targeting cynomolgus CD3 and human MCSP (SEQ ID NOs 3, 5, 35, 37) was analyzed for its potential to up-regulate the surface activation marker CD25 on CD8⁺ T cells in the presence of tumor target cells. Briefly, human MCSP-expressing MV-3 tumor target cells were harvested with Cell Dissociation Buffer, washed and resuspended in DMEM containing 2% FCS and 1% GlutaMax. 30 000 cells per well were plated in a round-bottom 96-well plate and the respective antibody dilution was added at the indicated concentrations (FIG. 25). The bispecific construct and the different IgG controls were adjusted to the same molarity. Cynomolgus PBMC effector cells, isolated from blood of two healthy animals, were added to obtain a final E:T ratio of 3:1. After an incubation for 43 h at 37° C., 5% CO₂, the cells were centrifuged at 350×g for 5 min and washed twice with PBS, containing 0.1% BSA. Surface staining for CD8 (Miltenyi Biotech #130-080-601) and CD25 (BD #557138) was performed according to the supplier's suggestions. Cells were washed twice with 150 μl/well PBS containing 0.1% BSA and fixed for 15 min at 4° C., using 100 μl/well fixation buffer (BD #554655). After centrifugation, the samples were resuspended in 200 μl/well PBS with 0.1% BSA and analyzed using a FACS CantoII machine (Software FACS Diva).

As depicted in FIG. 25, the bispecific construct induces concentration-dependent up-regulation of CD25 on CD8⁺ T cells only in the presence of target cells. The anti cyno CD3 IgG (clone FN-18) is also able to induce up-regulation of CD25 on CD8⁺ T cells, without being crosslinked (see data obtained with cyno Nestor). There is no hyperactivation of cyno T cells with the maximal concentration of the bispecific construct (in the absence of target cells).

In another experiment, the CD3-MCSP “2+1 IgG Crossfab, linked light chain” (see SEQ ID NOs 3, 5, 29, 179) was compared to the CD3-MCSP “2+1 IgG Crossfab” (see SEQ ID NOs 3, 5, 29, 33) for its potential to up-regulate the early activation marker CD69 or the late activation marker CD25 on CD8⁺ T cells in the presence of tumor target cells. Primary human PBMCs (isolated as described above) were incubated with the indicated concentrations of bispecific constructs for at least 22 h in the presence or absence of MCSP-positive Colo38 target cells. Briefly, 0.3 million primary human PBMCs were plated per well of a flat-bottom 96-well plate, containing the MCSP-positive target cells (or medium). The final effector to target cell (E:T) ratio was 10:1. The cells were incubated with the indicated concentration of the bispecific constructs and controls for the indicated incubation times at 37° C., 5% CO₂. The effector cells were stained for CD8, and CD69 or CD25 and analyzed by FACS CantoII.

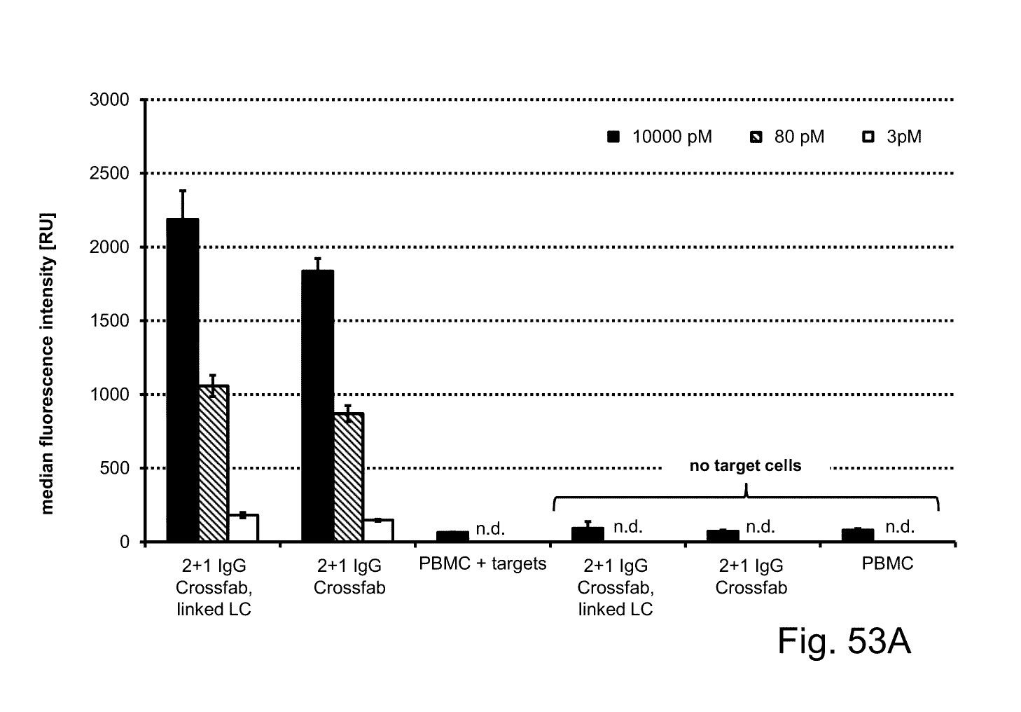

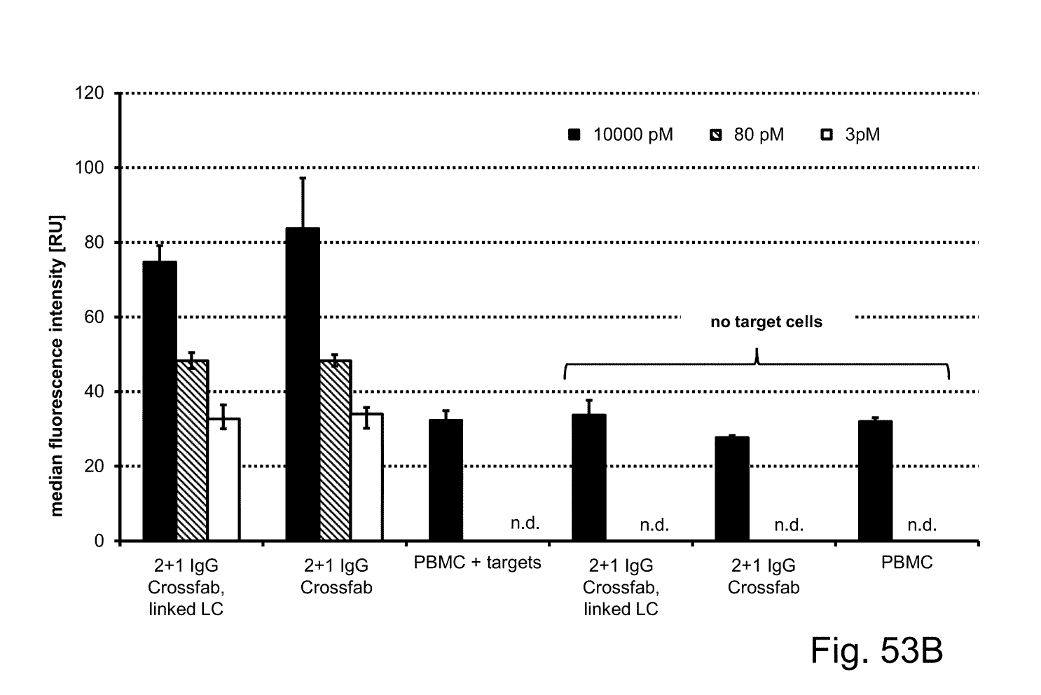

FIG. 53 shows the result of this experiment. There were no significant differences detected for CD69 (A) or CD25 up-regulation (B) between the two 2+1 IgG Crossfab molecules (with or without the linked light chain).

In yet another experiment, the CD3/MCSP “2+1 IgG Crossfab” (see SEQ ID NOs 3, 5, 29, 33) and “1+1 IgG Crossfab” (see SEQ ID NOs 5, 29, 33, 181) constructs were compared to the “1+1 CrossMab” construct (see SEQ ID NOs 5, 23, 183, 185) for their potential to up-regulate CD69 or CD25 on CD4⁺ or CD8⁺ T cells in the presence of tumor target cells. The assay was performed as described above, in the presence of absence of human MCSP expressing MV-3 tumor cells, with an incubation time of 24 h.

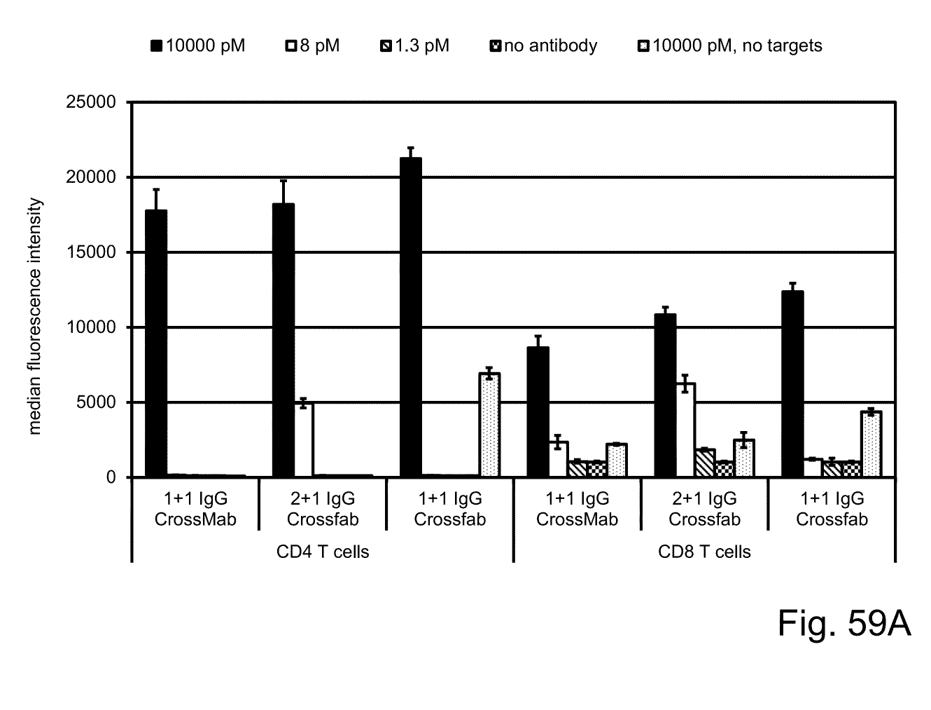

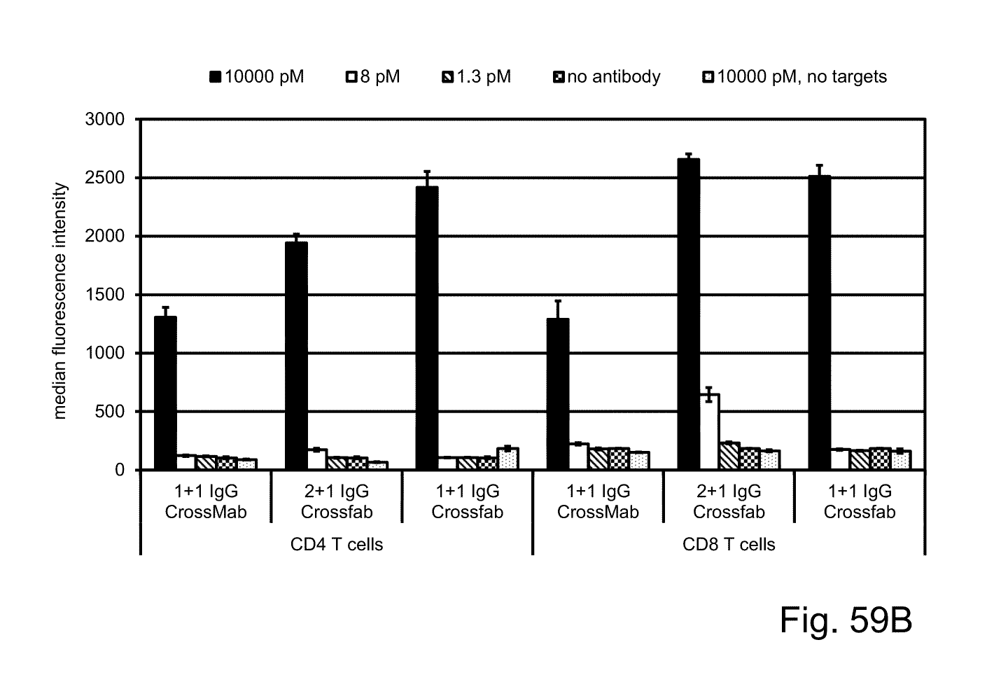

As shown in FIG. 59, the “1+1 IgG Crossfab” and “2+1 IgG Crossfab” constructs induced more pronounced upregulation of activation markers than the “1+1 CrossMab” molecule.

In a final experiment, the CD3/MCSP “2+1 IgG Crossfab” (see SEQ ID NOs 5, 23, 215, 217) and “2+1 IgG Crossfab, inverted” (see SEQ ID NOs 5, 23, 215, 219) constructs were assessed for their potential to up-regulate CD25 on CD4⁺ or CD8⁺ T cells from two different cynomolgus monkeys in the presence of tumor target cells. The assay was performed as described above, in the presence of absence of human MCSP expressing MV-3 tumor cells, with an E:T ratio of 3:1 and an incubation time of about 41 h.

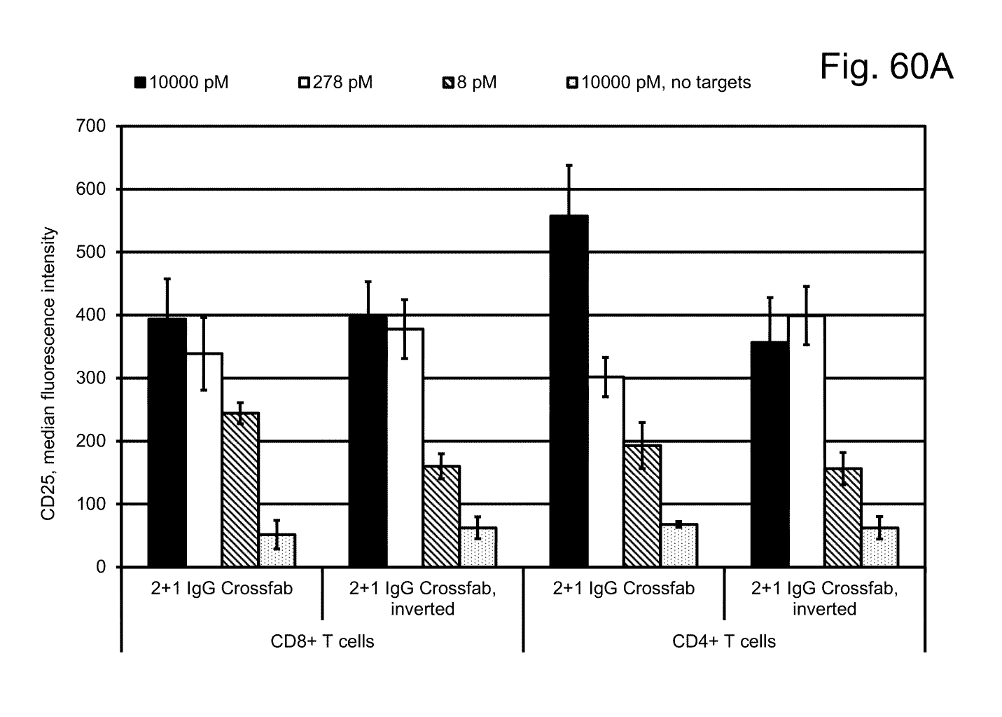

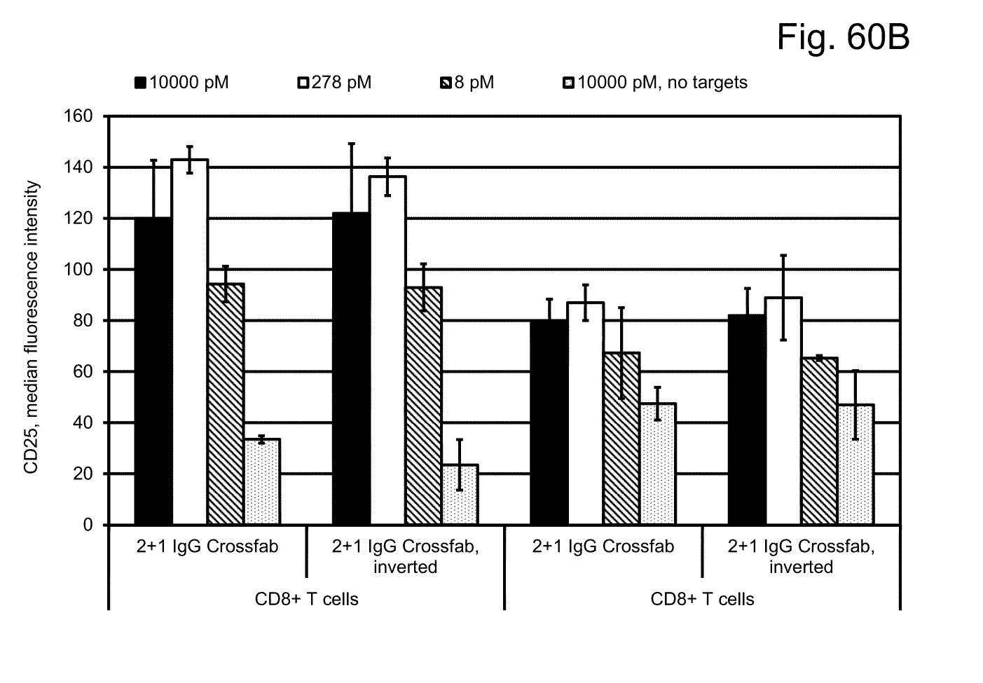

As shown in FIG. 60, both constructs were able to up-regulate CD25 on CD4⁺ and CD8⁺ T cells in a concentration-dependent manner, without significant difference between the two formats. Control samples without antibody and without target cells gave a comparable signal to the samples with antibody but no targets (not shown).

### Example 5

### Interferon-γ Secretion Upon Activation of Human Pan T Cells with CD3 Bispecific Constructs

Purified “2+1 IgG scFab” targeting human MCSP and human CD3 (SEQ ID NOs 5, 17, 19) was analyzed for its potential to induce T cell activation in the presence of human MCSP-positive U-87MG cells, measured by the release of human interferon (IFN)-γ into the supernatant. As controls, anti-human MCSP and anti-human CD3 IgGs were used, adjusted to the same molarity. Briefly, huMCSP-expressing U-87MG glioblastoma astrocytoma target cells (ECACC 89081402) were harvested with Cell Dissociation Buffer, washed and resuspended in AIM-V medium (Invitrogen #12055-091). 20 000 cells per well were plated in a round-bottom 96-well-plate and the respective antibody dilution was added to obtain a final concentration of 1 nM. Human pan T effector cells, isolated from Buffy Coat, were added to obtain a final E:T ratio of 5:1. After an overnight incubation of 18.5 h at 37° C., 5% CO₂, the assay plate was centrifuged for 5 min at 350×g and the supernatant was transferred into a fresh 96-well plate. Human IFN-γ levels in the supernatant were measured by ELISA, according to the manufacturer's instructions (BD OptEIA human IFN-γ ELISA Kit II from Becton Dickinson, #550612).

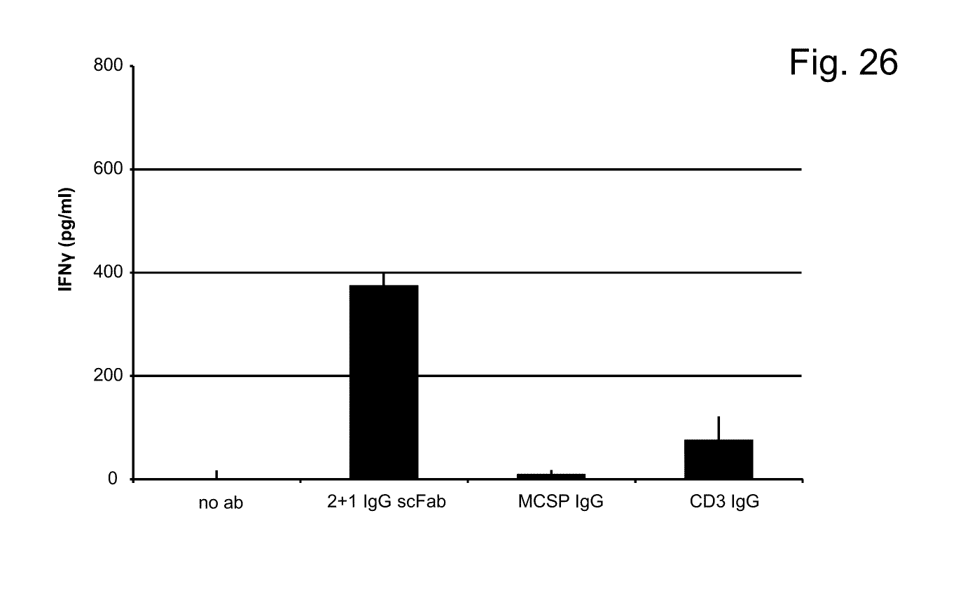

As depicted in FIG. 26, the reference IgGs show no to weak induction of IFN-γ secretion, whereas the “2+1 IgG scFab” construct is able to activate human T cells to secrete IFN-γ.

### Example 6

### Re-Directed T Cell Cytotoxicity Mediated by Cross-Linked Bispecific Constructs Targeting CD3 on T Cells and MCSP or EGFR on Tumor Cells (LDH Release Assay)

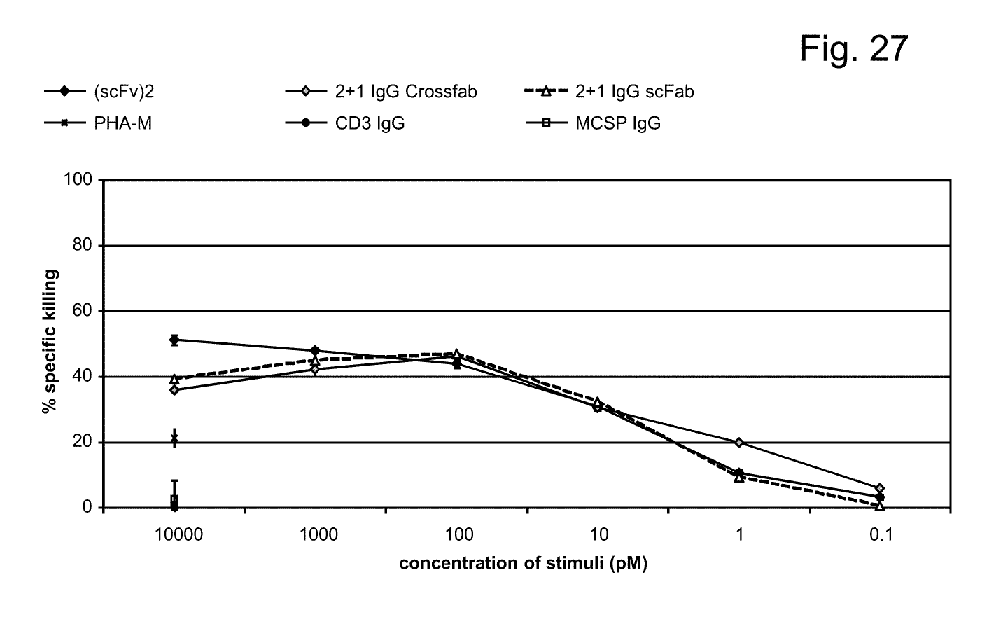

In a first series of experiments, bispecific constructs targeting CD3 and MCSP were analyzed for their potential to induce T cell-mediated apoptosis in tumor target cells upon crosslinkage of the construct via binding of the antigen binding moieties to their respective target antigens on cells (FIGS. 27-38).

In one experiment purified “2+1 IgG scFab” (SEQ ID NOs 5, 21, 23) and “2+1 IgG Crossfab” (SEQ ID NOs 3, 5, 29, 33) constructs targeting human CD3 and human MCSP, and the corresponding “(scFv)₂” molecule, were compared. Briefly, huMCSP-expressing MDA-MB-435 human melanoma target cells were harvested with Cell Dissociation Buffer, washed and resuspended in AIM-V medium (Invitrogen #12055-091). 30 000 cells per well were plated in a round-bottom 96-well plate and the respective dilution of the construct was added at the indicated concentration. All constructs and corresponding control IgGs were adjusted to the same molarity. Human pan T effector cells were added to obtain a final E:T ratio of 5:1. As a positive control for the activation of human pan T cells, 1 μg/ml PHA-M (Sigma #L8902; mixture of isolectins isolated from Phaseolus vulgaris) was used. For normalization, maximal lysis of the target cells (=100%) was determined by incubation of the target cells with a final concentration of 1% Triton X-100. Minimal lysis (=0%) refers to target cells co-incubated with effector cells, but without any construct or antibody. After an overnight incubation of 20 h at 37° C., 5% CO₂, LDH release of apoptotic/necrotic target cells into the supernatant was measured with the LDH detection kit (Roche Applied Science, #11 644 793 001), according to the manufacturer's instructions.

As depicted in FIG. 27, both “2+1” constructs induce apoptosis in target cells comparable to the “(scFv)₂” molecule.

Further, purified “2+1 IgG Crossfab” (SEQ ID NOs 3, 5, 29, 33) and “2+1 IgG scFab” constructs differing in their Fc domain, as well as the “(scFv)₂” molecule, were compared. The different mutations in the Fc domain (L234A+L235A (LALA), P329G and/or N297D, as indicated) reduce or abolish the (NK) effector cell function induced by constructs containing a wild-type (wt) Fc domain. Experimental procedures were as described above.

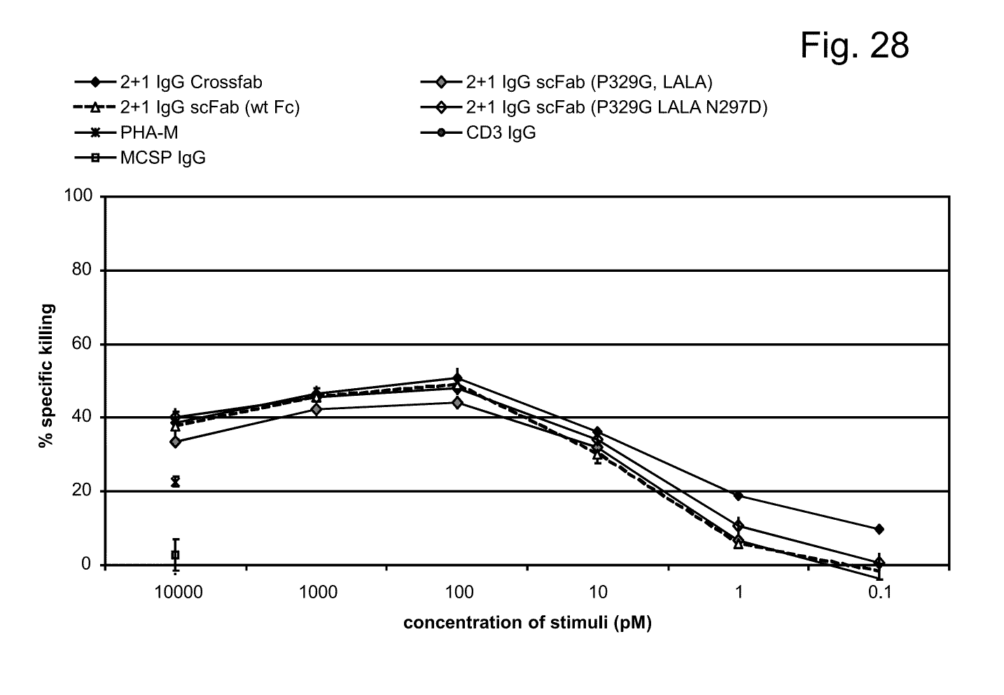

FIG. 28 shows that all constructs induce apoptosis in target cells comparable to the “(scFv)₂” molecule.

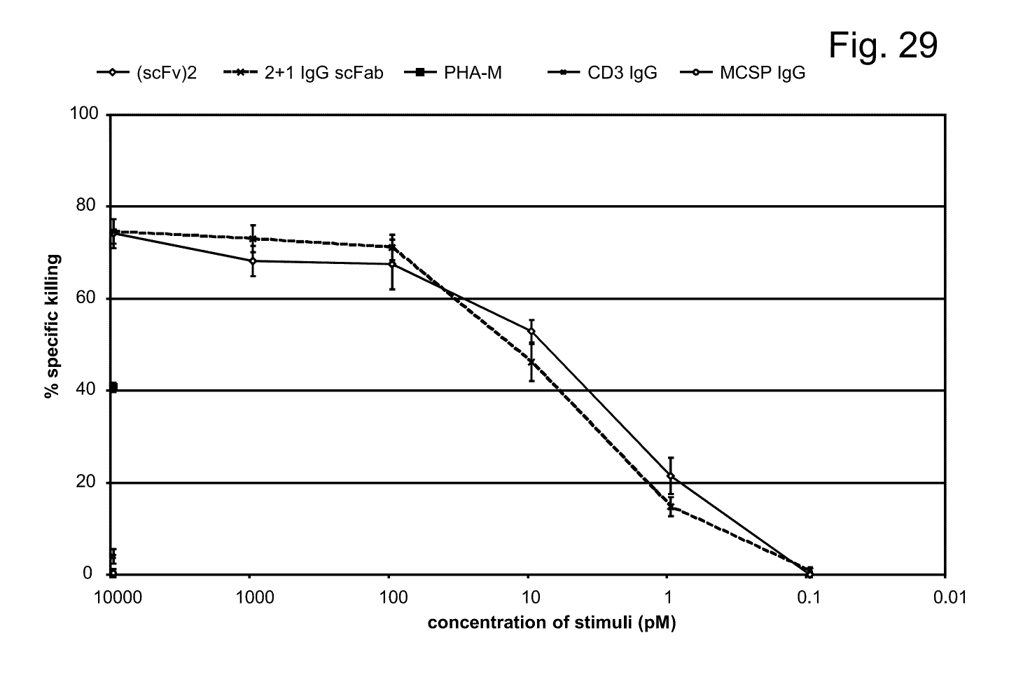

FIG. 29 shows the result of a comparison of the purified “2+1 IgG scFab” (SEQ ID NOs 5, 17, 19) and the “(scFv)₂” molecule for their potential to induce T cell-mediated apoptosis in tumor target cells. Experimental procedures were as described above, using huMCSP-expressing Colo-38 human melanoma target cells at an E:T ratio of 5:1, and an overnight incubation of 18.5 h. As depicted in the figure, the “2+1 IgG scFab” construct shows comparable cytotoxic activity to the “(scFv)₂” molecule.

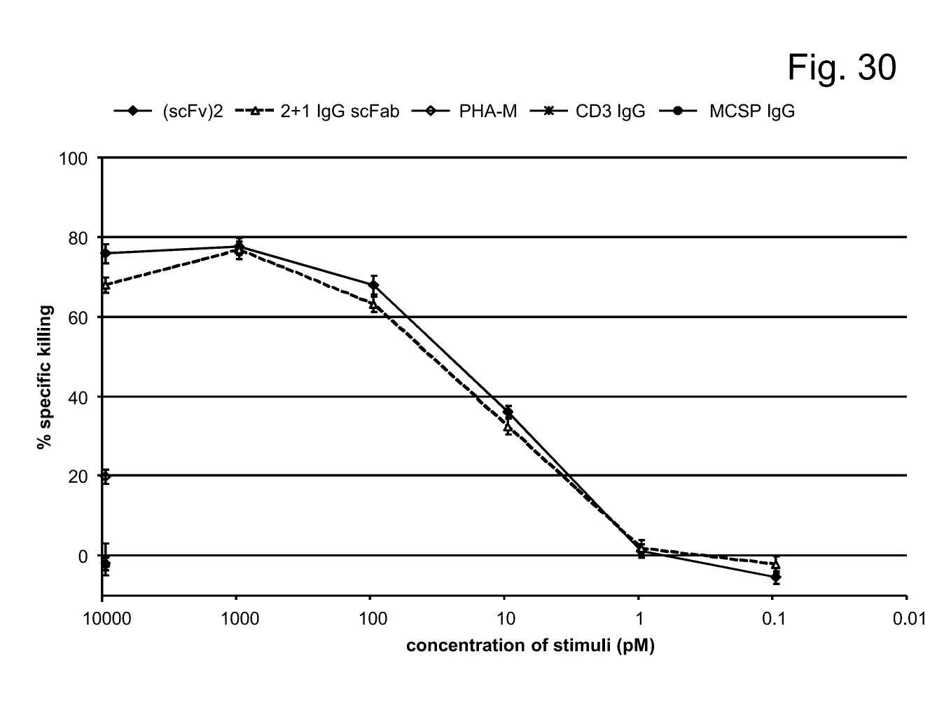

Similarly, FIG. 30 shows the result of a comparison of the purified “2+1 IgG scFab” construct (SEQ ID NOs 5, 17, 19) and the “(scFv)₂” molecule, using huMCSP-expressing Colo-38 human melanoma target cells at an E:T ratio of 5:1 and an incubation time of 18 h. As depicted in the figure, the “2+1 IgG scFab” construct shows comparable cytotoxic activity to the (scFv)₂ molecule.

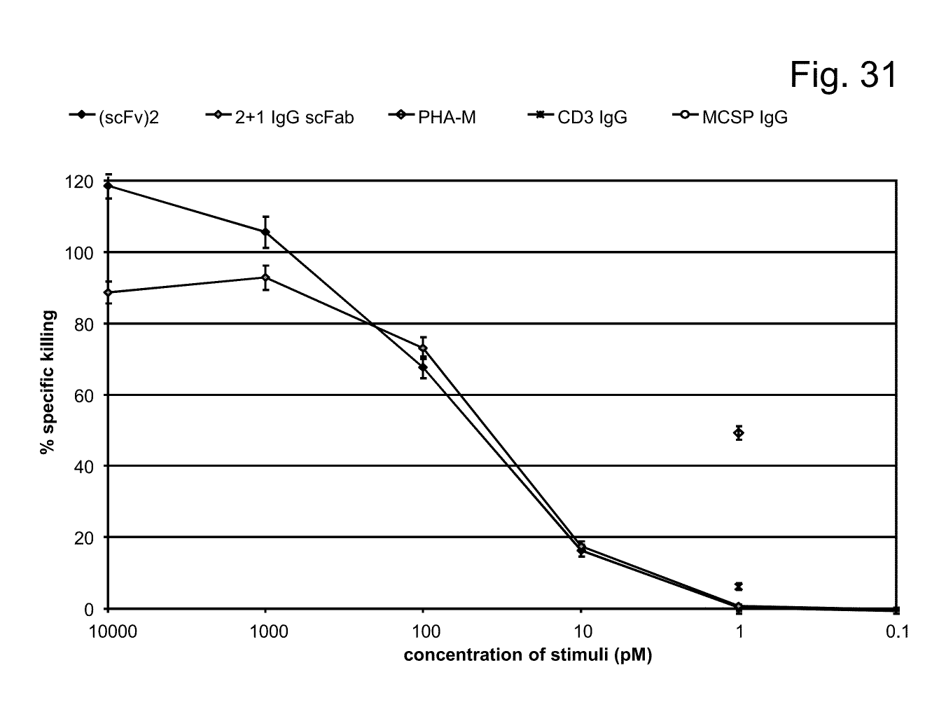

FIG. 31 shows the result of a comparison of the purified “2+1 IgG scFab” construct (SEQ ID NOs 5, 17, 19) and the “(scFv)₂” molecule, using huMCSP-expressing MDA-MB-435 human melanoma target cells at an E:T ratio of 5:1 and an overnight incubation of 23.5 h. As depicted in the figure, the construct induces apoptosis in target cells comparably to the “(scFv)₂” molecule. The “2+1 IgG scFab” construct shows reduced efficacy at the highest concentrations.

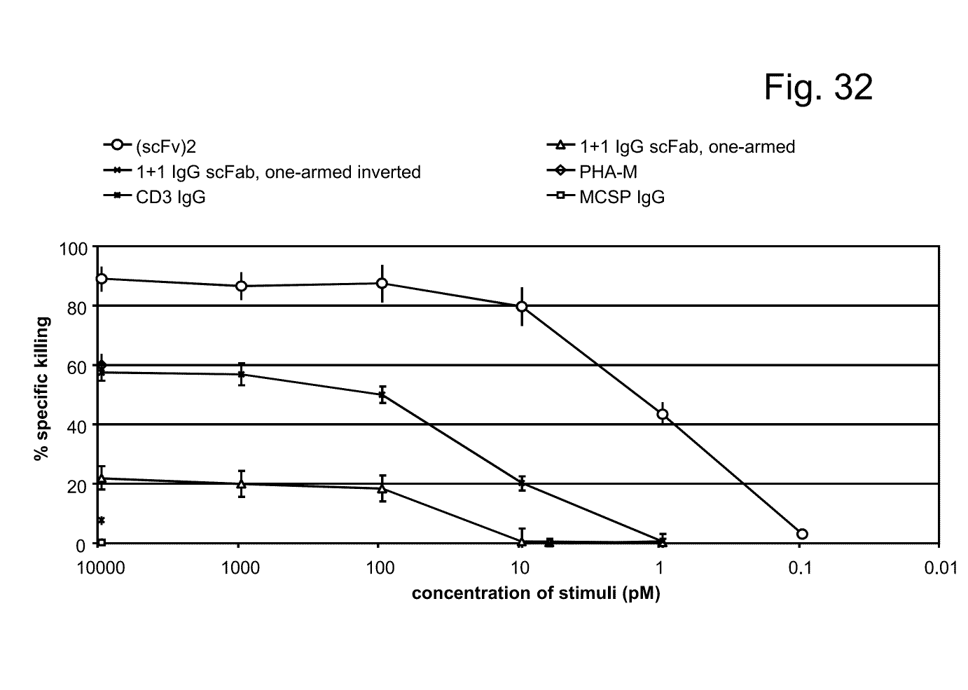

Furthermore, different bispecific constructs that are monovalent for both targets, human CD3 and human MCSP, as well as the corresponding “(scFv)₂” molecule were analyzed for their potential to induce T cell-mediated apoptosis. FIG. 32 shows the results for the “1+1 IgG scFab, one-armed” (SEQ ID NOs 1, 3, 5) and “1+1 IgG scFab, one-armed inverted” (SEQ ID NOs 7, 9, 11) constructs, using huMCSP-expressing Colo-38 human melanoma target cells at an E:T ratio of 5:1, and an incubation time of 19 h. As depicted in the figure, both “1+1” constructs are less active than the “(scFv)₂” molecule, with the “1+1 IgG scFab, one-armed” molecule being superior to the “1+1 IgG scFab, one-armed inverted” molecule in this assay.

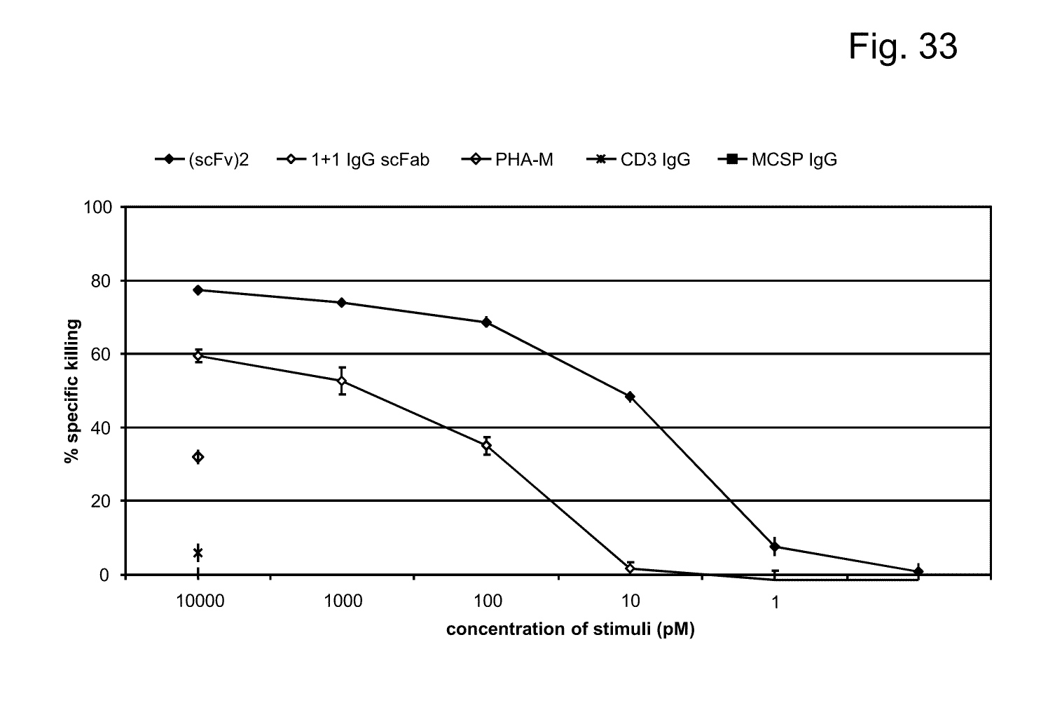

FIG. 33 shows the results for the “1+1 IgG scFab” construct (SEQ ID NOs 5, 21, 213), using huMCSP-expressing Colo-38 human melanoma target cells at an E:T ratio of 5:1, and an incubation time of 20 h. As depicted in the figure, the “1+1 IgG scFab” construct is less cytotoxic than the “(scFv)₂” molecule.

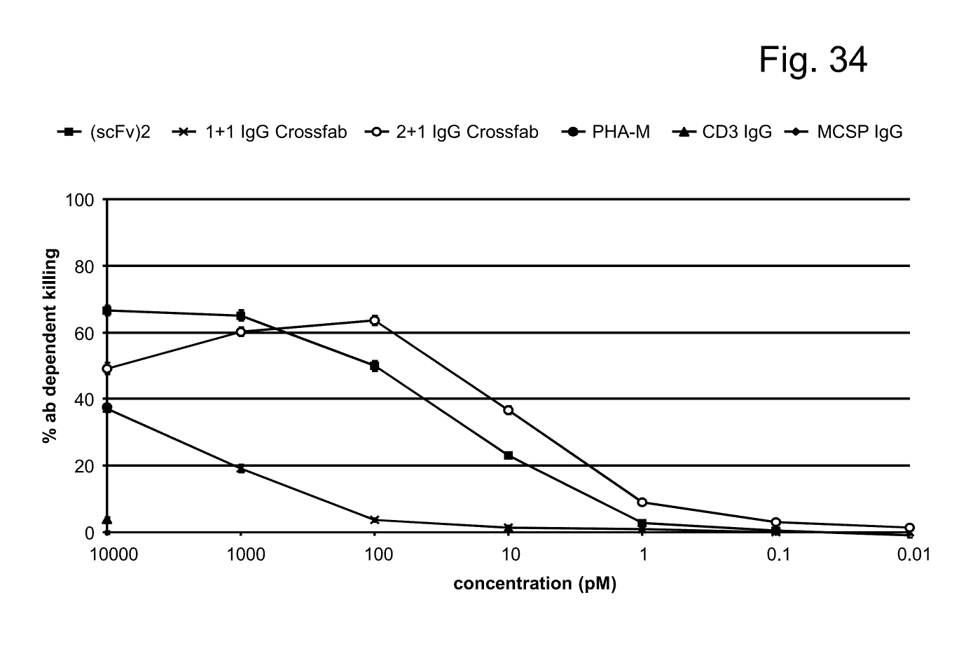

In a further experiment the purified “2+1 IgG Crossfab” (SEQ ID NOs 3, 5, 29, 33), the “1+1 IgG Crossfab” (SEQ ID NOs 5, 29, 31, 33) and the “(scFv)₂” molecule were analyzed for their potential to induce T cell-mediated apoptosis in tumor target cells upon crosslinkage of the construct via binding of both target antigens, CD3 and MCSP, on cells. huMCSP-expressing MDA-MB-435 human melanoma cells were used as target cells, the E:T ratio was 5:1, and the incubation time 20 h. The results are shown in FIG. 34. The “2+1 IgG Crossfab” construct induces apoptosis in target cells comparably to the “(scFv)₂” molecule. The comparison of the mono- and bivalent “IgG Crossfab” formats clearly shows that the bivalent one is much more potent.

In yet another experiment, the purified “2+1 IgG Crossfab” (SEQ ID NOs 3, 5, 29, 33) construct was analyzed for its potential to induce T cell-mediated apoptosis in different (tumor) target cells. Briefly, MCSP-positive Colo-38 tumor target cells, mesenchymal stem cells (derived from bone marrow, Lonza #PT-2501 or adipose tissue, Invitrogen #R7788-115) or pericytes (from placenta; PromoCell #C-12980), as indicated, were harvested with Cell Dissociation Buffer, washed and resuspended in AIM-V medium (Invitrogen #12055-091). 30 000 cells per well were plated in a round-bottom 96-well plate and the respective antibody dilution was added at the indicated concentrations. Human PBMC effector cells isolated from fresh blood of a healthy donor were added to obtain a final E:T ratio of 25:1. After an incubation of 4 h at 37° C., 5% CO₂, LDH release of apoptotic/necrotic target cells into the supernatant was measured with the LDH detection kit (Roche Applied Science, #11 644 793 001), according to the manufacturer's instructions.

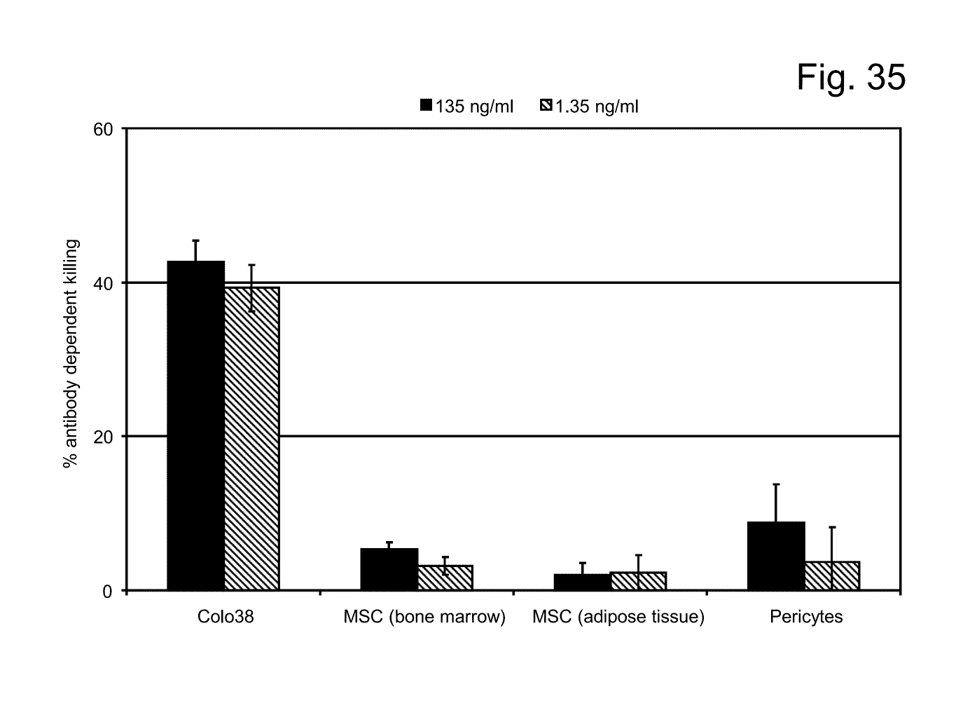

As depicted in FIG. 35, significant T-cell mediated cytotoxicity could be observed only with Colo-38 cells. This result is in line with Colo-38 cells expressing significant levels of MCSP, whereas mesenchymal stem cells and pericytes express MCSP only very weakly.

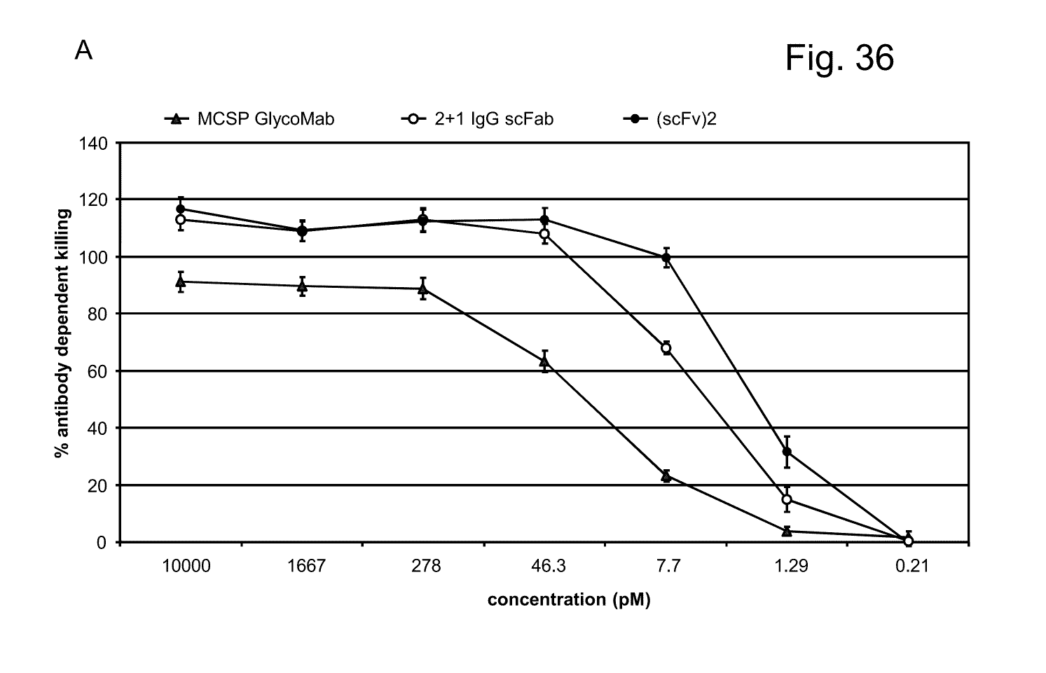

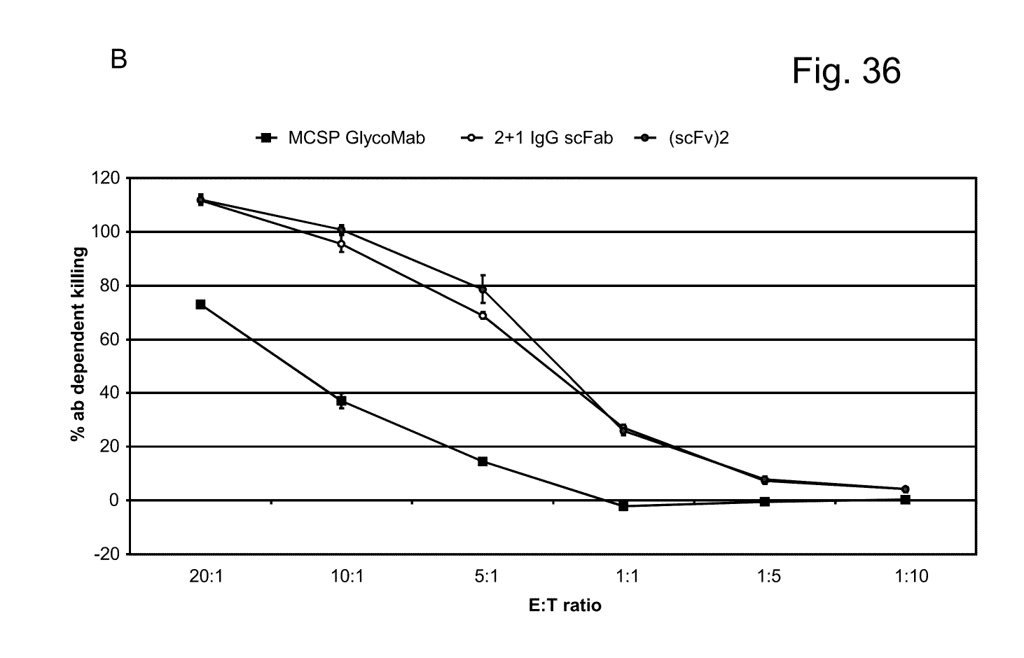

The purified “2+1 IgG scFab” (SEQ ID NOs 5, 17, 19) construct and the “(scFv)₂” molecule were also compared to a glycoengineered anti-human MCSP IgG antibody, having a reduced proportion of fucosylated N-glycans in its Fc domain (MCSP GlycoMab). For this experiment huMCSP-expressing Colo-38 human melanoma target cells and human PBMC effector cells were used, either at a fixed E:T ratio of 25:1 (FIG. 36A), or at different E:T ratios from 20:1 to 1:10 (FIG. 36B). The different molecules were used at the concentrations indicated in FIG. 36A, or at a fixed concentration of 1667 pM (FIG. 36B). Read-out was done after 21 h incubation. As depicted in FIGS. 36 A and B, both bispecific constructs show a higher potency than the MSCP GlycoMab.

In another experiment, purified “2+1 IgG Crossfab” targeting cynomolgus CD3 and human MCSP (SEQ ID NOs 3, 5, 35, 37) was analyzed. Briefly, human MCSP-expressing MV-3 tumor target cells were harvested with Cell Dissociation Buffer, washed and resuspended in DMEM containing 2% FCS and 1% GlutaMax. 30 000 cells per well were plated in a round-bottom 96-well plate and the respective dilution of construct or reference IgG was added at the concentrations indicated. The bispecific construct and the different IgG controls were adjusted to the same molarity. Cynomolgus PBMC effector cells, isolated from blood of healthy cynomolgus, were added to obtain a final E:T ratio of 3:1. After incubation for 24 h or 43 h at 37° C., 5% CO₂, LDH release of apoptotic/necrotic target cells into the supernatant was measured with the LDH detection kit (Roche Applied Science, #11 644 793 001), according to the manufacturer's instructions.

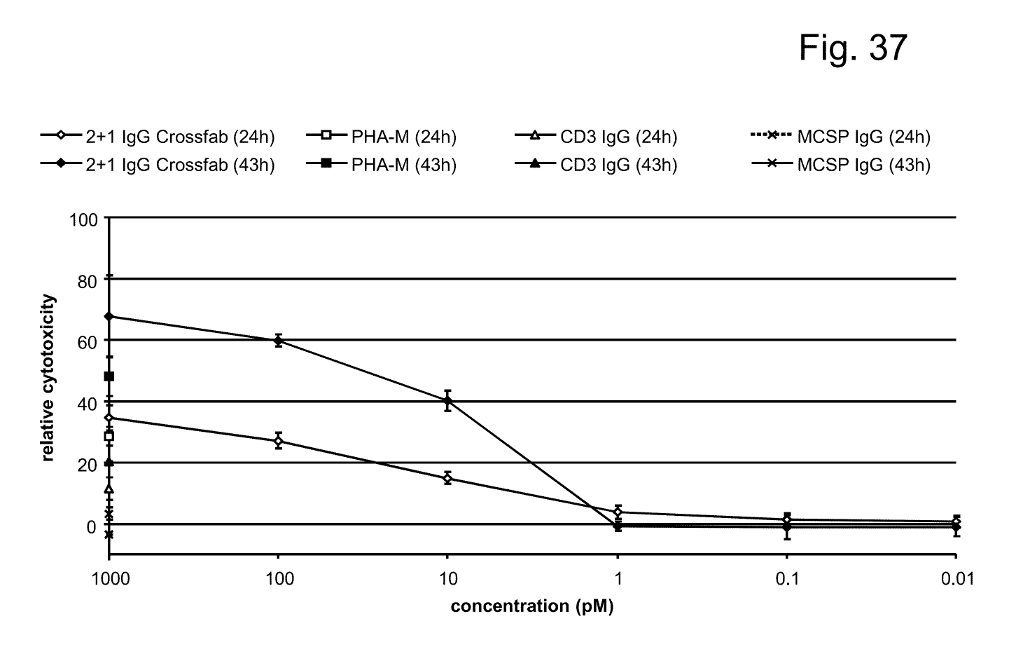

As depicted in FIG. 37, the bispecific construct induces concentration-dependent LDH release from target cells. The effect is stronger after 43 h than after 24 h. The anti-cynoCD3 IgG (clone FN-18) is also able to induce LDH release of target cells without being crosslinked.

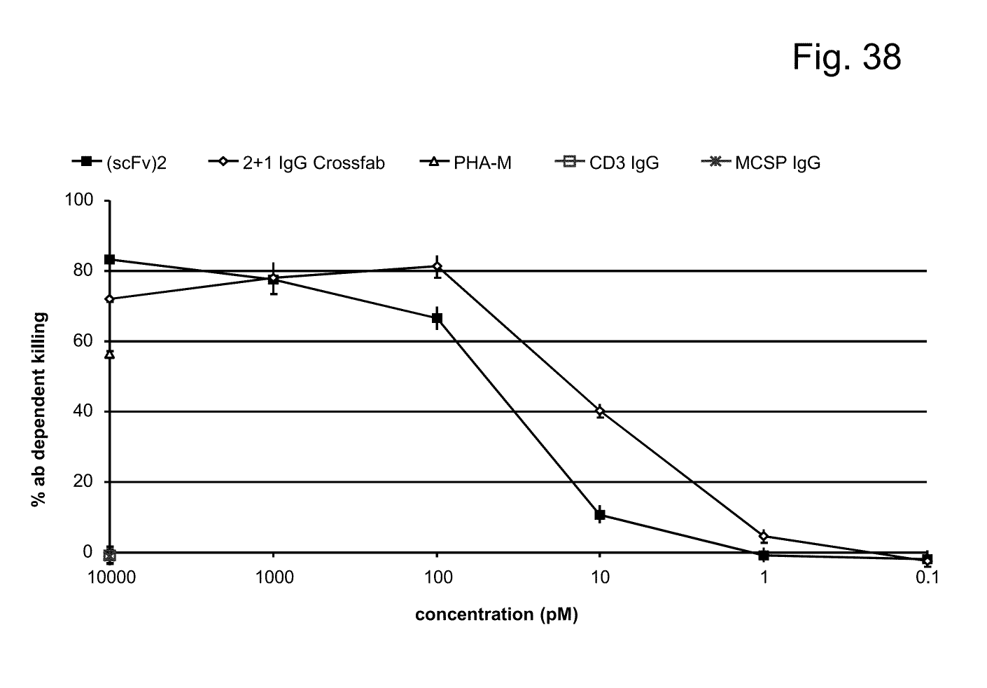

FIG. 38 shows the result of a comparison of the purified “2+1 IgG Crossfab” (SEQ ID NOs 3, 5, 29, 33) and the “(scFv)₂” construct, using MCSP-expressing human melanoma cell line (MV-3) as target cells and human PBMCs as effector cells with an E:T ratio of 10:1 and an incubation time of 26 h. As depicted in the figure, the “2+1 IgG Crossfab” construct is more potent in terms of EC50 than the “(scFv)₂” molecule.

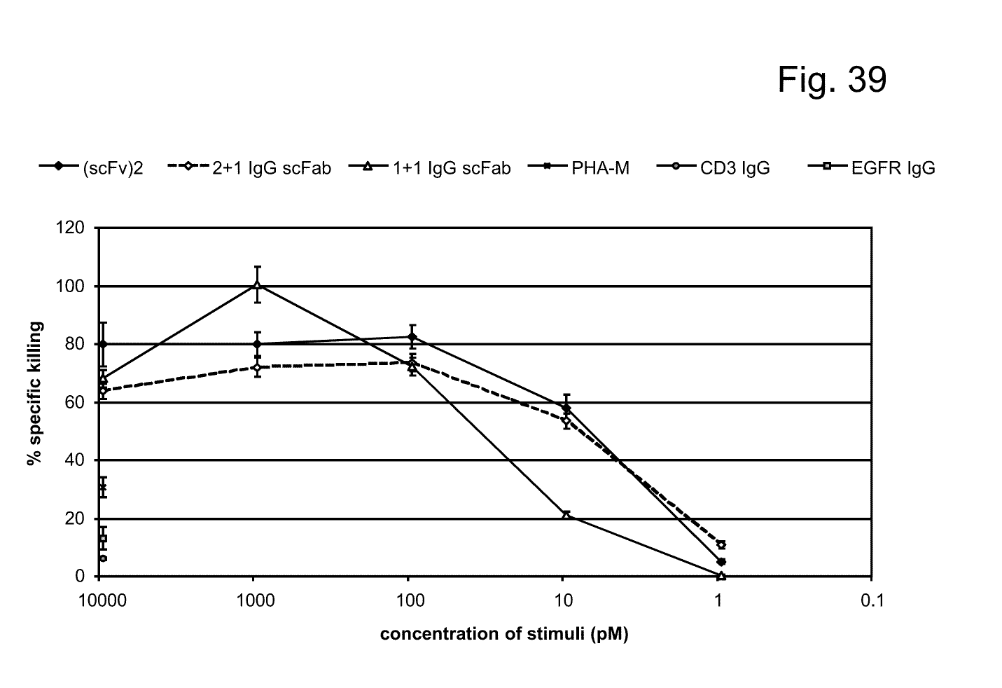

In a second series of experiments, bispecific constructs targeting CD3 and EGFR were analyzed for their potential to induce T cell-mediated apoptosis in tumor target cells upon crosslinkage of the construct via binding of the antigen binding moieties to their respective target antigens on cells (FIGS. 39-41).

In one experiment purified “2+1 IgG scFab” (SEQ ID NOs 45, 47, 53) and “1+1 IgG scFab” (SEQ ID NOs 47, 53, 213) constructs targeting CD3 and EGFR, and the corresponding “(scFv)₂” molecule, were compared. Briefly, human EGFR-expressing LS-174T tumor target cells were harvested with trypsin, washed and resuspended in AIM-V medium (Invitrogen #12055-091). 30 000 cells per well were plated in a round-bottom 96-well-plate and the respective antibody dilution was added at the indicated concentrations. All constructs and controls were adjusted to the same molarity. Human pan T effector cells were added to obtain a final E:T ratio of 5:1. As a positive control for the activation of human pan T cells, 1 μg/ml PHA-M (Sigma #L8902) was used. For normalization, maximal lysis of the target cells (=100%) was determined by incubation of the target cells with a final concentration of 1% Triton X-100. Minimal lysis (=0%) refers to target cells co-incubated with effector cells, but without any construct or antibody. After an overnight incubation of 18 h at 37° C., 5% CO₂, LDH release of apoptotic/necrotic target cells into the supernatant was measured with the LDH detection kit (Roche Applied Science, #11 644 793 001), according to the manufacturer's instructions.

As depicted in FIG. 39, the “2+1 IgG scFab” construct shows comparable cytotoxic activity to the “(scFv)₂” molecule, whereas the “1+1 IgG scFab” construct is less active.

In another experiment the purified “1+1 IgG scFab, one-armed” (SEQ ID NOs 43, 45, 47), “1+1 IgG scFab, one-armed inverted” (SEQ ID NOs 11, 49, 51), “1+1 IgG scFab” (SEQ ID NOs 47, 53, 213), and the “(scFv)₂” molecule were compared. Experimental conditions were as described above, except for the incubation time which was 21 h.

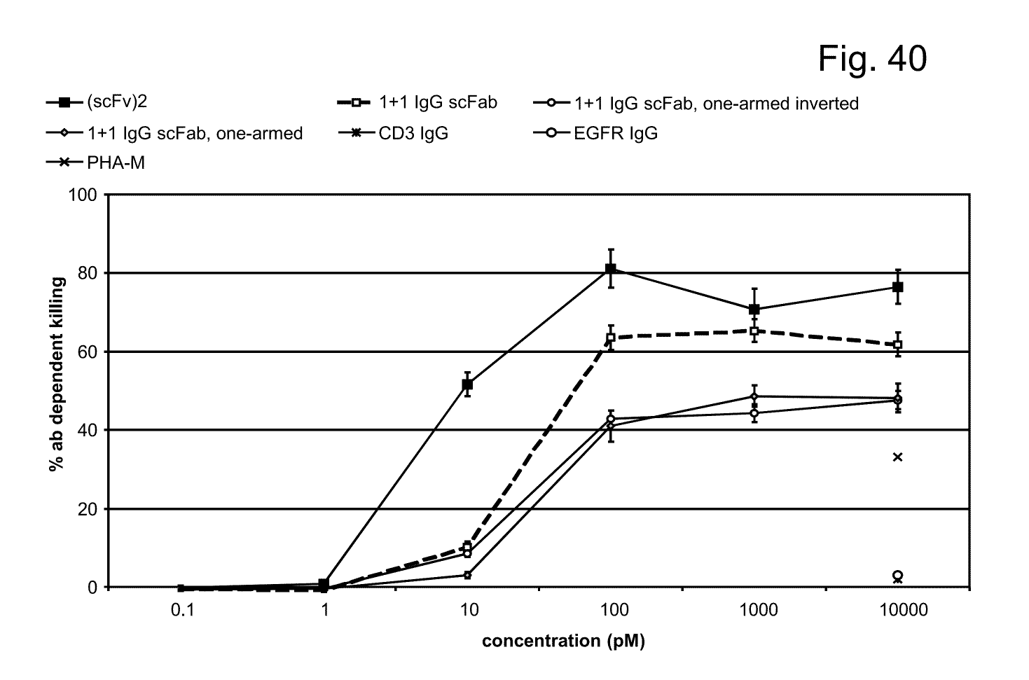

As depicted in FIG. 40, the “1+1 IgG scFab” construct shows a slightly lower cytotoxic activity than the “(scFv)₂” molecule in this assay. Both “1+1 IgG scFab, one-armed (inverted)” constructs are clearly less active than the “(scFv)₂” molecule.

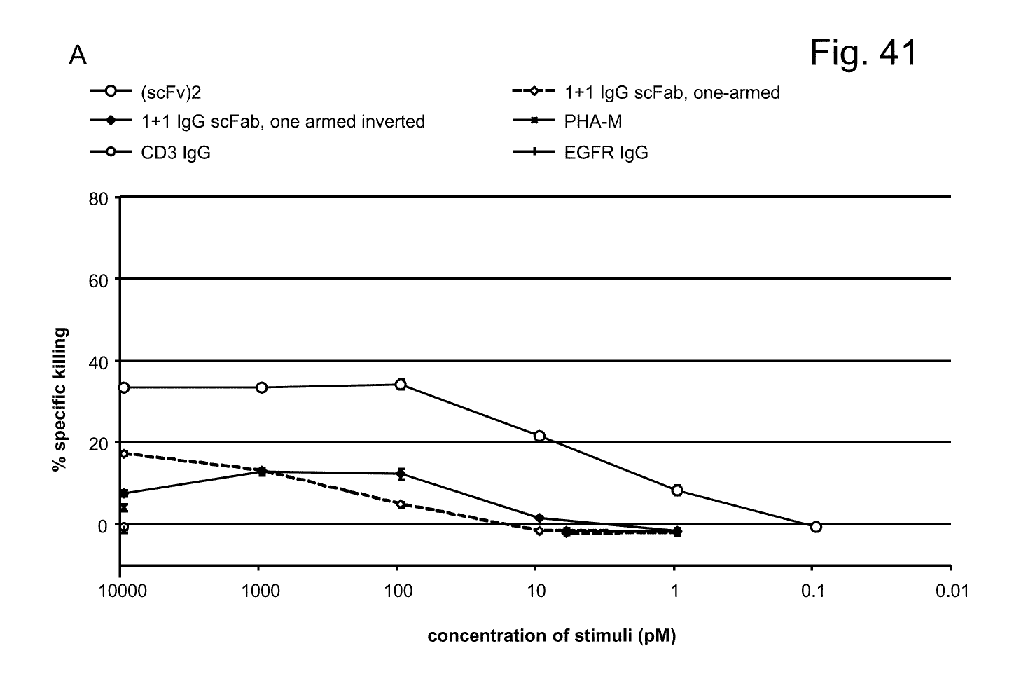

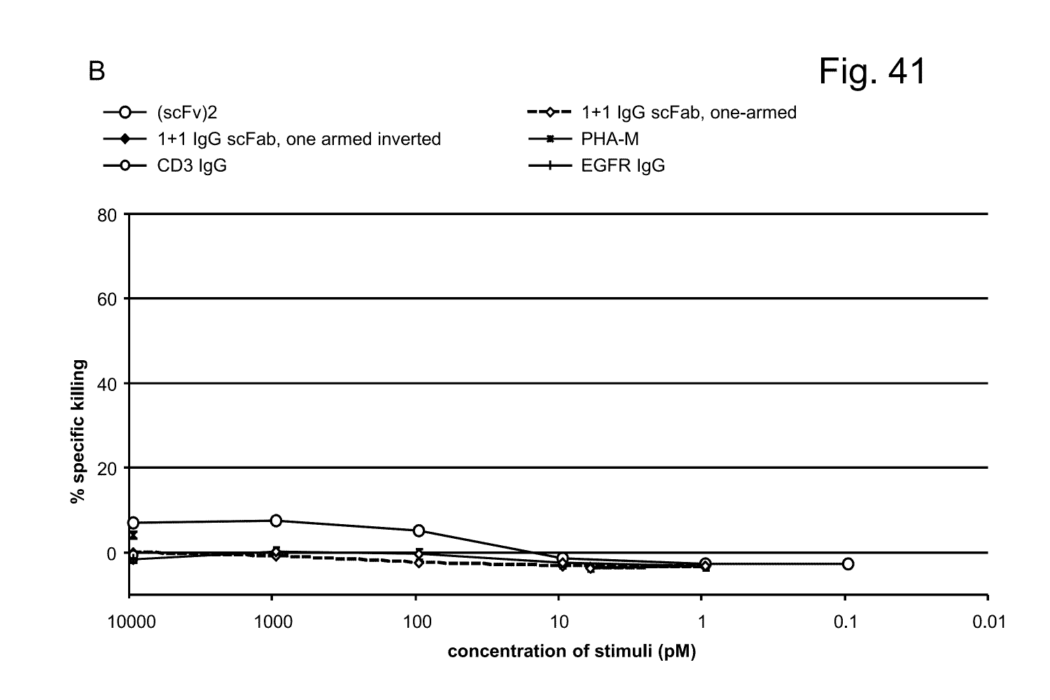

In yet a further experiment the purified “1+1 IgG scFab, one-armed” (SEQ ID NO 43, 45, 47) and “1+1 IgG scFab, one-armed inverted” (SEQ ID NOs 11, 49, 51) constructs and the “(scFv)₂” molecule were compared. The incubation time in this experiment was 16 h, and the result is depicted in FIG. 41. Incubated with human pan T cells, both “1+1 IgG scFab, one-armed (inverted)” constructs are less active than the “(scFv)₂” molecule, but show concentration-dependent release of LDH from target cells (FIG. 41A). Upon co-cultivation of the LS-174T tumor cells with naive T cells isolated from PBMCs, the constructs had only a basal activity—the most active among them being the “(scFv)₂” molecule (FIG. 41B).

In a further experiment, purified “1+1 IgG scFab, one-armed inverted” (SEQ ID NOs 11, 51, 55), “1+1 IgG scFab” (57, 61, 213), and “2+1 IgG scFab” (57, 59, 61) targeting CD3 and Fibroblast Activation Protein (FAP), and the corresponding “(scFv)₂” molecule were analyzed for their potential to induce T cell-mediated apoptosis in human FAP-expressing fibroblasts GM05389 cells upon crosslinkage of the construct via binding of both targeting moieties to their respective target antigens on the cells. Briefly, human GM05389 target cells were harvested with trypsin on the day before, washed and resuspended in AIM-V medium (Invitrogen #12055-091). 30 000 cells per well were plated in a round-bottom 96-well plate and incubated overnight at 37° C., 5% CO₂ to allow the cells to recover and adhere. The next day, the cells were centrifuged, the supernatant was discarded and fresh medium, as well as the respective dilution of the constructs or reference IgGs was added at the indicated concentrations. All constructs and controls were adjusted to the same molarity. Human pan T effector cells were added to obtain a final E:T ratio of 5:1. As a positive control for the activation of human pan T cells, 5 μg/ml PHA-M (Sigma #L8902) was used. For normalization, maximal lysis of the target cells (=100%) was determined by incubation of the target cells with a final concentration of 1% Triton X-100.

Minimal lysis (=0%) refers to target cells co-incubated with effector cells, but without any construct or antibody. After an additional overnight incubation of 18 h at 37° C., 5% CO₂, LDH release of apoptotic/necrotic target cells into the supernatant was measured with the LDH detection kit (Roche Applied Science, #11 644 793 001), according to the manufacturer's instructions.

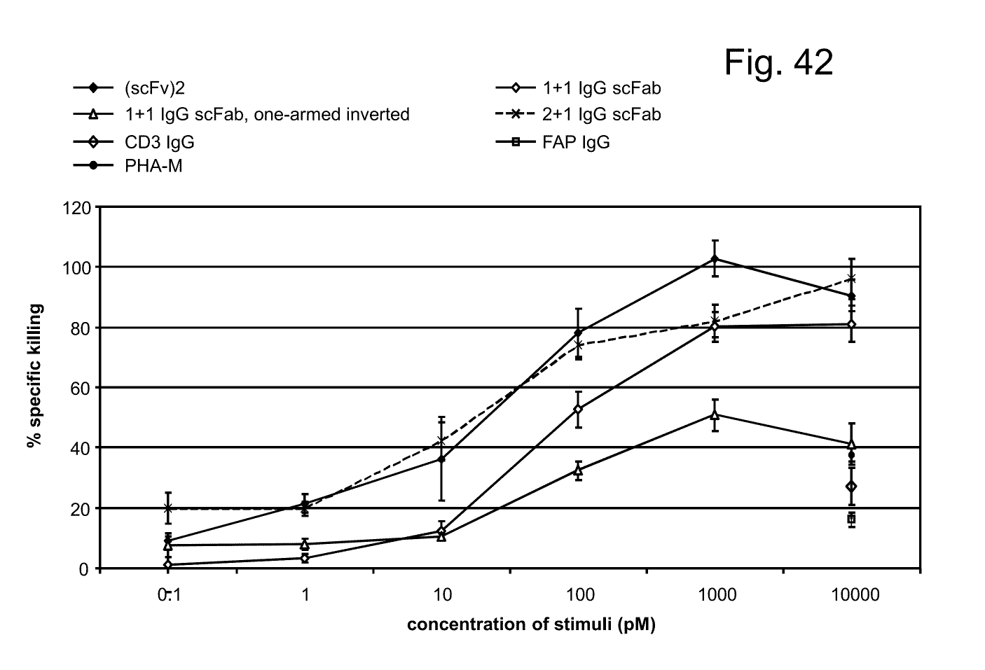

As depicted in FIG. 42, the “2+1 IgG scFab” construct shows comparable cytotoxic activity to the “(scFv)₂” molecule in terms of EC50 values. The “1+1 IgG scFab, one-armed inverted” construct is less active than the other constructs tested in this assay.

In another set of experiments, the CD3/MCSP “2+1 IgG Crossfab, linked light chain” (see SEQ ID NOs 3, 5, 29, 179) was compared to the CD3/MCSP “2+1 IgG Crossfab” (see SEQ ID NOs 3, 5, 29, 33). Briefly, target cells (human Colo-38, human MV-3 or WM266-4 melanoma cells) were harvested with Cell Dissociation Buffer on the day of the assay (or with trypsin one day before the assay was started), washed and resuspended in the appropriate cell culture medium (RPMI1640, including 2% FCS and 1% Glutamax). 20 000-30 000 cells per well were plated in a flat-bottom 96-well plate and the respective antibody dilution was added as indicated (triplicates). PBMCs as effector cells were added to obtain a final effector-to-target cell (E:T) ratio of 10:1. All constructs and controls were adjusted to the same molarity, incubation time was 22 h. Detection of LDH release and normalization was done as described above.

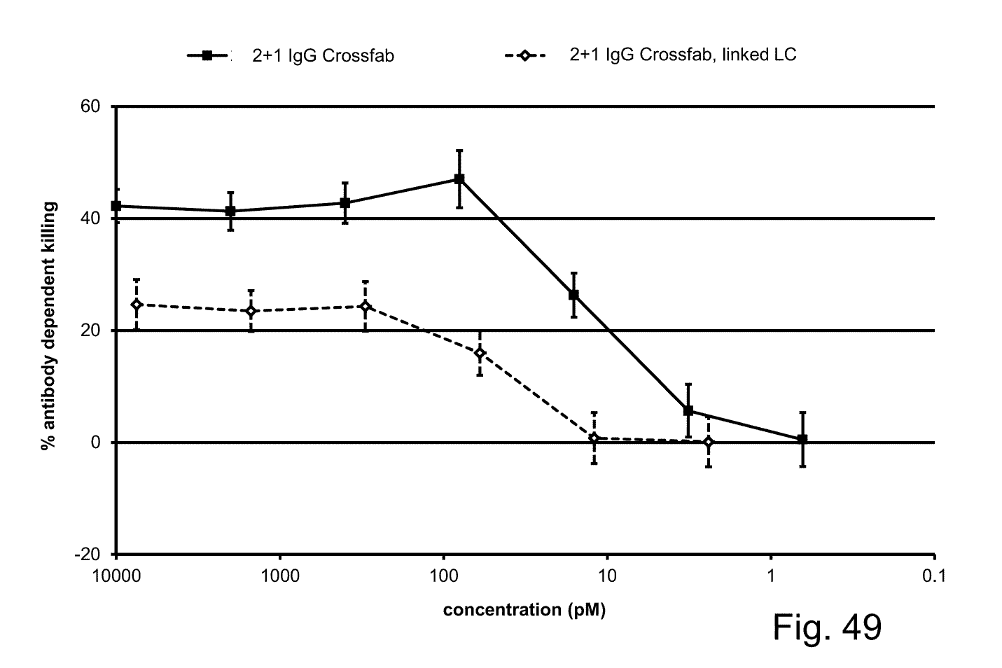

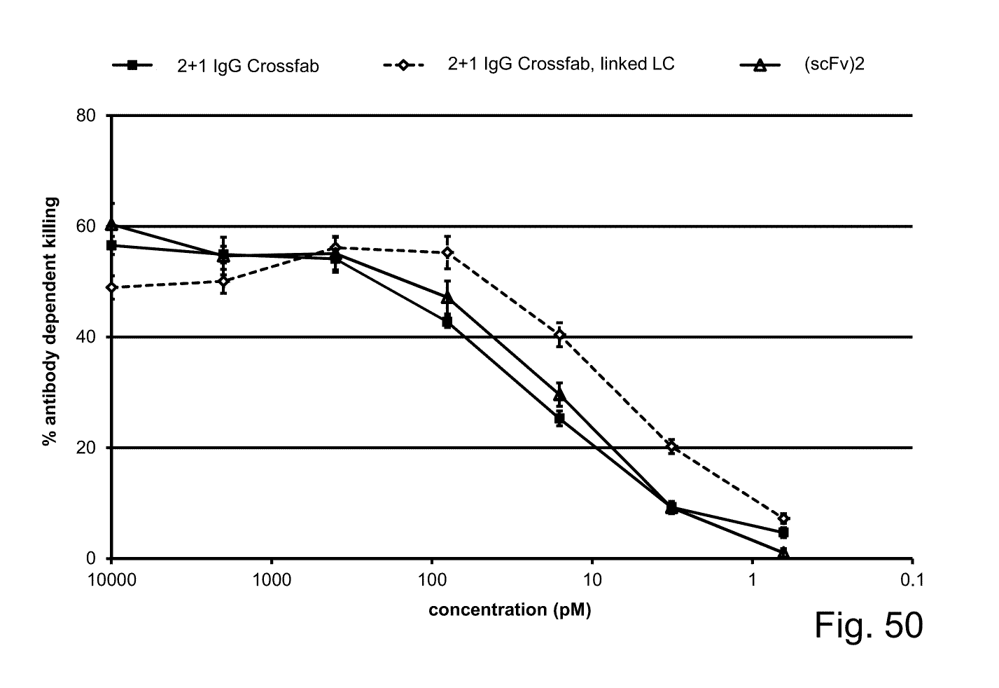

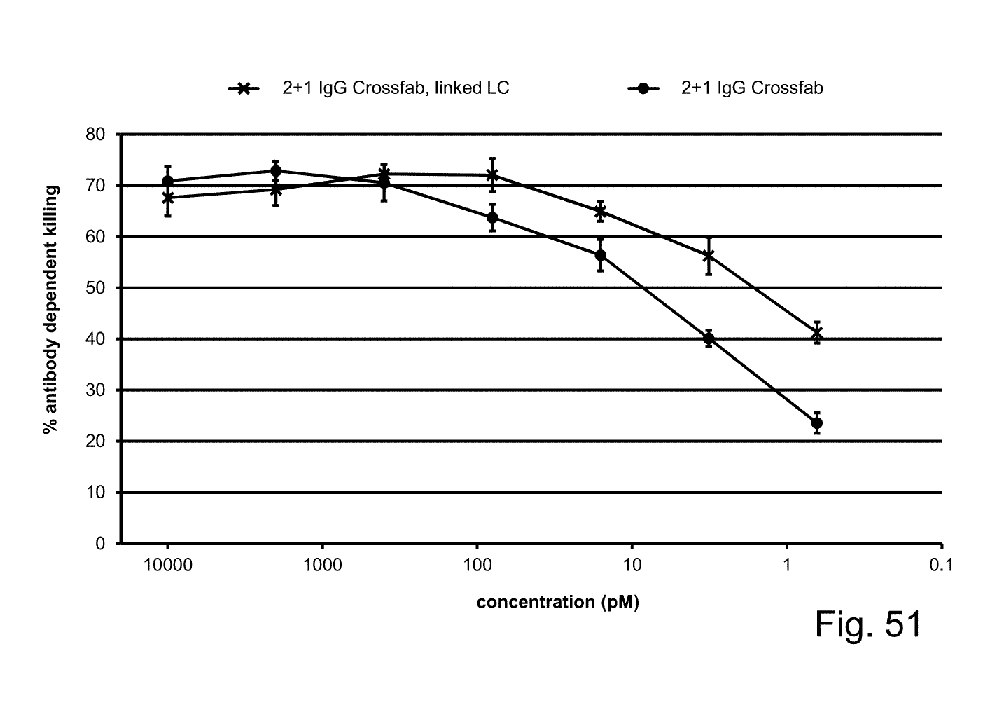

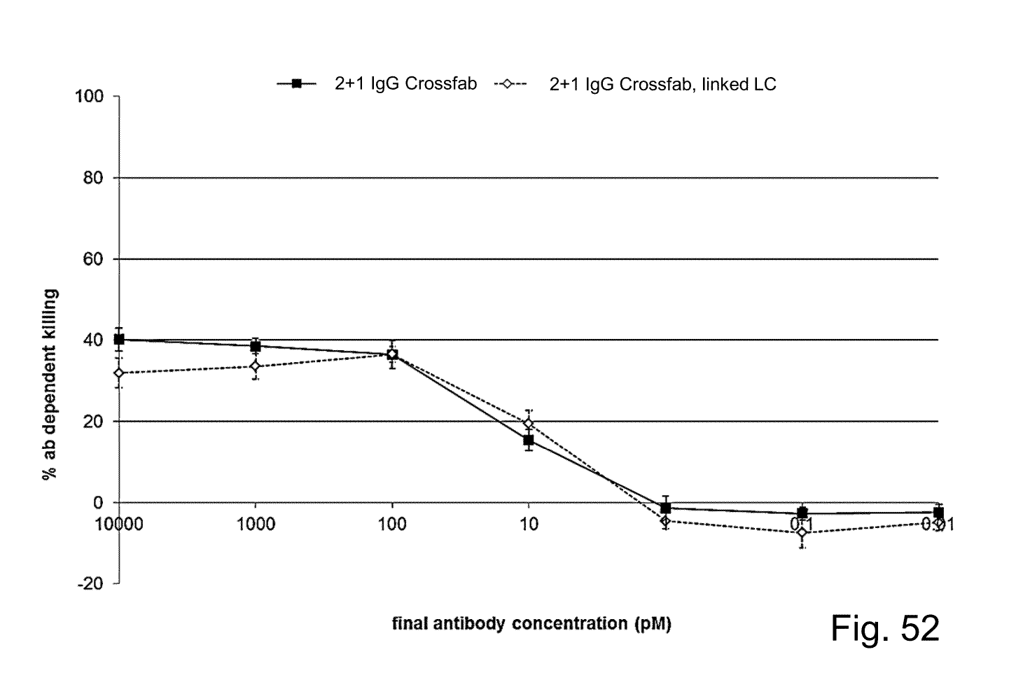

FIGS. 49 to 52 show the result of four assays performed with MV-3 melanoma cells (FIG. 49), Colo-38 cells (FIGS. 50 and 51) or WM266-4 cells (FIG. 52). As shown in FIG. 49, the construct with the linked light chain was less potent compared to the one without the linked light chain in the assay with MV-3 cells as target cells. As shown in FIGS. 50 and 51, the construct with the linked light chain was more potent compared to the one without the linked light chain in the assays with high MCSP expressing Colo-38 cells as target cells. Finally, as shown in FIG. 52, there was no significant difference between the two constructs when high MCSP-expressing WM266-4 cells were used as target cells.

In another experiment, two CEA-targeting “2+1 IgG Crossfab, inverted” constructs were compared, wherein in the Crossfab fragment either the V regions (VL/VH, see SEQ ID NOs 33, 63, 65, 67) or the C regions (CL/CH1, see SEQ ID NOs 65, 67, 183, 197) were exchanged. The assay was performed as described above, using human PBMCs as effector cells and human CEA-expressing target cells. Target cells (MKN-45 or LS-174T tumor cells) were harvested with trypsin-EDTA (LuBiosciences #25300-096), washed and resuspended in RPMI1640 (Invitrogen #42404042), including 1% Glutamax (LuBiosciences #35050087) and 2% FCS. 30 000 cells per well were plated in a round-bottom 96-well plate and the bispecific constructs were added at the indicated concentrations. All constructs and controls were adjusted to the same molarity. Human PBMC effector cells were added to obtain a final E:T ratio of 10:1, incubation time was 28 h. EC50 values were calculated using the GraphPad Prism 5 software.

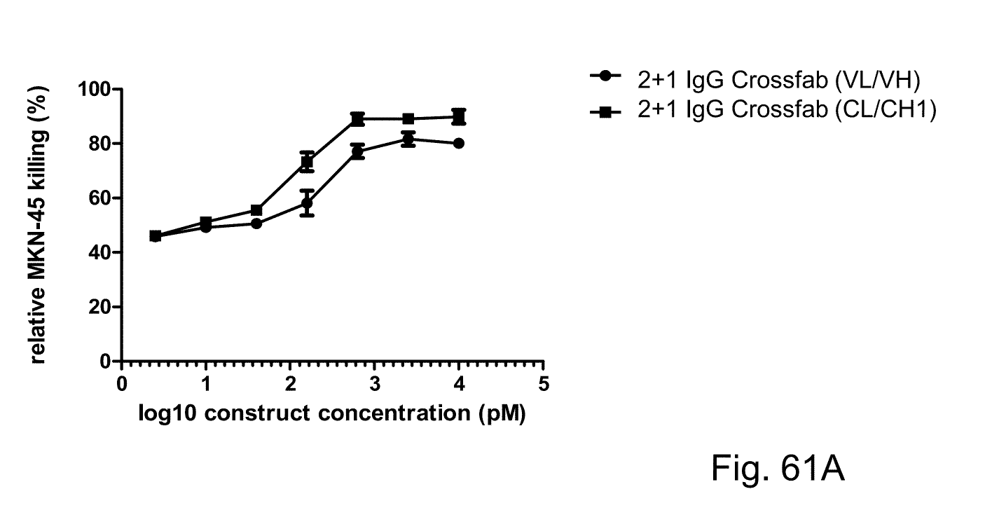

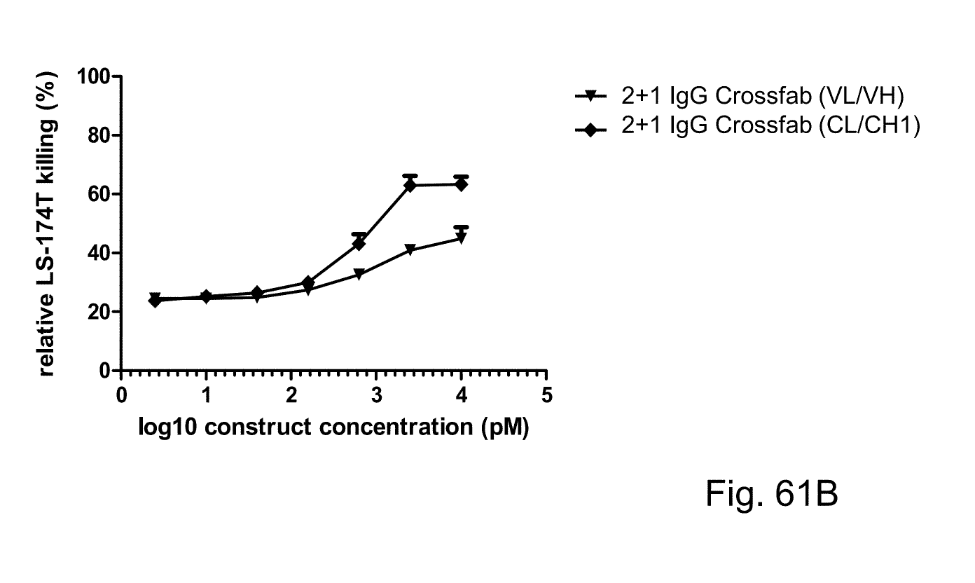

As shown in FIG. 61, the construct with the CL/CH1 exchange shows slightly better activity on both target cell lines than the construct with the VL/VH exchange. Calculated EC50 values were 115 and 243 pM on MKN-45 cells, and 673 and 955 pM on LS-174T cells, for the CL/CH1-exchange construct and the VL/VH-exchange construct, respectively.

Similarly, two MCSP-targeting “2+1 IgG Crossfab” constructs were compared, wherein in the Crossfab fragment either the V regions (VL/VH, see SEQ ID NOs 33, 189, 191, 193) or the C regions (CL/CH1, see SEQ ID NOs 183, 189, 193, 195) were exchanged. The assay was performed as described above, using human PBMCs as effector cells and human MCSP-expressing target cells. Target cells (WM266-4) were harvested with Cell Dissociation Buffer (LuBiosciences #13151014), washed and resuspended in RPMI1640 (Invitrogen #42404042), including 1% Glutamax (LuBiosciences #35050087) and 2% FCS. 30 000 cells per well were plated in a round-bottom 96-well plate and the constructs were added at the indicated concentrations. All constructs and controls were adjusted to the same molarity. Human PBMC effector cells were added to obtain a final E:T ratio of 10:1, incubation time was 26 h. EC50 values were calculated using the GraphPad Prism 5 software.

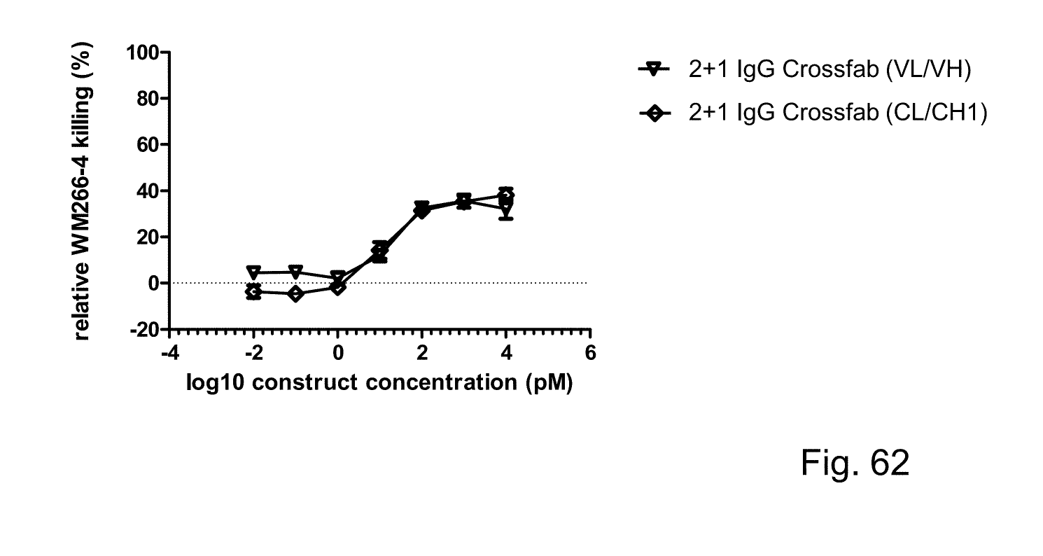

As depicted in FIG. 62, the two constructs show comparable activity, the construct with the CL/CH1 exchange having a slightly lower EC50 value (12.9 pM for the CL/CH1-exchange construct, compared to 16.8 pM for the VL/VH-exchange construct).

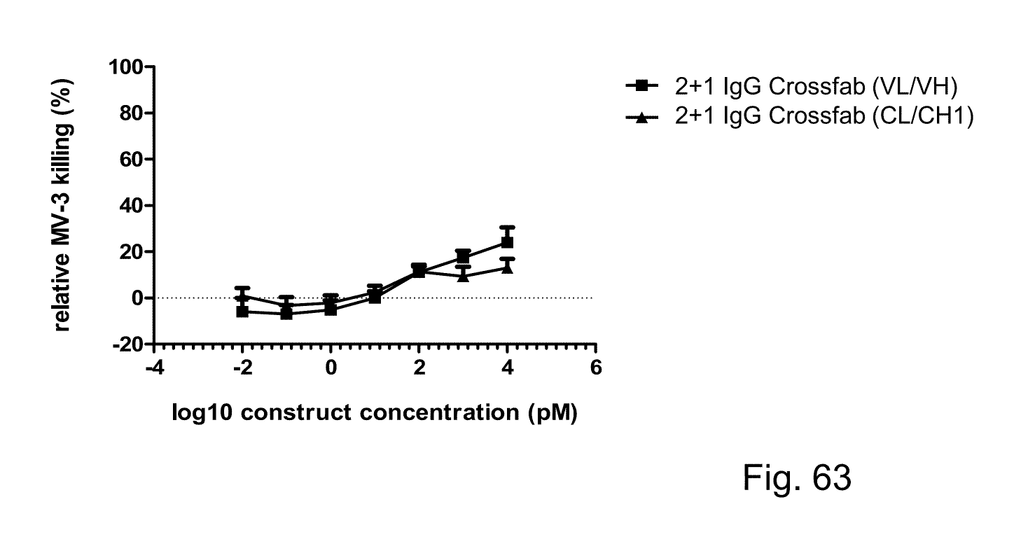

FIG. 63 shows the result of a similar assay, performed with human MCSP-expressing MV-3 target cells. Again, both constructs show comparable activity, the construct with the CL/CH1 exchange having a slightly lower EC50 value (approximately 11.7 pM for the CL/CH1-exchange construct, compared to approximately 82.2 pM for the VL/VH-exchange construct). Exact EC50 values could not be calculated, since the killing curves did not reach a plateau at high concentrations of the compounds.

In a further experiment, the CD3/MCSP “2+1 IgG Crossfab” (see SEQ ID NOs 3, 5, 29, 33) and “1+1 IgG Crossfab” (see SEQ ID NOs 5, 29, 33, 181) constructs were compared to the CD3/MCSP “1+1 CrossMab” (see SEQ ID NOs 5, 23, 183, 185). The assay was performed as described above, using human PBMCs as effector cells and WM266-4 or MV-3 target cells (E:T ratio=10:1) and an incubation time of 21 h.

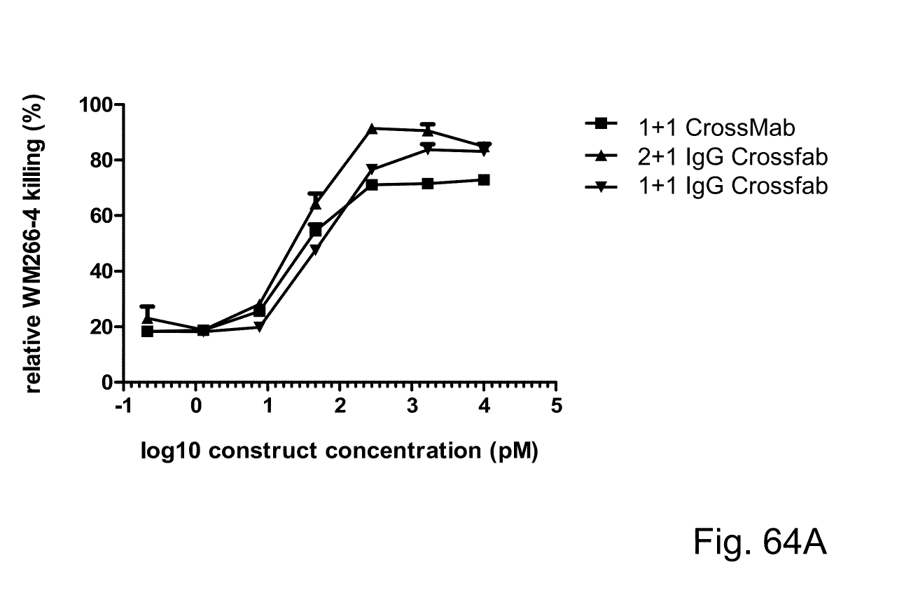

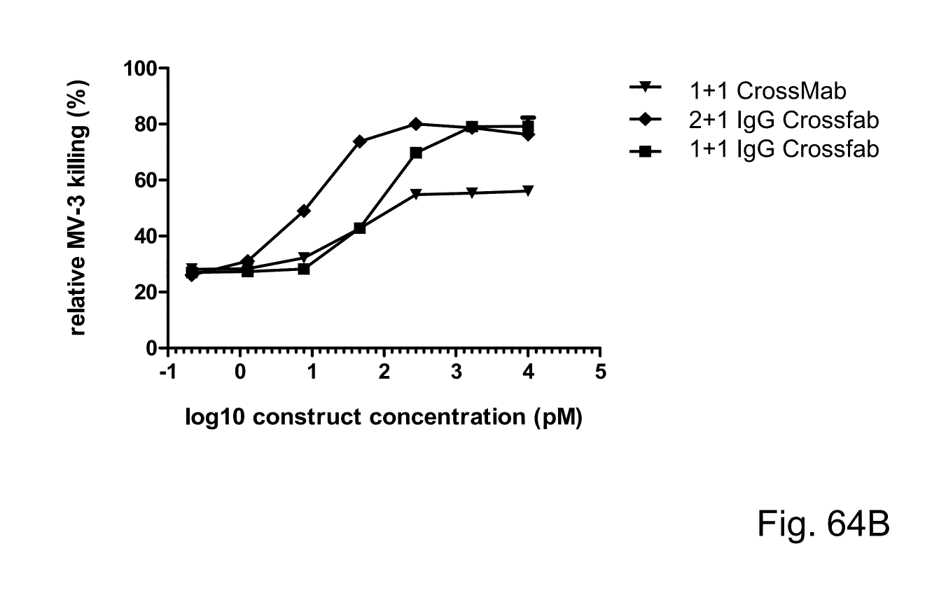

As shown in FIG. 64, the “2+1 IgG Crossfab” construct is the most potent molecule in this assay, followed by the “1+1 IgG Crossfab” and the “1+1 CrossMab”. This ranking is even more pronounced with MV-3 cells, expressing medium levels of MCSP, compared to high MCSP expressing WM266-4 cells. The calculated EC50 values on MV-3 cells were 9.2, 40.9 and 88.4 pM, on WM266-4 cells 33.1, 28.4 and 53.9 pM, for the “2+1 IgG Crossfab”, the “1+1 IgG Crossfab” and the “1+1 CrossMab”, respectively.

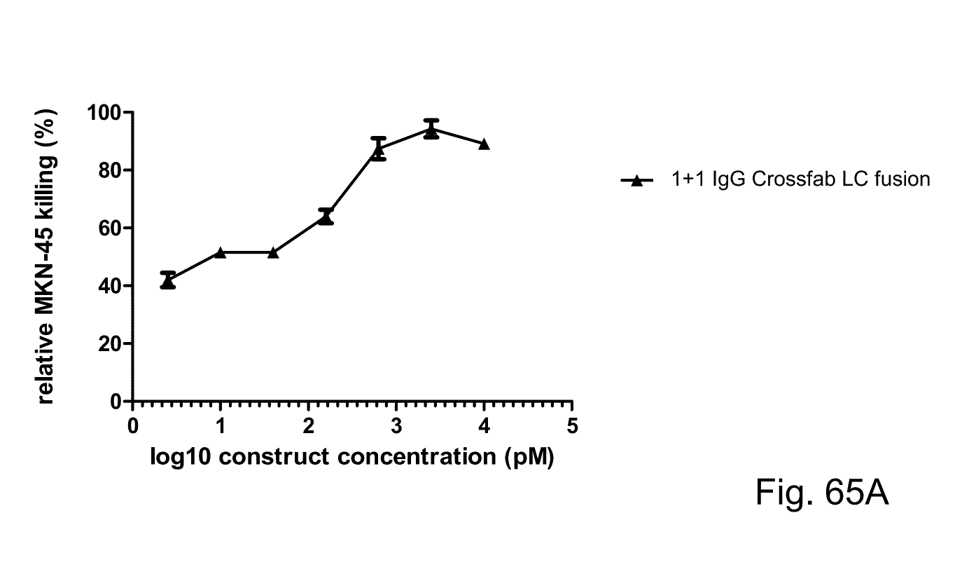

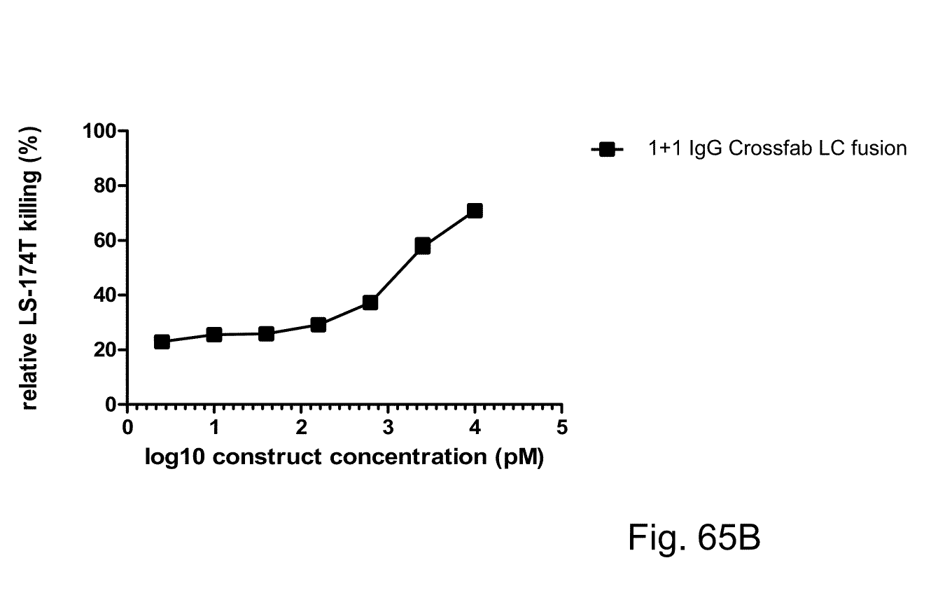

In a further experiment, different concentrations of the “1+1 IgG Crossfab LC fusion” construct (SEQ ID NOs 183, 209, 211, 213) were tested, using MKN-45 or LS-174T tumor target cells and human PBMC effector cells at an E:T ratio of 10:1 and an incubation time of 28 hours. As shown in FIG. 65, the “1+1 IgG Crossfab LC fusion” construct induced apoptosis in MKN-45 target cells with a calculated EC50 of 213 pM, whereas the calculated EC50 is 1.56 nM with LS-174T cells, showing the influence of the different tumor antigen expression levels on the potency of the bispecific constructs within a certain period of time.

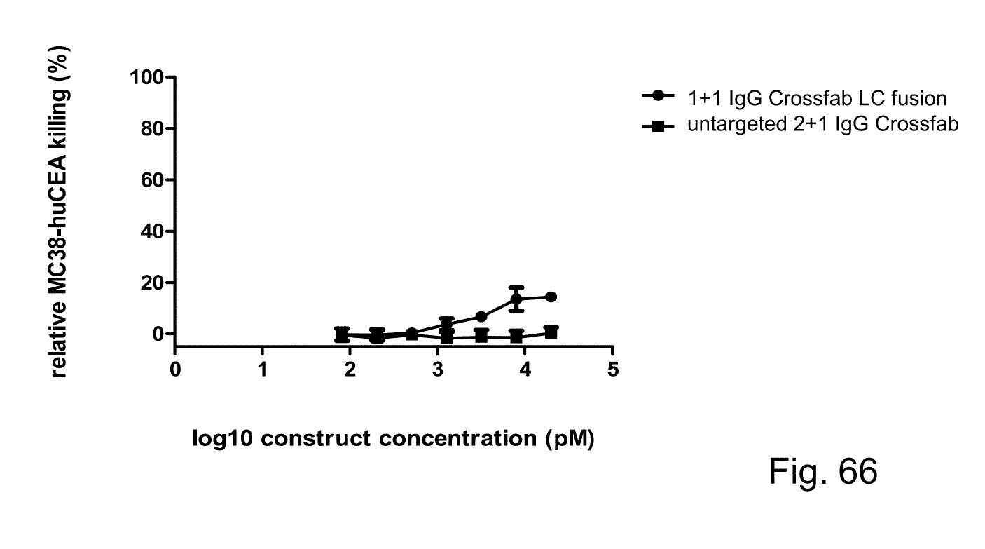

In yet another experiment, the “1+1 IgG Crossfab LC fusion” construct (SEQ ID NOs 183, 209, 211, 213) was compared to a untargeted “2+1 IgG Crossfab” molecule. MC38-huCEA tumor cells and human PBMCs (E:T ratio=10:1) and an incubation time of 24 hours were used. As shown in FIG. 66, the “1+1 IgG Crossfab LC fusion” construct induced apoptosis of target cells in a concentration-dependent manner, with a calculated EC50 value of approximately 3.2 nM. In contrast, the untargeted “2+1 IgG Crossfab” showed antigen-independent T cell-mediated killing of target cells only at the highest concentration.

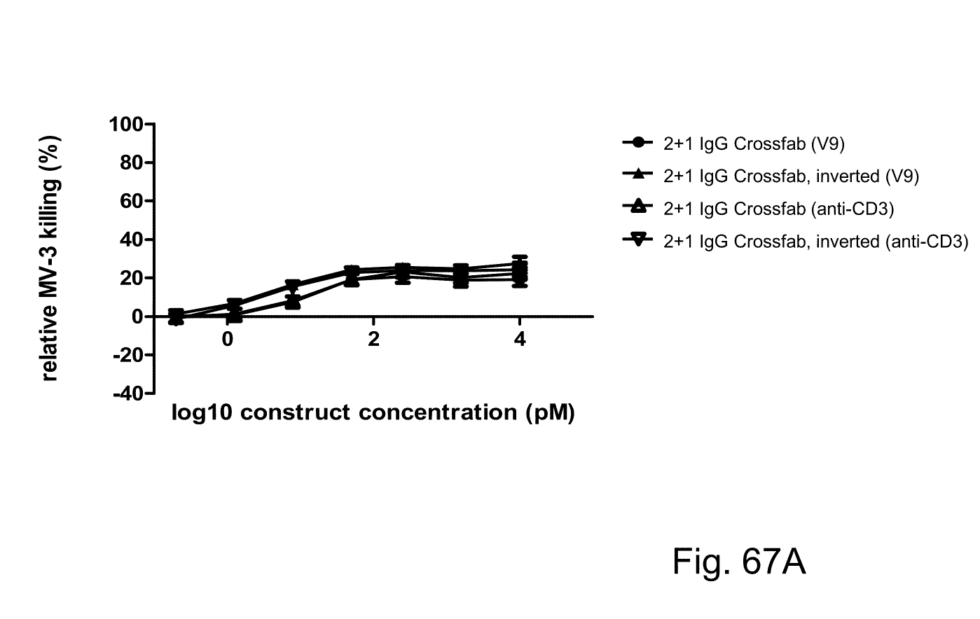

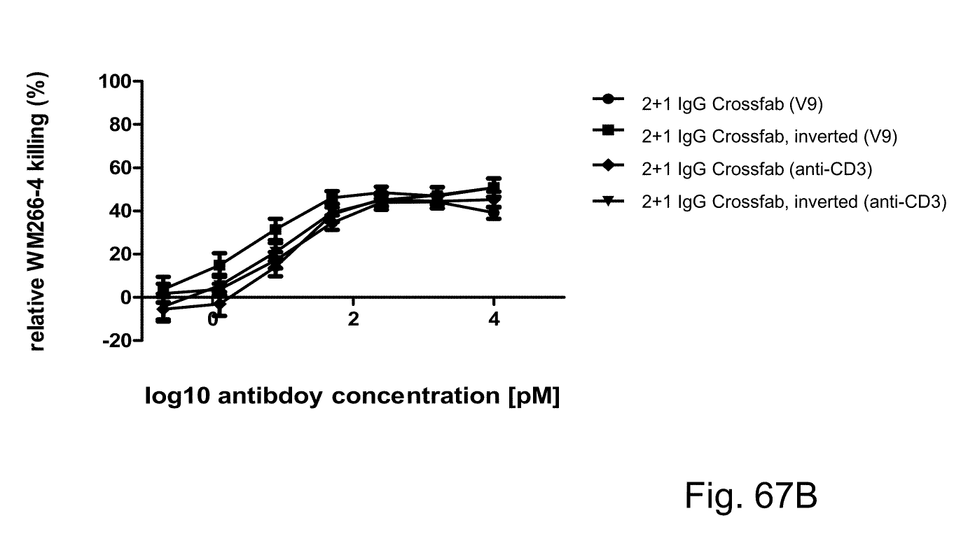

In a final experiment, the “2+1 IgG Crossfab (V9)” (SEQ ID NOs 3, 5, 29, 33), the “2+1 IgG Crossfab, inverted (V9)” (SEQ ID NOs 5, 23, 183, 187), the “2+1 IgG Crossfab (anti-CD3)” (SEQ ID NOs 5, 23, 215, 217), the “2+1 IgG Crossfab, inverted (anti-CD3)” (SEQ ID NOs 5, 23, 215, 219) were compared, using human MCSP-positive MV-3 or WM266-4 tumor cells and human PBMCs (E:T ratio=10:1), and an incubation time of about 24 hours. As depicted in FIG. 67, the T cell-mediated killing of the “2+1 IgG Crossfab, inverted” constructs seems to be slightly stronger or at least equal to the one induced by the “2+1 IgG Crossfabt” constructs for both CD3 binders. The calculated EC50 values were as follows:

| | 2 + 1 IgG Crossfab | 2 + 1 IgG Crossfab | 2 + 1 IgG Crossfab | 2 + 1 IgG Crossfab, |
|---|---|---|---|---|
| EC50 [pM] | (V9) | inverted (V9) | (anti-CD3) | inverted (anti-CD3) |
| MV-3 | 10.0 | 4.1 | 11.0 | 3.0 |
| WM266-4 | 12.4 | 3.7 | 11.3 | 7.1 |

### Example 7

### CD107a/b Assay

Purified “2+1 IgG scFab” construct (SEQ ID NOs 5, 17, 19) and the “(scFv)₂” molecule, both targeting human MCSP and human CD3, were tested by flow cytometry for their potential to up-regulate CD107a and intracellular perforin levels in the presence or absence of human MCSP-expressing tumor cells.

Briefly, on day one, 30 000 Colo-38 tumor target cells per well were plated in a round-bottom 96-well plate and incubated overnight at 37° C., 5% CO₂ to let them adhere. Primary human pan T cells were isolated on day 1 or day 2 from Buffy Coat, as described.

On day two, 0.15 million effector cells per well were added to obtain a final E:T ratio of 5:1. FITC-conjugated CD107a/b antibodies, as well as the different bispecific constructs and controls are added. The different bispecific molecules and antibodies were adjusted to same molarities to obtain a final concentration of 9.43 nM. Following a 1 h incubation step at 37° C., 5% CO₂, monensin was added to inhibit secretion, but also to neutralize the pH within endosomes and lysosomes. After an additional incubation time of 5 h, cells were stained at 4° C. for 30 min for surface CD8 expression. Cells were washed with staining buffer (PBS/0.1% BSA), fixed and permeabilized for 20 min using the BD Cytofix/Cytoperm Plus Kit with BD Golgi Stop (BD Biosciences #554715). Cells were washed twice using 1×BD Perm/Wash buffer, and intracellular staining for perforin was performed at 4° C. for 30 min. After a final washing step with 1×BD Perm/Wash buffer, cells were resuspended in PBS/0.1% BSA and analyzed on FACS CantoII (all antibodies were purchased from BD Biosciences or BioLegend).

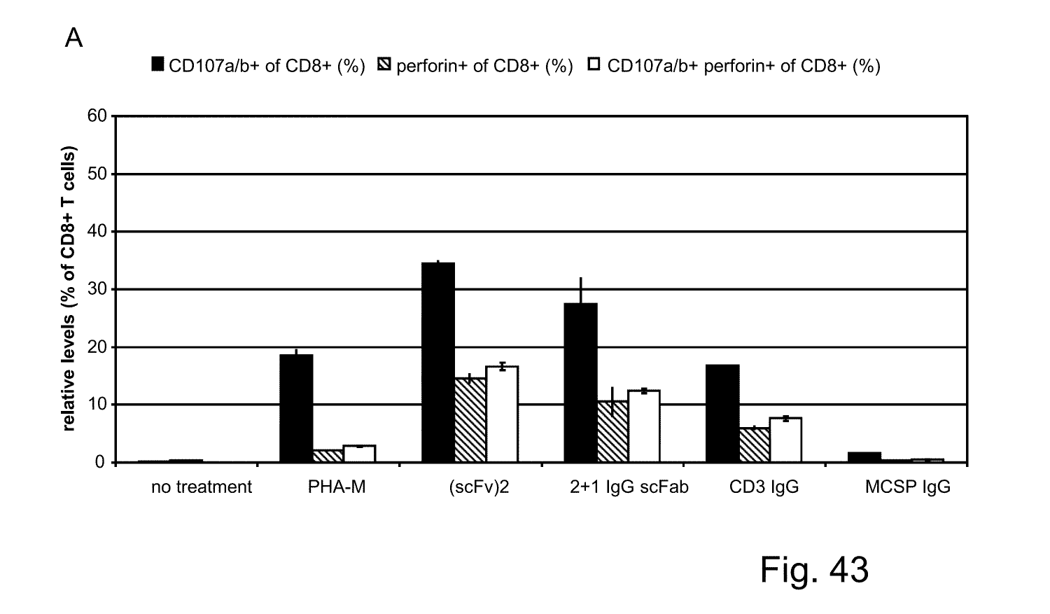

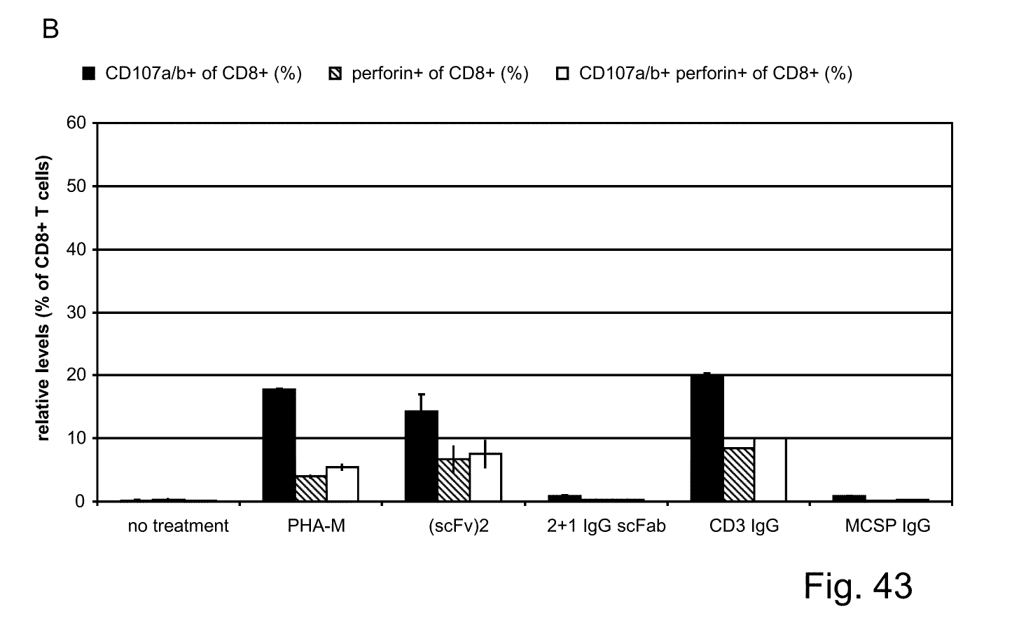

Gates were set either on all CD107a/b positive, perforin-positive or double-positive cells, as indicated (FIG. 43). The “2+1 IgG scFab” construct was able to activate T cells and up-regulate CD107a/b and intracellular perforin levels only in the presence of target cells (FIG. 43A), whereas the “(scFv)₂” molecule shows (weak) induction of activation of T cells also in the absence of target cells (FIG. 43B). The bivalent reference anti-CD3 IgG results in a lower level of activation compared to the “(scFv)₂” molecule or the other bispecific construct.

### Example 8

### Proliferation Assay

The purified “2+1 IgG scFab” (SEQ ID NOs 5, 17, 19) and “(scFv)₂” molecules, both targeting human CD3 and human MCSP, were tested by flow cytometry for their potential to induce proliferation of CD8⁺ or CD4⁺ T cells in the presence and absence of human MCSP-expressing tumor cells.

Briefly, freshly isolated human pan T cells were adjusted to 1 million cells per ml in warm PBS and stained with 1 μM CFSE at room temperature for 10 minutes. The staining volume was doubled by addition of RPMI1640 medium, containing 10% FCS and 1% GlutaMax. After incubation at room temperature for further 20 min, the cells were washed three times with pre-warmed medium to remove remaining CFSE. MCSP-positive Colo-38 cells were harvested with Cell Dissociation buffer, counted and checked for viability. Cells were adjusted to 0.2×10⁶ (viable) cells per ml in AIM-V medium, 100 μl of this cell suspension were pipetted per well into a round-bottom 96-well plate (as indicated). 50 μl of the (diluted) bispecific constructs were added to the cell-containing wells to obtain a final concentration of 1 nM. CFSE-stained human pan T effector cells were adjusted to 2×10⁶ (viable) cells per ml in AIM-V medium. 50 μl of this cell suspension was added per well of the assay plate (see above) to obtain a final E:T ratio of 5:1. To analyze whether the bispecific constructs are able to activate T cells only in the presence of target cells, expressing the tumor antigen huMCSP, wells were included that contained 1 nM of the respective bispecific molecules as well as PBMCs, but no target cells. After incubation for five days at 37° C., 5% CO₂, cells were centrifuged (5 min, 350×g) and washed twice with 150 μl/well PBS, including 0.1% BSA. Surface staining for CD8 (mouse IgG1, κ; clone HIT8a; BD #555635), CD4 (mouse IgG1, κ; clone RPA-T4; BD #560649), or CD25 (mouse IgG1, κ; clone M-A251; BD #555434) was performed at 4° C. for 30 min, according to the supplier's suggestions. Cells were washed twice with 150 μl/well PBS containing 0.1% BSA, resuspended in 200 μl/well PBS with 0.1% BSA, and analyzed using a FACS CantoII machine (Software FACS Diva). The relative proliferation level was determined by setting a gate around the non-proliferating cells and using the cell number of this gate relative to the overall measured cell number as the reference.

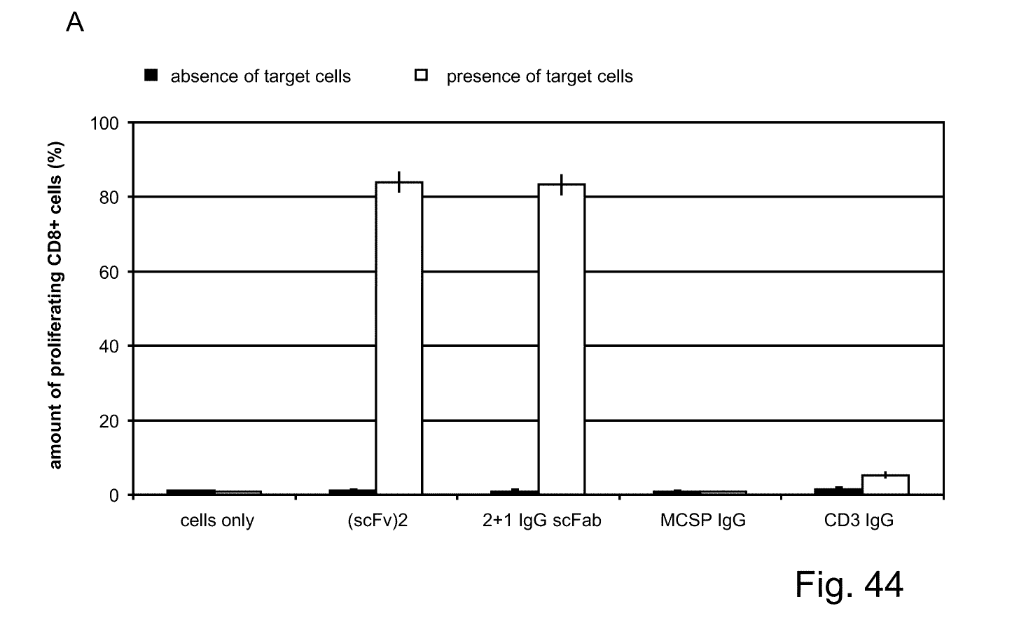

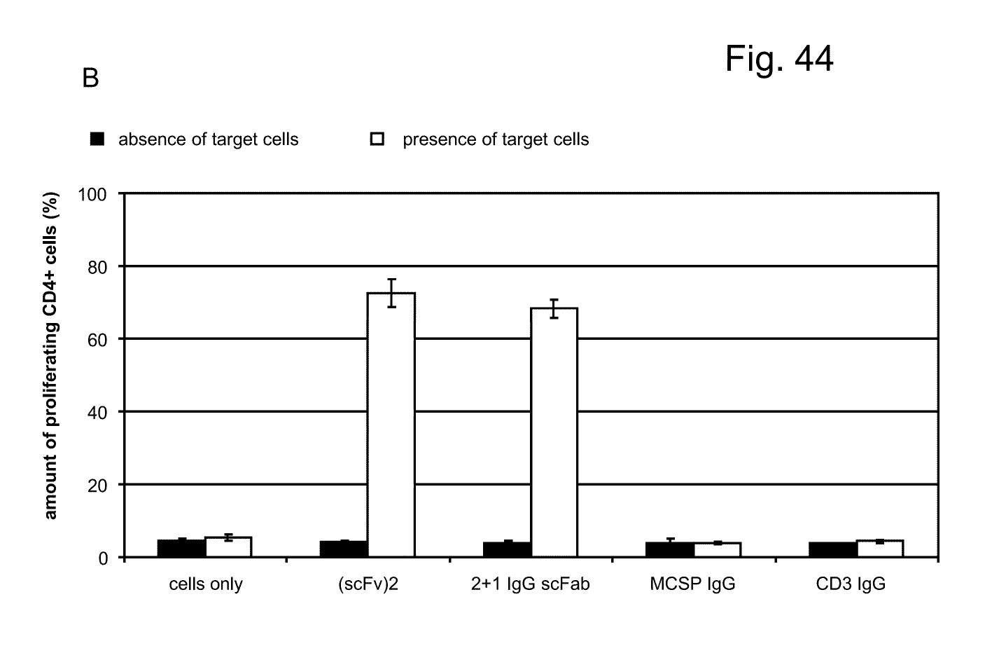

FIG. 44 shows that all constructs induce proliferation of CD8⁺ T cells (A) or CD4⁺ T cells (B) only in the presence of target cells, comparably to the “(scFv)₂” molecule. In general, activated CD8⁺ T cells proliferate more than activated CD4⁺ T cells in this assay.

### Example 9

### Cytokine Release Assay

The purified “2+1 IgG scFab” construct (SEQ ID NOs 5, 17, 19) and the “(scFv)₂” molecule, both targeting human MCSP and human CD3, were analyzed for their ability to induce T cell-mediated de novo secretion of cytokines in the presence or absence of tumor target cells.

Briefly, human PBMCs were isolated from Buffy Coats and 0.3 million cells were plated per well into a round-bottom 96-well plate. Colo-38 tumor target cells, expressing human MCSP, were added to obtain a final E:T-ratio of 10:1. Bispecific constructs and IgG controls were added at 1 nM final concentration and the cells were incubated for 24 h at 37° C., 5% CO₂. The next day, the cells were centrifuged for 5 min at 350×g and the supernatant was transferred into a new deep-well 96-well-plate for the subsequent analysis. The CBA analysis was performed according to manufacturer's instructions for FACS CantoII, using the Human Th1/Th2 Cytokine Kit II (BD #551809).

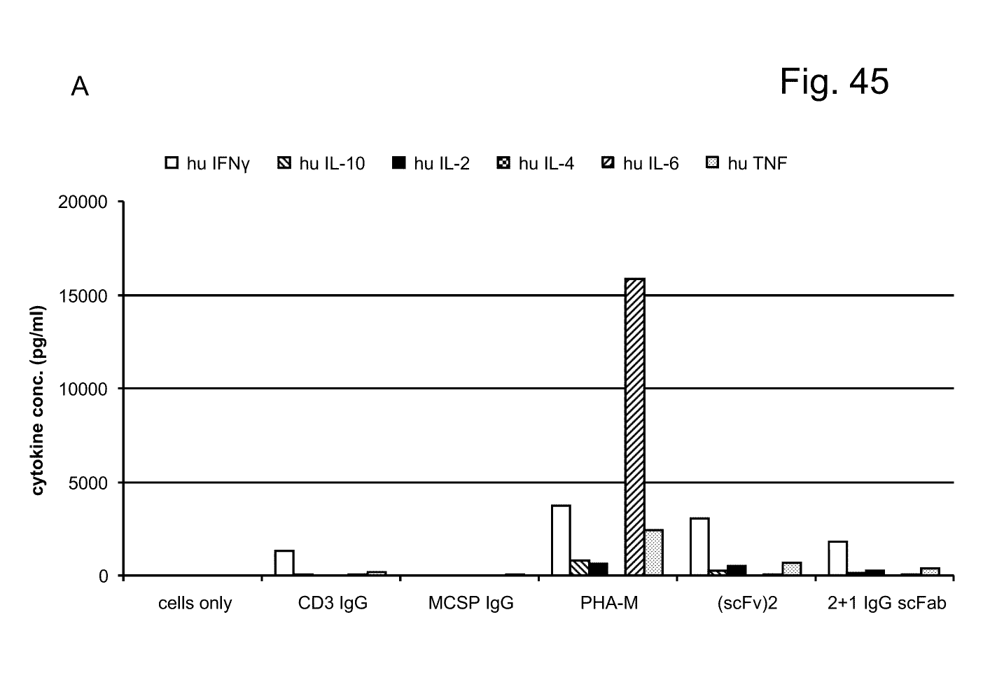

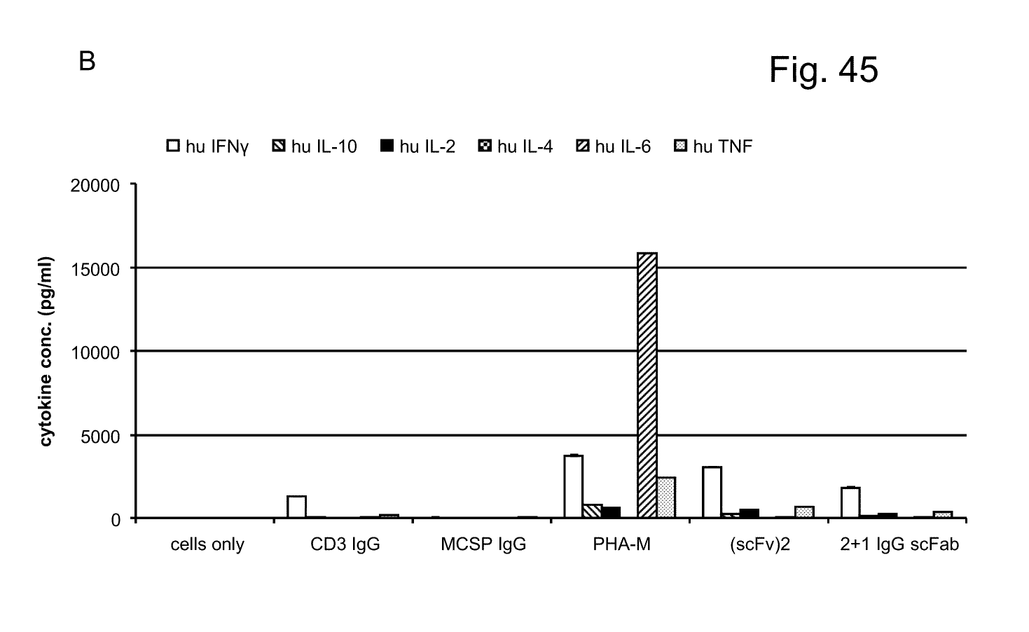

FIG. 45 shows levels of the different cytokine measured in the supernatant. In the presence of target cells the main cytokine secreted upon T cell activation is IFN-γ. The “(scFv)₂” molecule induces a slightly higher level of IFN-γ than the “2+1 IgG scFab” construct. The same tendency might be found for human TNF, but the overall levels of this cytokine were much lower compared to IFN-γ. There was no significant secretion of Th2 cytokines (IL-10 and IL-4) upon activation of T cells in the presence (or absence) of target cells. In the absence of Colo-38 target cells, only very weak induction of TNF secretion was observed, which was highest in samples treated with the “(scFv)₂” molecule.

In a second experiment, the following purified bispecific constructs targeting human MCSP and human CD3 were analyzed: the “2+1 IgG Crossfab” construct (SEQ ID NOs 3, 5, 29, 33), the “(scFv)₂” molecule, as well as different “2+1 IgG scFab” molecules comprising either a wild-type or a mutated (LALA, P329G and/or N297D, as indicated) Fc domain. Briefly, 280 μl whole blood from a healthy donor were plated per well of a deep-well 96-well plate. 30 000 Colo-38 tumor target cells, expressing human MCSP, as well as the different bispecific constructs and IgG controls were added at 1 nM final concentration. The cells were incubated for 24 h at 37° C., 5% CO₂ and then centrifuged for 5 min at 350×g. The supernatant was transferred into a new deep-well 96-well-plate for the subsequent analysis. The CBA analysis was performed according to manufacturer's instructions for FACS CantoII, using the combination of the following CBA Flex Sets: human granzyme B (BD #560304), human IFN-γ Flex Set (BD #558269), human TNF Flex Set (BD #558273), human IL-10 Flex Set (BD #558274), human IL-6 Flex Set (BD #558276), human IL-4 Flex Set (BD #558272), human IL-2 Flex Set (BD #558270).

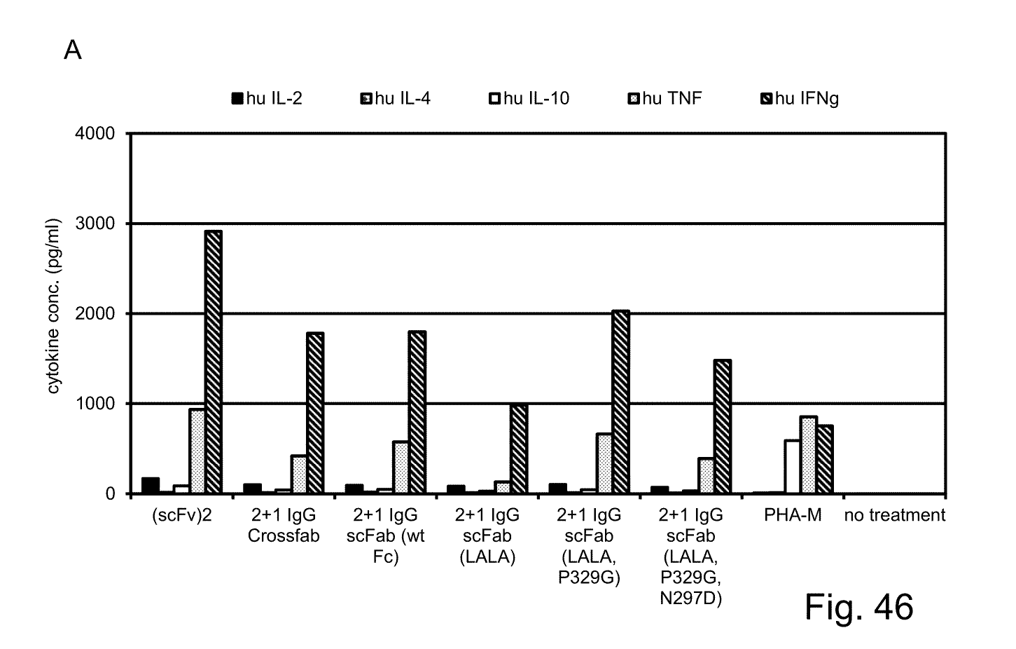

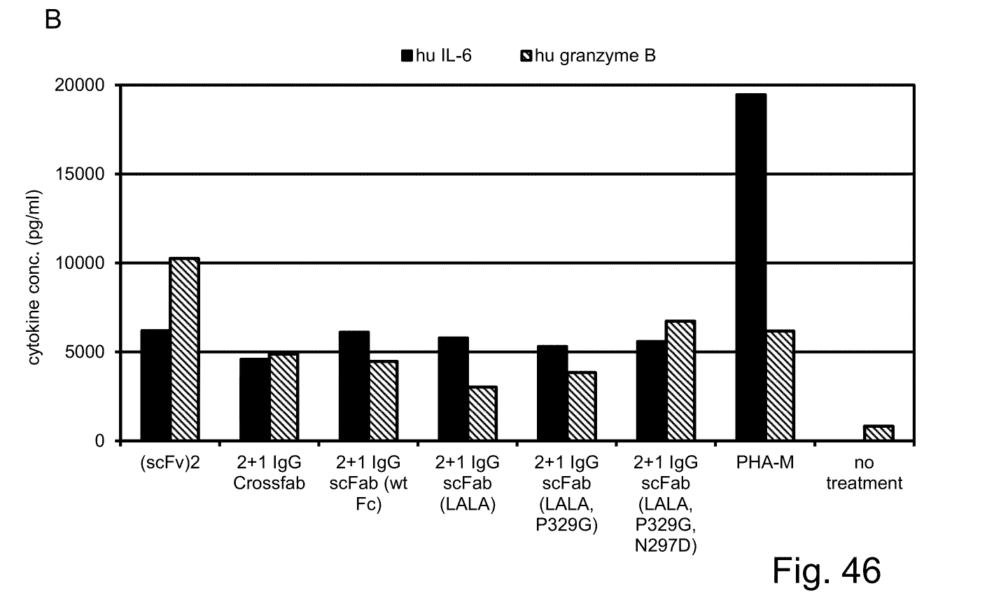

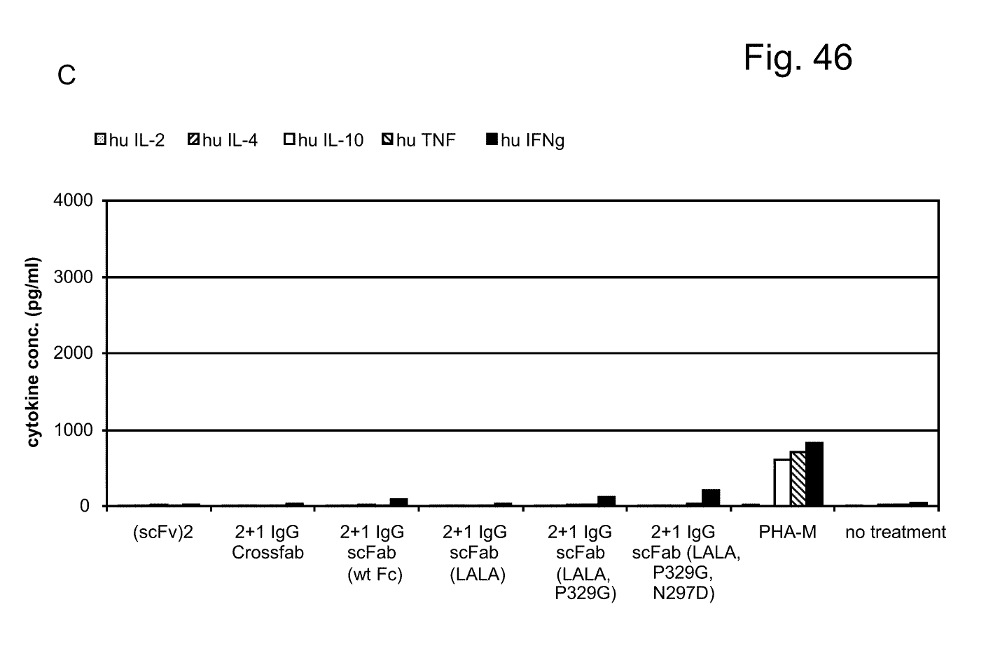

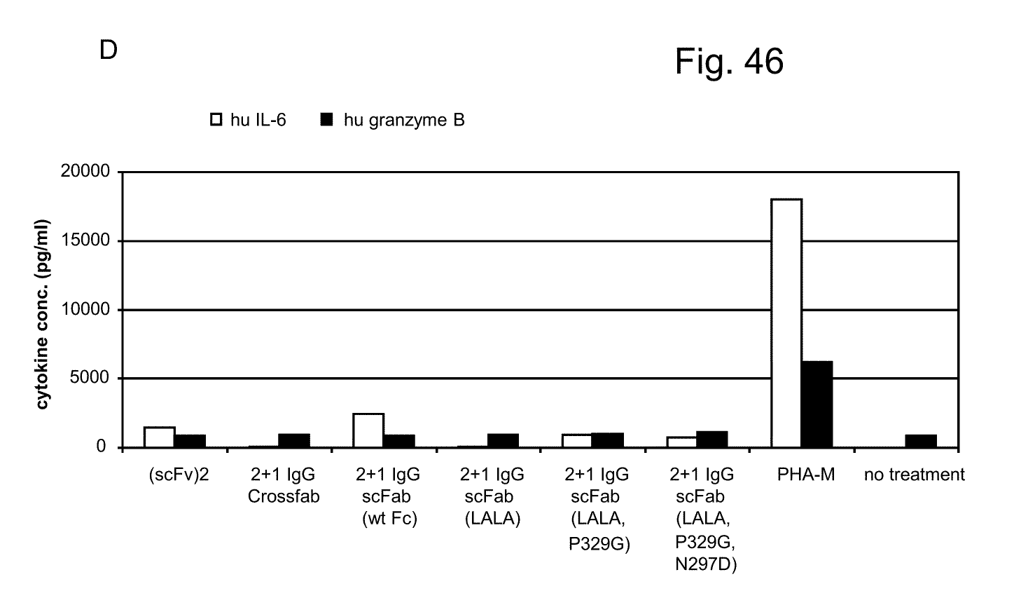

FIG. 46 shows the levels of the different cytokine measured in the supernatant. The main cytokine secreted in the presence of Colo-38 tumor cells was IL-6, followed by IFN-γ. In addition, also the levels of granzyme B strongly increased upon activation of T cells in the presence of target cells. In general, the “(scFv)₂” molecule induced higher levels of cytokine secretion in the presence of target cells (FIG. 46, A and B). There was no significant secretion of Th2 cytokines (IL-10 and IL-4) upon activation of T cells in the presence (or absence) of target cells.

In this assay, there was a weak secretion of IFN-γ, induced by different “2+1 IgG scFab” constructs, even in the absence of target cells (FIG. 46, C and D). Under these conditions, no significant differences could be observed between “2+1 IgG scFab” constructs with a wild-type or a mutated Fc domain.

### Example 10

### Affinity Maturation of Anti-MCSP Antibody M4-3/ML2

Affinity maturation was performed via the oligonucleotide-directed mutagenesis procedure. For this the heavy chain variant M4-3, and the light chain variant ML2 were cloned into a phagemid vector, similar to those described by Hoogenboom, (Hoogenboom et al., Nucleic Acids Res. 1991, 19, 4133-4137). Residues to be randomized were identified by first generating a 3D model of that antibody via classical homology modeling and then identifying the solvent accessible residues of the complementary determining regions (CDRs) of heavy and light chain. Oligonucleotides with randomization based on trinucleotide synthesis as shown in Table 4 were purchased from Ella Biotech (Munich, Germany). Three independent sublibraries were generated via classical PCR, and comprised randomization in CDR-H1 together with CDR-H2, or CDR-L1 together with CDR-L2. CDR-L3 was randomized in a separate approach. The DNA fragments of those libraries were cloned into the phagemid via restriction digest and ligation, and subsequently electroporated into TG1 bacteria.

### Library Selection

The antibody variants thus generated were displayed in a monovalent fashion from filamentous phage particles as fusions to the gene III product of M13 packaged within each particle. The phage-displayed variants were then screened for their biological activities (here: binding affinity) and candidates that have one or more improved activities were used for further development. Methods for making phage display libraries can be found in Lee et al., J. Mol. Biol. (2004) 340, 1073-1093.

Selections with all affinity maturation libraries were carried out in solution according to the following procedure: 1. binding of ˜1012 phagemid particles of each affinity maturation libraries to 100 nM biotinylated hu-MCSP(D3 domain)-avi-his (SEQ ID NO: 376) for 0.5 h in a total volume of 1 ml, 2. capture of biotinylated hu-MCSP(D3 domain)-avi-his and specifically bound phage particles by addition of 5.4×10⁷ streptavidin-coated magnetic beads for 10 min, 3. washing of beads using 5-10×1 ml PBS/Tween-20 and 5-10×1 ml PBS, 4. elution of phage particles by addition of 1 ml 100 mM TEA (triethylamine) for 10 min and neutralization by adding 500 μl 1M Tris/HCl pH 7.4 and 5. re-infection of exponentially growing E. coli TG1 bacteria, infection with helper phage VCSM13 and subsequent PEG/NaCl precipitation of phagemid particles to be used in subsequent selection rounds. Selections were carried out over 3-5 rounds using either constant or decreasing (from 10⁻⁷ M to 2×10⁻⁹ M) antigen concentrations. In round 2, capture of antigen-phage complexes was performed using neutravidin plates instead of streptavidin beads. Specific binders were identified by ELISA as follows: 100 μl of 10 nM biotinylated hu-MCSP(D3 domain)-avi-his per well were coated on neutravidin plates. Fab-containing bacterial supernatants were added and binding Fabs were detected via their Flag-tags by using an anti-Flag/HRP secondary antibody. ELISA-positive clones were bacterially expressed as soluble Fab fragments in 96-well format and supernatants were subjected to a kinetic screening experiment by SPR-analysis using ProteOn XPR36 (BioRad). Clones expressing Fabs with the highest affinity constants were identified and the corresponding phagemids were sequenced.

TABLE 4
(excluded were always Cys and Met. Lys was excluded on top in those cases where
the oligonucleotide was a reverse primer)

| Position | Randomization |
|---|---|
| Heavy chain | |
| CDR1 | |
| Ser31 | S (40%), rest (60%, 4% each) |
| Gly32 | G (40%), rest (60%, 4% each). |
| Tyr33 | Y (40%), rest (60%, 4% each) |
| Tyr34 | Y (40%), rest (60%, 4% each) |
| CDR2 | |
| Tyr50 | Y 40%, (F, W, L, A, I, 30%, 6% each), rest (30%, 2.5% each) |
| Thr52 | T (60%), rest (40%, 2.5% each) |
| Tyr53 | Y (40%), rest (60%, 3.8% each) |
| Asp54 | D (40%), rest (60%, 3.8% each) |
| Ser56 | S (40%), rest (60%, 3.8% each) |
| Light chain | |
| CDR1 | |
| Gln27 | Q (40%), (E, D, N, S, T, R, 40%, 6.7% each), rest (total 20%, 2.2% each) |
| Gly28 | G (40%), (N, T, S, Q, Y, D, E, 40%, 5.7% each), rest (20%, 2.5% each) |
| Asn31 | N (40%), (S, T, G, Q, Y, D, E, R, 50%, 6.3% each), rest (10%, 1.4% each) |
| Tyr32 | Y (40%), (W, S, R, 30%, 10% each), rest (30%, 2.3% each) |
| CDR2 | |
| Tyr50 | Y (70%), (E, R, K, A, Q, T, S, D, G, W, F, 30%, 2.7% each) |
| Thr51 | T (50%), (S, A, G, N, Q, V, 30%, 5% each), rest (20%, 2% each) |
| Ser52 | S (50%), rest (50%, 3.1% each) |
| Ser53 | S (40%), (N, T, Q, Y, D, E, I, 40%, 5.7% each), rest (20%, 2.2% each) |
| CDR3 | |
| Tyr91 | Y (50%), rest (50%, 3.1% each) |
| Ser92 | S (50%), (N, Q, T, A, G 25%, 5% each), rest (25%, 2.3% each) |
| Lys93 | K (50%), S (5%), T (5%), N (5%), rest (35%, 2.7% each) |
| Leu94 | L (50%), (Y, F, S, I, A, V, 30%, 5% each), rest (20%, 2% each) |
| Pro95 | P (50%), (S, A, 20%, 10% each), rest (30%, 2.1% each) |
| Trp96 | W 50%, (Y, R, L, 15%, 5% each), rest (35%, 2.5% each) |

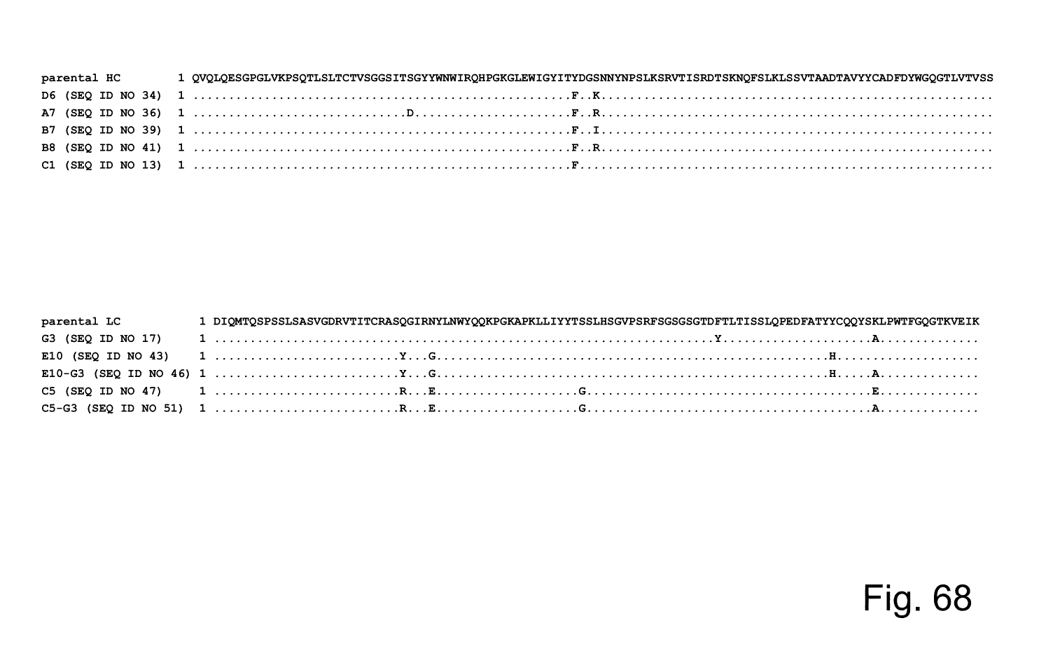

FIG. 68 shows an alignment of affinity matured anti-MCSP clones compared to the non-matured parental clone (M4-3 ML2). Heavy chain randomization was performed only in the CDR1 and 2. Light chain randomization was performed in CDR1 and 2, and independently in CDR3.

During selection, a few mutations in the frameworks occurred like F71Y in clone G3 or Y87H in clone E10.

### Production and Purification of Human IgG₁

The variable region of heavy and light chain DNA sequences of the affinity matured variants were subcloned in frame with either the constant heavy chain or the constant light chain pre-inserted into the respective recipient mammalian expression vector. The antibody expression was driven by an MPSV promoter and carries a synthetic polyA signal sequence at the 3′ end of the CDS. In addition each vector contained an EBV OriP sequence.

The molecule was produced by co-transfecting HEK293-EBNA cells with the mammalian expression vectors using polyethylenimine (PEI). The cells were transfected with the corresponding expression vectors in a 1:1 ratio. For transfection HEK293 EBNA cells were cultivated in suspension serum-free in CD CHO culture medium. For the production in 500 ml shake flask, 400 million HEK293 EBNA cells were seeded 24 hours before transfection. For transfection cells were centrifuged for 5 min at 210×g, supernatant was replaced by pre-warmed 20 ml CD CHO medium. Expression vectors were mixed in 20 ml CD CHO medium to a final amount of 200 μg DNA. After addition of 540 μl PEI solution, the mixture was vortexed for 15 s and subsequently incubated for 10 min at room temperature. Afterwards cells were mixed with the DNA/PEI solution, transferred to a 500 ml shake flask and incubated for 3 hours at 37° C. in an incubator with a 5% CO₂ atmosphere. After incubation time 160 ml F17 medium was added and cells were cultivated for 24 hours. One day after transfection 1 mM valproic acid and 7% Feed 1 (Lonza) was added. After 7 days cultivation supernatant was collected for purification by centrifugation for 15 min at 210×g, the solution was sterile filtered (0.22 μm filter) and sodium azide in a final concentration of 0.01% w/v was added, and kept at 4° C.

The secreted protein was purified from cell culture supernatants by affinity chromatography using Protein A. Supernatant was loaded on a HiTrap Protein A HP column (CV=5 mL, GE Healthcare) equilibrated with 40 ml 20 mM sodium phosphate, 20 mM sodium citrate, 0.5 M sodium chloride, pH 7.5. Unbound protein was removed by washing with at least 10 column volumes 20 mM sodium phosphate, 20 mM sodium citrate, 0.5 M sodium chloride, pH 7.5. Target protein was eluted during a gradient over 20 column volumes from 20 mM sodium citrate, 0.5 M sodium chloride, pH 7.5 to 20 mM sodium citrate, 0.5 M sodium chloride, pH 2.5. Protein solution was neutralized by adding 1/10 of 0.5 M sodium phosphate, pH 8. Target protein was concentrated and filtrated prior loading on a HiLoad Superdex 200 column (GE Healthcare) equilibrated with 20 mM histidine, 140 mM sodium chloride solution of pH 6.0.

The protein concentration of purified protein samples was determined by measuring the optical density (OD) at 280 nm, using the molar extinction coefficient calculated on the basis of the amino acid sequence. Purity and molecular weight of molecules were analyzed by CE-SDS analyses in the presence and absence of a reducing agent. The Caliper LabChip GXII system (Caliper Life Sciences) was used according to the manufacturer's instruction. 2 μg sample was used for analyses. The aggregate content of antibody samples was analyzed using a TSKgel G3000 SW XL analytical size-exclusion column (Tosoh) in 25 mM K₂HPO₄, 125 mM NaCl, 200 mM L-arginine monohydrochloride, 0.02% (w/v) NaN₃, pH 6.7 running buffer at 25° C.

TABLE 5
Production and purification of affinity matured anti-MCSP IgGs

| | | HMW | LMW | Monomer |
|---|---|---|---|---|
| Construct | Yield [mg/l] | [%] | [%] | [%] |
| M4-3(C1) ML2(G3) | 43.9 | 0 | 0 | 100 |
| M4-3(C1) ML2(E10) | 59.5 | 0 | 0 | 100 |
| M4-3(C1) ML2(C5) | 68.9 | 0 | 0.8 | 99.2 |

### Affinity Determination

### ProteOn Analysis

K_{D} was measured by surface plasmon resonance using a ProteOn XPR36 machine (BioRad) at 25° C. with anti-human F(ab′)2 fragment specific capture antibody (Jackson ImmunoResearch #109-005-006) immobilized by amine coupling on CM5 chips and subsequent capture of Fabs from bacterial supernatant or from purified Fab preparations. Briefly, carboxymethylated dextran biosensor chips (CM5, GE Healthcare) were activated with N-ethyl-N′-(3-dimethylaminopropyl)-carbodiimide hydrochloride (EDC) and N-hydroxysuccinimide (NHS) according to the supplier's instructions. Anti-human F(ab′)2 fragment specific capture antibody was diluted with 10 mM sodium acetate, pH 5.0 at 50 μg/ml before injection at a flow rate of 10 μl/minute to achieve approximately up to 10.000 response units (RU) of coupled capture antibody. Following the injection of the capture antibody, 1 M ethanolamine is injected to block unreacted groups. For kinetics measurements, Fabs from bacterial supernatant or purified Fabs were injected at a flow rate of 10 μl/minute for 300 s and a dissociation of 300 s for capture baseline stabilization. Capture levels were in the range of 100-500 RU. In a subsequent step, human MCSP(D3 domain)-avi-his analyte was injected either as a single concentration or as a concentration series (depending of clone affinity in a range between 100 nM and 250 pM) diluted into HBS-EP+ (GE Healthcare, 10 mM HEPES, 150 mM NaCl, 3 mM EDTA, 0.05% Surfactant P20, pH 7.4) at 25° C. at a flow rate of 50 μl/min. The surface of the sensorchip was regenerated by injection of glycine pH 1.5 for 30 s at 90 μl/min followed by injection of NaOH for 20 s at the same flow rate. Association rates (kₒₙ) and dissociation rates (k_{off}) were calculated using a simple one-to-one Langmuir binding model (ProteOn XPR36 Evaluation Software or Scrubber software (BioLogic)) by simultaneously fitting the association and dissociation sensorgrams. The equilibrium dissociation constant (K_{D}) was calculated as the ratio k_{off}/kₒₙ. This data was used to determine the comparative binding affinity of the affinity matured variants with the parental antibody. Table 6a shows the data generated from these assays.

G3, E10, C5 for the light chain, and D6, A7, B7, B8, C1 for the heavy chain were chosen for conversion into human IgG₁ format. Since CDR1 and 2 of the light chain were randomized independent from CDR3, the obtained CDRs were combined during IgG conversion.

In the IgG format affinities were measured again to the human MCSP antigen (SEQ ID NO: 376), in addition also to the cynomolgus homologue (SEQ ID NO: 375).

The method used was exactly as described for the Fab fragments, just using purified IgG from mammalian production.

TABLE 6a
MCSP affinity matured clones: Proteon data.

| | | | | Human | Cyno |
|---|---|---|---|---|---|
| | | | | MCSP | MCSP |
| | Human | Human | Cyno | IgG K_{D} | IgG K_{D} |
| | MCSP | MCSP | MCSP | Comparative binding | |
| | Fab K_{D} | IgG K_{D} | IgG K_{D} | affinity— | |
| Variant | Proteon generated affinity data | | | Fold increase over parent | |
| Parental M4-3/ML2 | 5*10⁻⁹ | 2*10⁻⁹ | 2*10⁻⁹ | | |
| M4-3/ML2(G3) | 4*10⁻¹⁰ | 3*10⁻¹⁰ | 6*10⁻¹⁰ | 6.7 | 3.3 |
| M4-3/ML2(E10) | 7*10⁻¹⁰ | 1*10⁻⁹ | 2*10⁻⁹ | 2.0 | 1.0 |
| M4-3/ML2(E10/G3) | | 4*10⁻¹⁰ | 9*10⁻¹⁰ | 5.0 | 2.2 |
| M4-3/ML2(C5) | 7*10⁻¹⁰ | 4*10⁻¹⁰ | 1*10⁻⁹ | 5.0 | 2.0 |
| M4-3/ML2(C5/G3) | | 7*10⁻¹⁰ | 1*10⁻⁹ | 2.9 | 2.0 |
| M4-3(D6)/ML2 | 2*10⁻⁹ | 4*10⁻¹⁰ | 1*10⁻⁹ | 5.0 | 2.0 |
| M4-3(A7)/ML2 | 2*10⁻¹¹ | 8*10⁻¹⁰ | 1*10⁻⁹ | 2.5 | 2.0 |
| M4-3(B7)/ML2 | | 5*10⁻¹⁰ | 7*10⁻¹⁰ | 4.0 | 2.9 |
| M4-3(B8)/ML2 | 3*10⁻¹⁰ | 9*10⁻¹⁰ | 1*10⁻⁹ | 2.2 | 2.0 |
| M4-3(C1)/ML2 | 6*10⁻¹⁰ | 9*10⁻¹⁰ | 8*10⁻¹⁰ | 2.2 | 2.5 |
| M4-3(C1)/ML2(G3) | | 7*10⁻¹¹ | 2*10⁻¹⁰ | 28.6 | 10.0 |
| M4- | | 5*10⁻¹⁰ | 6*10⁻¹⁰ | 4.0 | 3.3 |
| 3(C1)/ML2(E10) | | | | | |
| M4-3(A7)/ML2(G3) | | 7*10⁻¹¹ | 2*10⁻¹⁰ | 28.6 | 10.0 |
| M4- | | 3*10⁻¹⁰ | 7*10⁻¹⁰ | 6.7 | 2.9 |
| 3(A7)/ML2(E10) | | | | | |
| M4-3(C1)/ML2(C5) | | 2*10⁻¹⁰ | 3*10⁻¹⁰ | 10.0 | 6.7 |
| M4-(A7)/ML2(C5) | | 7*10⁻¹⁰ | 2*10⁻¹⁰ | 28.6 | 10.0 |

### Affinity Determination by Surface Plasmon Resonance (SPR) Using Biacore T200

Surface plasmon resonance (SPR) experiments to determine the affinity and the avidity of the affinity matured IgGs were performed on a Biacore T200 at 25° C. with HBS-EP as running buffer (0.01 M HEPES pH 7.4, 0.15 M NaCl, 3 mM EDTA, 0.005% Surfactant P20, Biacore, Freiburg/Germany).

For analyzing the avidity of the interaction of different anti-MCSP IgGs to human and cynomolgus MCSP D3 direct coupling of around 9,500 resonance units (RU) of the anti-Penta His antibody (Qiagen) was performed on a CM5 chip at pH 5.0 using the standard amine coupling kit (Biacore, Freiburg/Germany). Antigens were captured for 60 s at 30 nM with 10 μl/min respectively. IgGs were passed at a concentration of 0.0064-100 nM with a flowrate of 30 μl/min through the flow cells over 280 s. The dissociation was monitored for 180 s. Bulk refractive index differences were corrected for by subtracting the response obtained on reference flow cell. Here, the IgGs were flown over a surface with immobilized anti-Penta His antibody but on which HBS-EP has been injected rather than human MCSP D3 or cynomolgus MCSP D3. For affinity measurements IgGs were captured on a CM5 sensorchip surface with immobilized anti human Fc. Capture IgG was coupled to the sensorchip surface by direct immobilization of around 9,500 resonance units (RU) at pH 5.0 using the standard amine coupling kit (Biacore, Freiburg/Germany). IgGs are captured for 25 s at 10 nM with 30 μl/min. Human and cynomolgus MCSP D3 were passed at a concentration of 2-500 nM with a flowrate of 30 μl/min through the flow cells over 120 s. The dissociation was monitored for 60 s. Association and dissociation for concentration 166 and 500 nM was monitored for 1200 and 600 s, respectively. Bulk refractive index differences were corrected for by subtracting the response obtained on reference flow cell. Here, the antigens were flown over a surface with immobilized anti-human Fc antibody but on which HBS-EP has been injected rather than anti-MCSP IgGs.

Kinetic constants were derived using the Biacore T200 Evaluation Software (vAA, Biacore AB, Uppsala/Sweden), to fit rate equations for 1:1 Langmuir binding by numerical integration.

Higher affinity to human and cynomolgus MCSP D3 were confirmed by surface plasmon resonance measurements using Biacore T200. In addition, avidity measurements showed an up to 3-fold increase in bivalent binding (Table 6b).

TABLE 6b
Affinity and avidity of anti MCSP IgGs to human
MCSP-D3 and cynomolgus MCSP-D3.

| K_{D} in nM | Human MCSP D3 | | Cynomolgus MCSP D3 | |
|---|---|---|---|---|
| T = 25° C. | Affinity | Avidity | Affinity | Avidity |
| M4-3(C1) ML2(G3) | 1.8 | 0.0045 | 1.4 | 0.0038 |
| M4-3(C1) ML2(E10) | 4.6 | 0.0063 | 3.8 | 0.0044 |
| M4-3(C1) ML2(C5) | 1.8 | 0.0046 | 1.3 | 0.0044 |
| M4-3 ML2 (parental) | 8.6 | 0.0090 | 11.4 | 0.0123 |

### Example 11

### Preparation of MCSP TCB (2+1 Crossfab-IgG P329G LALA Inverted) Containing M4-3(C1) ML2(G3) as Anti MCSP Antibody and Humanized CH2527 as Anti CD3 Antibody

The variable region of heavy and light chain DNA sequences were subcloned in frame with either the constant heavy chain or the constant light chain pre-inserted into the respective recipient mammalian expression vector. The antibody expression was driven by an MPSV promoter and carries a synthetic polyA signal sequence at the 3′ end of the CDS. In addition each vector contains an EBV OriP sequence.

The molecule was produced by co-transfecting HEK293-EBNA cells with the mammalian expression vectors using polyethylenimine (PEI). The cells were transfected with the corresponding expression vectors in a 1:2:1:1 ratio (“vector heavy chain Fc(hole)”:“vector light chain”:“vector light chain Crossfab”:“vector heavy chain Fc(knob)-FabCrossfab”).

For transfection HEK293 EBNA cells were cultivated in suspension serum-free in CD CHO culture medium. For the production in 500 ml shake flask 400 million HEK293 EBNA cells were seeded 24 hours before transfection. For transfection cells were centrifuged for 5 min at 210×g, supernatant was replaced by pre-warmed 20 ml CD CHO medium. Expression vectors were mixed in 20 ml CD CHO medium to a final amount of 200 μg DNA. After addition of 540 μl PEI solution the mixture was vortexed for 15 s and subsequently incubated for 10 min at room temperature. Afterwards cells were mixed with the DNA/PEI solution, transferred to a 500 ml shake flask and incubated for 3 hours at 37° C. in an incubator with a 5% CO₂ atmosphere. After incubation time 160 ml F17 medium was added and cell were cultivated for 24 hours. One day after transfection 1 mM valproic acid and 7% Feed 1 (Lonza) was added. After 7 days cultivation supernatant was collected for purification by centrifugation for 15 min at 210×g, the solution was sterile filtered (0.22 μm filter) and sodium azide in a final concentration of 0.01% w/v was added, and kept at 4° C.

The secreted protein was purified from cell culture supernatants by affinity chromatography using Protein A. Supernatant was loaded on a HiTrap Protein A HP column (CV=5 mL, GE Healthcare) equilibrated with 40 ml 20 mM sodium phosphate, 20 mM sodium citrate, 0.5 M sodium chloride, pH 7.5. Unbound protein was removed by washing with at least 10 column volumes 20 mM sodium phosphate, 20 mM sodium citrate, 0.5 M sodium chloride, pH 7.5. Target protein was eluted during a gradient over 20 column volumes from 20 mM sodium citrate, 0.5 M sodium chloride, pH 7.5 to 20 mM sodium citrate, 0.5 M sodium chloride, pH 2.5. Protein solution was neutralized by adding 1/10 of 0.5 M sodium phosphate, pH 8. Target protein was concentrated and filtrated prior loading on a HiLoad Superdex 200 column (GE Healthcare) equilibrated with 20 mM histidine, 140 mM sodium chloride solution of pH 6.0.

The protein concentration of purified protein samples was determined by measuring the optical density (OD) at 280 nm, using the molar extinction coefficient calculated on the basis of the amino acid sequence.

Purity and molecular weight of molecules were analyzed by CE-SDS analyses in the presence and absence of a reducing agent. The Caliper LabChip GXII system (Caliper lifescience) was used according to the manufacturer's instruction. 2 μg sample was used for analyses.

The aggregate content of antibody samples was analyzed using a TSKgel G3000 SW XL analytical size-exclusion column (Tosoh) in 25 mM K₂HPO₄, 125 mM NaCl, 200 mM L-arginine monohydrochloride, 0.02% (w/v) NaN₃, pH 6.7 running buffer at 25° C.

TABLE 7a
Summary production and purification of MCSP TCB.

| | | | Aggregate | | | |
|---|---|---|---|---|---|---|
| | | | after 1^{st} | | | |
| | Titer | Yield | purification | HMW | LMW | Monomer |
| Construct | [mg/l] | [mg/l] | step [%] | [%] | [%] | [%] |
| MCSP | 157 | 0.32 | 32 | 3.3 | 0 | 96.7 |
| TCB | | | | | | |

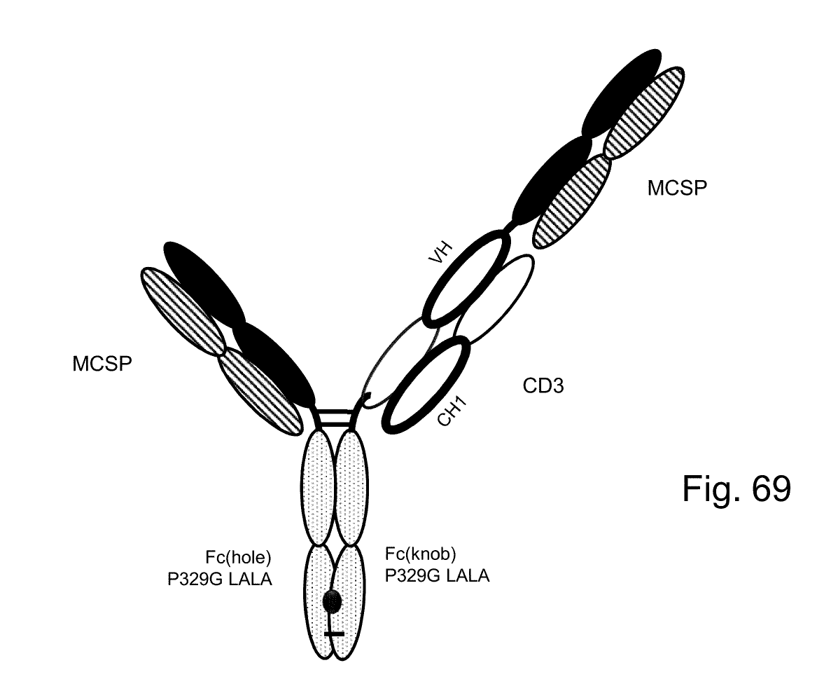

FIG. 69 shows a schematic drawing of the MCSP TCB (2+1 Crossfab-IgG P329G LALA inverted) molecule.

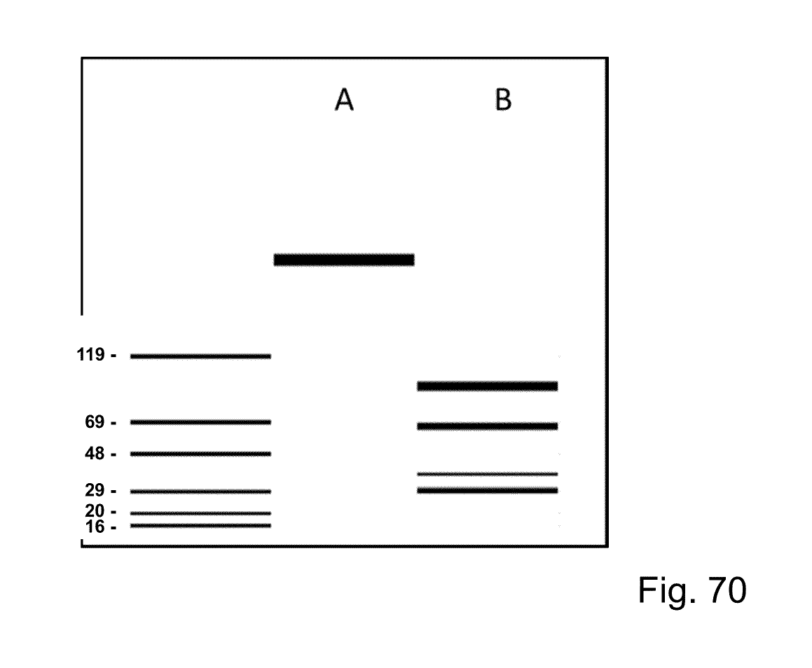

FIG. 70 and Table 7b show CE-SDS analyses of a MCSP TCB (2+1 Crossfab-IgG P329G LALA inverted) molecule (SEQ ID NOs: 278, 319, 320 and 321).

TABLE 7b
CE-SDS analyses of MCSP TCB.

| | Peak | kDa | Corresponding Chain |
|---|---|---|---|
| MCSP TCB non reduced (A) | 1 | 206.47 | |
| MCSP TCB reduced (B) | 1 | 29.15 | Light chain ML2 (C1) |
| | 2 | 37.39 | Light chain huCH2527 |
| | 3 | 66.07 | Fc(hole) |
| | 4 | 94.52 | Fc(knob) |

### Example 12

### Preparation of CEA TCB (2+1 Crossfab-IgG P329G LALA Inverted) Containing CH1A1a 98/99 2F1 as Anti CEA Antibody and Humanized CH2527 as Anti CD3 Antibody

The variable region of heavy and light chain DNA sequences were subcloned in frame with either the constant heavy chain or the constant light chain pre-inserted into the respective recipient mammalian expression vector. The antibody expression was driven by an MPSV promoter and carries a synthetic polyA signal sequence at the 3′ end of the CDS. In addition each vector contains an EBV OriP sequence.

The molecule was produced by co-transfecting HEK293 EBNA cells with the mammalian expression vectors using polyethylenimine (PEI). The cells were transfected with the corresponding expression vectors in a 1:2:1:1 ratio (“vector heavy chain Fc(hole)”:“vector light chain”:“vector light chain Crossfab”:“vector heavy chain Fc(knob)-FabCrossfab”).

For transfection HEK293 EBNA cells were cultivated in suspension serum-free in CD CHO culture medium. For the production in 500 ml shake flask 400 million HEK293 EBNA cells were seeded 24 hours before transfection. For transfection cells were centrifuged for 5 min at 210×g, supernatant was replaced by pre-warmed 20 ml CD CHO medium. Expression vectors were mixed in 20 ml CD CHO medium to a final amount of 200 μg DNA. After addition of 540 μl PEI solution the mixture was vortexed for 15 s and subsequently incubated for 10 min at room temperature. Afterwards cells were mixed with the DNA/PEI solution, transferred to a 500 ml shake flask and incubated for 3 hours by 37° C. in an incubator with a 5% CO₂ atmosphere. After incubation time 160 ml F17 medium was added and cell were cultivated for 24 hours. One day after transfection 1 mM valproic acid and 7% Feed 1 (Lonza) was added. After 7 days cultivation supernatant was collected for purification by centrifugation for 15 min at 210×g, the solution was sterile filtered (0.22 μm filter) and sodium azide in a final concentration of 0.01% w/v was added, and kept at 4° C.

The secreted protein was purified from cell culture supernatants by affinity chromatography using Protein A. Supernatant was loaded on a HiTrap Protein A HP column (CV=5 mL, GE Healthcare) equilibrated with 40 ml 20 mM sodium phosphate, 20 mM sodium citrate, 0.5 M sodium chloride, pH 7.5. Unbound protein was removed by washing with at least 10 column volumes 20 mM sodium phosphate, 20 mM sodium citrate, 0.5 M sodium chloride, pH 7.5. Target protein was eluted during a gradient over 20 column volumes from 20 mM sodium citrate, 0.5 M sodium chloride, pH 7.5 to 20 mM sodium citrate, 0.5 M sodium chloride, pH 2.5. Protein solution was neutralized by adding 1/10 of 0.5 M sodium phosphate, pH 8. Target protein was concentrated and filtrated prior loading on a HiLoad Superdex 200 column (GE Healthcare) equilibrated with 20 mM histidine, 140 mM sodium chloride solution of pH 6.0.

The protein concentration of purified protein samples was determined by measuring the optical density (OD) at 280 nm, using the molar extinction coefficient calculated on the basis of the amino acid sequence.

Purity and molecular weight of molecules were analyzed by CE-SDS analyses in the presence and absence of a reducing agent. The Caliper LabChip GXII system (Caliper lifescience) was used according to the manufacturer's instructions. 2 μg sample was used for analyses.

The aggregate content of antibody samples was analyzed using a TSKgel G3000 SW XL analytical size-exclusion column (Tosoh) in 25 mM K₂HPO₄, 125 mM NaCl, 200 mM L-arginine monohydrochloride, 0.02% (w/v) NaN₃, pH 6.7 running buffer at 25° C.

TABLE 8
Summary production and purification of CEA TCB.

| | | | Aggregate | | | |
|---|---|---|---|---|---|---|
| | | | after 1^{st} | | | |
| | Titer | Yield | purification | HMW | LMW | Monomer |
| Construct | [mg/l] | [mg/l] | step [%] | [%] | [%] | [%] |
| CEA TCB | 66 | 0.31 | 21.5 | 8.1 | 4.4 | 87.5 |

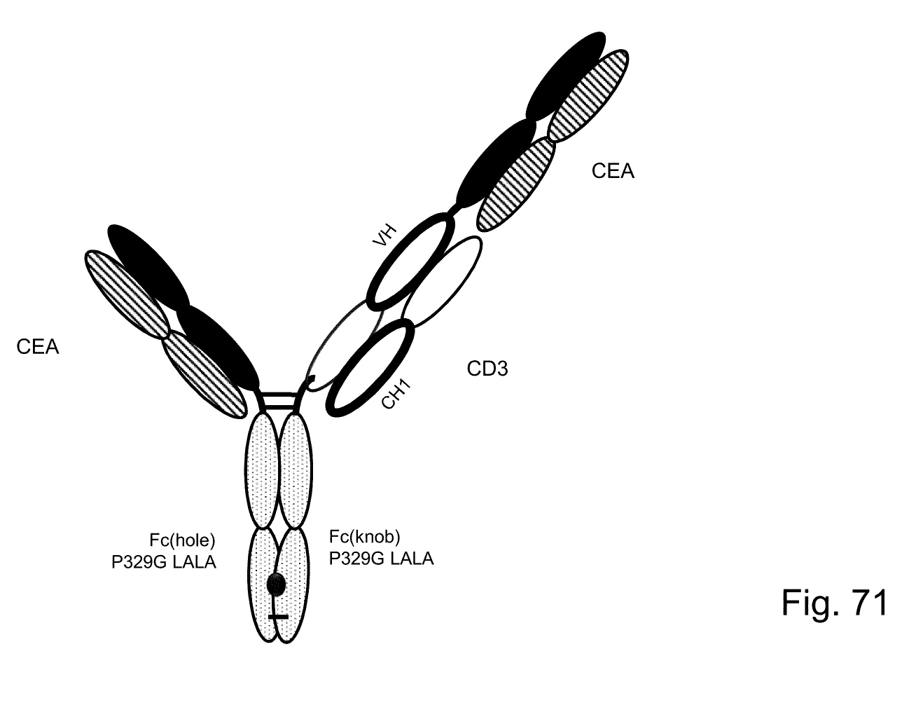

FIG. 71 shows a schematic drawing of CEA TCB (2+1 Crossfab-IgG P329G LALA inverted) molecule.

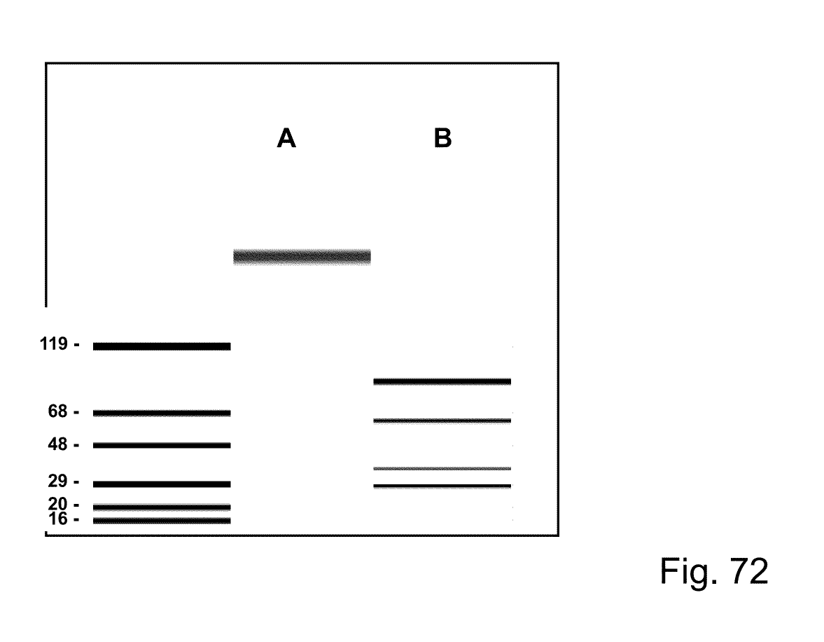

FIG. 72 and Table 9 show CE-SDS analyses of a CEA TCB (2+1 Crossfab-IgG P329G LALA inverted) molecule (SEQ ID NOs: 288, 322, 323 and 324).

TABLE 9
CE-SDS analyses of CEA TCB.

| | Peak | kDa | Corresponding Chain |
|---|---|---|---|
| CEA TCB non reduced (A) | 1 | 205.67 | Correct molecule |
| CEA TCB reduced (B) | 1 | 28.23 | Light chain CH1A1A |
| | | | 98/99 x 2F1 |
| | 2 | 36.31 | Light chain CH2527 |
| | 3 | 63.48 | Fc(hole) |
| | 4 | 90.9 | Fc(knob) |

In an alternative purification method, the CEA TCB was captured from harvested and clarified fermentation supernatant by Protein A affinity chromatography (MabSelect SuRe). The Protein A eluate was then submitted to cation exchange chromatography (Poros 50 HS) and subsequently fractionated and analyzed by means of SE-HPLC and capillary electrophoresis. The product containing fractions were pooled and subjected to hydrophobic interaction chromatography (Butyl-Sepharose 4FF) at room temperature in a bind-elute mode. The eluate therefrom was then fractionated and analyzed by means of SE-HPLC and capillary electrophoresis. The product containing fractions were pooled and subsequently anion exchange chromatography (Q-Sepharose FF) in flow-through mode was performed. The material obtained using this purification method had a monomer content of >98%.

### Example 13

### Binding of MCSP TCB to MCSP- and CD3-Expressing Cells

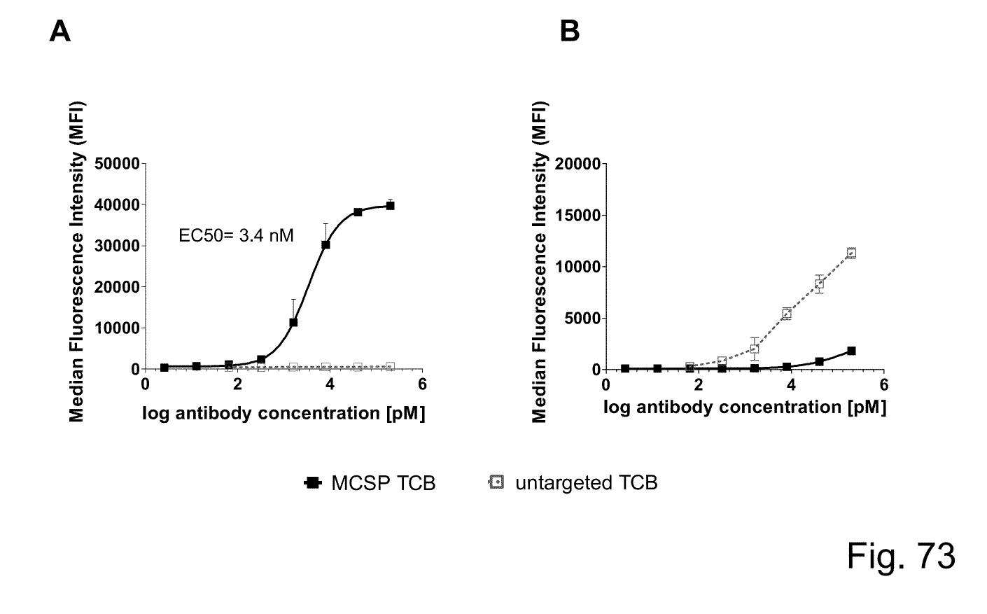

The binding of MCSP TCB was tested on a MCSP-expressing human malignant melanoma cell line (A375) and a CD3-expressing immortalized T lymphocyte line (Jurkat). Briefly, cells were harvested, counted, checked for viability and resuspended at 2×10⁶ cells/ml in FACS buffer (100 μl PBS 0.1% BSA). 100 μl cell suspension (containing 0.2×10⁶ cells) were incubated in round-bottom 96-well plate for 30 min at 4° C. with increasing concentrations of the MCSP TCB (2.6 pM-200 nM), washed twice with cold PBS 0.1% BSA, re-incubated for further 30 min at 4° C. with the PE-conjugated AffiniPure F(ab′)2 Fragment goat anti-human IgG Fcγ Fragment Specific secondary antibody (Jackson Immuno Research Lab PE #109-116-170), washed twice with cold PBS 0.1% BSA and immediately analyzed by FACS using a FACS CantoII (Software FACS Diva) by gating live, DAPI-negative, cells. Binding curves were obtained using GraphPadPrism5 (FIG. 73A, binding to A375 cells, EC₅₀=3381 pM; FIG. 73B, binding to Jurkat cells).

### Example 14

### T-Cell Killing Induced by MCSP TCB Antibody

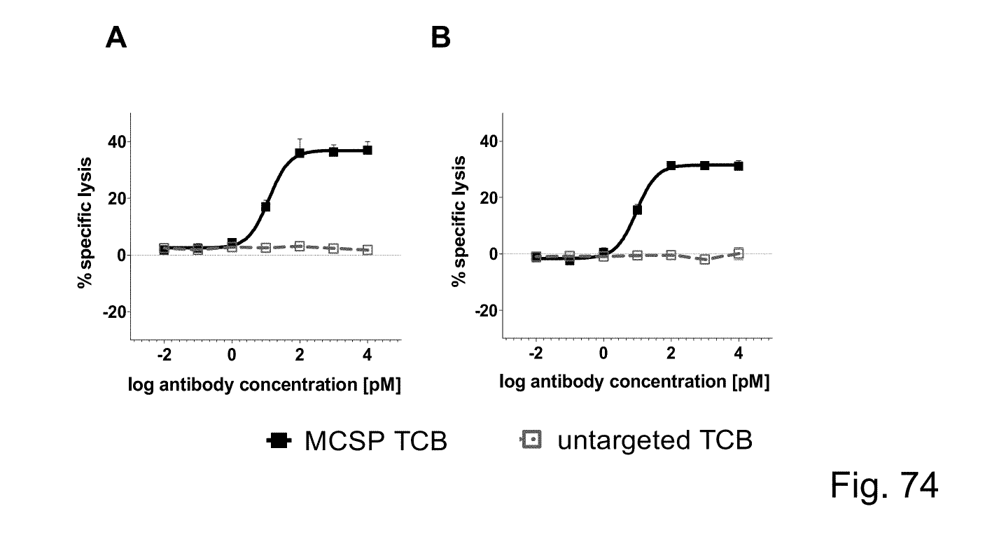

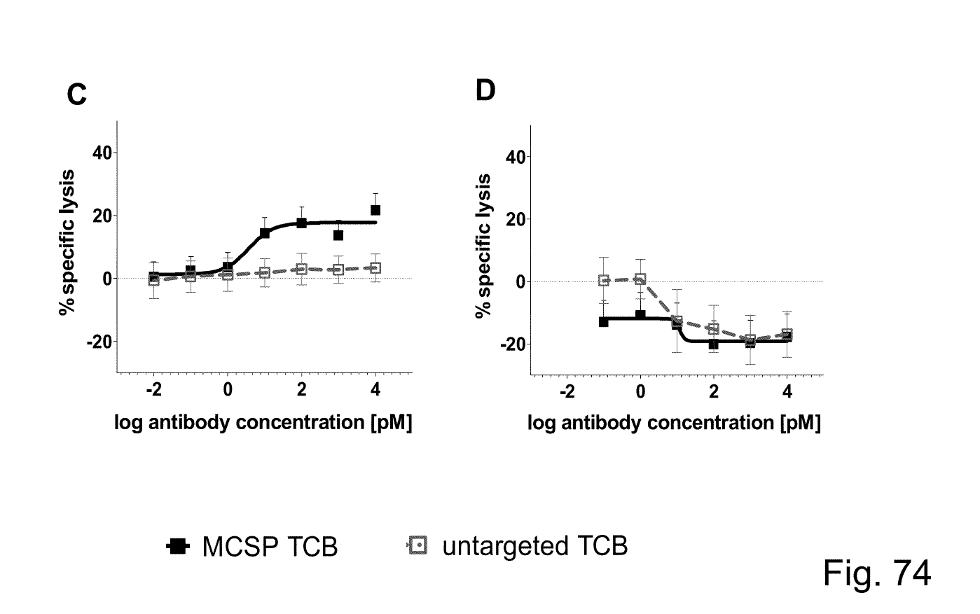

T-cell killing mediated by MCSP TCB antibody was assessed using a panel of tumor cell lines expressing different levels of MCSP (A375=MCSP high, MV-3=MSCP medium, HCT-116=MCSP low, LS180=MCSP negative). Briefly, target cells were harvested with Trypsin/EDTA, washed, and plated at density of 25 000 cells/well using flat-bottom 96-well plates. Cells were left to adhere overnight. Peripheral blood mononuclear cells (PBMCs) were prepared by Histopaque density centrifugation of enriched lymphocyte preparations (buffy coats) obtained from healthy human donors. Fresh blood was diluted with sterile PBS and layered over Histopaque gradient (Sigma, #H8889). After centrifugation (450×g, 30 minutes, room temperature), the plasma above the PBMC-containing interphase was discarded and PBMCs transferred in a new falcon tube subsequently filled with 50 ml of PBS. The mixture was centrifuged (400×g, 10 minutes, room temperature), the supernatant discarded and the PBMC pellet washed twice with sterile PBS (centrifugation steps 350×g, 10 minutes). The resulting PBMC population was counted automatically (ViCell) and stored in RPMI1640 medium containing 10% FCS and 1% L-alanyl-L-glutamine (Biochrom, K0302) at 37° C., 5% CO₂ in cell incubator until further use (no longer than 24 h). For the killing assay, the antibody was added at the indicated concentrations (range of 1 pM-10 nM in triplicates). PBMCs were added to target cells at final effector to target (E:T) ratio of 10:1. Target cell killing was assessed after 24 h of incubation at 37° C., 5% CO₂ by quantification of LDH released into cell supernatants by apoptotic/necrotic cells (LDH detection kit, Roche Applied Science, #11 644 793 001). Maximal lysis of the target cells (=100%) was achieved by incubation of target cells with 1% Triton X-100. Minimal lysis (=0%) refers to target cells co-incubated with effector cells without bispecific construct. The results show that MCSP TCB induced a strong and target-specific killing of MCSP-positive target cell lines with no killing of MCSP-negative cell lines (FIG. 74, A-D). The EC₅₀ values related to the killing assays, calculated using GraphPadPrism5 are given in Table 10.

TABLE 10
EC₅₀ values (pM) for T-cell mediated killing of MCSP-
expressing tumor cells induced by MCSP TCB antibody.

| Cell line | MCSP receptor copy number | EC₅₀ [pM] |
|---|---|---|
| A375 | 387 058 | 12.3 |
| MV-3 | 260 000 | 9.4 |
| HCT-116 | 36770 | 3.7 |
| LS180 | Negative | n.d. |

### Example 15

### CD25 and CD69 Upregulation on CD8⁺ and CD4⁺ Effector Cells after T Cell Killing of MCSP-Expressing Tumor Cells Induced by MCSP TCB Antibody

Activation of CD8⁺ and CD4⁺ T cells after T-cell killing of MCSP-expressing MV-3 tumor cells mediated by the MCSP TCB antibody was assessed by FACS analysis using antibodies recognizing the T cell activation markers CD25 (late activation marker) and CD69 (early activation marker). The antibody and the killing assay conditions were essentially as described above (Example 14), using the same antibody concentration range (1 pM-10 nM in triplicates), E:T ratio 10:1 and an incubation time 24 h.

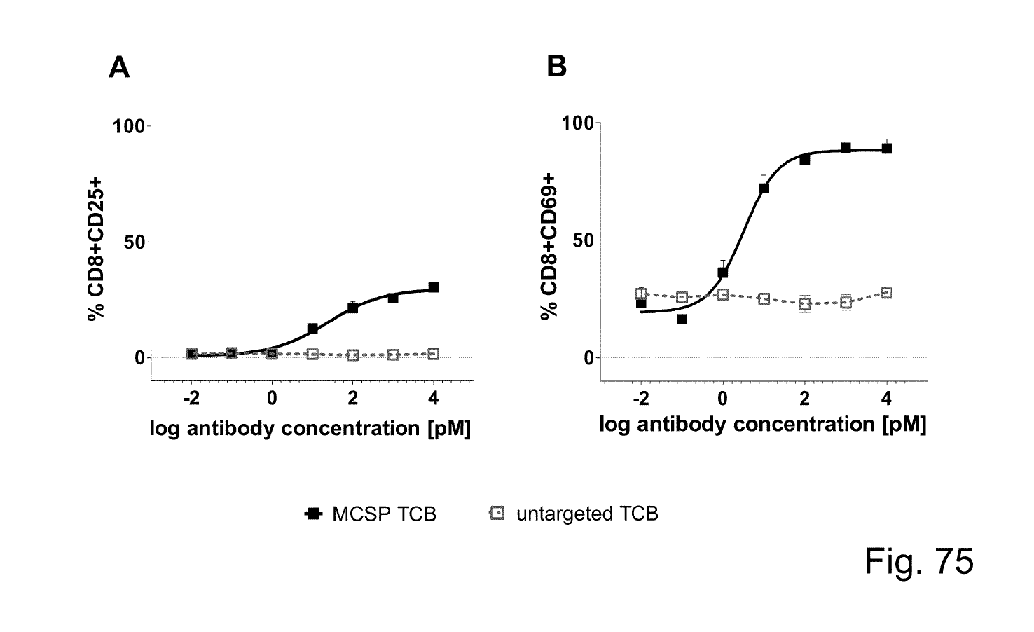

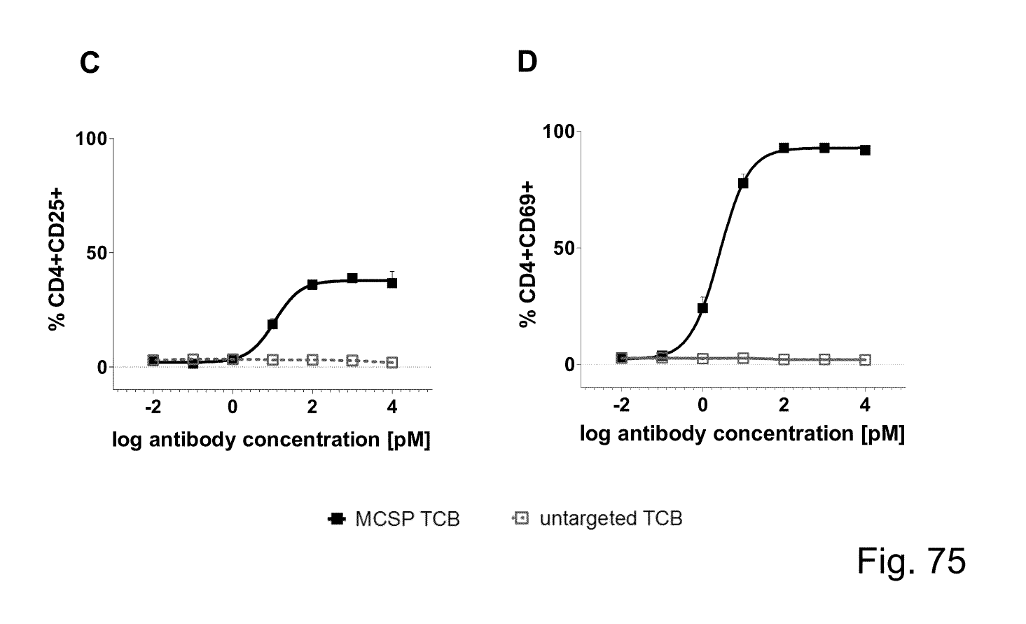

After the incubation, PBMCs were transferred to a round-bottom 96-well plate, centrifuged at 350×g for 5 min and washed twice with PBS containing 0.1% BSA. Surface staining for CD8 (FITC anti-human CD8, BD #555634), CD4 (PECy7 anti-human CD4, BD #557852), CD69 (PE anti-human CD69, Biolegend #310906) and CD25 (APC anti-human CD25, BD #555434) was performed according to the suppliers' indications. Cells were washed twice with 150 μl/well PBS containing 0.1% BSA and fixed for 15 min at 4° C. using 100 μl/well fixation buffer (BD #554655). After centrifugation, the samples were resuspended in 200 μl/well PBS 0.1% BSA containing DAPI to exclude dead cells for the FACS measurement. Samples were analyzed at BD FACS Fortessa. The results show that MCSP TCB induced a strong and target-specific upregulation of activation markers (CD25, CD69) on CD8⁺ T cells (FIG. 75 A, B) and CD4⁺ T cells (FIG. 75 C, D) after killing.

### Example 16

### Cytokine Secretion by Human Effector Cells after T Cell-Killing of MCSP-Expressing Tumor Cells Induced by MCSP TCB Antibody

Cytokine secretion by human PBMCs after T-cell killing of MCSP-expressing MV-3 tumor cells induced by the MCSP TCB antibody was assessed by FACS analysis of cell supernatants after the killing assay.

The same antibody was used and the killing assay was performed essentially as described above (Example 14 and 15), using an E:T ratio of 10:1 and an incubation time of 24 h.

At the end of the incubation time, the plate was centrifuged for 5 min at 350×g, the supernatant transferred in a new 96-well plate and stored at −20° C. until subsequent analysis. Granzyme B, TNFα, IFN-γ, IL-2, IL-4 and IL-10 secreted into in cell supernatants were detected using the BD CBA Human Soluble Protein Flex Set, according to manufacturer's instructions on a FACS CantoII. The following kits were used: BD CBA human Granzyme B BD CBA human Granzyme B Flex Set #BD 560304; BD CBA human TNF Flex Set #BD 558273; BD CBA human IFN-γ Flex Set #BD 558269; BD CBA human IL-2 Flex Set #BD 558270; BD CBA human IL-4 Flex Set #BD 558272; BD CBA human IL-10 Flex Set #BD 558274.

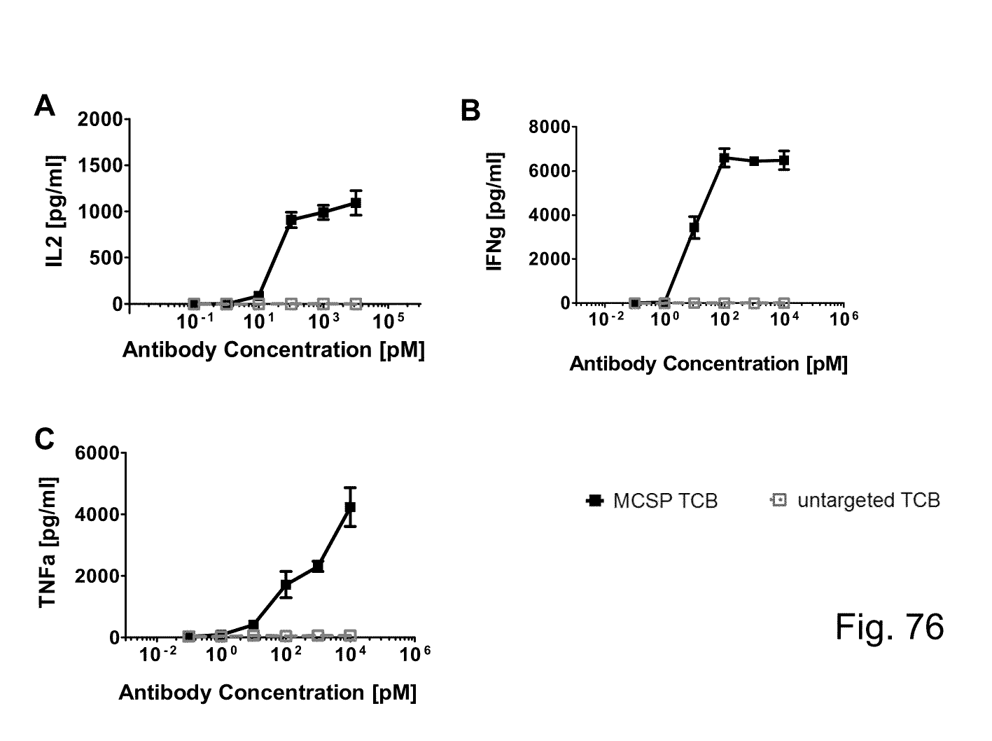

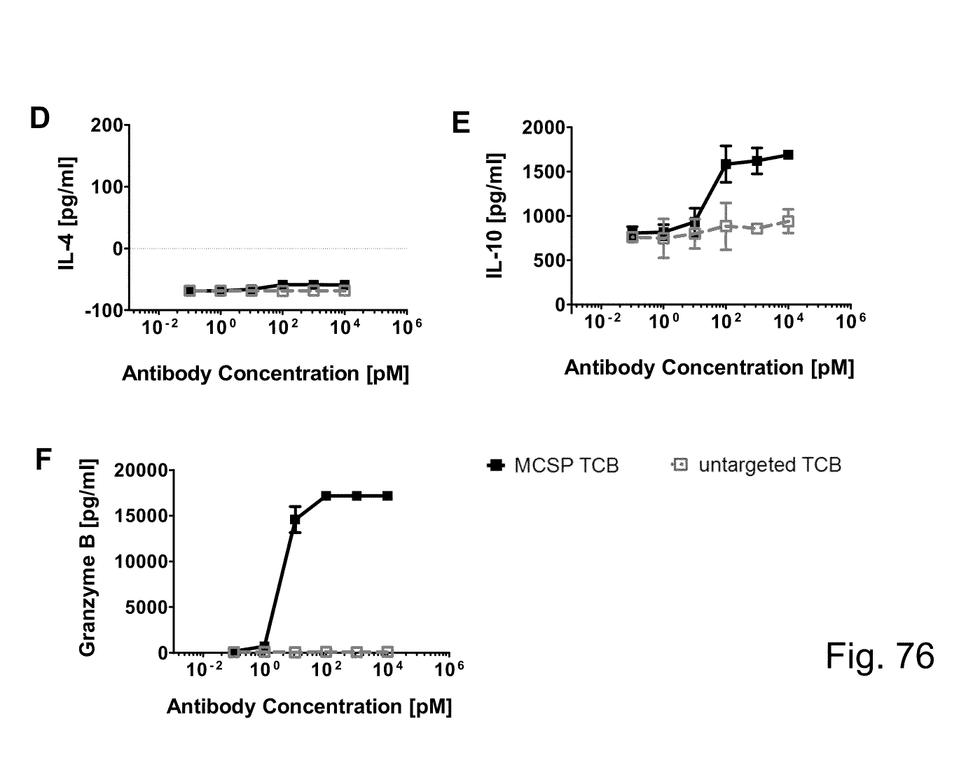

The results show that MCSP TCB induced secretion of IL-2, IFN-γ, TNFα, Granzyme B and IL-10 (but no IL-4) upon killing (FIG. 76, A-F).

Taken together, these examples show that the MCSP CD3 bispecific antibody
Showed a good binding to MCSP-positive A375 cells
Induced a strong and target-specific killing of MCSP-positive target cell lines, and no killing of MCSP-negative cell lines
Induced a strong and target-specific upregulation of activation markers (CD25, CD69) on CD8⁺ and CD4⁺ T cells after killing
Induced secretion of IL-2, IFN-γ, TNFα, Granzyme B and IL-10 (no IL-4) upon killing.

### Example 17

### Binding of CEA TCB to CEA- and CD3-Expressing Cells

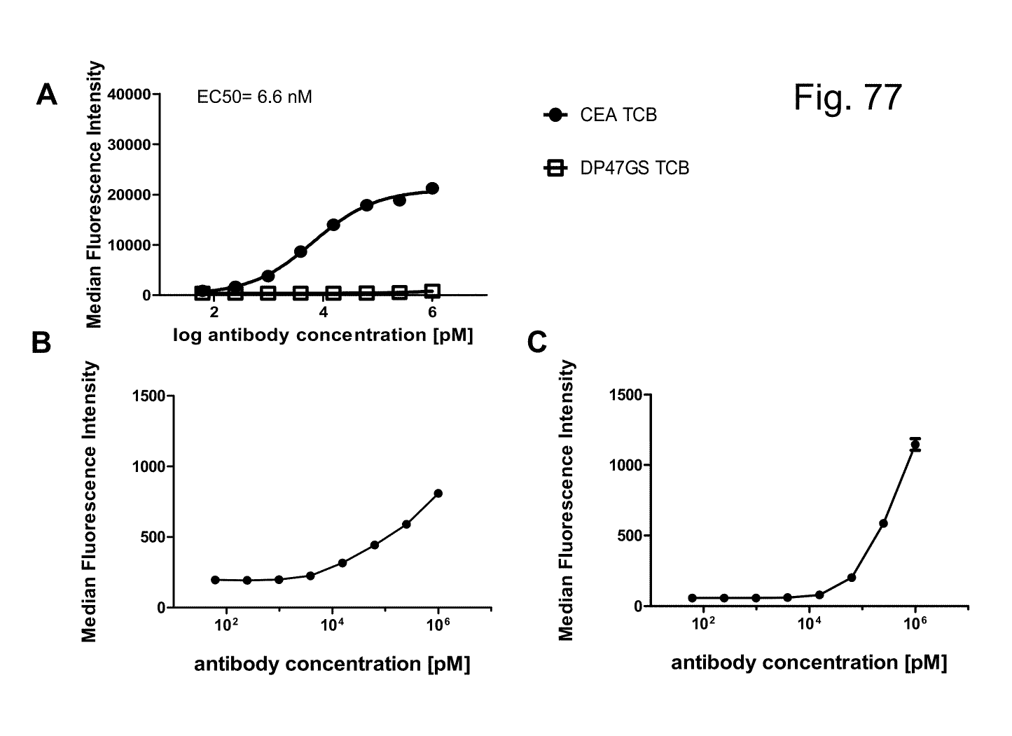

The binding of CEA TCB was tested on transfected CEA-expressing lung adenocarcinoma cells (A549-huCEA) and CD3-expressing immortalized human and cynomolgus T lymphocyte lines (Jurkat and HSC-F, respectively). An untargeted TCB (SEQ ID NOs:325, 326, 327 and 328; see example 33) was used as control. Briefly, cells were harvested, counted, checked for viability and resuspended at 2×10⁶ cells/ml in FACS buffer (100 μl PBS 0.1% BSA). 100 μl cell suspension (containing 0.2×10⁶ cells) were incubated in round-bottom 96-well plate for 30 min at 4° C. with increasing concentrations of the CEA TCB (61 pM-1000 nM), washed twice with cold PBS 0.1% BSA, re-incubated for further 30 min at 4° C. with the FITC-conjugated AffiniPure F(ab′)2 Fragment goat anti-human IgG F(ab′)2 Fragment Specific secondary antibody (Jackson Immuno Research Lab FITC #109-096-097), washed twice with cold PBS 0.1% BSA and immediately analyzed by FACS using a FACS CantoII or Fortessa (Software FACS Diva) by gating live, PI-negative, cells. Binding curves were obtained using GraphPadPrism5 (FIG. 77A, binding to A549 cells (EC₅₀ 6.6 nM); FIG. 77B, binding to Jurkat cells; FIG. 77C, binding to HSC-F cells).

### Example 18

### T Cell-Mediated Killing of CEA-Expressing Tumor Target Cells Induced by CEA TCB Antibody

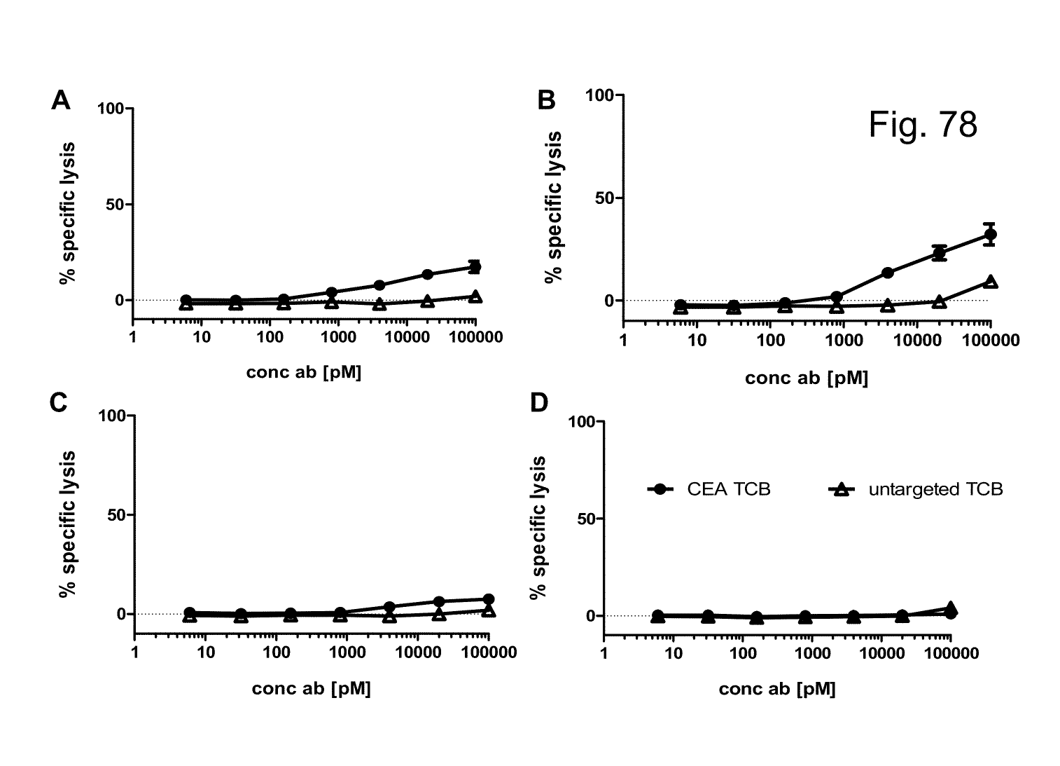

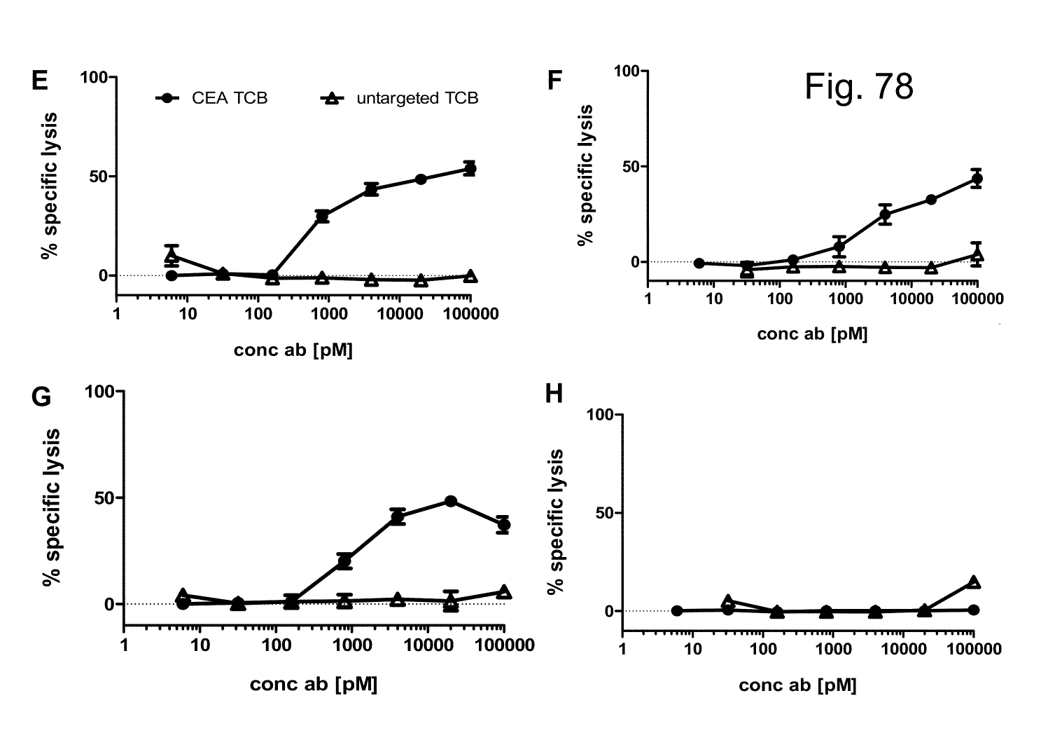

T cell-mediated killing of target cells induced by CEA TCB antibody was assessed on HPAFII (high CEA), BxPC-3 (medium CEA) and ASPC-1 (low CEA) human tumor cells. HCT-116 (CEA negative tumor cell line) and the untargeted TCB were used as negative controls. Human PBMCs were used as effectors and killing detected 24 h and 48 h after incubation with the bispecific antibody. Briefly, target cells were harvested with Trypsin/EDTA, washed, and plated at density of 25 000 cells/well using flat-bottom 96-well plates. Cells were left to adhere overnight. Peripheral blood mononuclear cells (PBMCs) were prepared by Histopaque density centrifugation of enriched lymphocyte preparations (buffy coats) obtained from healthy human donors. Fresh blood was diluted with sterile PBS and layered over Histopaque gradient (Sigma, #H8889). After centrifugation (450×g, 30 minutes, room temperature), the plasma above the PBMC-containing interphase was discarded and PBMCs transferred in a new falcon tube subsequently filled with 50 ml of PBS. The mixture was centrifuged (400×g, 10 minutes, room temperature), the supernatant discarded and the PBMC pellet washed twice with sterile PBS (centrifugation steps 350×g, 10 minutes). The resulting PBMC population was counted automatically (ViCell) and kept in RPMI1640 medium containing 10% FCS and 1% L-alanyl-L-glutamine (Biochrom, K0302) in cell incubator (37° C., 5% CO₂) until further use (no longer than 24 h). For the killing assay, the antibodies were added at indicated concentrations (range of 6 pM-100 nM in triplicates). PBMCs were added to target cells at the final E:T ratio of 10:1. Target cell killing was assessed after 24 h and 48 h of incubation by quantification of LDH (lactate dehydrogenase) released into cell supernatants by apoptotic/necrotic cells (LDH detection kit, Roche Applied Science, #11 644 793 001). Maximal lysis of the target cells (=100%) was achieved by incubation of target cells with 1% Triton X-100. Minimal lysis (=0%) refers to target cells co-incubated with effector cells without bispecific antibody. The results show that CEA TCB induced a strong and target-specific killing of CEA-positive target cells (FIG. 78, A-H). The EC₅₀ values related to the killing assays, calculated using GraphPadPrism5 are given in Table 11.

TABLE 11
CEA receptor copy number and EC₅₀ values
(pM) for T-cell mediated killing of CEA-expressing
tumor cells induced by CEA TCB antibody.

| | CEA receptor | EC50 [pM] |
|---|---|---|
| Cell line | copy number | 48 h |
| HPAFII | 120 000-205 000 | 667 |
| BxPC-3 | 41 000 | 3785 |
| ASPC1 | 3500-8000 | 846 |

### Example 19

### T Cell Proliferation and Activation 5 Days after CEA TCB-Mediated Killing of CEA-Expressing Tumor Target Cells

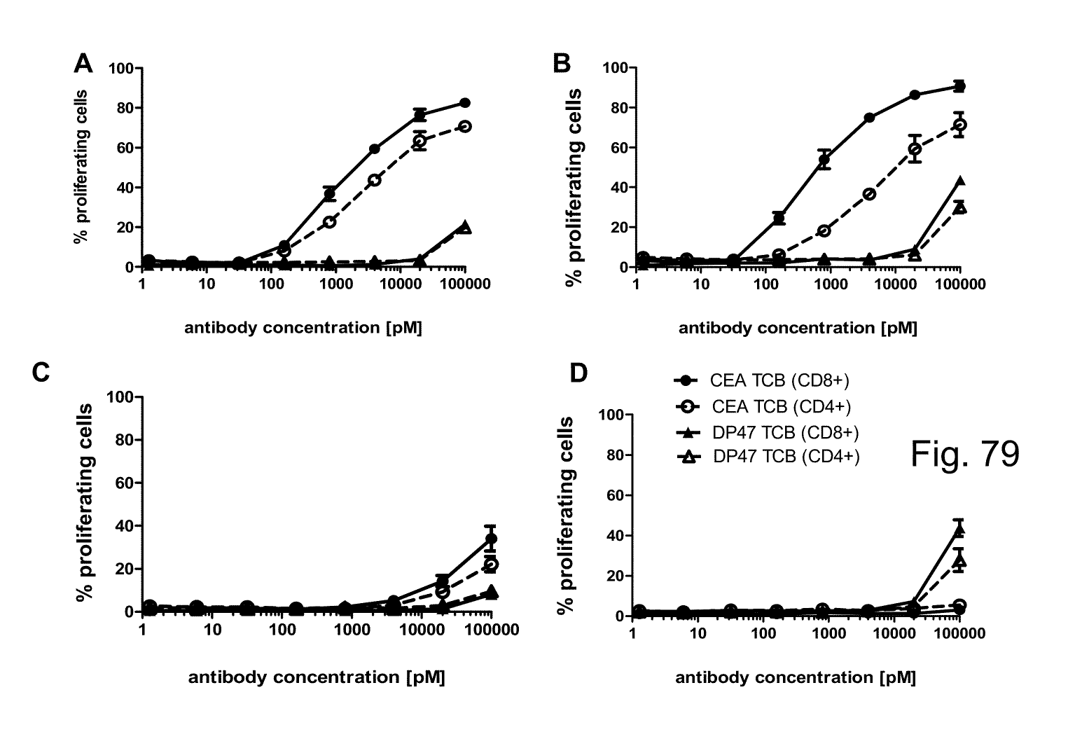

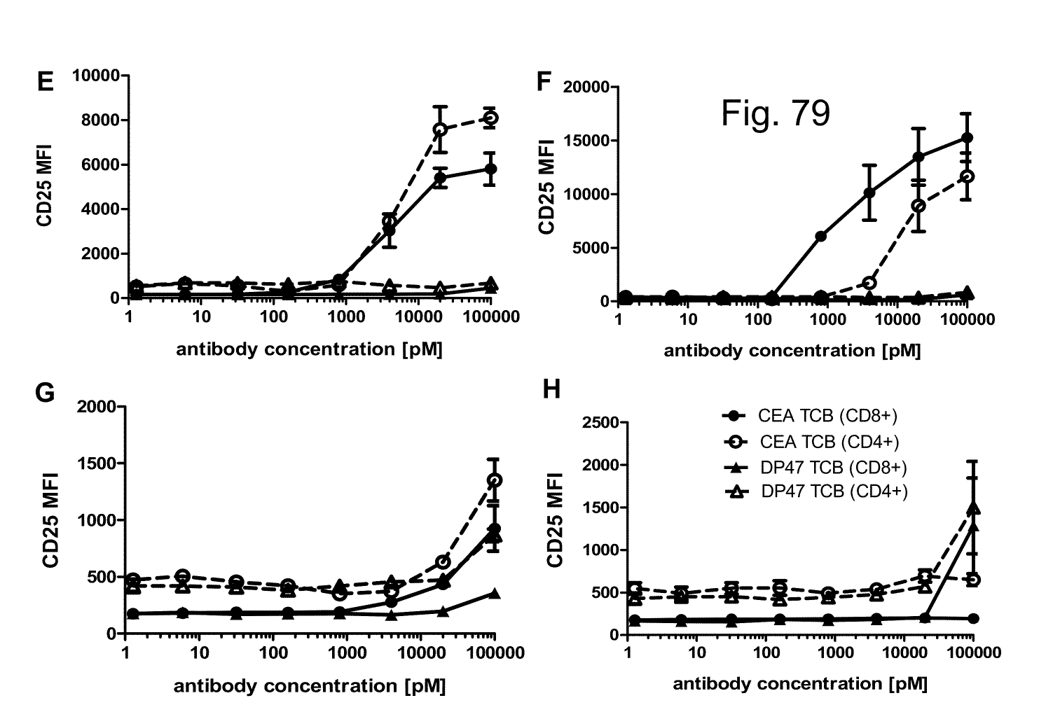

T cell proliferation and activation was detected 5 days after CEA TCB-mediated killing of CEA-expressing tumor target cells assessed on HPAFII (high CEA), BxPC-3 (medium CEA) and ASPC-1 (low CEA) cells. HCT-116 (CEA negative tumor cell line) and the untargeted TCB were used as negative controls. The experimental conditions for the proliferation assay were similar to the ones described in Example 18, but only 10 000 target cells were plated per well of a 96-flat bottom well plate. To assess T cell proliferation, freshly-isolated PBMCs were labeled using CFSE (Sigma #21888). Briefly, CFSE stock solution was diluted to obtain a working solution of 100 μM. 90×10⁶ PBMC cells were re-suspended in 90 ml pre-warmed PBS and supplemented with 90 μl of the CFSE working solution. Cells were mixed immediately and incubated 15 min at 37° C. 10 ml of pre-warmed FCS were added to cells to stop the reaction. The cells were centrifuged for 10 min at 400 g, re-suspended in 50 ml medium and incubated for 30 min at 37° C. After incubation, cells were washed once with warm medium, counted, re-suspended in medium and added to target cells for the killing assay and subsequent measurement of cell proliferation and activation at an E:T of 10:1. Proliferation was assessed 5 days after killing on CD4 and CD8 positive T cells by quantification of the CFSE dye dilution. CD25 expression was assessed on the same T cell subsets using the anti-human CD25 antibody. Briefly, after centrifugation (400×g for 4 min), cells were resuspended, washed with FACS buffer and incubated with 25 μl of the diluted CD4/CD8/CD25 antibody mix for 30 min at 4° C. (APC/Cy7 anti-human CD4 #317418, APC anti-human CD8 #301014, PE/Cy7 anti-human CD25 #302612). Cells were then washed three times to remove the unbound antibody, and finally resuspended in 200 μl FACS buffer containing propidium iodide (PI) to exclude dead cells for the FACS measurement. Fluorescence was measured using BD FACS CantoII. The results show that the CEA TCB induced a strong and target-specific proliferation of CD8⁺ and CD4⁺ T cells (FIG. 79, A-D) as well as their activation as detected by up-regulation of the CD25 activation marker (FIG. 79, E-H).

### Example 20

### Cytokine Secretion by Human Effector Cells after T Cell-Mediated Killing of CEA-Expressing Tumor Cells Induced by CEA TCB

Cytokine secretion by human PBMCs after T cell-mediated killing of CEA-expressing MKN45 tumor cells induced by the CEA TCB was assessed by FACS analysis (CBA kit) of cell supernatants 48 h after killing.

The experimental conditions were identical to the ones described in Example 18. At the end of the incubation time, the plate was centrifuged for 5 min at 350×g, the supernatant transferred into a new 96-well plate and stored at −20° C. until subsequent analysis. (A) IFN-γ, (B) TNFα, (C) Granzyme B, (D) IL-2, (E) IL-6 and (F) IL-10 secreted into cell supernatants were detected using the BD CBA Human Soluble Protein Flex Set, according to the manufacturer's instructions on a FACS CantoII. The following kits were used: BD CBA human IL-2 BD Flex Set #BD 558270; BD CBA human Granzyme B BD Flex Set #BD 560304; BD CBA human TNF Flex Set #BD 558273; BD CBA human IFN-γ Flex Set #BD 558269; BD CBA human IL-4 Flex Set #BD 558272; BD CBA human IL-10 Flex Set #BD 558274.

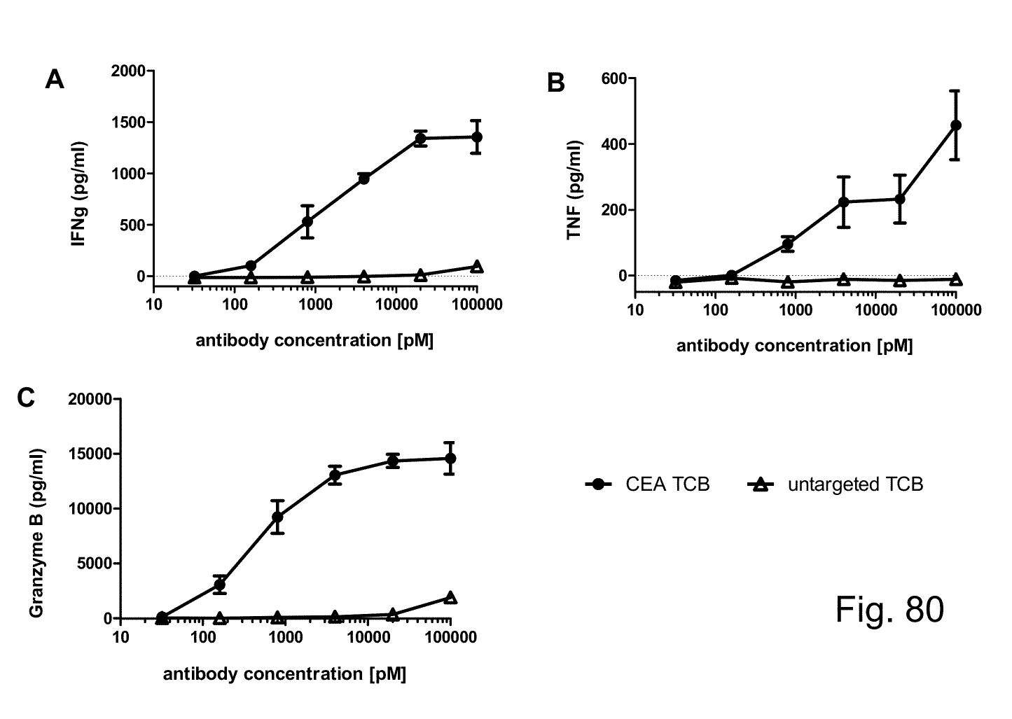

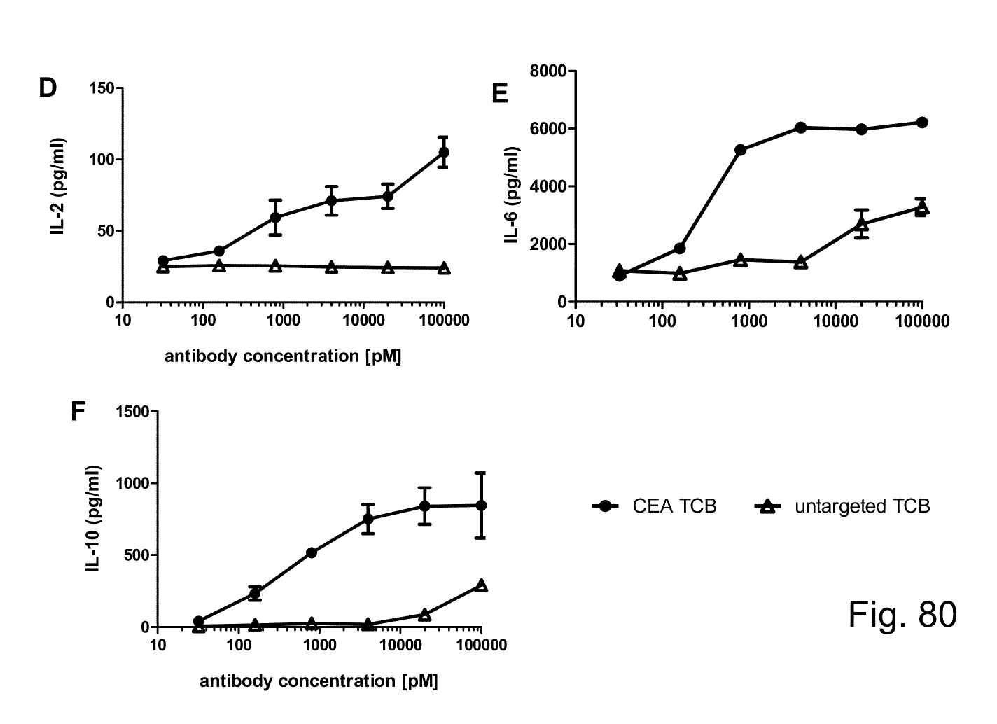

The results show that the CEA TCB mediated killing (but not the killing mediated by untargeted TCB control) induced secretion of IFN-γ, TNFα, Granzyme B, IL-2, IL-6 and IL-10 (FIG. 80, A-F).

### Example 21

### T Cell-Mediated Killing of Target Cells in Presence of Increasing Concentrations of Shed CEA (sCEA)

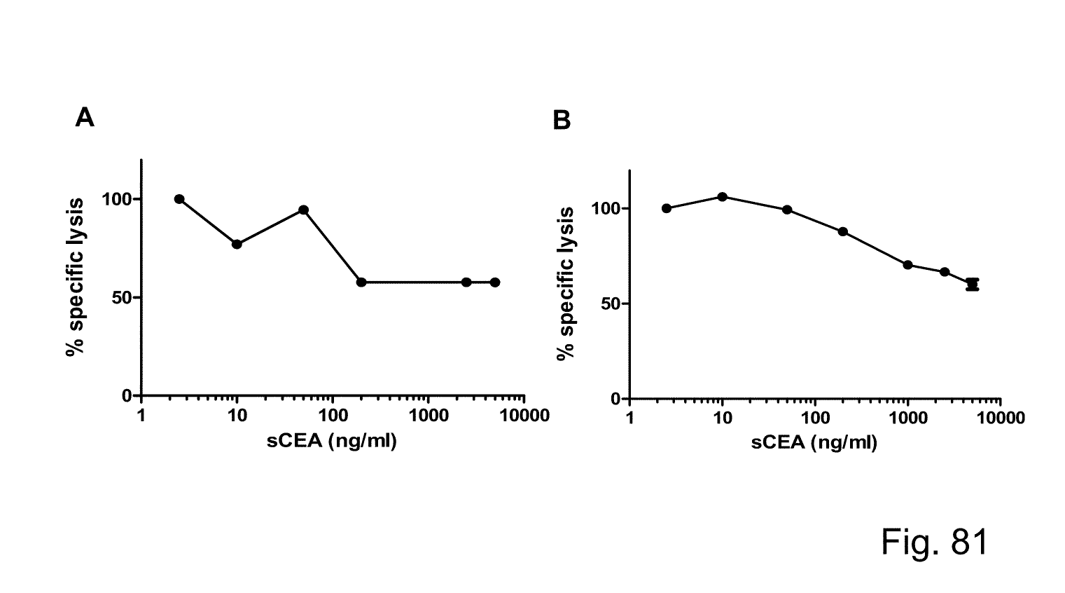

T cell-mediated killing of CEA-expressing tumor target cells (LS 180) induced by CEA TCB antibody in presence of increasing concentrations of shed CEA (sCEA 2.5 ng/ml-5 μg/ml) was assessed. Human PBMCs were used as effector cells and killing detected 24 h and 48 h after incubation with the bispecific antibody and sCEA. Briefly, target cells were harvested with Trypsin/EDTA, washed, and plated at density of 25 000 cells/well using flat-bottom 96-well plates. Cells were left to adhere overnight. Peripheral blood mononuclear cells (PBMCs) were prepared by Histopaque density centrifugation of enriched lymphocyte preparations (buffy coats) obtained from healthy human donors. Fresh blood was diluted with sterile PBS and layered over Histopaque gradient (Sigma, #H8889). After centrifugation (450×g, 30 minutes, room temperature), the plasma above the PBMC-containing interphase was discarded and PBMCs transferred in a new Falcon tube subsequently filled with 50 ml of PBS. The mixture was centrifuged (400×g, 10 minutes, room temperature), the supernatant discarded and the PBMC pellet washed twice with sterile PBS (centrifugation steps 350×g, 10 minutes). The resulting PBMC population was counted automatically (ViCell) and kept in RPMI1640 medium containing 10% FCS and 1% L-alanyl-L-glutamine (Biochrom, K0302) in cell incubator (37° C., 5% CO₂) until further use (no longer than 24 h). For the killing assay, the CEA TCB antibody was used at a fixed concentration of 1 nM and sCEA was spiked into the experiment at a concentration range of 2.5 ng-5 μg/ml. PBMCs were added to target cells at the final E:T ratio of 10:1. Target cell killing was assessed after 24 h and 48 h of incubation by quantification of LDH (lactate dehydrogenase) released into cell supernatants by apoptotic/necrotic cells (LDH detection kit, Roche Applied Science, #11 644 793 001). Maximal lysis of the target cells (=100%) was achieved by incubation of target cells with 1% Triton X-100. Minimal lysis (=0%) refers to target cells co-incubated with effector cells without bispecific antibody. The killing mediated by CEA TCB in absence of sCEA was set at 100% and the killing obtained in presence of increasing concentrations of sCEA was normalized to it. Results show that sCEA had only a minor impact on CEA TCB-mediated killing of CEA-expressing target cells (FIG. 81 A, B). No effect on T cell killing was detected up to 0.2 μg/ml of sCEA. The sCEA concentrations above 0.2 μg/ml had only a minor impact on overall killing (10-50% reduction).

### Example 22

### T Cell-Mediated Killing of Target Cells Using Human and Cynomolgus PBMCs as Effector Cells

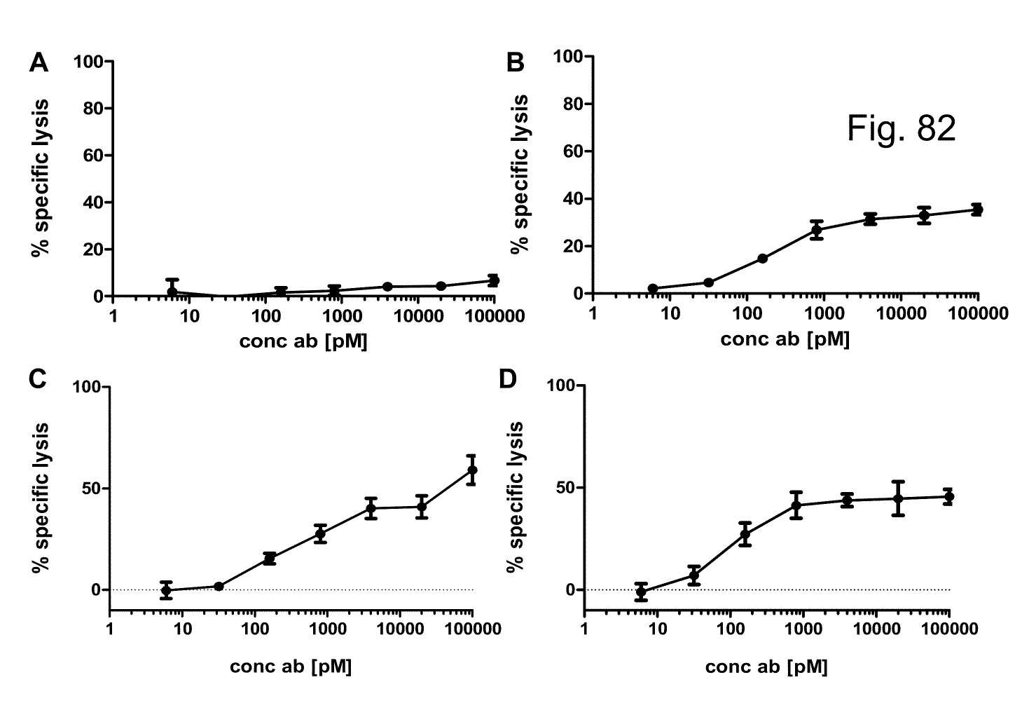

T cell-mediated killing of A549 (lung adenocarcinoma) cells overexpressing human CEA (A549-hCEA), assessed 21 h and 40 h after incubation with CEA TCB antibody and human PBMCs or cynomolgus PBMCs as effector cells was assessed. Briefly, target cells were harvested with Trypsin/EDTA, washed, and plated at density of 25 000 cells/well using flat-bottom 96-well plates. Cells were left to adhere for several hours. Peripheral blood mononuclear cells (PBMCs) were prepared by Histopaque density centrifugation of enriched lymphocyte preparations (buffy coats) obtained from healthy human donors or healthy cynomolgus monkey. For the later, a 90% Histopaque-PBS density gradient was used. Fresh blood was diluted with sterile PBS and layered over Histopaque gradient (Sigma, #H8889). After centrifugation (450×g, 30 minutes, room temperature for human PBMCs, respective 520×g, 30 min, room temperature for cynomolgus PBMCs), the plasma above the PBMC-containing interphase was discarded and PBMCs transferred in a new Falcon tube subsequently filled with 50 ml of PBS. The mixture was centrifuged (400×g, 10 minutes, room temperature), the supernatant discarded and the PBMC pellet washed twice with sterile PBS (centrifugation steps 350×g, 10 minutes). For the preparation of the cynomolgus PBMCs, an additional low-speed centrifugation step was performed at 150×g for 15 min. The resulting PBMC population was counted automatically (ViCell) and kept in RPMI1640 medium containing 10% FCS and 1% L-alanyl-L-glutamine (Biochrom, K0302) in cell incubator (37° C., 5% CO₂) until further use (up to 4 h). For the killing assay, the antibodies were added at indicated concentrations (range of 6 pM-100 nM in triplicates). PBMCs were added to target cells at the final E:T ratio of 10:1. Target cell killing was assessed after 21 h and 40 h of incubation by quantification of LDH (lactate dehydrogenase) released into cell supernatants by apoptotic/necrotic cells (LDH detection kit, Roche Applied Science, #11 644 793 001). Maximal lysis of the target cells (=100%) was achieved by incubation of target cells with 1% Triton X-100. Minimal lysis (=0%) refers to target cells co-incubated with effector cells without bispecific antibody. Results show that CEA TCB mediates target-specific killing of CEA-positive target cells using both human (FIG. 82, A, C) and cynomolgus (FIG. 82, B, D) effector cells (PBMCs). The EC₅₀ values related to 40 h of killing, calculated using GraphPadPrism5 are 306 pM for human PBMCs and 102 pM for cynomolgus PBMCs.

### Example 23

### T Cell-Mediated Killing of CEA-Expressing Human Colorectal Cancer Cell Lines Induced by CEA TCB Antibody

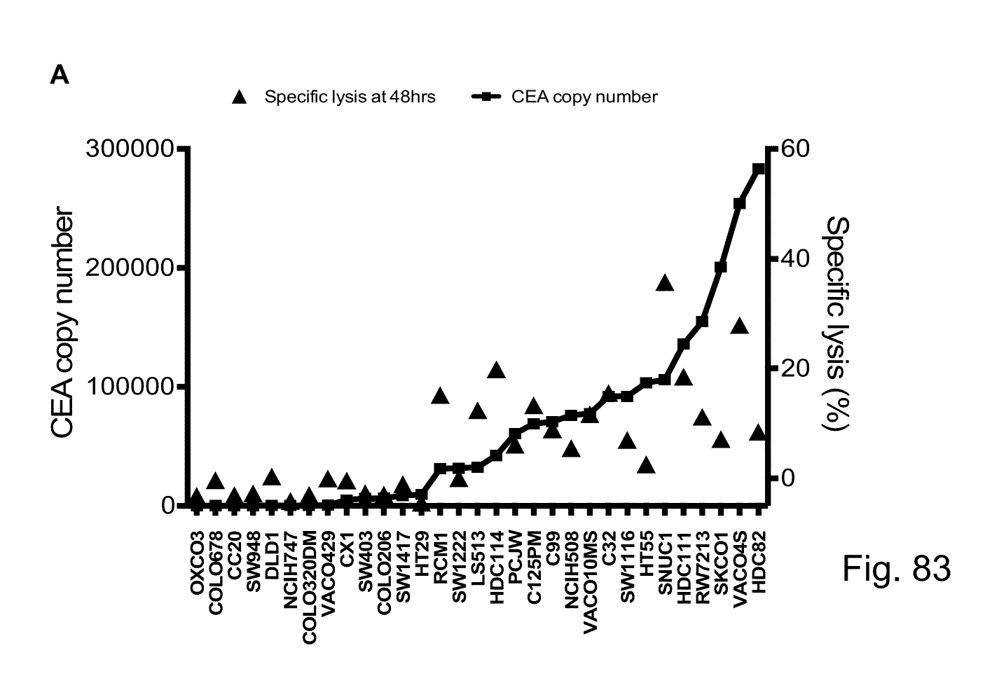

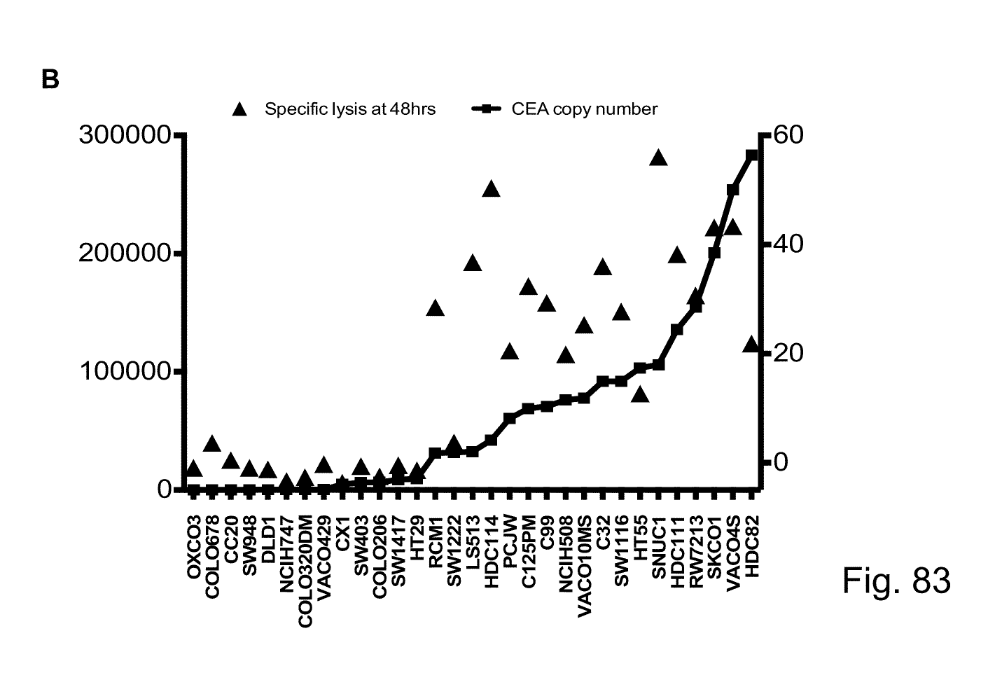

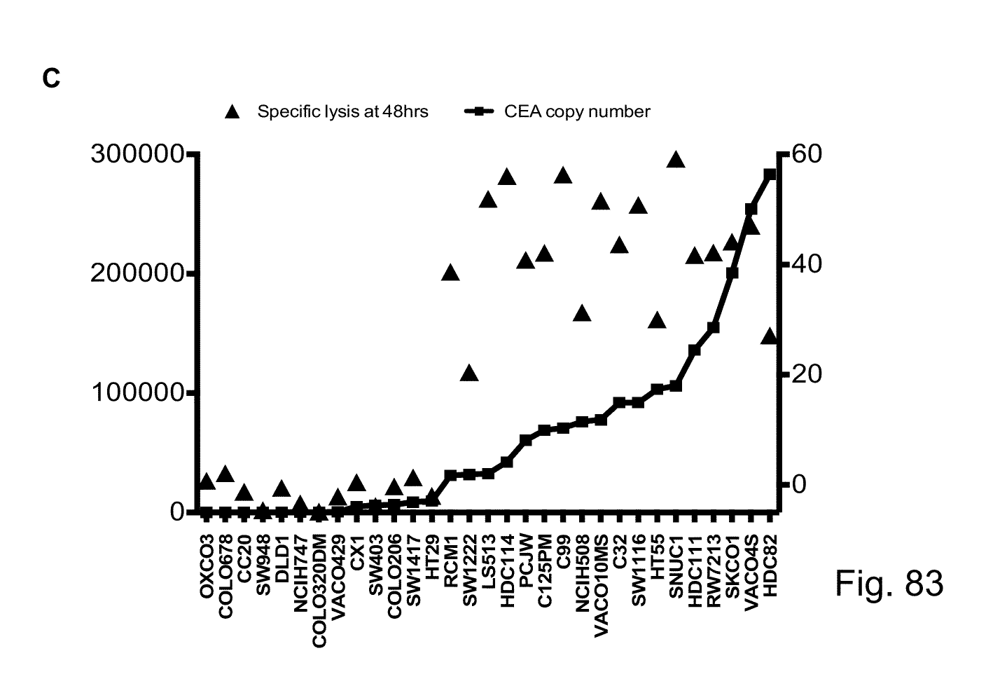

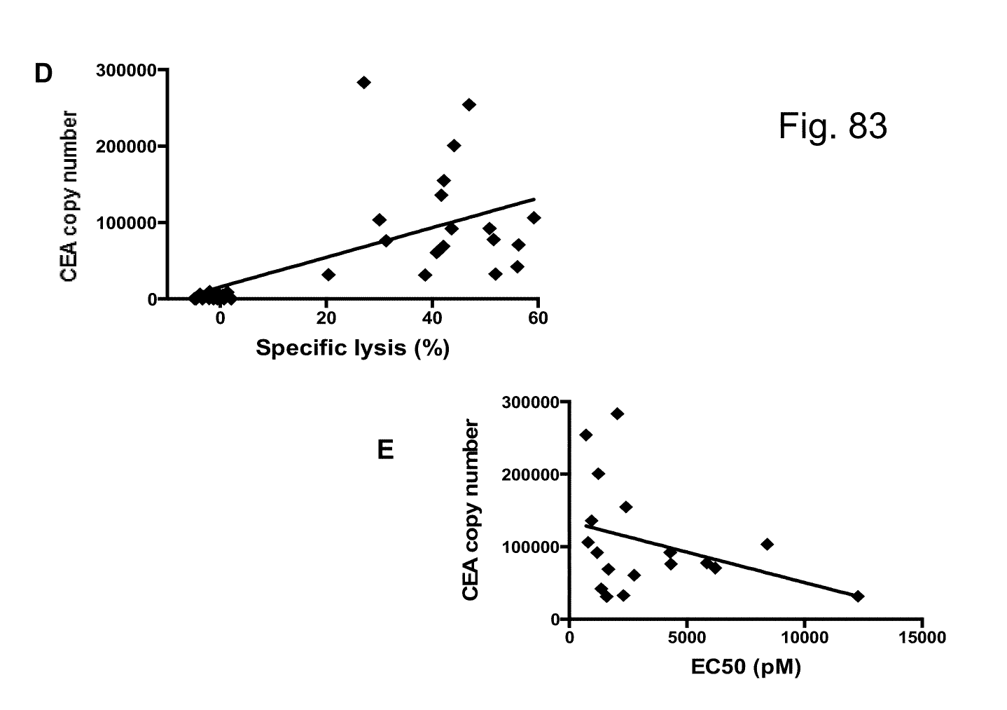

T cell-mediated killing of CEA-expressing human colorectal cancer cell lines 48 h after incubation with human PBMCs and CEA TCB antibody at 0.8 nM, 4 nM and 20 nM was assessed. Briefly, PBMCs were isolated from leukocyte cones obtained from single healthy donors. Cells were diluted with PBS (1:10) and layered on Lymphoprep in 50 mL Falcon tubes. After centrifugation (1800 rpm for 25 min), the PBMC layer was withdrawn from the interface and washed 4x with PBS. PBMCs were counted, frozen in 10% DMSO in FCS under controlled-rate freezing conditions at 40×10⁶ cells/mL and stored in liquid nitrogen until further use. For the T-cell killing assay, tumor cells were plated directly into 96-well plates from frozen stocks. Cells were warmed quickly and transferred immediately into pre-warmed medium, centrifuged, and re-suspended in complete medium (DMEM, Iscoves or RPMI-1640, all supplemented with 10% FCS and 1% penicillin/streptomycin) and plated at a density of 2.5×10⁴ cells/well. Plates were then incubated at 37° C. in a humidified 10% CO₂ incubator and medium replaced the next day by 100 μL of RPMI 2% FCS with 1% glutamine and 50 μL CEA TCB (final concentrations ranging from 6.4 to 20000 pM, 1:5 titration steps, in duplicate wells for each condition). Fresh-thawed PBMCs were used for the assay (thawed from frozen vials within 2 hours of the assay start) and 50 μL (3×10⁵) was added to each well to give an effector:target (E:T) ratio of 10:1. Triton X100 (50 μL of 4%) was added to 150 μL of target cells to obtain maximum release values. Plates were incubated at 37° C. for 48 h and the killing activity determined using the Lactose Dehydrogenase Cytotoxicity Detection Kit (Roche) in accordance with the manufacturer's instructions. Percentage of specific cell lysis was calculated as [sample release−spontaneous release]/[maximum release−spontaneous release]×100. FIG. 83, A-C shows the correlation between CEA expression (receptor copy number quantified using QIFIKIT, see below) and % killing for 31 colorectal cancer cell lines (listed on x axis). FIG. 83, D shows the correlation between CEA expression and % specific lysis at 20 nM of CEA TCB (Spearman correlation=0.7289, p<0.0001, n=31), indicating that tumor cells displaying high CEA receptor copy numbers (>50 000) are efficiently lysed by CEA TCB whereas a cluster of cells displaying low CEA receptor copy numbers (<10 000) are not being lysed by CEA TCB under the same experimental conditions. FIG. 83, E shows the correlation between CEA expression and EC₅₀ of CEA TCB. Although the correlation is not statistically significant (Spearman correlation=−0.3994, p=0.1006, R²=0.1358) the graph clearly shows a pattern of better CEA TCB potency (i.e. lower EC₅₀ values) on tumor cell lines expressing high CEA receptor copy numbers.

For the analysis of CEA surface expression on cancer cell lines, the Qifikit (DakoCytomation, Glostrup, Denmark) was used to calibrate the fluorescent signals and determine the number of binding sites per cell. Cells were incubated on ice for 30 min with a mouse anti-human CEACAM5 monoclonal antibody (0.5 μg for 5×105 cells, clone: CI-P83-1, sc-23928, Santa Cruz), washed twice with PBS1X-BSA 0.1% followed by a 45 min incubation with polyclonal fluorescein isothiocyanate-conjugated goat anti-mouse antibody provided with the Qifikit. Dead cells were excluded from the analysis using 4′,6-diamidino-2-phenylindole (DAPI) staining Samples were analysed on a CyAn™ ADP Analyzer (Beckman Coulter). All mean fluorescence intensities (MFIs) were obtained after data analyses using Summit 4.3 software. These MFIs were used to determine the relative number of antibody binding sites on the cell lines (named as CEA copy number on the results) using the equation obtained from the calibration curve (Qifikit calibration beads).

The colorectal cancer cell lines used for the T-cell killing assays and CEA surface expression quantification were seeded from cryovials. The method used to maintain the frozen stock was as described in Bracht et al. (Bracht et al. (2010), Br J Cancer 103, 340-346).

### Example 24

### In Vivo Anti-Tumor Efficacy of CEA TCB in a LS174T-Fluc2 Human Colon Carcinoma Co-Grafted with Human PBMC (E:T Ratio 5:1)

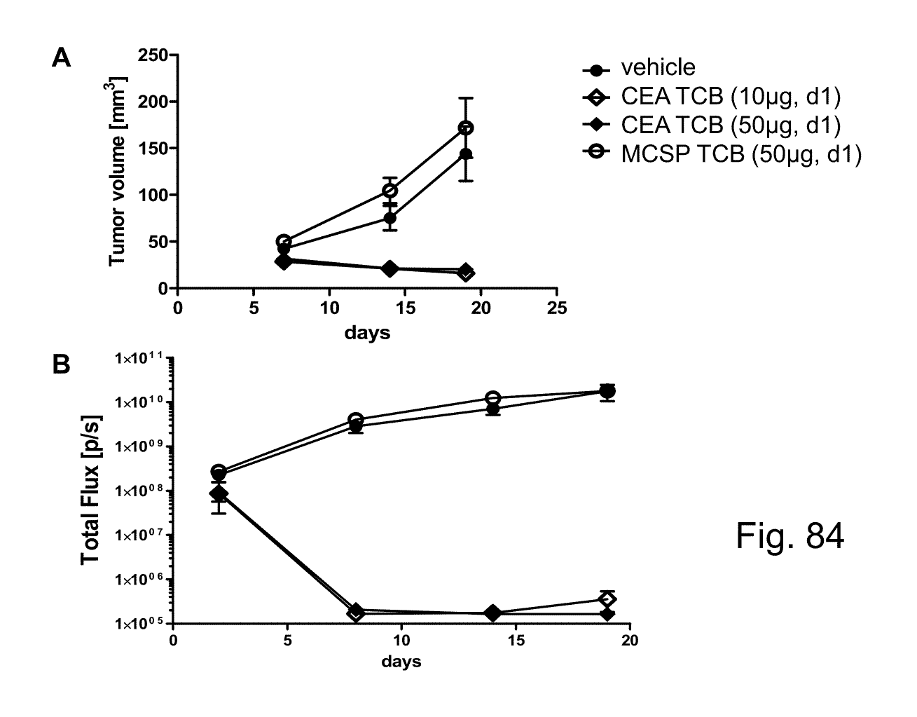

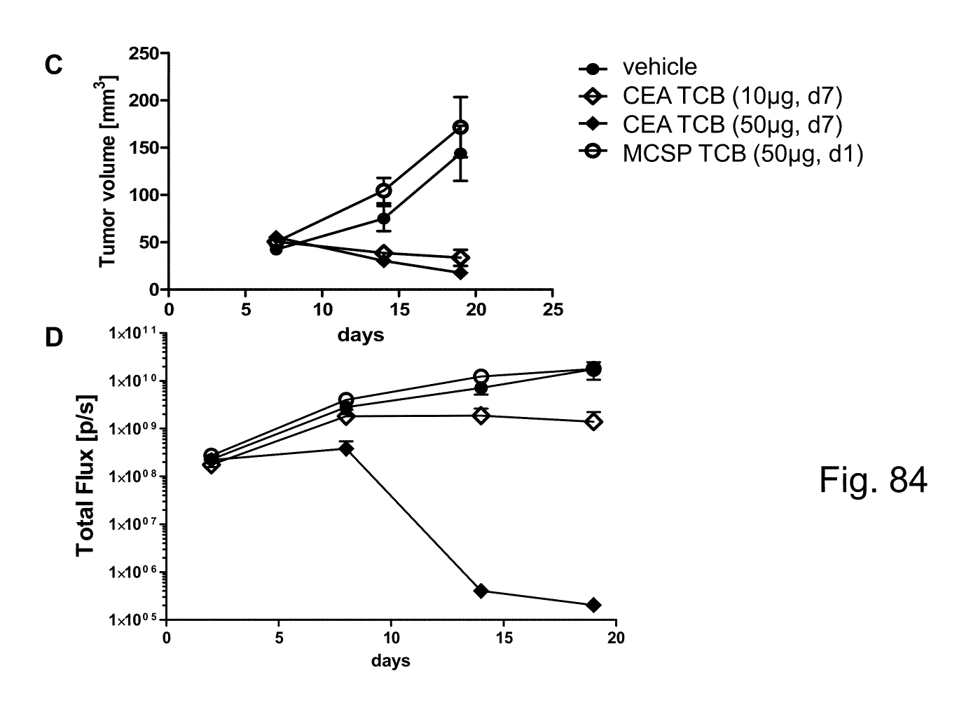

NOG (NOD/Shi-scid/IL-2Rγnull) mice (n=12) were injected subcutaneously with 1×10⁶ LS174T-fluc2 cells pre-mixed with human PBMC in a total volume of 100 μl in PBS, E:T ratio 5:1. LS174T-fluc2 cells have been engineered to express luciferase, which allows monitoring tumor progression by bioluminescence (BLI) in a non-invasive and highly sensitive manner. To assess early and delayed treatment effects, mice received bi-weekly i.v. injections of either 0.5 or 2.5 mg/kg of the CEA TCB starting at day 1 (early treatment) or day 7 (delayed treatment) after tumor cell/PBMCs co-grafting s.c. As a control, one group of mice received bi-weekly i.v. injections of 2.5 mg/kg of a control TCB that had the same format as CEA TCB (in this case the MCSP TCB served as untargeted control since LS174T-fluc2 cells do not express MCSP), and an extra control group received only PBS (vehicle) starting at day 1. Tumor volume was measured once a week by digital caliper. Furthermore, mice were injected i.p. once weekly with D-Luciferin and the bioluminescent light emission of living tumor cells was measured with IVIS Spectrum (Perkin Elmer). Treatment was administered until 19 days after tumor cell inoculation, which corresponds to the day of study termination. The results of the experiment are shown in FIG. 84 A-D. Results show average and SEM from 12 mice of tumor volume measured by caliper (A and C) and by bioluminescence (Total Flux, B and D) in the different study groups ((A, B) early treatment, (C, D) delayed treatment).

### Example 25

### In Vivo Anti-Tumor Efficacy of CEA TCB in a LS174T-Fluc2 Human Colon Carcinoma Co-Grafted with Human PBMC (E:T Ratio 1:1)

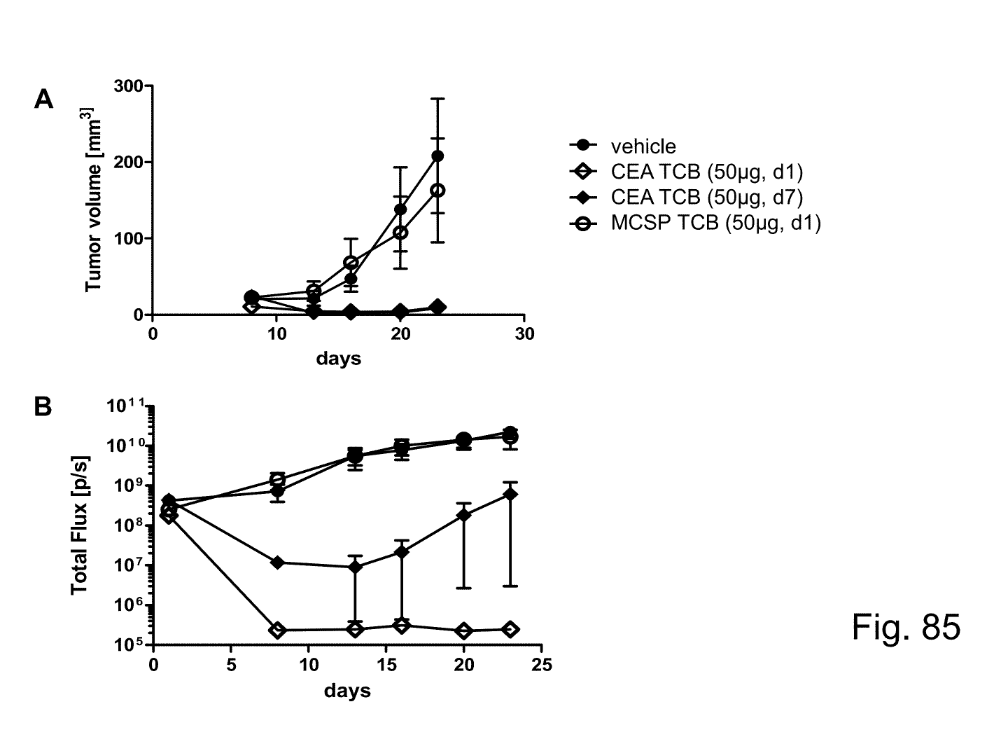

NOG (NOD/Shi-scid/IL-2Rγnull) mice (n=10) were injected subcutaneously with 1×10⁶ LS174T-fluc2 cells (see Example 24) pre-mixed with human PBMC in a total volume of 100 μl in PBS, E:T ratio 1:1. To assess early and delayed treatment effects, mice received bi-weekly i.v. injections of 2.5 mg/kg of the CEA TCB starting at day 1 (early treatment) or day 7 (delayed treatment) after tumor cell inoculation. As control, one group of mice received bi-weekly i.v. injections of 2.5 mg/kg of the MCSP TCB (see also Example 24), and an extra control group received only PBS (vehicle) starting at day 1. Tumor volume was measured once weekly by digital caliper. Furthermore, mice were injected i.p. once weekly with D-Luciferin and the bioluminescent light emission of living tumor cells was measured with IVIS Spectrum (Perkin Elmer). Treatment was administered until 23 days after tumor cell inoculation, which corresponds to the day of study termination. The results of the experiment are shown in FIG. 85. Results show average and SEM of tumor volume measured by caliper (A) as well as by bioluminescence (B) in the different study groups (n=10).

### Example 26

### In Vivo Efficacy of Murinized CEA TCB in a Panco2-huCEA Orthotopic Tumor Model in Immunocompetent huCD3ε/huCEA Transgenic Mice

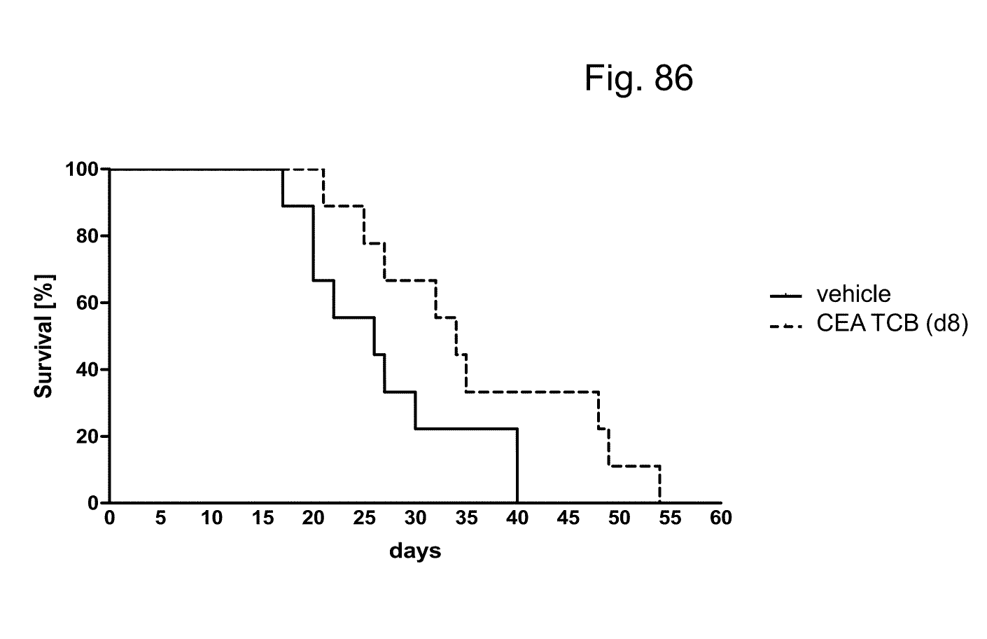

huCD3ε/huCEA transgenic mice (n=10) received an intra-pancreatic injection of 2×10⁵ Panco2-huCEA cells in a total volume of 10 μl in PBS. As murine cells do not express CEA, the murine pancreatic carcinoma cell line Panco2 was engineered to overexpress human CEA as the target antigen for the CEA TCB. Mice were injected twice weekly i.v. with 0.5 mg/kg of the murinized CEA TCB or PBS as a control group (vehicle) and survival was monitored. Animals were controlled daily for clinical symptoms and detection of adverse effects. Termination criteria for animals were visible sickness: scruffy fur, arched back, breathing problems, impaired locomotion. The result as overall survival is shown in FIG. 86. Result shows percent of surviving animals per time point. The significance of the treatment group to the PBS control group was compared using a paired Student t test (p=0.078).

### Example 27

### Affinity of the CEA TCB to CEA and CD3 by Surface Plasmon Resonance (SPR)

Surface plasmon resonance (SPR) experiments were performed on a Biacore T100 at 25° C. with HBS-EP as running buffer (0.01 M HEPES pH 7.4, 0.15 M NaCl, 3 mM EDTA, 0.005% Surfactant P20, Biacore, Freiburg/Germany).

For affinity measurements CEA TCB was captured on a CM5 sensorchip surface with immobilized anti human Fab (GE Healthcare #28-9583-25). Capture IgG was coupled to the sensorchip surface by direct immobilization of around 10,000 resonance units (RU) at pH 5.0 using the standard amine coupling kit (Biacore, Freiburg/Germany).

To analyze the interaction to human CD3c stalk-Fc (knob)-Avi/CD3δ-stalk-Fc(hole) (SEQ ID NOs 378 and 379, respectively), CEA TCB was captured for 30 s at 50 nM with 10 μl/min. CD3ε/CD3δ was passed at a concentration of 0.68-500 nM with a flowrate of 30 μl/min through the flow cells over 360 s. The dissociation was monitored for 360 s.

The K_{D} value of the interaction between CEA TCB and the recombinant tumor target antigen human NABA-avi-his (containing the B3 domain of human CEA (CEACAM5) surrounded by the N, A1 and A2 domain of human CEACAM1 with a C-terminal avi 6his tag; see SEQ ID NO: 377) was determined by capturing the TCB molecule for 40 s at 10 μl/min. The antigen was flown over the flow cell for 240 s in a concentration range from 0.68 to 500 nM at a flow rate of 30 μl/min. The dissociation was measured over 240 s.

Bulk refractive index differences were corrected for by subtracting the response obtained on a reference flow cell. Here, the antigens were flown over a surface with immobilized anti-human Fab antibody but on which HBS-EP has been injected rather than CEA.

Kinetic constants were derived using the Biacore T200 Evaluation Software (vAA, Biacore AB, Uppsala/Sweden), to fit rate equations for 1:1 Langmuir binding by numerical integration. The half-life (t_{1/2}) of the interaction was calculated using following formula: t_{1/2}=ln 2/k_{off}.

The CEA TCB binds to the tumor target and CD3ε/CD3δ in the nM-range with K_{D} values of 62 nM for the human NABA and 75.3 nM for the human CD3ε/CD3δ. The half-life of the monovalent binding to NABA is 5.3 minutes, the half-life of the binding to CD3ε/CD3δ is 5.7 minutes. The kinetic values are summarized in Table 12.

TABLE 12
Affinity of CEA TCB to human NABA and human CD3ε/CD3δ (T = 25° C.).

| Antigen | TCB | kₒₙ [1/Ms] | k_{off} [1/s] | t_{1/2} [min] | K_{D} [nM] |
|---|---|---|---|---|---|
| Human | CEA TCB | 3.49 × 10⁴ | 2.18 × 10⁻³ | 5.3 | 62.4 |
| NABA | | | | | |
| Human | CEA TCB | 2.69 × 10⁴ | 2.03 × 10⁻³ | 5.7 | 75.3 |
| CD3ε/CD3δ | | | | | |

### Example 28

### Affinity of the MSCP TCB to MCSP and CD3 by Surface Plasmon Resonance (SPR)

Surface plasmon resonance (SPR) experiments were performed on a Biacore T100 at 25° C. with HBS-EP as running buffer (0.01 M HEPES pH 7.4, 0.15 M NaCl, 3 mM EDTA, 0.005% Surfactant P20, Biacore, Freiburg/Germany).

For affinity measurements MCSP TCB was captured on a CM5 sensorchip surface with immobilized anti human Fab (GE Healthcare #28-9583-25). Capture IgG was coupled to the sensorchip surface by direct immobilization of around 7,500 resonance units (RU) at pH 5.0 using the standard amine coupling kit (Biacore, Freiburg/Germany). MCSP TCB was captured for 60 s at 30 nM with 10 μl/min. Human and cynomolgus MCSP D3 (see SEQ ID NOs 376 and 375, respectively) were passed at a concentration of 0.024-50 nM with a flowrate of 30 μl/min through the flow cells over 90 s. The concentration range for human and cynomolgus CD3ε stalk-Fc (knob)-Avi/CD3δ-stalk-Fc(hole) was 1.17-600 nM. Since the interaction with murine MCSP (SEQ ID NO: 380) was expected to be weak the concentration range for this antigen was chosen between 3.9 and 500 nM. The dissociation for all interactions was monitored for 120 s. Bulk refractive index differences were corrected for by subtracting the response obtained on a reference flow cell. Here, the antigens were flown over a surface with immobilized anti-human Fab antibody but on which HBS-EP has been injected rather than MCSP TCB.

Kinetic constants were derived using the Biacore T200 Evaluation Software (vAA, Biacore AB, Uppsala/Sweden), to fit rate equations for 1:1 Langmuir binding by numerical integration. The interaction for the MCSP TCB with the murine MCSP D3 was determined in steady state. The half-life (t_{1/2}) of the interaction was calculated using following formula: t_{1/2}=ln 2/k_{off}.

The MCSP TCB binds to the tumor target in pM-range with K_{D} values of 0.15 nM for the human and 0.12 nM for the cynomolgus antigen. Recombinant CD3ε/CD3δ is bound by the MCSP TCB with a K_{D} value of 78 nM (human) and 104 nM (cynomolgus). The half-life of the monovalent binding is up to 260 minutes for the tumor target and 2.9 minutes for the CD3e/CD3d. Upon affinity maturation the MCSP antibody obtained some binding to recombinant murine MCSP D3. K_{D} value for this interaction is in mM range (1.6 mM). The kinetic values are summarized in Table 13.

TABLE 13
Affinity of MCSP TCB to the human, cynomolgus and murine MCSP D3
and human and cynomolgus CD3ε/CD3δ (T = 25° C.).

| | kₒₙ [1/Ms] | k_{off} [1/s] | t_{1/2} [min] | K_{D} [nM] |
|---|---|---|---|---|
| Human MCSP D3 | 3.89 × 10⁵ | 5.63 × 10−⁵ | 205 | 0.15 |
| Cynomolgus MCSP D3 | 3.70 × 10⁵ | 4.39 × 10−⁵ | 263 | 0.12 |
| Murine MCSP D3 | nd | nd | nd | 1570* |
| Human CD3ε/CD3δ | 4.99 × 10⁴ | 3.92 × 10⁻³ | 2.9 | 78.7 |
| Cynomolgus CD3ε/CD3δ | 4.61 × 10⁴ | 4.78 × 10⁻³ | 2.4 | 104 |

*determined by steady state measurement

### Example 29

### Thermal Stability of CEA TCB

Thermal stability of the CEA TCB was monitored by Dynamic Light Scattering (DLS). 30 μg of filtered protein sample with a protein concentration of 0.5 mg/ml was applied in duplicate to a Dynapro plate reader (Wyatt Technology Corporation; USA). The temperature was ramped from 25 to 75° C. at 0.05° C./min, with the radius and total scattering intensity being collected.

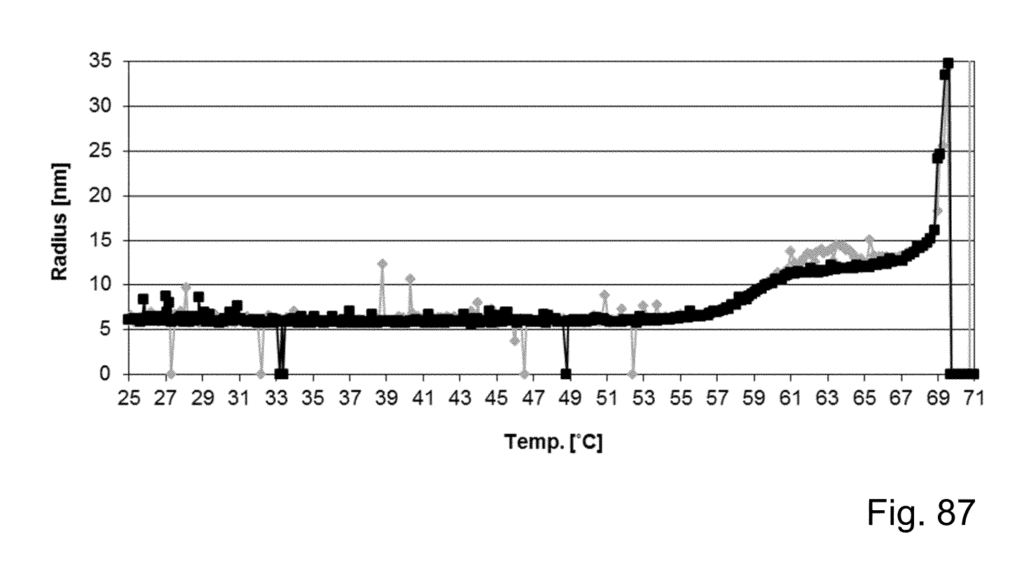

The result is shown in FIG. 87. The aggregation temperature of the CEA TCB was measured at 55° C.

### Example 30

### Thermal Stability of MCSP TCB

Thermal stability of the MCSP TCB was monitored by Dynamic Light Scattering (DLS). 30 μg of filtered protein sample with a protein concentration of 0.5 mg/ml was applied in duplicate to a Dynapro plate reader (Wyatt Technology Corporation; USA). The temperature was ramped from 25 to 75° C. at 0.05° C./min, with the radius and total scattering intensity being collected.

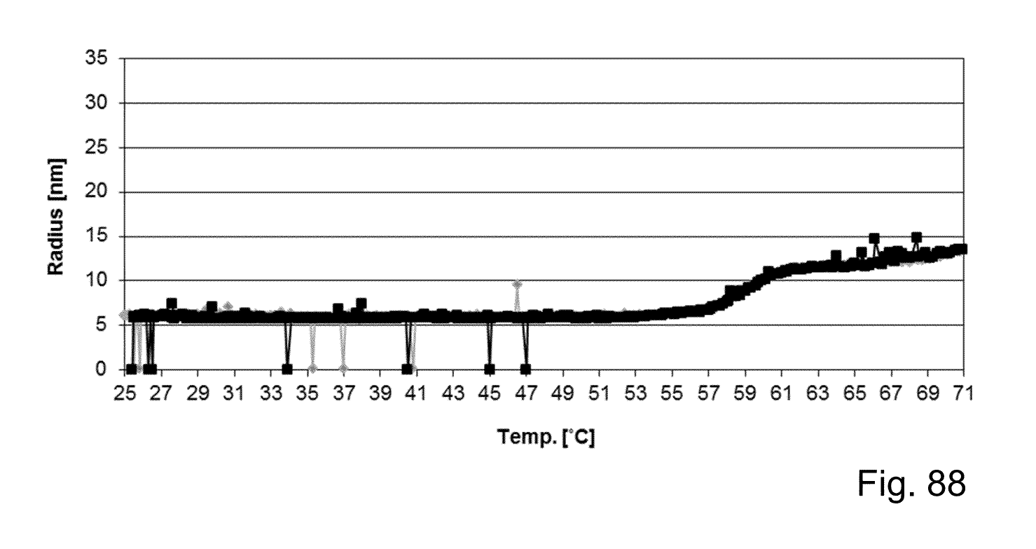

The result is shown in FIG. 88. The aggregation temperature of the MCSP TCB was measured at 55° C.

### Example 31

### T Cell-Mediated Killing of MCSP-Expressing Tumor Target Cells Induced by MCSP TCB and MCSP 1+1 CrossMab Antibodies

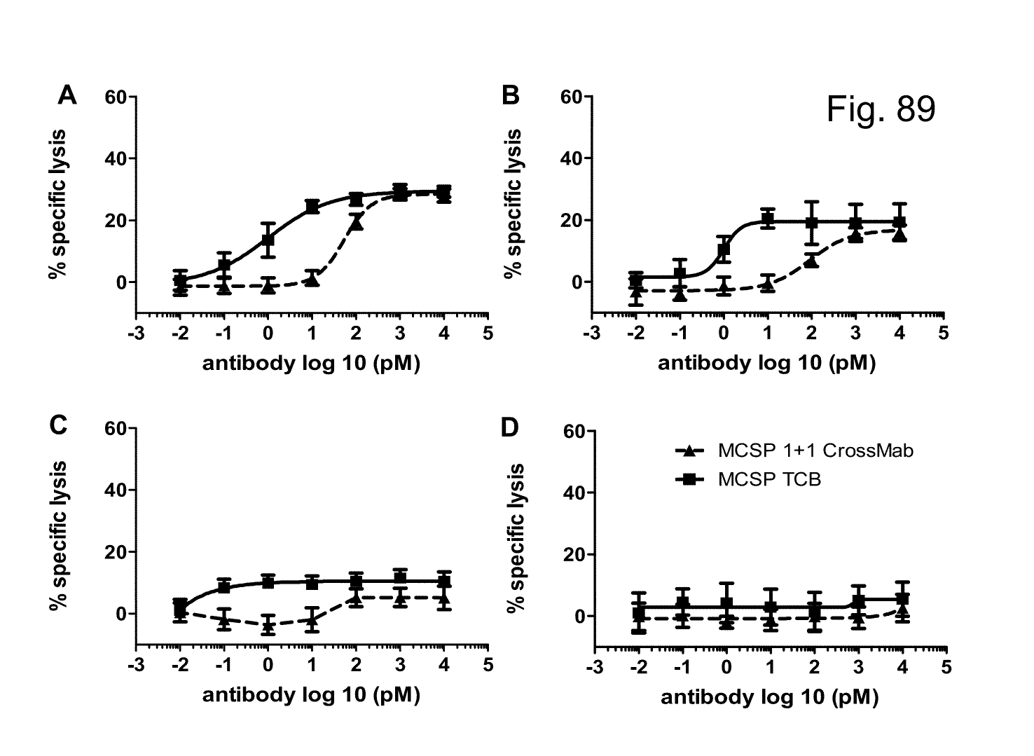

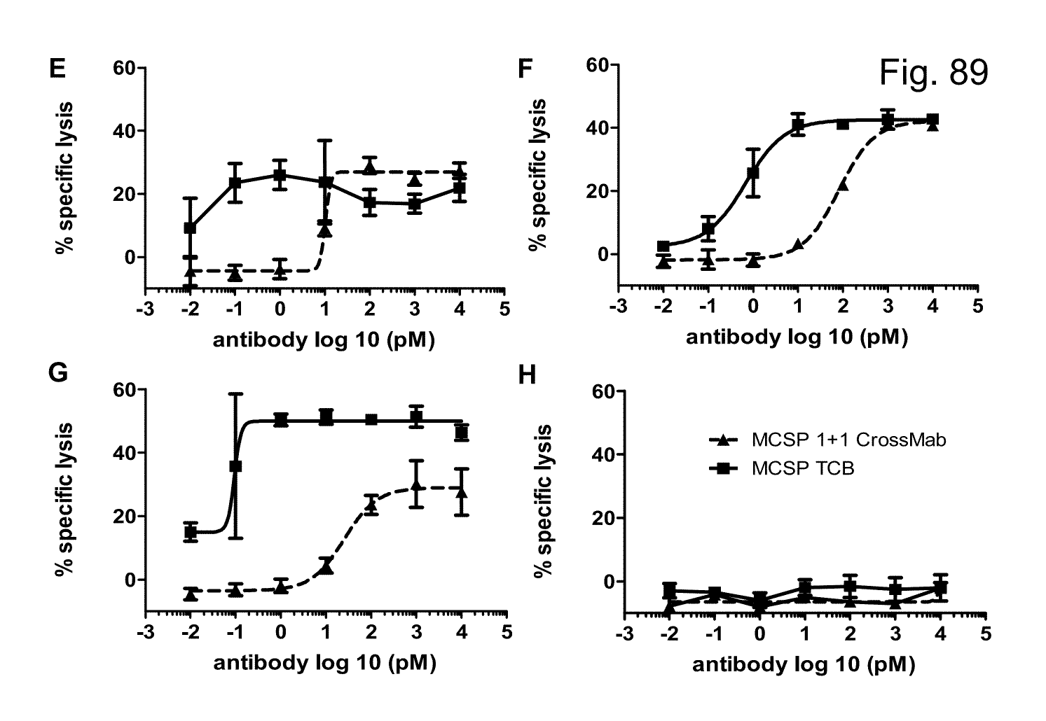

T cell-mediated killing of target cells induced by MCSP TCB and MCSP 1+1 CrossMab TCB (a T cell activating bispecific antibody having the same CD3 and MCSP binding sequences as the MCSP TCB, with the molecular format shown in FIG. 1D) antibodies was assessed on A375 (high MCSP), MV-3 (medium MCSP) and HCT-116 (low MCSP) tumor target cells. LS180 (MCSP negative tumor cell line) was used as negative control. Tumor cell killing was assessed 24 h and 48 h post incubation of target cells with the antibodies and effector cells (human PBMCs). Briefly, target cells were harvested with Trypsin/EDTA, washed, and plated at density of 25 000 cells/well using flat-bottom 96-well plates. Cells were left to adhere overnight. Peripheral blood mononuclear cells (PBMCs) were prepared by Histopaque density centrifugation of enriched lymphocyte preparations (buffy coats) obtained from healthy human donors. Fresh blood was diluted with sterile PBS and layered over Histopaque gradient (Sigma, #H8889). After centrifugation (450×g, 30 minutes, room temperature), the plasma above the PBMC-containing interphase was discarded and PBMCs transferred in a new Falcon tube subsequently filled with 50 ml of PBS. The mixture was centrifuged (400×g, 10 minutes, room temperature), the supernatant discarded and the PBMC pellet washed twice with sterile PBS (centrifugation steps 350×g, 10 minutes). The resulting PBMC population was counted automatically (ViCell) and kept in RPMI1640 medium containing 10% FCS and 1% L-alanyl-L-glutamine (Biochrom, K0302) in cell incubator (37° C., 5% CO₂) until further use (no longer than 24 h). For the killing assay, the antibodies were added at indicated concentrations (range of 0.01 pM-10 nM in triplicates). PBMCs were added to target cells at the final E:T ratio of 10:1. Target cell killing was assessed after 24 h and 48 h of incubation by quantification of LDH (lactate dehydrogenase) released into cell supernatants by apoptotic/necrotic cells (LDH detection kit, Roche Applied Science, #11 644 793 001). Maximal lysis of the target cells (=100%) was achieved by incubation of target cells with 1% Triton X-100. Minimal lysis (=0%) refers to target cells co-incubated with effector cells without bispecific antibody. The results show that MCSP TCB antibody is more potent than the MCSP 1+1 CrossMab TCB as it induced stronger killing of MCSP-positive target cells at both time points and on all tumor target cells (FIG. 89 A-H). The EC₅₀ values related to killing assays, calculated using GraphPadPrism5, are given in Table 14.

TABLE 14
MCSP receptor copy number and EC₅₀ values
(pM) for T-cell mediated killing of MCSP-expressing tumor
cells induced by MCSP TCB antibody (n.d. = not determined).

| | MCSP receptor | EC50 [pM] | EC50 [pM] |
|---|---|---|---|
| Cell line | copy number | 24 h | 48h |
| A375 | 387 058 | 0.1 | n.d. |
| MV-3 | 260 000 | 1.0 | 0.7 |
| HCT-116 | 36770 | ~6.2e−008 | ~0.09 |
| LS180 | negative | ~764 | n.d. |

### Example 32

### CD25 and CD69 Upregulation on CD8⁺ and CD4⁺ Effector Cells after T Cell-Mediated Killing of MCSP-Expressing Tumor Cells Induced by MCSP TCB and MCSP 1+1 CrossMab Antibodies

Activation of CD8⁺ and CD4⁺ T cells after T-cell killing of MCSP-expressing tumor cells (A375 and MV-3) mediated by the MCSP TCB and MCSP 1+1 CrossMab antibodies was assessed by FACS analysis using antibodies recognizing T cell activation markers CD25 (late activation marker) and CD69 (early activation marker). The antibody and the killing assay conditions were essentially as described above (Example 31), using the same antibody concentration range (0.01 pM-10 nM in triplicates), E:T ratio 10:1 and an incubation time of 48 h.

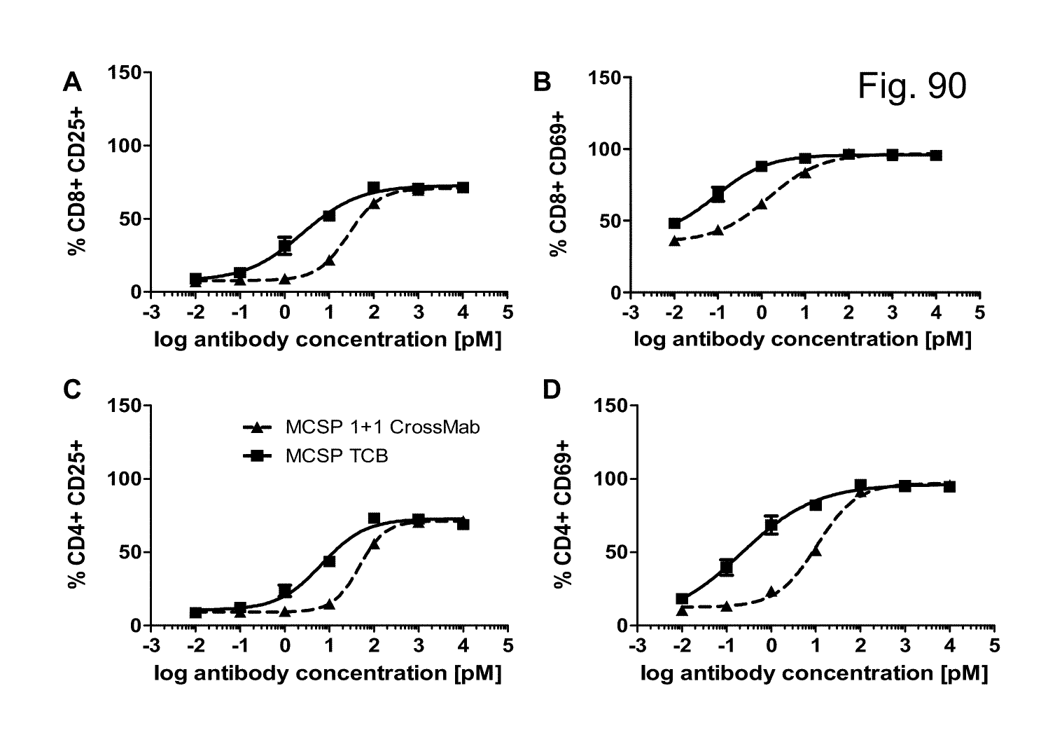

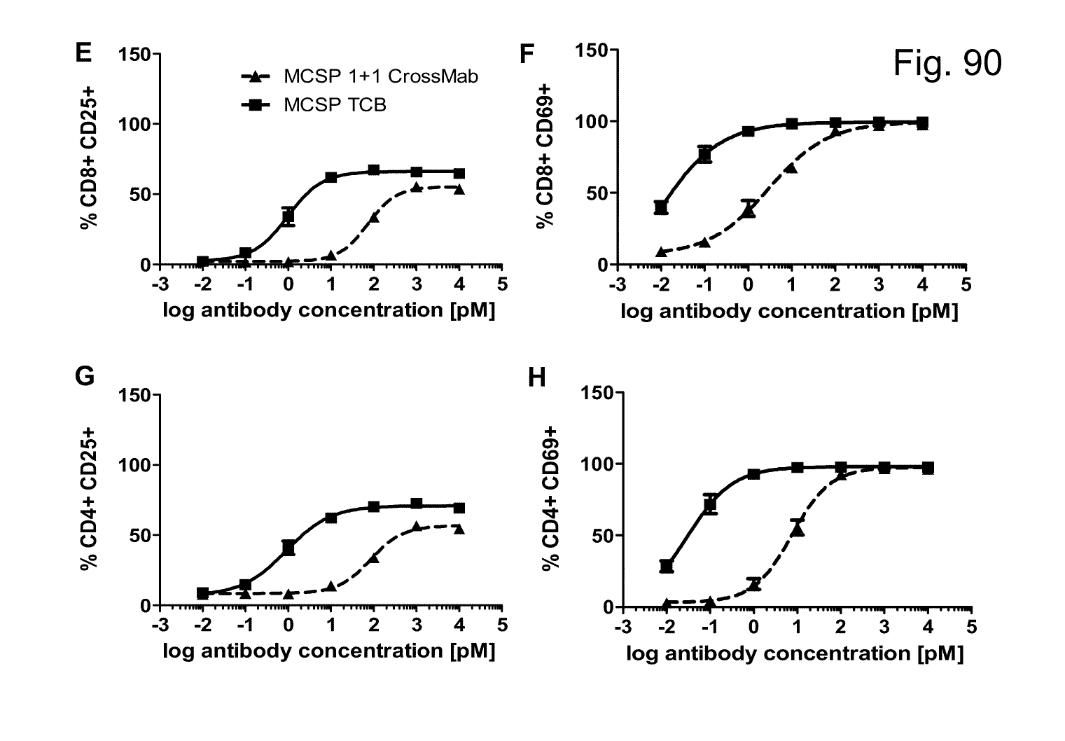

After the incubation, PBMCs were transferred to a round-bottom 96-well plate, centrifuged at 350×g for 5 min and washed twice with PBS containing 0.1% BSA. Surface staining for CD8 (FITC anti-human CD8, BD #555634), CD4 (PECy7 anti-human CD4, BD #557852), CD69 (PE anti-human CD69, Biolegend #310906) and CD25 (APC anti-human CD25, BD #555434) was performed according to the suppliers' indications. Cells were washed twice with 150 μl/well PBS containing 0.1% BSA and fixed for 15 min at 4° C. using 100 μl/well fixation buffer (BD #554655). After centrifugation, the samples were resuspended in 200 μl/well PBS 0.1% BSA containing DAPI to exclude dead cells for the FACS measurement. Samples were analyzed at BD FACS Fortessa. The results show that MCSP TCB induced a strong and target-specific upregulation of activation markers (CD25, CD69) on CD8⁺ T cells (FIG. 90 A, B (for A375 cells) and E, F (for MV-3 cells)) and CD4⁺ T cells (FIG. 90 C, D (for A375 cells) and G, H (for MV-3 cells)) after killing. As for the killing results, the activation of T cells was stronger with MCSP TCB than with MCSP 1+1 CrossMab.

### Example 33

### Preparation of DP47 GS TCB (2+1 Crossfab-IgG P329G LALA Inverted=“Untargeted TCB”) Containing DP47 GS as Non Binding Antibody and Humanized CH2527 as Anti CD3 Antibody

The “untargeted TCB” was used as a control in the above experiments. The bispecific antibody engages CD3c but does not bind to any other antigen and therefore cannot crosslink T cells to any target cells (and subsequently cannot induce any killing). It was therefore used as negative control in the assays to monitor any unspecific T cell activation.

The variable region of heavy and light chain DNA sequences were subcloned in frame with either the constant heavy chain or the constant light chain pre-inserted into the respective recipient mammalian expression vector. The antibody expression is driven by an MPSV promoter and carries a synthetic polyA signal sequence at the 3′ end of the CDS. In addition each vector contains an EBV OriP sequence.

The molecule was produced by co-transfecting HEK293 EBNA cells with the mammalian expression vectors using polyethylenimine (PEI). The cells were transfected with the corresponding expression vectors in a 1:2:1:1 ratio (“vector heavy chain Fc(hole)”:“vector light chain”:“vector light chain Crossfab”:“vector heavy chain Fc(knob)-FabCrossfab”).

For transfection HEK293 EBNA cells were cultivated in suspension serum-free in CD CHO culture medium. For the production in 500 ml shake flask 400 million HEK293 EBNA cells were seeded 24 hours before transfection. For transfection cells were centrifuged for 5 min at 210×g, supernatant is replaced by pre-warmed 20 ml CD CHO medium. Expression vectors were mixed in 20 ml CD CHO medium to a final amount of 200 μg DNA. After addition of 540 μl PEI solution the mixture was vortexed for 15 s and subsequently incubated for 10 min at room temperature. Afterwards cells were mixed with the DNA/PEI solution, transferred to a 500 ml shake flask and incubated for 3 hours at 37° C. in an incubator with a 5% CO₂ atmosphere. After the incubation time 160 ml F17 medium was added and cell were cultivated for 24 hours. One day after transfection 1 mM valproic acid and 7% Feed 1 (Lonza) was added. After 7 days cultivation supernatant was collected for purification by centrifugation for 15 min at 210×g, the solution was sterile filtered (0.22 μm filter) and sodium azide in a final concentration of 0.01% w/v was added, and kept at 4° C.

The secreted protein was purified from cell culture supernatants by affinity chromatography using Protein A. Supernatant was loaded on a HiTrap Protein A HP column (CV=5 mL, GE Healthcare) equilibrated with 40 ml 20 mM sodium phosphate, 20 mM sodium citrate, 0.5 M sodium chloride, pH 7.5. Unbound protein was removed by washing with at least 10 column volumes 20 mM sodium phosphate, 20 mM sodium citrate, 0.5 M sodium chloride, pH 7.5. Target protein was eluted during a gradient over 20 column volumes from 20 mM sodium citrate, 0.5 M sodium chloride, pH 7.5 to 20 mM sodium citrate, 0.5 M sodium chloride, pH 2.5. Protein solution was neutralized by adding 1/10 of 0.5 M sodium phosphate, pH 8. Target protein was concentrated and filtrated prior loading on a HiLoad Superdex 200 column (GE Healthcare) equilibrated with 20 mM histidine, 140 mM sodium chloride solution of pH 6.0.

The protein concentration of purified protein samples was determined by measuring the optical density (OD) at 280 nm, using the molar extinction coefficient calculated on the basis of the amino acid sequence.

Purity and molecular weight of molecules were analyzed by CE-SDS analyses in the presence and absence of a reducing agent. The Caliper LabChip GXII system (Caliper lifescience) was used according to the manufacturer's instruction. 2 μg sample was used for analyses.

The aggregate content of antibody samples was analyzed using a TSKgel G3000 SW XL analytical size-exclusion column (Tosoh) in 25 mM K₂HPO₄, 125 mM NaCl, 200 mM L-arginine monohydrochloride, 0.02% (w/v) NaN₃, pH 6.7 running buffer at 25° C.

TABLE 15
Summary production and purification of DP47 GS TCB.

| | | | Aggregate | | | |
|---|---|---|---|---|---|---|
| | | | after 1^{st} | | | |
| | Titer | Yield | purification | HMW | LMW | Monomer |
| Construct | [mg/l] | [mg/l] | step [%] | [%] | [%] | [%] |
| DP47 | 103.7 | 8.04 | 8 | 2.3 | 6.9 | 91.8 |
| GS TCB | | | | | | |

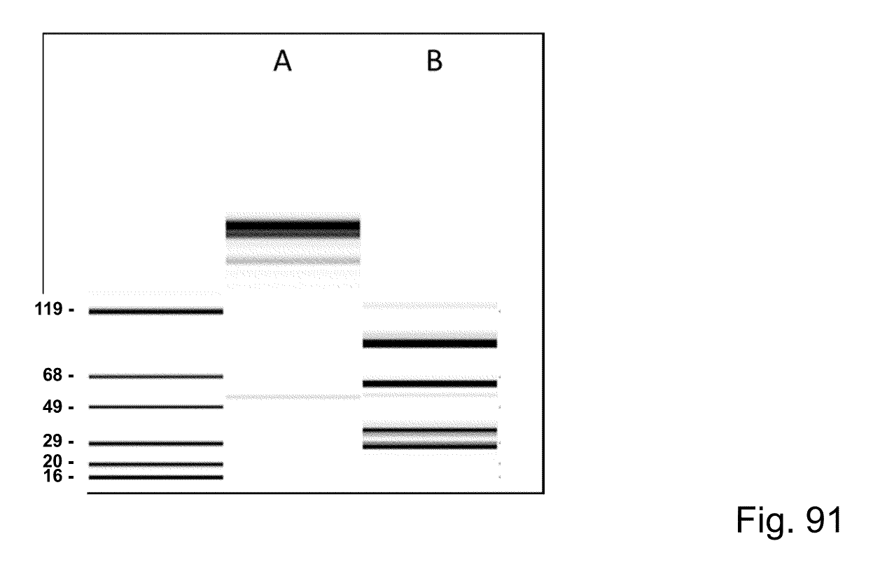

FIG. 91 and Table 16 show CE-SDS analyses of the DP47 GS TCB (2+1 Crossfab-IgG P329G LALA inverted) containing DP47 GS as non-binding antibody and humanized CH2527 as anti-CD3 antibody. (SEQ ID NOs:325, 326, 327 and 328).

TABLE 16
CE-SDS analyses of DP47 GS TCB.

| | Peak | kDa | Corresponding Chain |
|---|---|---|---|
| DP47 GS TCB | 1 | 165.22 | Molecule with 2 missing light chains |
| non reduced (A) | | | |
| | 2 | 181.35 | Molecule with 1 missing light chain |
| | 3 | 190.58 | Correct molecule without N-linked |
| | | | glycosylation |
| | 4 | 198.98 | Correct molecule |
| DP47 GS TCB | 1 | 27.86 | Light chain DP47 GS |
| reduced (B) | | | |
| | 2 | 35.74 | Light chain huCH2527 |
| | 3 | 63.57 | Fc(hole) |
| | 4 | 93.02 | Fc(knob) |

### Example 34

### Preparation of a Knob-in-Hole MCSP TCB hIgG₄ S228P/L325E Containing M4-3(C1) ML2(G3) as Anti-MCSP Antibody and Humanized CH2527 as Anti-CD3 Antibody

A knob-in-hole TCB with a human IgG₄ Fc region containing the mutations S228P and L325E (SPLE) was constructed. This TCB has M4-3(C1) ML2(G3) as anti-MCSP antibody and humanized CH2527 as anti-CD3 antibody.

The variable region of heavy and light chain DNA sequences were subcloned in frame with either the constant heavy chain or the constant light chain pre-inserted into the respective recipient mammalian expression vector. The antibody expression was driven by an MPSV promoter and carries a synthetic polyA signal sequence at the 3′ end of the CDS. In addition each vector contains an EBV OriP sequence.

The molecule was produced by co-transfecting HEK293-EBNA cells with the mammalian expression vectors using polyethylenimine (PEI). The cells were transfected with the corresponding expression vectors in a 1:1:2:1 ratio (“vector heavy chain Fc(hole)”:“vector light chain Crossfab”:“vector light chain”:“vector heavy chain Fc(knob) FabCrossfab”).

For transfection HEK293 EBNA cells were cultivated in suspension serum-free in CD CHO culture medium. For the production in 500 ml shake flask 400 million HEK293 EBNA cells were seeded 24 hours before transfection. For transfection cells were centrifuged for 5 min at 210×g, supernatant was replaced by pre-warmed 20 ml CD CHO medium. Expression vectors were mixed in 20 ml CD CHO medium to a final amount of 200 μg DNA. After addition of 540 μl PEI solution the mixture was vortexed for 15 s and subsequently incubated for 10 min at room temperature. Afterwards cells were mixed with the DNA/PEI solution, transferred to a 500 ml shake flask and incubated for 3 hours at 37° C. in an incubator with a 5% CO₂ atmosphere.

After incubation time 160 ml F17 medium was added and cells were cultivated for 24 hours. One day after transfection 1 mM valproic acid and 7% Feed 1 (Lonza) was added. After 7 days cultivation supernatant was collected for purification by centrifugation for 15 min at 210×g, the solution was sterile filtered (0.22 μm filter) and sodium azide in a final concentration of 0.01% w/v was added, and kept at 4° C.

The secreted protein was purified from cell culture supernatants by affinity chromatography using Protein A. Supernatant was loaded on a 4 ml column packed with POROS® MabCapture™ A Perfusion Chromatography®Media (CV=4 mL, Applied Biosystems) equilibrated with 36 ml 20 mM sodium phosphate, 20 mM sodium citrate, 0.5 M sodium chloride, 0.01% v/v Tween-20, pH 7.5. Unbound protein was removed by washing with at least 10 column volumes 20 mM sodium phosphate, 20 mM sodium citrate, 0.5 M sodium chloride, 0.01% v/v Tween-20, pH 7.5. Target protein was eluted during a gradient over 20 column volumes from 20 mM sodium citrate, 0.5 M sodium chloride, pH 7.5 to 20 mM sodium citrate, 0.5 M sodium chloride, 0.01% v/v Tween-20, pH 2.5. Protein solution was neutralized by adding 1/10 of 0.5 M sodium phosphate, pH 8. Target protein was concentrated and filtrated prior loading on a HiLoad Superdex 200 column (GE Healthcare) equilibrated with 20 mM histidine, 140 mM sodium chloride, 0.01% v/v Tween-20 solution of pH 6.0.

The protein concentration of purified protein samples was determined by measuring the optical density (OD) at 280 nm, using the molar extinction coefficient calculated on the basis of the amino acid sequence. Purity and molecular weight of molecules were analyzed by CE-SDS analyses in the presence and absence of a reducing agent. The Caliper LabChip GXII system (Caliper Lifescience) was used according to the manufacturer's instruction. 2 μg sample was used for analyses. The aggregate content of antibody samples was analyzed using a TSKgel G3000 SW XL analytical size-exclusion column (Tosoh) in 25 mM K₂HPO₄, 125 mM NaCl, 200 mM L-arginine monohydrochloride, 0.02% (w/v) NaN₃, pH 6.7 running buffer at 25° C.

Human IgG₄ carrying a Fc with knob into hole and SPLE mutation can be used to generate heterodimeric T cell bispecific molecules with good quality and high yield. Compared to T cell bispecifics based on hIgG₁ knob into hole P329G LALA these molecules seemed to be more stable as reflected in the higher yields and low aggregate content after the first purification step shown in Table 17.

TABLE 17
Yields, aggregate content after Protein A and final monomer content for MCSP TCB
hIgG₄ S228P/L325E.

| | | | Aggregate | LMW after | | | |
|---|---|---|---|---|---|---|---|
| | | | after 1^{st} | 1^{st} | | | |
| | Titer | Yield | purification | purification | HMW | LMW | Monomer |
| Construct | [mg/l] | [mg/l] | step [%] | step [%] | [%] | [%] | [%] |
| MCSP TCB hIgG₄ | 78.4 | 5.9 | 2.8 | 0 | 0 | 0 | 100 |
| S228P/L325E | | | | | | | |
| hIgG1 knob-in- | 157 | 0.32 | 32 | 0 | 3.3 | 0 | 96.7 |
| hole P329G | | | | | | | |
| LALA TCB | | | | | | | |

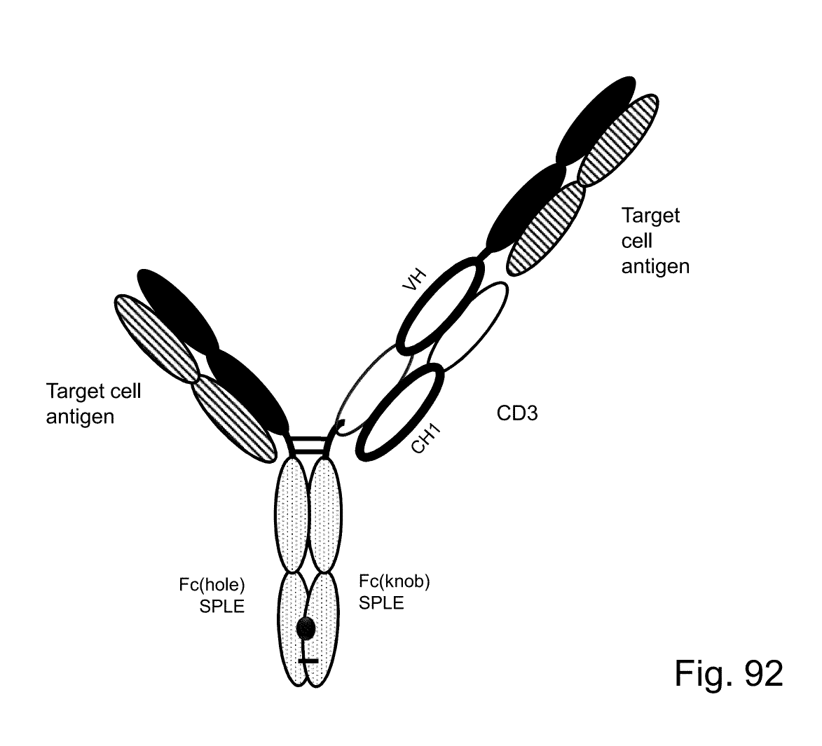

FIG. 92 shows a schematic drawing of the MCSP TCB hIgG₄ S228P/L325E (SEQ ID NOs: 278, 319, 369 and 370) molecule.

TABLE 18
CE-SDS analyses of MCSP TCB hIgG₄ S228P/L325E.

| | Peak | kDa | Corresponding Chain |
|---|---|---|---|
| MCSP TCB hIgG₄ S228P/L325E | 1 | 205.21 | Correct molecule |
| non reduced (A) | | | |
| MCSP TCB hIgG₄ S228P/L325E | 1 | 32 | Light chain ML2(G3) |
| reduced (B) | | | |
| | 2 | 40 | Light Chain huCH2527 |
| | 3 | 71 | Fab-Fc(hole SPLE) |
| | 4 | 99 | Fab-Crossfab-Fc(knob) |
| | | | SPLE |

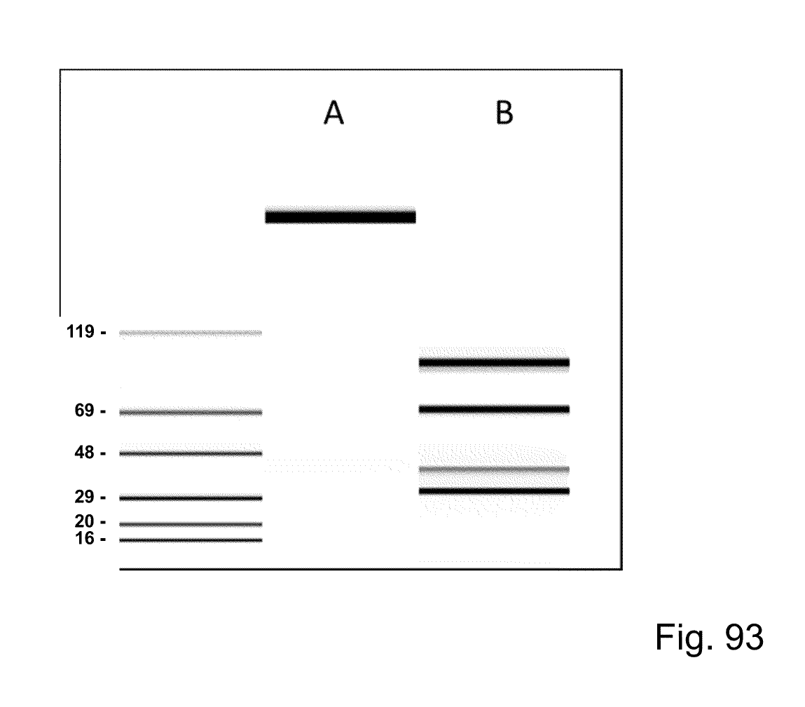

FIG. 93 and Table 18 show CE-SDS analyses of the MCSP TCB hIgG₄ S228P/L325E molecule (SEQ ID NOs: 278, 319, 369, and 370).

### Example 35

### Binding of MCSP TCB hIgG4 S228P/L325E to MCSP- and CD3-Expressing Cells

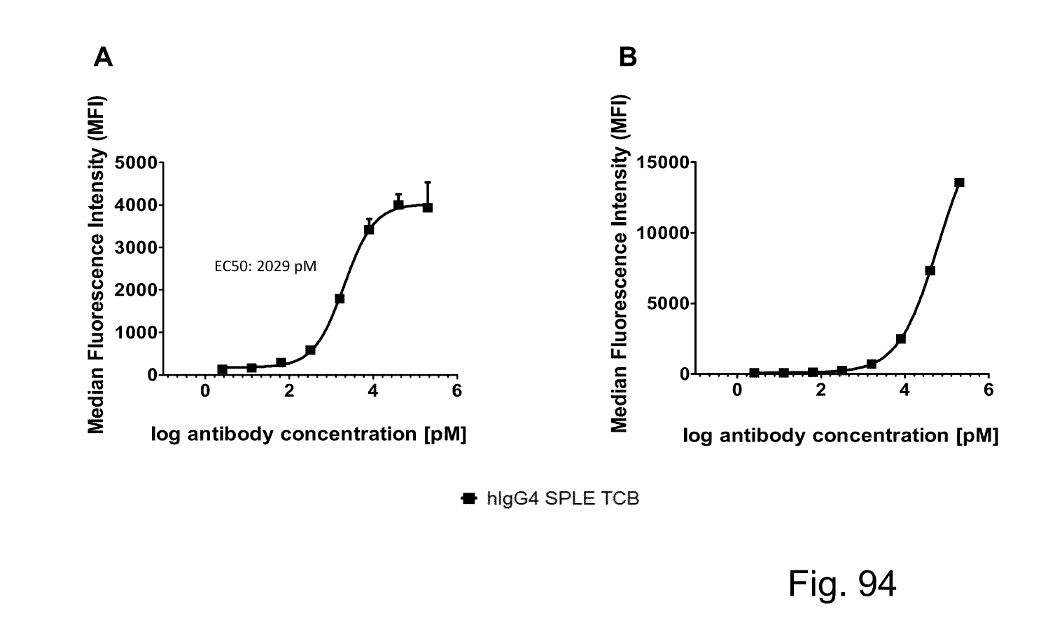

The binding of MCSP TCB hIgG4 S228P/L325E was tested on MCSP-expressing human melanoma cell line (MV-3) and CD3-expressing immortalized T lymphocyte line (Jurkat). Briefly, cells were harvested, counted, checked for viability and resuspended at 2×10⁶ cells/ml in FACS buffer (PBS 0.1% BSA). 100 μl of cell suspension (containing 0.2×10⁶ cells) were incubated in round-bottom 96-well plate for 30 min at 4° C. with increasing concentrations of the hIgG₄ SPLE TCB (3 pM-200 nM), washed twice with cold PBS 0.1% BSA, re-incubated for further 30 min at 4° C. with the PE-conjugated AffiniPure F(ab′)2 Fragment goat anti-human IgG Fcγ Fragment Specific secondary antibody (Jackson Immuno Research Lab PE #109-116-170), washed twice with cold PBS 0.1% BSA and immediately analyzed by FACS using a FACS CantoII (Software FACS Diva). Binding curves were obtained using GraphPadPrism5 (FIG. 94 A, binding to MV-3 cells, EC₅₀=2029 pM; FIG. 94 B, binding to Jurkat cells).

### Example 36

### T-Cell Killing Induced by MCSP TCB hIgG₄ S228P/L325E

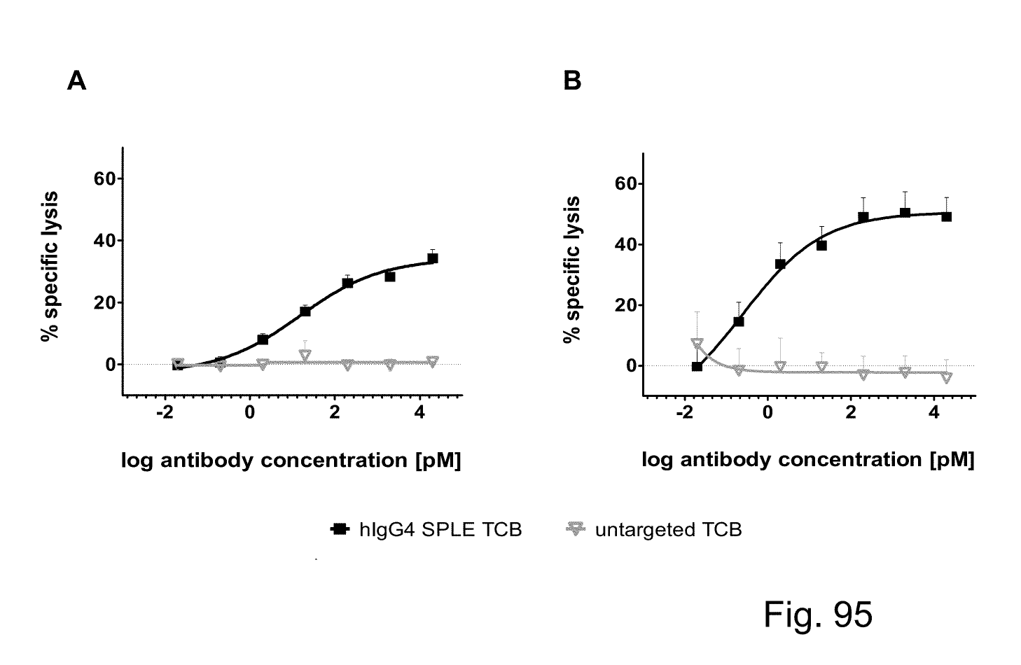

T-cell killing mediated by MCSP TCB hIgG₄ S228P/L325E was assessed using MCSP-expressing human melanoma tumor cells (MV-3) and human PBMCs at 24 hours and 48 hours of incubation. Briefly, target cells were harvested with Trypsin/EDTA, washed, and plated at a density of 25 000 cells/well using flat-bottom 96-well plates. Cells were left to adhere overnight. Peripheral blood mononuclear cells (PBMCs) were prepared by Histopaque density centrifugation of enriched lymphocyte preparations (buffy coats) obtained from healthy human donors. Fresh blood was diluted with sterile PBS and layered over Histopaque gradient (Sigma, #H8889). After centrifugation (450×g, 30 minutes, room temperature), the plasma above the PBMC-containing interphase was discarded and PBMCs transferred in a new Falcon tube subsequently filled with 50 ml of PBS. The mixture was centrifuged (400×g, 10 minutes, room temperature), the supernatant discarded and the PBMC pellet washed twice with sterile PBS (centrifugation steps 350×g, 10 minutes). The resulting PBMC population was counted automatically (ViCell) and stored in RPMI1640 medium containing 10% FCS and 1% L-alanyl-L-glutamine (Biochrom, K0302) at 37° C., 5% CO₂ in a cell incubator until further use (no longer than 24 h). For the killing assay, the antibody was added at the indicated concentrations (range of 0.02 pM-20 nM in triplicates). PBMCs were added to target cells at a final E:T ratio of 10:1. Target cell killing was assessed after 24 h and 48 h of incubation at 37° C., 5% CO₂ by quantification of LDH released into cell supernatants by apoptotic/necrotic cells (LDH detection kit, Roche Applied Science, #11 644 793 001). Maximal lysis of the target cells (=100%) was achieved by incubation of target cells with 1% Triton X-100. Minimal lysis (=0%) refers to target cells co-incubated with effector cells without bispecific construct. The results show that hIgG₄ SPLE TCB induced a strong and target-specific killing of MCSP-positive target cell lines (FIG. 95 A, B). The EC₅₀ values related to killing assays, calculated using GraphPadPrism5, are given in Table 19.

TABLE 19
EC₅₀ values (pM) for T-cell mediated killing of MCSP-expressing
tumor cells (MV-3) induced by MCSP TCB hIgG4 S228P/L325E.

| Cell line | EC₅₀ [pM] 24 h | EC₅₀ [pM] 48 h |
|---|---|---|
| MV-3 | 14.9 | 0.24 |

### Example 37

### Preparation of a Knob-in-Hole MCSP TCB hIgG₄ S228P/L325E P329GLALA Containing M4-3(C1) ML2(G3) as Anti-MCSP Antibody and Humanized CH2527 as Anti-CD3 Antibody

A knob-in-hole TCB with a human IgG₄ Fc region containing the mutations S228P and L325E (SPLE) and P329G LALA is constructed. This TCB has M4-3(C1) ML2(G3) as anti-MCSP antibody and humanized CH2527 as anti-CD3 antibody.

The variable region of heavy and light chain DNA sequences are subcloned in frame with either the constant heavy chain or the constant light chain pre-inserted into the respective recipient mammalian expression vector. The antibody expression is driven by an MPSV promoter and carries a synthetic polyA signal sequence at the 3′ end of the CDS. In addition each vector contains an EBV OriP sequence.

The molecule is produced by co-transfecting HEK293-EBNA cells with the mammalian expression vectors using polyethylenimine (PEI). The cells are transfected with the corresponding expression vectors in a 1:1:2:1 ratio (“vector heavy chain Fc(hole)”:“vector light chain Crossfab”:“vector light chain”:“vector heavy chain Fc(knob) FabCrossfab”).

For transfection HEK293 EBNA cells are cultivated in suspension serum-free in CD CHO culture medium. For the production in 500 ml shake flask 400 million HEK293 EBNA cells are seeded 24 hours before transfection. For transfection cells are centrifuged for 5 min at 210×g, supernatant is replaced by pre-warmed 20 ml CD CHO medium. Expression vectors are mixed in 20 ml CD CHO medium to a final amount of 200 μg DNA. After addition of 540 μl PEI solution the mixture is vortexed for 15 s and subsequently incubated for 10 min at room temperature. Afterwards cells are mixed with the DNA/PEI solution, transferred to a 500 ml shake flask and incubated for 3 hours by 37° C. in an incubator with a 5% CO₂ atmosphere. After incubation time 160 ml F17 medium is added and cell are cultivated for 24 hours. One day after transfection 1 mM valproic acid and 7% Feed 1 (Lonza) is added. After 7 days cultivation supernatant is collected for purification by centrifugation for 15 min at 210×g, the solution is sterile filtered (0.22 μm filter) and sodium azide in a final concentration of 0.01% w/v is added, and kept at 4° C.

The secreted protein is purified from cell culture supernatants by affinity chromatography using Protein A. Supernatant is loaded on a 4 ml column packed with POROS® MabCapture™ A Perfusion Chromatography®Media (CV=4 mL, Applied Biosystems) equilibrated with 36 ml 20 mM sodium phosphate, 20 mM sodium citrate, 0.5 M sodium chloride, 0.01% v/v Tween-20, pH 7.5. Unbound protein is removed by washing with at least 10 column volumes 20 mM sodium phosphate, 20 mM sodium citrate, 0.5 M sodium chloride, 0.01% v/v Tween-20, pH 7.5. Target protein is eluted during a gradient over 20 column volume from 20 mM sodium citrate, 0.5 M sodium chloride, pH 7.5 to 20 mM sodium citrate, 0.5 M sodium chloride, 0.01% v/v Tween-20, pH 2.5. Protein solution is neutralized by adding 1/10 of 0.5 M sodium phosphate, pH 8. Target protein is concentrated and filtrated prior to loading on a HiLoad Superdex 200 column (GE Healthcare) equilibrated with 20 mM histidine, 140 mM sodium chloride, 0.01% v/v Tween-20 solution of pH 6.0.

The protein concentration of purified protein samples is determined by measuring the optical density (OD) at 280 nm, using the molar extinction coefficient calculated on the basis of the amino acid sequence. Purity and molecular weight of molecules are analyzed by CE-SDS analyses in the presence and absence of a reducing agent. The Caliper LabChip GXII system (Caliper Lifescience) is used according to the manufacturer's instruction. 2 μg sample is used for analyses. The aggregate content of antibody samples was analyzed using a TSKgel G3000 SW XL analytical size-exclusion column (Tosoh) in 25 mM K₂HPO₄, 125 mM NaCl, 200 mM L-arginine monohydrochloride, 0.02% (w/v) NaN₃, pH 6.7 running buffer at 25° C.

### Example 38

### Binding of MCSP TCB hIgG₄ S228P/L325E P329G to MCSP- and CD3-Expressing Cells

The binding of MCSP TCB hIgG₄ S228P/L325E P329G is tested on MCSP-expressing human melanoma cell line (MV-3) and CD3-expressing immortalized T lymphocyte line (Jurkat). Briefly, cells are harvested, counted, checked for viability and resuspended at 2×10⁶ cells/ml in FACS buffer (PBS 0.1% BSA). 100 μl of cell suspension (containing 0.2×10⁶ cells) are incubated in round-bottom 96-well plate for 30 min at 4° C. with increasing concentrations of the hIgG₄ SPLE PG TCB (3 pM-200 nM), washed twice with cold PBS 0.1% BSA, re-incubated for further 30 min at 4° C. with the PE-conjugated AffiniPure F(ab′)2 Fragment goat anti-human IgG Fcγ Fragment Specific secondary antibody (Jackson Immuno Research Lab PE #109-116-170), washed twice with cold PBS 0.1% BSA and immediately analyzed by FACS using a FACS CantoII (Software FACS Diva). Binding curves are obtained using GraphPadPrism5.

### Example 39

### T-Cell Killing Induced by MCSP TCB hIgG4 S228P/L325E P329G

T-cell killing mediated by MCSP TCB hIgG₄ S228P/L325E is assessed using MCSP-expressing human melanoma tumor cells (MV-3) and human PBMCs at 24 hours and 48 hours of incubation. Briefly, target cells are harvested with Trypsin/EDTA, washed, and plated at density of 25 000 cells/well using flat-bottom 96-well plates. Cells are left to adhere overnight. Peripheral blood mononuclear cells (PBMCs) are prepared by Histopaque density centrifugation of enriched lymphocyte preparations (buffy coats) obtained from healthy human donors. Fresh blood is diluted with sterile PBS and layered over Histopaque gradient (Sigma, #H8889). After centrifugation (450×g, 30 minutes, room temperature), the plasma above the PBMC-containing interphase is discarded and PBMCs transferred in a new Falcon tube subsequently filled with 50 ml of PBS. The mixture is centrifuged (400×g, 10 minutes, room temperature), the supernatant discarded and the PBMC pellet washed twice with sterile PBS (centrifugation steps 350×g, 10 minutes). The resulting PBMC population is counted automatically (ViCell) and stored in RPMI1640 medium containing 10% FCS and 1% L-alanyl-L-glutamine (Biochrom, K0302) at 37° C., 5% CO₂ in cell incubator until further use (no longer than 24 h). For the killing assay, the antibody is added at the indicated concentrations (range of 0.02 pM-20 nM in triplicates). PBMCs are added to target cells at final E:T ratio of 10:1. Target cell killing is assessed after 24 h and 48 h of incubation at 37° C., 5% CO₂ by quantification of LDH released into cell supernatants by apoptotic/necrotic cells (LDH detection kit, Roche Applied Science, #11 644 793 001). Maximal lysis of the target cells (=100%) is achieved by incubation of target cells with 1% Triton X-100. Minimal lysis (=0%) refers to target cells co-incubated with effector cells without bispecific construct.

SEQ ID NOs: 371 and 372 are for IgG₄ SPLE PG heavy chains and SEQ ID NOs: 278 and 319 for light chains.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A T cell activating bispecific antigen binding molecule comprising a first and a second antigen binding moiety, one of which is a Fab molecule capable of specific binding to an activating T cell antigen and the other one of which is a Fab molecule capable of specific binding to a target cell antigen, and an IgG₄ Fc domain composed of a first and a second subunit capable of stable association;
wherein the first antigen binding moiety is
(a) a single chain Fab molecule wherein the Fab light chain and the Fab heavy chain are connected by a peptide linker, or
(b) a crossover Fab molecule wherein either the variable or the constant regions of the Fab light chain and the Fab heavy chain are exchanged.

2. The T cell activating bispecific antigen binding molecule of claim 1, comprising not more than one antigen binding moiety capable of specific binding to an activating T cell antigen.

3. The T cell activating bispecific antigen binding molecule of claim 1, wherein the first and the second antigen binding moiety are fused to each other, optionally via a peptide linker.

4. The T cell activating bispecific antigen binding molecule of claim 1, wherein the second antigen binding moiety is fused at the C-terminus of the Fab heavy chain to the N-terminus of the Fab heavy chain of the first antigen binding moiety.

5. The T cell activating bispecific antigen binding molecule of claim 1, wherein the first antigen binding moiety is fused at the C-terminus of the Fab heavy chain to the N-terminus of the Fab heavy chain of the second antigen binding moiety.

6. The T cell activating bispecific antigen binding molecule of claim 3, wherein the first antigen binding moiety is a crossover Fab molecule and the Fab light chain of the first antigen binding moiety and the Fab light chain of the second antigen binding moiety are fused to each other, optionally via a peptide linker.

7. The T cell activating bispecific antigen binding molecule claim 1, wherein the second antigen binding moiety of the T cell activating bispecific antigen binding molecule is fused at the C-terminus of the Fab light chain to the N-terminus of the Fab light chain of the first antigen binding moiety.

8. The T cell activating bispecific antigen binding molecule of claim 1, wherein the second antigen binding moiety of the T cell activating bispecific antigen binding molecule is fused at the C-terminus of the Fab heavy chain to the N-terminus of the first or the second subunit of the Fc domain.

9. The T cell activating bispecific antigen binding molecule of claim 1, wherein the first antigen binding moiety is fused at the C-terminus of the Fab heavy chain to the N-terminus of the first or second subunit of the Fc domain.

10. The T cell activating bispecific antigen binding molecule of claim 1, wherein the first and the second antigen binding moiety are each fused at the C-terminus of the Fab heavy chain to the N-terminus of one of the subunits of the Fc domain.

11. The T cell activating bispecific antigen binding molecule of claim 1, comprising a third antigen binding moiety which is a Fab molecule capable of specific binding to a target cell antigen.

12. The T cell activating bispecific antigen binding molecule of claim 11, wherein the third antigen binding moiety is fused at the C-terminus of the Fab heavy chain to the N-terminus of the first or second subunit of the Fc domain.

13. The T cell activating bispecific antigen binding molecule of claim 11, wherein the second and the third antigen binding moiety are each fused at the C-terminus of the Fab heavy chain to the N-terminus of one of the subunits of the Fc domain, and the first antigen binding moiety is fused at the C-terminus of the Fab heavy chain to the N-terminus of the Fab heavy chain of the second antigen binding moiety.

14. The T cell activating bispecific antigen binding molecule of claim 11, wherein the first and the third antigen binding moiety are each fused at the C-terminus of the Fab heavy chain to the N-terminus of one of the subunits of the Fc domain, and the second antigen binding moiety is fused at the C-terminus of the Fab heavy chain to the N-terminus of the Fab heavy chain of the first antigen binding moiety.

15. The T cell activating bispecific antigen binding molecule of claim 1 wherein the IgG₄ Fc domain is a human IgG₄ Fc domain.

16. The T cell activating bispecific antigen binding molecule of claim 1 wherein the IgG₄ Fc domain comprises an amino acid substitution at position S228 (Kabat numbering).

17. The T cell activating bispecific antigen binding molecule of claim 16, wherein said amino acid substitution is S228P.

18. The T cell activating bispecific antigen binding molecule of claim 1 wherein the Fc domain comprises a modification promoting the association of the first and the second subunit of the Fc domain.

19. The T cell activating bispecific antigen binding molecule of claim 18, wherein in the CH3 domain of the first subunit of the Fc domain an amino acid residue is replaced with an amino acid residue having a larger side chain volume, thereby generating a protuberance within the CH3 domain of the first subunit which is positionable in a cavity within the CH3 domain of the second subunit, and in the CH3 domain of the second subunit of the Fc domain an amino acid residue is replaced with an amino acid residue having a smaller side chain volume, thereby generating a cavity within the CH3 domain of the second subunit within which the protuberance within the CH3 domain of the first subunit is positionable.

20. The T cell activating bispecific antigen binding molecule of claim 1 wherein the Fc domain exhibits reduced binding affinity to an Fc receptor and/or reduced effector function, as compared to a native IgG₄ Fc domain.

21. The T cell activating bispecific antigen binding molecule of claim 1 wherein the Fc domain comprises one or more amino acid substitution that reduces binding to an Fc receptor and/or effector function.

22. The T cell activating bispecific antigen binding molecule of claim 21, wherein said one or more amino acid substitution is at one or more position selected from the group of L235 and P329 (Kabat numbering).

23. The T cell activating bispecific antigen binding molecule of claim 22, wherein said amino acid substitution at position L235 is L235E and said amino acid substitution at position P329 is P329G.

24. The T cell activating bispecific antigen binding molecule of claim 1 wherein each subunit of the IgG₄ Fc domain comprises the amino acid substitutions L235E and S228P.

25. The T cell activating bispecific antigen binding molecule of claim 24 wherein each subunit of the IgG₄ Fc domain comprises the amino acid substitutions L235E, S228P and P329G.

26. The T cell activating bispecific antigen binding molecule of claim 21, wherein the Fc receptor is an Fcγ receptor.

27. The T cell activating bispecific antigen binding molecule of claim 21, wherein the effector function is antibody-dependent cell-mediated cytotoxicity (ADCC).

28. The T cell activating bispecific antigen binding molecule of claim 1 wherein the activating T cell antigen is CD3.

29. The T cell activating bispecific antigen binding molecule of claim 28, wherein the first antigen binding moiety comprises the heavy chain CDR1 of SEQ ID NO: 270, the heavy chain CDR2 of SEQ ID NO: 271, the heavy chain CDR3 of SEQ ID NO: 272, the light chain CDR1 of SEQ ID NO: 274, the light chain CDR2 of SEQ ID NO: 275, and the light chain CDR3 of SEQ ID NO: 276.

30. The T cell activating bispecific antigen binding molecule of claim 28, wherein the first antigen binding moiety comprises a heavy chain variable region sequence that is at least about 95%, 96%, 97%, 98%, 99% or 100% identical to SEQ ID NO: 269 and a light chain variable region sequence that is at least about 95%, 96%, 97%, 98%, 99% or 100% identical to SEQ ID NO: 273.

31. The T cell activating bispecific antigen binding molecule of claim 1 wherein the target cell antigen is selected from the group consisting of: Melanoma-associated Chondroitin Sulfate Proteoglycan (MCSP), Epidermal Growth Factor Receptor (EGFR), CD19, CD20, CD33, Carcinoembryonic Antigen (CEA) and Fibroblast Activation Protein (FAP).

32. The T cell activating bispecific antigen binding molecule of claim 1 wherein the target cell antigen is Carcinoembryonic Antigen (CEA).

33. The T cell activating bispecific antigen binding molecule of claim 32, wherein the second antigen binding moiety comprises the heavy chain CDR1 of SEQ ID NO: 290, the heavy chain CDR2 of SEQ ID NO: 291, the heavy chain CDR3 of SEQ ID NO: 292, the light chain CDR1 of SEQ ID NO: 294, the light chain CDR2 of SEQ ID NO: 295 and the light chain CDR3 of SEQ ID NO: 296.

34. The T cell activating bispecific antigen binding molecule of claim 32, wherein the second antigen binding moiety comprises a heavy chain variable region sequence that is at least about 95%, 96%, 97%, 98%, 99% or 100% identical to the amino acid sequence of SEQ ID NO: 289 and a light chain variable region sequence that is at least about 95%, 96%, 97%, 98%, 99% or 100% identical to the amino acid sequence of SEQ ID NO: 293.

35. The T cell activating bispecific antigen binding molecule of claim 1, wherein the target cell antigen is Melanoma-associated Chondroitin Sulfate Proteoglycan (MCSP).

36. The T cell activating bispecific antigen binding molecule of claim 35, wherein the second antigen binding moiety comprises the heavy chain CDR1 of SEQ ID NO: 280, the heavy chain CDR2 of SEQ ID NO: 281, the heavy chain CDR3 of SEQ ID NO: 282, the light chain CDR1 of SEQ ID NO: 284, the light chain CDR2 of SEQ ID NO: 285 and the light chain CDR3 of SEQ ID NO: 286.

37. The T cell activating bispecific antigen binding molecule of claim 35, wherein the second antigen binding moiety comprises a heavy chain variable region sequence that is at least about 95%, 96%, 97%, 98%, 99% or 100% identical to the amino acid sequence of SEQ ID NO: 279 and a light chain variable region sequence that is at least about 95%, 96%, 97%, 98%, 99% or 100% identical to the amino acid sequence of SEQ ID NO: 283.

38. A T cell activating bispecific antigen binding molecule comprising a first antigen binding moiety capable of specific binding to CD3, a second antigen binding moiety capable of binding to MCSP, and an IgG₄ Fc domain, wherein the antigen binding molecule comprises the amino acid sequences of SEQ ID NOs 278, 319, 369 and 370.

39. A T cell activating bispecific antigen binding molecule comprising a first antigen binding moiety capable of specific binding to CD3, a second antigen binding moiety capable of binding to MCSP, and an IgG₄ Fc domain, wherein the antigen binding molecule comprises the amino acid sequences of SEQ ID NOs 278, 319, 371 and 372.

40. An isolated polynucleotide encoding the T cell activating bispecific antigen binding molecule of claim 1 or a fragment thereof.

41. An isolated polynucleotide encoding the T cell activating bispecific antigen binding molecule of claim 38 or a fragment thereof.

42. An isolated polynucleotide encoding the T cell activating bispecific antigen binding molecule of claim 39 or a fragment thereof.

43. A polypeptide encoded by the isolated polynucleotide of claim 40.

44. A vector, particularly an expression vector, comprising the isolated polynucleotide of claim 40.

45. A host cell comprising the expression vector of claim 44.

46. A method of producing a T cell activating bispecific antigen binding molecule, comprising the steps of a) culturing the host cell of claim 45 under conditions suitable for the expression of the T cell activating bispecific antigen binding molecule and b) recovering the T cell activating bispecific antigen binding molecule.

47. A T cell activating bispecific antigen binding molecule produced by the method of claim 46.

48. A pharmaceutical composition comprising the T cell activating bispecific antigen binding molecule of claim 1 and a pharmaceutically acceptable carrier.

49. A pharmaceutical composition comprising the T cell activating bispecific antigen binding molecule of claim 38 and a pharmaceutically acceptable carrier.

50. A pharmaceutical composition comprising the T cell activating bispecific antigen binding molecule of claim 39 and a pharmaceutically acceptable carrier.

51. A pharmaceutical composition comprising the T cell activating bispecific antigen binding molecule of claim 47 and a pharmaceutically acceptable carrier.

52. The T cell activating bispecific antigen binding molecule of claim 1 for use as a medicament.

53. The T cell activating bispecific antigen binding molecule of claim 38 for use as a medicament.

54. The T cell activating bispecific antigen binding molecule of claim 39 for use as a medicament

55. The T cell activating bispecific antigen binding molecule of claim 47 for use as a medicament

56. The T cell activating bispecific antigen binding molecule of claim 1 for use in the treatment of a disease in an individual in need thereof.

57. The T cell activating bispecific antigen binding molecule of claim 56, wherein the disease is cancer.

58. The T cell activating bispecific antigen binding molecule of claim 38 for use in the treatment of a disease in an individual in need thereof.

59. The T cell activating bispecific antigen binding molecule of claim 39 for use in the treatment of a disease in an individual in need thereof.

60. The T cell activating bispecific antigen binding molecule of claim 47 for use in the treatment of a disease in an individual in need thereof.

61. A method of treating a disease in an individual, comprising administering to said individual a therapeutically effective amount of a composition comprising the T cell activating bispecific antigen binding molecule of claim 1 in a pharmaceutically acceptable form.

62. The method of claim 61, wherein said disease is cancer.

63. A method of treating a disease in an individual, comprising administering to said individual a therapeutically effective amount of a composition comprising the T cell activating bispecific antigen binding molecule of claim 38 in a pharmaceutically acceptable form.

64. A method of treating a disease in an individual, comprising administering to said individual a therapeutically effective amount of a composition comprising the T cell activating bispecific antigen binding molecule of claim 39 in a pharmaceutically acceptable form.

65. A method of treating a disease in an individual, comprising administering to said individual a therapeutically effective amount of a composition comprising the T cell activating bispecific antigen binding molecule of claim 47 in a pharmaceutically acceptable form.

66. A method for inducing lysis of a target cell, comprising contacting a target cell with the T cell activating bispecific antigen binding molecule of claim 1 in the presence of a T cell.

67. A method for inducing lysis of a target cell, comprising contacting a target cell with the T cell activating bispecific antigen binding molecule of claim 38 in the presence of a T cell.

68. A method for inducing lysis of a target cell, comprising contacting a target cell with the T cell activating bispecific antigen binding molecule of claim 39 in the presence of a T cell.

69. A method for inducing lysis of a target cell, comprising contacting a target cell with the T cell activating bispecific antigen binding molecule of claim 47 in the presence of a T cell.

70. A host cell comprising the isolated polynucleotide of claim 40.
